(19) United States
(12) Patent Application Publication
(10) Pub. No.: US 2026/0325654 A1
(43) Pub. Date: Oct. 22, 2026

(54) METHODS AND COMPOSITIONS FOR TARGETED PROTEIN DEGRADATION

(71) Applicant: Ranok Therapeutics (Hangzhou) Co. Ltd., Zhejiang (CN)

(72) Inventors: Chenghao Ying, Shanghai (CN); Guoqiang Wang, Zhejiang (CN); Zhiyong Wang, Zhejiang (CN); Zimo Yang, Jiangsu (CN); Jinhua Li, Zhejiang (CN); Yaya Wang, Shanghai (CN); Yan Dai, Zhejiang (CN); Weiwen Ying, Lexington, MA (US); Kevin P. Foley, Waltham, MA (US)

(21) Appl. No.: 19/481,933

(22) PCT Filed: May 9, 2024
(86) PCT No.: PCT/US2024/028674

§ 371 Date: Nov. 6, 2025

(30) Foreign Application Priority Data
May 9, 2023 (WO) PCT/CN2023/093091

(51) Int. Cl. A61K 47/55; A61K 47/54; A61P 35/00

(52) U.S. Cl. CPC A61K 47/55; A61K 47/545; A61P 35/00

(57) ABSTRACT

Provided are PROTACs comprised of a targeting moiety designed to bind KRAS(G12D/C/V) linked to a degrader-directing moiety designed to bind an E3 ubiquitin-ligase. Pharmaceutical compositions comprising the disclosed PROTACs and their use in treating cancer and related conditions are also provided.

### RELATED APPLICATIONS

This application claims the benefit of priority to international application No. PCT/CN2023/093091, filed May 9, 2023, the entire contents of which are incorporated herein by reference.

### BACKGROUND

Chemically induced, targeted protein degradation (TPD) is a new modality for small molecule drug development. Small molecules can be designed to induce and/or stabilize the direct physical interactions of target proteins with components of various cellular protein degradation pathways, thereby driving the degradation of the targeted proteins as a way to treat disease. Proteolysis-targeting chimeras (PROTACs) are an example of such small molecules that enable TPD of specific proteins, such as KRAS mutant oncogenes (Burslem and Crews, Cell, 2020, 181:102-114; Pettersson and Crews, Drug Discov Today Technol, 2019, 31:15-27).

In a cell, networks of protein components compete and cooperate to maintain and alter the proteome in order to carry-out specific biological processes. Proteostasis is this dynamic regulation protein synthesis, folding, trafficking and degradation inside and outside of cells. Protein degradation is especially critical for proper cell function including proliferation, differentiation, and death (Lang et al, Arch Toxicol, 2021, 95:1943-1970). In cancer protein degradation is often dysregulated with tumor suppressors degraded while oncogenes are stabilized (Hanahan and Weinberg, Cell, 2011, 144:646-74).

The ubiquitin-proteasome system (UPS) is the primary pathway cells utilize to degrade and metabolically recycle proteins (Yu and Matouschek, Annu Rev Biophys, 2017, 46:149-173; Navon and Ciechanover, J Biol Chem, 2009, 284:33713-33718). Covalent attachment of the globally expressed 76 amino acid-residue peptide, ubiquitin, marks a substrate protein for degradation. The process of ubiquitination involves a series of enzymatic hand-offs. Initially, ubiquitin is attached to an E1 ubiqutin-activating enzyme. Once activated, ubiquitin is next transferred from E1 to an E2 ubibuitin-conjugating enzyme. Finally, one of several hundred E3 ubiquitin-ligase enzyme complex components, such as cereblon (CRBN) and von Hippel Lindau (VHL) (Bricelj et al, Front Chem, 2021, 9:707317), facilitate the transfer of ubiquitin to a lysine on the substrate protein. Additional ubiquitin peptides may then be attached to lysines on each ubiquitin peptide creating a polyubiquitin chain that directs the substrate protein to the proteosome, a multi-subunit protein degrading enzyme complex. For some protein substrates, ubiquitination may also affect a protein's activity, subcellular localization and protein-protein interaction profile (Zou et al., Int J Mol Sci, 2021, 22:5754; Amm et al., Biochim Biophys Acta, 2014, 1843:182-196).

The CRBN and VHL proteins function as critical components in Cullin RING E3 ubiquitin-ligase complexes (Cai and Wang, Cell Div, 2016, 11; Cheng et al., Biochim Biophys Acta Rev Cancer, 2019, 1871:138-159). Both CRBN and VHL are widely expressed across tissues types and evolutionarily conserved among vertebrates. Normally, CRBN has been observed to coordinate the ubiquitination and degradation of ion channels, the MEIS2 developmental transcription factor, the AMPK metabolic-regulating kinase, and glutamine synthase (Jo et al., J Neurochem, 2005, 94:1212-24, Hohberger and Enz, FEBS Lett, 2009, 583:633-7; Fischer et al., Nature, 2014, 12:49-53, Lee et al., J Biol Chem, 2014, 289:23343-52; Nguyen, et al., Mol Cell, 61:809-20). CRBN can also be induced to degrade transcription factors IKZF1 and IKZF3 along with Casein kinase 1A1 by immunomodulatory compounds (Kronke et al. Science, 2014, 343:301-5; Petzold et al., Nature, 2016, 532:127-30). VHL normally ubiquitinates hypoxia-inducible factor 1a (HIF1A), the primary transcription factor responsible for promoting angiogenesis (Kaelin, Nat Rev Cancer, 2008, 8:865-73).

PROTACs are bifunctional molecules that contain two different drug moieties held together by a linker. By design these PROTAC molecules can simultaneously bind both a target protein and an E3 ubiquitin-ligase. Within cells this creates ternary complexes composed of target proteins and E3 ubiquitin-ligases that are held together by PROTAC molecules. This induced proximity of the target proteins and E3 ubiquitin-ligases results in the ubiquitination of the target proteins and subsequent degradation by the proteasome. If the target protein is an oncogene this degradation may result in the death of the cancer cell or the inability to proliferate (Pettersson and Crews, Drug Discov Today Technol, 2019, 31:15-27; Bondeson et al., Cell Chem Biol, 2018, 25:78-87; Gadd et al., Nat Chem Biol, 2017, 13:514-521; Zengerle et al., ACS Chem Biol, 2015, 10:1770-1777).

Compared to conventional biochemical enzyme inhibitors, PROTACs possess several advantages. For example, PROTACs are able to work sub-stoichiometrically by inducing multiple rounds of degradation of target proteins. This is presumably due to PROTAC molecules being released from the proteosome degraded protein to bind another target protein and E3 ubiquitin ligase. This leads to a greater potency compared to each isolated moiety binding to its respective target. Moreover, synthesis and recovery of the target protein function is slower for PROTACs than observed for biochemical inhibitors. PROTACs may also possess improved target selectivity over single moiety biochemical inhibitors. Finally, PROTACs can deplete target proteins that are not responsive to biochemical inhibition by binding accessible pockets that do not affect the biochemical activity of the target but still permit their degradation (Pettersson and Crews, Drug Discov Today Technol, 2019, 31:15-27; Ding et al., Trends Pharmacol Sci, 2020, 41:464-474).

There is a need to develop improved PROTAC agents that direct the degradation of specific proteins in cancer and other diseases. The Kirsten rat sarcoma virus homolog (KRAS) is small GTPase and among the most frequently mutated genes in human cancers (Pylayeva-Gupta et al., Nat Rev Cancer, 2011, 11:761-774). Mutations that lock KRAS in an active GTP-bound state reprograms cells for perpetual proliferation by continuously stimulating the RAF-MAPK and PI3K-AKT-MTOR pro-growth signaling pathways (Kerk et al., Nat Rev Cancer, 2021, 21:510-525; Nussinov et al., Cancer Res, 2018, 78:593-602). KRAS mutated from glycine (G) at the 12th codon to aspartate (D) creates the chronically active KRAS(G12D) oncogene observed in 6.8% of cancers cases analyzed by next-generation sequencing (Zhou et al., Pathol Oncol Res, 2020, 26:2835-2837). In tumor type specific studies, KRAS(G12D) is associated with poor clinical outcomes and observed in 17% of lung, 14.3% of colorectal, and 48% of pancreatic tumors (Aredo et al., Lung Cancer, 2019, 133:144-150; Olmedillas-López et al., World J Gastroenterol, 2017, 23 (39): 7087-709; Miglio et al., Pathol Res Pract, 2014, 210:307-11; Gou et al., Br J Cancer, 2020, 22:857-867). Historically, oncogenic KRAS mutants have been considered undruggable (McCormick F, Biochem J, 2019, 476:356-74), however the G12D/G12C/G12V mutants provide a unique moiety binding space due to the encoding of an aspartic acid (D)/an cysteine (C)/an valine (V) in place of glycine (G). This allows for cancer cells that are driven by the KRAS(G12D)/KRAS(G12C)/KRAS(G12V) mutants to be selectively targeted and degraded by PROTAC agents. It is therefore desirable to develop PROTAC agents directed at degrading KRAS(G12D) or KRAS(G12C) or KRAS(G12V) in cancer.

### SUMMARY

Provided herein are PROTACs comprised of a targeting moiety designed to bind KRAS(G12D/G12C/G12V) linked to a degrader-directing moiety designed to bind an E3 ubiquitin-ligase, e.g., CRBN or VHL. Such compounds include those having the Formula I:

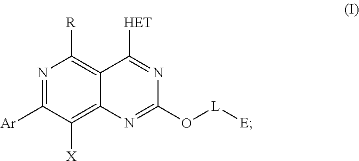

and pharmaceutically acceptable salts thereof. Compositions comprising the disclosed compounds of Formula I as well as methods for their manufacture and their biochemical activity are also provided. In one aspect, the disclosed compounds induce targeted oncogenic protein degradation in a tumor-selective fashion and are useful in the treatment of cancer and related conditions.

### DETAILED DESCRIPTION

### 1. General Description of Compounds

Provided herein are PROTAC compounds having the Formula I:

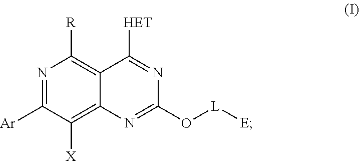

or a pharmaceutically acceptable salt thereof, wherein,
HET is an optionally substituted heterocyclyl;
Ar is an optionally substituted aryl or optionally substituted heteroaryl; R is selected from hydrogen, (C₁-C₄)alkyl, (C₁-C₄)haloalkyl, (C₁-C₄)alkoxy, deuterated(C₁-C₄)alkoxy, (C₁-C₄)haloalkoxy, (C₁-C₄)alkynyl, (C₁-C₄)alkenyl, halo, (C₃-C₆)cycloalkyl, —O(C₃-C₆)cycloalkyl, cyano, NH₂, —NH(C₁-C₄)alkyl, —N[(C₁-C₄)alkyl]₂, —P(O)[(C₁-C₄)alkyl]₂, and —S(C₁-C₄)alkyl, wherein said (C₃-C₆)cycloalkyl and said (C₃-C₆)cycloalkyl of —O(C₃-C₆)cycloalkyl are optionally substituted with 1 to 3 groups selected from halo, (C₁-C₄)alkyl, (C₁-C₄)alkoxy, halo(C₁-C₄)alkoxy, and cyano;
X is hydrogen or halo;
L is a linker; and
E is a chemical moiety that targets E3 ligase.

### 2. Definitions

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.”

As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values. The term “substantially” means more than 50%, preferably more than 80%, and most preferably more than 90% or 95%.

As used herein the term “comprising” or “comprises” are used in reference to compositions, methods, and respective component(s) thereof, that are present in a given embodiment, yet open to the inclusion of unspecified elements.

As used herein the term “consisting essentially of” refers to those elements required for a given embodiment. The term permits the presence of additional elements that do not materially affect the basic and novel or functional characteristic(s) of that embodiment of the disclosure.

The term “consisting of” refers to compositions, methods, and respective components thereof as described herein, which are exclusive of any element not recited in that description of the embodiment.

As used herein, the term “alkyl” means a saturated straight chain or branched non-cyclic hydrocarbon having, unless specified otherwise, from 1 to 10 carbon atom e.g., (C₁-C₆)alkyl or (C₁-C₄)alkyl. Representative straight chain alkyls include methyl, ethyl, n-propyl, n-butyl, n-pentyl, n-hexyl, n-heptyl, n-octyl, n-nonyl and n-decyl; while saturated branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like.

As used herein, the term “alkynyl” means a saturated straight chain or branched non-cyclic hydrocarbon having, unless specified otherwise, from 2 to 10 carbon atoms (e.g., (C₂-C₆)alkynyl or (C₂-C₄)alkynyl) and having at least one carbon-carbon triple bond. Representative straight chain and branched alkynyls include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl, and the like.

As used herein, the term “cycloalkyl” means a saturated, monocyclic alkyl radical having from e.g., 3 to 10 carbon atoms (e.g., from 4 to 6 carbon atoms). Representative cycloalkyls include cyclopropyl, cyclobutyl, cyclopentyl, cyclohexyl, cycloheptyl, cyclooctyl, cyclononyl, and cyclodecanyl.

The term “oxo” refers to the group ═O.

As used herein, the term “haloalkyl” means and alkyl group in which one or more (including all) the hydrogen radicals are replaced by a halo group, wherein each halo group is independently selected from —F, —Cl, —Br, and —I. Representative haloalkyl groups include trifluoromethyl, bromomethyl, 1,2-dichloroethyl, 4-iodobutyl, 2-fluoropentyl, and the like.

“Alkoxy” means an alkyl radical attached through an oxygen linking atom, represented by —O-alkyl. For example, “(C₁-C₄)alkoxy” includes methoxy, ethoxy, proproxy, and butoxy.

“Haloalkoxy” is a haloalkyl group which is attached to another moiety via an oxygen atom such as, e.g., —OCHF₂ or —OCF₃.

The term “aryl” used alone or as part of a larger moiety as in “aralkyl”, “aralkoxy”, or “aryloxyalkyl”, refers to monocyclic and bicyclic carbon ring systems having a total of six to 10 ring members, wherein at least one ring in the system is aromatic. Examples include, but are not limited to phenyl, naphthyl, anthracyl and the like. It will be understood that when specified, optional substituents on an aryl group may be present on any substitutable position.

As used herein, the term “heterocyclyl” means a 4- to 12-membered monocyclic or polycyclic saturated or partially unsaturated heterocyclic ring (e.g., bridged bicyclic) containing 1 to 4 heteroatoms independently selected from N, O, and S. The heterocycle may be attached via any heteroatom or carbon atom, as valency permits. Representative heterocycles include morpholinyl, thiomorpholinyl, pyrrolidinonyl, pyrrolidinyl, piperidinyl, piperazinyl, oxiranyl, dioxanyl, oxetanyl, dihydrofuranyl, dihydropyranyl, isoindolinyl, dihydropyridinyl, tetrahydrofuranyl, tetrahydropyranyl, tetrahydropyrindinyl, tetrahydropyrimidinyl, diazabicyclooctanyl, hexahydropyrrolizinyl, and the like. Optional substituents on a heterocyclyl group may be present on any substitutable position and, include, e.g., the position at which the heterocyclyl is attached.

As used herein, the term “heteroaryl” means a 5- to 12-membered aromatic radical containing 1-4 heteroatoms selected from N, O, and S. A heteroaryl group may be mono- or bicyclic. The heteroaryl may be attached via any heteroatom or carbon atom, as valency permits. Representative heteroaryl groups include pyridyl, furanyl, thienyl, pyrrolyl, oxazolyl, imidazolyl, thiazolyl, isoxazolyl, quinolinyl, pyrazolyl, isothiazolyl, pyridazinyl, pyrimidinyl, pyrazinyl, triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, benzothienyl, and the like. Optional substituents on a heteroaryl group may be present on any substitutable position and, include, e.g., the position at which the heteroaryl is attached.

As used herein, the term “halogen” or “halo” means F, Cl, Br or I.

The term “linker” or “tether,” used interchangeably, refers to a chemical moiety that joins two other moieties (e.g., a first binding moiety and a second binding moiety). A linker can covalently join a first binding moiety and a second binding moiety. In one aspect, the linker is uncleavable in vivo. In one aspect, the linker comprises one or more cyclic ring systems. In another aspect, the linker comprises an alkyl chain optionally substituted by and/or interrupted with one or more chemical groups. In one aspect, the linker comprises optimal spatial and chemical properties to effectuate optimal therapeutic activity. In one aspect, the linker does not interfere with the ability of the first binding moiety and/or the second binding moiety to bind their respective targets (e.g., CRBN or VHL and KRAS(G12D/C/V)). In one aspect, the linker alters the ability of the first binding moiety and/or the second binding moiety to bind their respective targets (e.g., CRBN or VHL and KRAS(G12D/C/V)).

The term “KRAS” refers to the protein product of the KRAS proto-oncogene, GTPase gene.

The term “KRAS(G12D/C/V)” refers to the protein product of the KRAS gene carrying a mutation that results in the glycine amino acid at position 12 of KRAS being replaced by a aspartate (D), cysteine (C), or valine (V).

The term “CRBN” refers to the E3-ubiquitin ligase also known as cereblon, MRT2 and MRT2A along with all its isoforms and splice variants.

The term “VHL” refers to the E3-ubiquitin ligase also known as RCA1, VHL1, pVHL, and HRCA along with all of its isoforms and splice variants.

When used in connection to describe a chemical group that may have multiple points of attachment, a hyphen (-) designates the point of attachment of that group to the variable to which it is defined. For example, —NH(C₁-C₄)alkyl means that the point of attachment for this group occurs on the nitrogen atom.

A hash bond as in “ [symbol] ” represents the point at which the depicted group is attached to the defined variable.

The compounds described herein may have chiral centers and/or geometric centers (E- and Z-isomers). It will be understood that the present disclosure encompasses all stereoisomers and geometric isomers. Tautomeric forms of the compounds described herein are also part of the present disclosure.

When the stereochemistry of a disclosed compound is named or depicted by structure, the named or depicted stereoisomer is at least 60%, 70%, 80%, 90%, 99% or 99.9% by weight pure relative to all of the other stereoisomers. Percent by weight pure relative to all of the other stereoisomers is the ratio of the weight of one stereoisomer over the weight of the depicted stereoisomer plus the weight of the other stereoisomers.

For use in medicines, the pharmaceutically acceptable salts of the disclosed compounds refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Compounds of the present teachings with acidic groups such as carboxylic acids can form pharmaceutically acceptable salts with pharmaceutically acceptable base(s). Suitable pharmaceutically acceptable basic salts include e.g., ammonium salts, alkali metal salts (such as sodium and potassium salts) and alkaline earth metal salts (such as magnesium and calcium salts). Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, benzoates and salts with amino acids such as glutamic acid.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Any compositions or methods provided herein can be combined with one or more of any of the other compositions and methods provided herein.

As used herein, the term “subject” refers to human and non-human animals, including veterinary subjects. The term “non-human animal” includes all vertebrates, e.g., mammals and non-mammals, such as non-human primates, mice, rabbits, sheep, dog, cat, horse, cow, chickens, amphibians, and reptiles. In a preferred embodiment, the subject is a human and may be referred to as a patient.

As used herein, the terms “treat,” “treating” or “treatment” refer, preferably, to an action to obtain a beneficial or desired clinical result including, but not limited to, alleviation or amelioration of one or more signs or symptoms of a disease or condition, diminishing the extent of disease, stability (i.e., not worsening) of the state of disease, amelioration or palliation of the disease state, diminishing rate of or time to progression, and remission (whether partial or total). “Treatment” can also mean prolonging survival as compared to expected survival in the absence of treatment. Treatment does not need to be curative.

A “therapeutically effective amount” is that amount sufficient to treat a disease in a subject. A therapeutically effective amount can be administered in one or more administrations. In one aspect, a therapeutically effective amount refers to a dosage of from about 0.01 to about 100 mg/kg body weight/day.

The terms “administer,” “administering” or “administration” include any method of delivery of a pharmaceutical composition or agent into a subject's system or to a particular region in or on a subject. In certain embodiments of the invention, an agent is administered intravenously, intramuscularly, subcutaneously, intradermally, intranasally, orally, transcutaneously, or mucosally. In a preferred embodiment, an agent is administered intravenously. In another preferred embodiment, an agent is administered orally. Administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, intratumoral delivery, etc.

### 3. Compounds

In a first embodiment, the compound of Formula I or a pharmaceutically acceptable salt thereof is of the Formula Ia:

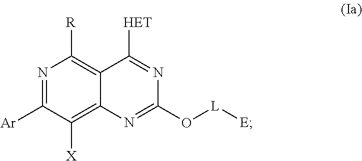

wherein R is selected from (C₁-C₄)alkyl, (C₁-C₄)haloalkyl, (C₁-C₄)alkoxy, deuterated(C₁-C₄)alkoxy, (C₁-C₄)haloalkoxy, (C₁-C₄)alkynyl, (C₁-C₄)alkenyl, halo, (C₃-C₆)cycloalkyl, —O(C₃-C₆)cycloalkyl, cyano, NH₂, —NH(C₁-C₄)alkyl, —N[(C₁-C₄)alkyl]₂, —P(O)[(C₁-C₄)alkyl]₂, and —S(C₁-C₄)alkyl, wherein said (C₃-C₆)cycloalkyl and said (C₃-C₆)cycloalkyl of —O(C₃-C₆)cycloalkyl are optionally substituted with 1 to 3 groups selected from halo, (C₁-C₄)alkyl, (C₁-C₄)alkoxy, halo(C₁-C₄)alkoxy, and cyano, wherein the remaining varaiables are as described above for Formula I.

In a second embodiment, R in the compound of Formula I or Ia is slected from (C₁-C₄)alkyl, (C₁-C₄)alkoxy, deuterated(C₁-C₄)alkoxy, (C₁-C₄)alkynyl, (C₁-C₄)alkenyl, OH, O(C₃-C₆)cycloalkyl, NH₂, —NH(C₁-C₄)alkyl, and —N[(C₁-C₄)alkyl]₂, wherein the remaining variables are as described above for Formula I or Ia. Alternatively, as part of a second embodiment, R in the compound of Formula I or Ia is selected from ethyl, —CHCH₂; —CCH, NH₂, NHMe, NMe₂, OCH₃, OCD₃, OCF₃, OEt, OCH(CH₃)₂, and, O(cyclopropyl), wherein the remaining variables are as described above for Formula I or Ia. In another alternative, as part of a second embodiment, R in the compound of Formula I or Ia is selected from (C₁-C₄)alkoxy, deuterated(C₁-C₄)alkoxy, and OH, wherein the remaining variables are as described above for Formula I or Ia. In another alternative, as part of a second embodiment, R in the compound of Formula I or Ia is selected from OCH₃, OH, and OCD₃, wherein the remaining variables are as described above for Formula I or Ia.

In a third embodiment, the compound of Formula I or Ia is of the Formula II:

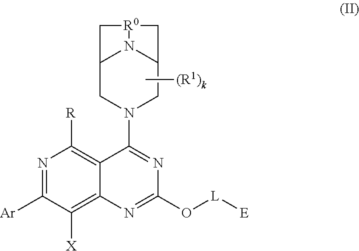

or a pharmaceutically acceptable salt thereof, wherein
R¹ is selected from halo, (C₁-C₄)alkyl, hydroxy(C₁-C₄)alkyl, cyano (C₁-C₄)alkyl, —CH(═O), —C(O)₂H, —C(O)₂ (C₁-C₄)alkyl, C(O)₂NH₂, —C(O)₂NH(C₁-C₄)alkyl, —C(O)₂N [(C₁-C₄)alkyl]₂, and 5- to 6-membered optionally substituted heteroaryl;
k is 0, 1, 2, or 3;
R⁰ is selected from hydrogen, CN, —C(O)CN, —C(O)R^{a′}, —C(O)NHR^{a′}, and (C₁-C₄)alkyl, wherein said (C₁-C₄)alkyl is optionally substituted with 1 to 3 groups selected from CN, OH, (C₃-C₆)cycloalkyl, halo, O(C₁-C₄)alkyl, (C₂-C₄)alkenyl, (C₂-C₄)alkynyl, —COR^{a′}, and —C(O)OR^{a′}; and
R^{a′} is slected from (C₁-C₄)alkyl and (C₁-C₄)alkenyl, wherein said alkyl is opt substituted with 1 to 3 groups selected from CN and halo, wherein the remaining variables are as described above for Formula I or Ia or the second embodiment.

In a fourth embodiment, R⁰ in Formula I, Ia, or II is selected from hydrogen-C(O)R^{a′}, —C(O)NHR^{a′}, and (C₁-C₄)alkyl, wherein said (C₁-C₄)alkyl is optionally substituted with 1 to 3 groups selected from halo, CN, OH, and (C₃-C₆)cycloakyl, wherein the remaining variables are as described above for Formula I, Ia, or II and/or any one of the third or fourth embodiments. In a fifth embodiment, R^{a′} is selected from (C₁-C₄)alkyl, (C₁-C₄)alkenyl, each of which are optionally substituted with 1 to 3 groups slected from CN and halo, wherein the remaining variables are as described above for Formula I, Ia, or II and/or any one of the third or fourth embodiments. Alternatively, as part of a fourth embodiment, R⁰ in Formula I, Ia, or II is selected from hydrogen, C(O)CH₂CH₃, C(O)CF₃, CH₃, CH₂CN, (CH₂)₂CN, CH₂CH(CH₃), CH₂CF₃, (CH₂)₂OH, CH₂(cylopropyl), C(O)CH₂CN, C(O)CH(CH₃)CN, C(O)CH(CH₂CH₃)CN, C(O)(CH₂)₂CN, C(O)CH₂CH(CH₃)CN, C(O)CH(CH₃)CH₂CN, C(O)CH(CN) CH₂CF₃, C(O)NHCH₂CN, C(O)NHCH₂CF₃, and C(O)CHCH₂, wherein the remaining variables are as described above for Formula I, Ia, or II and/or any one of the third or fourth embodiments. In another alternative, as part of a fourth embodiment, R⁰ in Formula I, Ia, or II is selected from —CH₂CN, —CH₂CH₂OH, —CH₂CH₂OMe, and CH₂C(O)Me, wherein the remaining variables are as described above for Formula I, Ia, or II and/or any one of the third or fourth embodiments.

In a sixth embodiment, k in Formula I, Ia, or II is 0, wherein the remaining variables are as described above for Formula I, Ia, or II and/or any one of the third to fifth embodiments.

In a seventh embodiment, X in Formula I, Ia, or II is hydrogen or fluoro, wherein the remaining variables are as described above for Formula I, Ia, or II and/or any one of the third to sixth embodiments. Alternatively, as part of a seventh embodiment X in Formula I, Ia, or II is fluoro, wherein the remaining variables are as described above for Formula I, Ia, or II and/or any one of the third to sixth embodiments.

In an eighth embodiment, Ar in Formula I, Ia, or II is an optionally substituted phenyl or optionally substituted naphthyl, wherein the remaining variables are as described above for Formula I, Ia, or II and/or any one of the third to seventh embodiments. Alternatively, as part of an eighth embodiment, Ar in Formula I, Ia, or II is phenyl or naphthalenyl, each of which are optionally substituted with one to three groups independently selected from R^{A}, wherein R^{A} is selected from halo, (C₁-C₄)alkyl, (C₂-C₄)alkynyl, (C₂-C₄)alkynylNH₂, (C₁-C₄)alkoxy, halo(C₁-C₄)alkoxy, (C₁-C₄)alkylOH, OH, NH₂, —NH(C₁-C₄)alkyl, —N[(C₁-C₄)alkyl]₂, C(O)NH₂, C(O)NH(C₁-C₄)alkyl, C(O)[(C₁-C₄)alkyl]₂, —NHC(O)(C₁-C₄)alkyl, —N(C₁-C₄)alkylC(O)(C₁-C₄)alkyl, —NHC(O)O(C₁-C₄)alkyl, NHC(O)NH(C₁-C₄)alkyl, CN, —S(C₁-C₄)alkyl, —Shalo(C₁-C₄)alkyl, and (C₃-C₆)cycloalkyl, wherein said (C₁-C₄)alkyl and said (C₃-C₆)cycloalkyl are each optionally substituted with one to two groups selected from halo, (C₁-C₄)alkoxy, halo(C₁-C₄)alkoxy, OH, NH₂, —NH(C₁-C₄)alkyl, —N[(C₁-C₄)alkyl]₂, C(O)NH₂, C(O)NH(C₁-C₄)alkyl, C(O)[(C₁-C₄)alkyl]₂, —NHC(O)(C₁-C₄)alkyl, —N(C₁-C₄)alkyIC(O)(C₁-C₄)alkyl, —NHC(O)O(C₁-C₄)alkyl, NHC(O)NH(C₁-C₄)alkyl, CN, —S(C₁-C₄)alkyl, and —S(C₁-C₄)haloalkyl, wherein the remaining variables are as described above for Formula I, Ia, or II and/or any one of the third to seventh embodiments. In another alternative, as part of an eighth embodiment, Ar in Formula I, Ia, or II is naphthalenyl optionally substituted with one to three groups independently selected from R^{A}, wherein the remaining variables are as described above for Formula I, Ia, or II and/or any one of the third to seventh embodiments. In another alternative, as part of an eighth embodiment, R^{A} is selected from (C₂-C₄)alkynyl, halo, and OH, wherein the remaining variables are as described above for Formula I, Ia, or II and/or any one of the third to seventh embodiments. In another alternative, as part of an eighth embodiment, Ar in Formula I, Ia, or II is

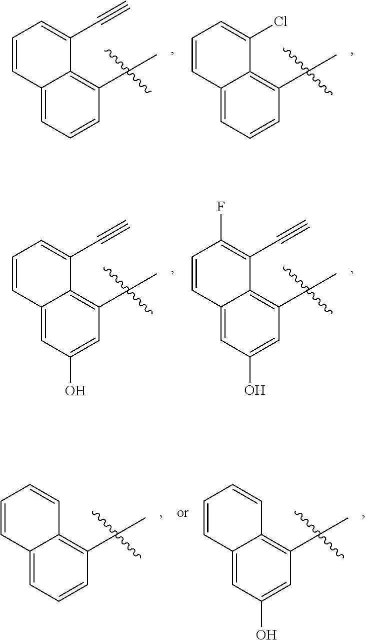 [SMILES: *c1cccc2cccc(C#C)c12 ; *c1cccc2cccc(Cl)c12 ; *c1cc(O)cc2cccc(C#C)c12 ; *c1cc(O)cc2ccc(F)c(C#C)c12 ; *c1cccc2ccccc12 ; *c1cc(O)cc2ccccc12]

wherein the remaining variables are as described above for Formula I, Ia, or II and/or any one of the third to seventh embodiments. In another alternative, as part of an eighth embodiment, Ar in Formula I, Ia, or II is

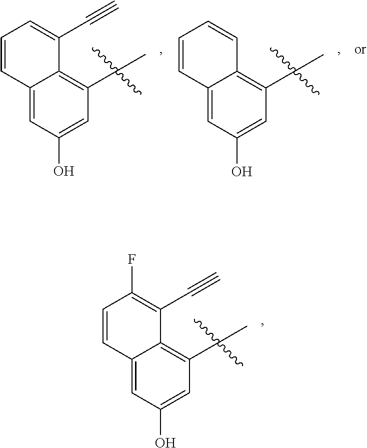 [SMILES: *c1cc(O)cc2cccc(C#C)c12 ; *c1cc(O)cc2ccccc12 ; *c1cc(O)cc2ccc(F)c(C#C)c12]

wherein the remaining variables are as described above for Formula I, Ia, or II and/or any one of the third to seventh embodiments. In another alternative, as part of an eighth embodiment, Ar in Formula I, Ia, or II is

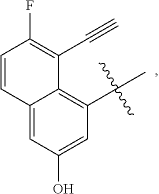 [SMILES: *c1cc(O)cc2ccc(F)c(C#C)c12]

wherein the remaining variables are as described above for Formula I, Ia, or II and/or any one of the third to seventh embodiments.

In a ninth embodiment, E in Formula I, Ia, or II is selected from a cereblon (CRBN) modulator and a von Hippel-Lindau (VHL) ligand, wherein the remaining variables are as described above for Formula I, Ia, or II and/or any one of the third to eighth embodiments. Alternatively, as part of a ninth embodiment, E in Formula I, Ia, or II is of the structural formula:

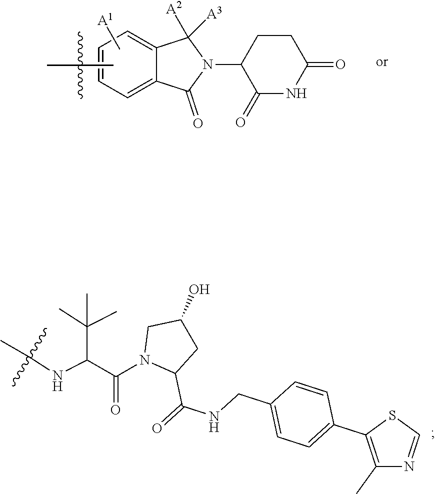

wherein A¹ is halo; and A² and A³ are both hydrogen or A² and A³ taken together form ═O, wherein the remaining variables are as described above for Formula I, Ia, or II and/or any one of the third to eighth embodiments. In another alternative, as part of a ninth embodiment, E in Formula I, Ia, or II is of the structural formula:

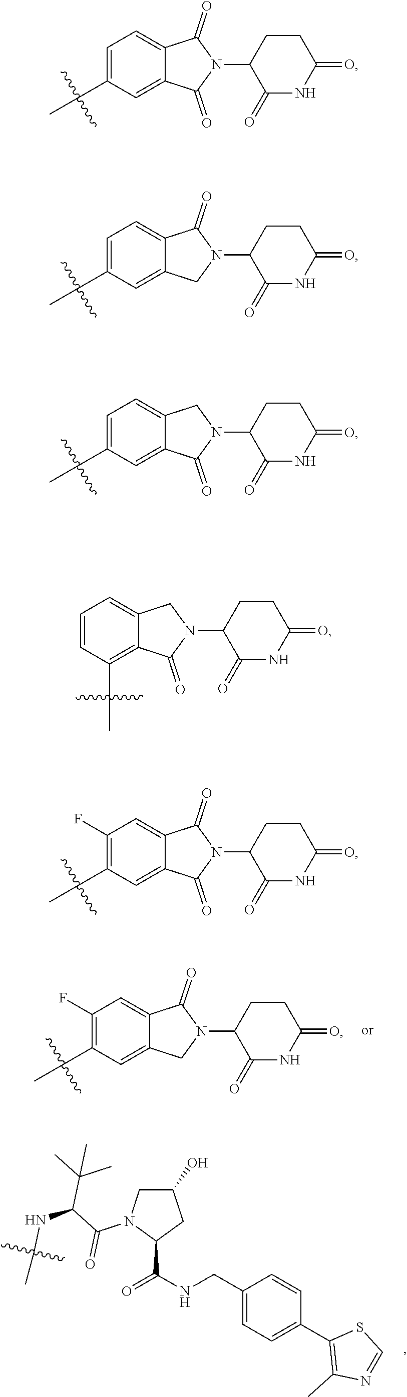

wherein the remaining variables are as described above for Formula I, Ia, or II and/or any one of the third to eighth embodiments. In another alternative, as part of a ninth embodiment, E in Formula I, Ia, or II is of the structural formula:

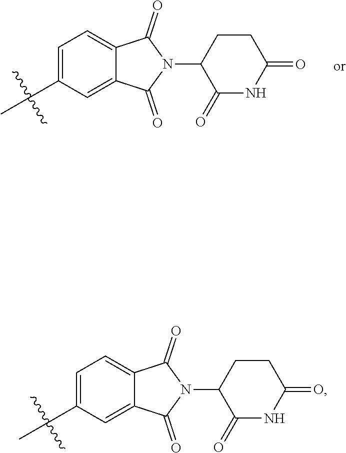 [SMILES: *c1ccc2c(c1)C(=O)N(C1CCC(=O)NC1=O)C2=O ; *c1ccc2c(c1)C(=O)N(C1CCC(=O)NC1=O)C2=O]

wherein the remaining variables are as described above for Formula I, Ia, or II and/or any one of the third to eighth embodiments.

In a tenth embodiment, L in Formula I, Ia, or II is -Het¹-X¹—*, *-Het¹-Het²-X¹, *—NR^{c}—X¹-Het¹-Het²-X², *—X¹-Het¹-X²-Het²-(CH₂)ₘO—, —NR^{c}—(CH₂)ₘ—X¹-Het¹-X²—*, —NR^{c}—(CH₂)ₘ—X¹—NR^{c}—(CH₂CH₂O)ₙ—*, —NR^{c}—(CH₂)ₘ—X¹—NR^{c}—(CH₂)ₚ—*, —NR^{c}—(CH₂)ₘ—X¹-Het¹-X²-Het²-X³—*, —O(CH₂)ₘ—X¹-Het¹-X²-Het²-X³—*, —O(CH₂)ₘ—X¹—NR^{c}—(CH₂)ₚ-Het¹-X²-Het²-X³—*, *—X¹—NR^{c}—(CH₂)ₘ-Het¹-X²-Het²-X³—(CH₂)ₚ—NR^{d}—(CH₂)ₚ—, —NR^{c}—(CH₂)ₘ—X¹—(CH) CH₃-Het¹-X²-Het³-X³—*, —NR^{c}—(CH₂)ₘ—X¹—(CH₂)ₚ-Het¹-X²-Het²-X³—*, —NR^{c}—(CH₂)ₘ—X¹—NR^{d}—(CH₂)ₚ-Het¹-X²-Het²-X³—*, —NR^{c}—(CH₂)ₘ—NR^{d}-X¹-Het¹-X²—*, *Het¹-X¹-Het²-X²—, *X¹-Het¹-X²—, *(CH₂CH₂O)ₙ—NR^{c}—X¹-Het¹-Het²-X²—, *Het¹-X¹-Het²-X²-Het³-X³, *X¹-Het¹-X²-Het²-X³—, *-Het¹-X¹—NR^{c}-Het²-X²—, *X¹-Het¹-Het²-X²—, *X¹—(CH₂)ₘO-Het¹-X²—, *X¹—(CH₂)ₘNR^{c}-X²-Het¹-Het²-X², *-Het¹-X¹-Het²-X²—O—, *—O(CH₂)ₘ-Het¹-(CH₂)ₚ—O(CH₂)ₘ—NR^{c}—X²—*, *-Het¹-O—(CH₂)ₘ—X¹-Het²-X², *-Het¹-O—(CH₂)ₘ—X¹—NR^{c}—(CH₂CH₂O), (CH₂)ₘ-Het²-X²—, *-Het¹-X¹—NR^{c}—(CH₂)ₘ—, *-Het¹-X¹-Het²-Het³-X²—, *-Het¹-X¹—NR^{c}—(CH₂CH₂O) n (CH₂)ₘ—, *-Het¹-X¹—NR^{c}—(CH₂CH₂O) n Het²-(CH₂)ₘ—X²—, *-Het¹-X¹—NR^{c}—(CH₂CH₂O)ₙ—, *-Het¹-X¹—NR^{c}—(CH₂)ₘ-Het²-X²-Het³-(CH₂)ₘ—, *-Het¹-X¹-Het²-(CH₂)ₘ-Het³-X²—, *-Het¹-X¹-Het²-, *-Het¹-X¹—NR^{c}—, *-Het¹-X¹—NR^{c}—(CH₂)ₘ-Phe-X²-Het²-(CH₂)ₘ—, *-Het¹-X¹-Het²-Het³, *-Het¹-X¹-Het²-(CH₂)ₘ-Het³-X²—(CH₂)ₚ—NR^{c}—(CH₂)ₘ—, *-Het¹-X¹-Het²-(CH₂)ₘ-Het³-(CH₂)ₘ—O—, *-Het¹-X¹-Het²-(CH₂)ₘ-Het³-(CH₂)ₚ—NR^{c}—(CH₂)ₘ—, *-Het¹-X¹-Het²-(CH₂CH₂O)ₙ—, *-Het¹-X¹—(CH₂)ₘ-Het²-X²—, *—(CH₂CH₂O)ₒ—(CH₂)ₚ-Het¹-X¹-Het²-(CH₂CH₂O)ₙ, *—(CH₂CH₂O)ₙ—(CH₂)ₘ-Het¹-X¹-Het²-X², *-Het¹-X¹-Phe-X²—NR^{c}—X³—, *—(CH₂CH₂O)—(CH₂)ₚ-Het¹-X¹-Phe-X²—NR^{c}—(CH₂CH₂O)ₙ—, *—(CH₂CH₂O)ₙ—(CH₂)ₘ—NR^{c}-Phe-X¹—, *—(CH₂CH₂O), —(CH₂)ₚ—NR^{c}-Phe-(CH₂CH₂O)ₙ—, *—(CH₂CH₂O)ₒ—(CH₂)ₚ—NR^{c}—(CH₂CH₂O)ₙ—(CH₂)ₘ—, *—(CH₂CH₂O)ₙ—(CH₂)ₘ—NR^{c}—(CH₂CH₂O)ₙ—(CH₂)ₘ—C(O)—NR^{d}—(CH₂CH₂O)ₒ—(CH₂)ₚ—, *—(CH₂CH₂O), —(CH₂)ₚ—NR^{c}—(CH₂CH₂O)ₙ—(CH₂)ₘ-Het¹-X¹-Het²-X²—, *—(CH₂CH₂O)ₒ—(CH₂)ₚ—NR^{c}—(CH₂CH₂O)ₙ—(CH₂)ₘ-Het¹-X¹-Het²-X²—(CH₂CH₂O)ₒ, *—NR^{c}—(CH₂CH₂O)ₙ—(CH₂)ₘ-Phe-NH—X¹-Het¹-X², *—NR^{c}—(CH₂CH₂O)ₙ—(CH₂)ₘ-Phe-NH—X¹-Het¹-X²—(CH₂CH₂O)ₒ, *—(CH₂CH₂O)ₒ—(CH₂)ₚ—NR^{c}—(CH₂CH₂O)ₙ—(CH₂)ₘ-Phe-X¹—NR^{c}—(CH₂CH₂O)ₒ—(CH₂)ₚ—, *—(CH₂CH₂O), —(CH₂)ₚ—NR^{c}—(CH₂CH₂O)ₙ—(CH₂)ₘ-Het¹-X¹—, *—(CH₂CH₂O)ₒ—(CH₂)ₚ—NR^{c}—(CH₂CH₂O)ₙ—(CH₂)ₘ-Het¹-X¹—(CH₂CH₂O)ₙ—, *—(CH₂CH₂O)ₙ—(CH₂)ₘ—NR^{c}—(CH₂)ₘ—C(O)—NR^{d}-Het¹-X¹-Het²-(CH₂CH₂O)ₒ—(CH₂)ₚ, or *—NR^{c}—(CH₂)ₘ—C(O)—NR^{d}—(CH₂)ₘ-Het¹-X¹-Het²-X²—;
* indicates the point of attachment to E;
Het¹, Het², and Het³ are each independently phenyl, a 5- to 8-membered heterocyclyl, 5- to 7-membered heteroaryl, or a 3- to 6-membered cycloalkyl, each of which are optionally substituted with (C₁-C₄)alkyl;
Phe is phenyl;
X¹, X², and X³, are each independently C(O) or (CH₂)ᵣ;
R^{c} and R^{d} are each independently hydrogen or (C₁-C₄)alkyl; and m, n, o, p, and r are each independently integers selected from 0, 1, 2, 3, 4, 5, and 6, wherein the remaining variables are as described above for Formula I, Ia, or II and/or any one of the third to ninth embodiments. Alternatively, as part of a tenth embodiment, L in Formula I, Ia, or II is *-Het¹-Het²-X¹—, *—NR^{c}—X¹-Het¹-Het²-X², *Het¹-X¹-Het²-X²—, *X¹-Het¹-X², *(CH₂CH₂O)ₙ—NR^{c}—X¹-Het¹-Het²-X², *Het¹-X¹-Het²-X²-Het³-X³, *X¹-Het¹-X²-Het²-X³—, *-Het¹-X¹—NR^{c}-Het²-X², *X¹-Het¹-Het²-X²—, *X¹—(CH₂)ₘO-Het¹-X²—, *X¹—(CH₂)ₘNR^{c}-X²-Het¹-Het²-X²—, or *-Het¹-X¹-Het²-Het³-X²— wherein the remaining variables are as described above for Formula I, Ia, or II and/or any one of the third to ninth embodiments. Also as part of a tenth embodiment, Het¹, Het², and Het³ are each independently a 5- to 8-membered heterocyclyl, or a 3- to 6-membered cycloalkyl, each of which are optionally substituted with (C₁-C₄)alkyl wherein the remaining variables are as described above for Formula I, Ia, or II and/or any one of the third to ninth embodiments. Also as part of a tenth embodiment, m, n, o, p, and r are each independently integers selected from 0, 1, 2, or 3 wherein the remaining variables are as described above for Formula I, Ia, or II and/or any one of the third to ninth embodiments. Also as part of a tenth embodiment, L in Formula I, Ia, or II is selected from

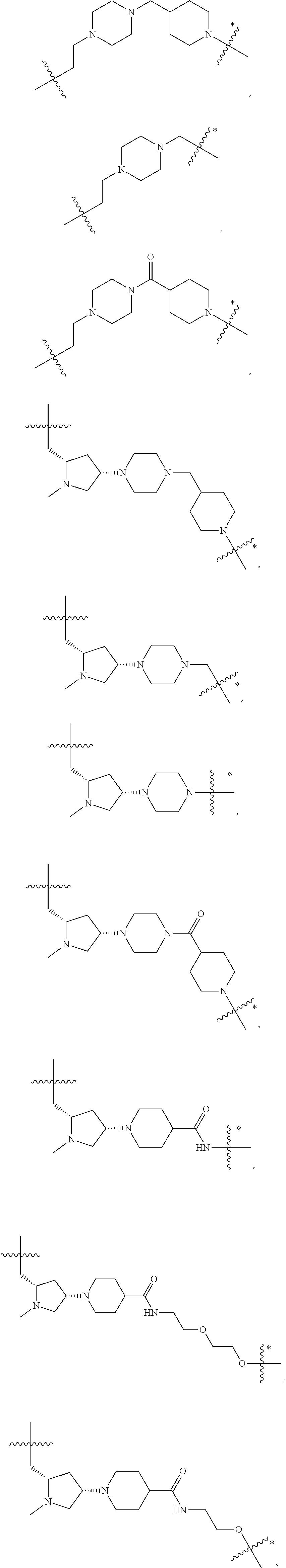

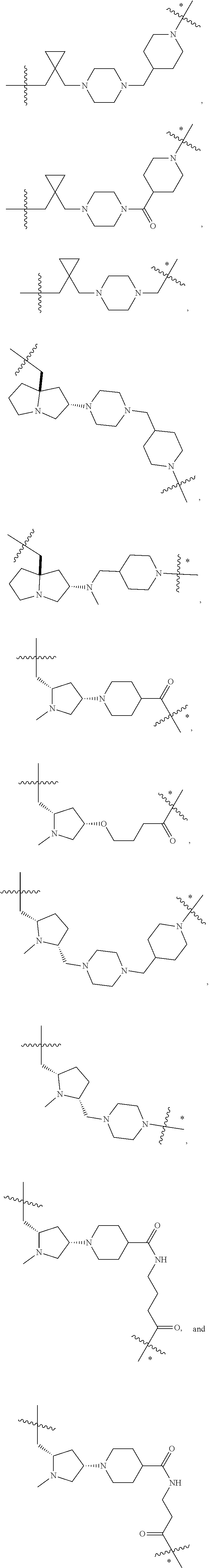

wherein the remaining variables are as described above for Formula I, Ia, or II and/or any one of the third to ninth embodiments. Also as part of a tenth embodiment, L in Formula I, Ia, or II is

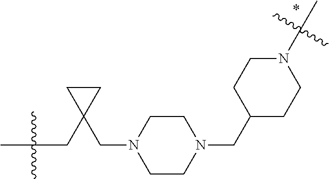 [SMILES: *CC1(CN2CCN(CC3CCN(*)CC3)CC2)CC1]

wherein the remaining variables are as described above for Formula I, Ia, or II and/or any one of the third to ninth embodiments.

Compounds having the Formula I, Ia, or II are further disclosed in the Exemplification and are included in the present disclosure. Pharmaceutically acceptable salts thereof as well as the neutral forms are included.

### 4. Uses, Formulation and Administration

Compounds and compositions described herein are generally useful as anticancer therapies. In one aspect, the disclosed compounds and compositions behave as proteolysis-targeting chimeras (PROTACs) in which one portion of the compounds is responsible for binding KRAS(G12D/C/V) and the other portion is responsible for binding to E3 ubiquitin-ligases, CRBN or VHL. Their mechanisms of action include, but are not limited to, degrading KRAS(G12D/C/V) and thereby impeding down-stream signals that may result in inhibition of cancer cell growth and/or induction of cancer cell death or other KRAS or KRAS(G12D/C/V) functions.

In one aspect, the disclosed compounds effectuate the degradation of KRAS(G12D/C/V).

In one aspect, the disclosed compounds may effectuate the activity of KRAS(G12D/C/V).

In one aspect, the disclosed compounds may effectuate the protein-protein interactions between KRAS(G12D/C/V) and upstream signaling component such as SOS1.

In one aspect, the disclosed compounds may effectuate the protein-protein interactions between KRAS(G12D/C/V) and downstream signaling components such as RAF1 or PI3K. Thus, provided herein are methods of treating conditions which are responsive to the degradation of KRAS(G12D/C/V) comprising administering to a subject in need thereof, a therapeutically effective amount of one or more compounds or compositions described herein. Also provided is the use of one or more compounds or compositions described herein in the manufacture of a medicament for treating conditions which are responsive to the degradation of KRAS(G12D/C/V). Further provided is the use of a compound or composition described herein for treating conditions which are responsive to the degradation of KRAS(G12D/C/V).

In one aspect, the condition treated by the present compounds and compositions is a cancer. The terms “cancer” or “tumor” are well known in the art and refer to the presence, e.g., in a subject, of cells possessing characteristics typical of cancer-causing cells, such as uncontrolled proliferation, immortality, metastatic potential, rapid growth and proliferation rate, decreased cell death/apoptosis, and certain characteristic morphological features. Cancer cells are often in the form of a solid tumor. However, cancer also includes non-solid tumors, e.g., blood tumors, e.g., leukemia, wherein the cancer cells are derived from bone marrow. As used herein, the term “cancer” includes pre-malignant as well as malignant cancers. Cancers include, but are not limited to, acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, Burkitt's lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin and non-Hodgkin), malignancies and hyperproliferative disorders of the bladder, breast, colon, lung, ovaries, pancreas, prostate, skin, and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, and Wilms' tumor. Other cancers include primary cancer, metastatic cancer, oropharyngeal cancer, hypopharyngeal cancer, liver cancer, gall bladder cancer, bile duct cancer, small intestine cancer, urinary tract cancer, kidney cancer, urothelium cancer, female genital tract cancer, uterine cancer, gestational trophoblastic disease, male genital tract cancer, seminal vesicle cancer, testicular cancer, germ cell tumors, endocrine gland tumors, thyroid cancer, adrenal cancer, pituitary gland cancer, hemangioma, sarcoma arising from bone and soft tissues, Kaposi's sarcoma, nerve cancer, ocular cancer, meningial cancer, glioblastomas, neuromas, neuroblastomas, Schwannomas, solid tumors arising from hematopoietic malignancies such as leukemias, metastatic melanoma, recurrent or persistent ovarian epithelial cancer, fallopian tube cancer, primary peritoneal cancer, gastrointestinal stromal tumors, colorectal cancer, gastric cancer, melanoma, glioblastoma multiforme, non-squamous non-small-cell lung cancer, malignant glioma, epithelial ovarian cancer, primary peritoneal serous cancer, metastatic liver cancer, neuroendocrine carcinoma, refractory malignancy, triple negative breast cancer, HER2-amplified breast cancer, nasopharageal cancer, oral cancer, biliary tract, hepatocellular carcinoma, squamous cell carcinomas of the head and neck (SCCHN), non-medullary thyroid carcinoma, recurrent glioblastoma multiforme, neurofibromatosis type 1, CNS cancer, liposarcoma, leiomyosarcoma, salivary gland cancer, mucosal melanoma, acral/lentiginous melanoma, paraganglioma, pheochromocytoma, advanced metastatic cancer, solid tumor, triple negative breast cancer, colorectal cancer, sarcoma, melanoma, renal carcinoma, endometrial cancer, thyroid cancer, rhabdomysarcoma, multiple myeloma, ovarian cancer, glioblastoma, gastrointestinal stromal tumor, mantle cell lymphoma, and refractory malignancy.

“Solid tumor,” as used herein, is understood as any pathogenic tumor that can be palpated or detected using imaging methods as an abnormal growth having three dimensions. A solid tumor is differentiated from a blood tumor such as leukemia. However, cells of a blood tumor are derived from bone marrow; therefore, the tissue producing the cancer cells is a solid tissue that can be hypoxic.

“Tumor tissue” or “tumorous tissue” are understood as cells, extracellular matrix, and other naturally occurring components associated with the solid tumor.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the composition.

### Abbreviation

DMF: dimethylformaldehyde
CH₂Cl₂: dichloromethane
EtOAc: ethyl acetate
PE: petroleum ether
THF: Tetrahydrofuran
MeOH: Methanol
CDI: 1,1′-Carbonylbis-1H-imidazole
DIEA: N,N-Diisopropylethylamine
HOBt: 1-Hydroxybenzotriazole hydrate
EDCI: 1-Ethyl-3-(3-dimethylaminopropyl) carbodiimide
HATU: 1-[Bis(dimethylamino)methylene]-1H-1,2,3-triazolo[4,5-b]pyridinium 3-oxide hexafluorophosphate
TFA: Trifluoroacetic Acid
BINAP: 2,2′-bis(diphenylphosphino)-1,1′-binaphthyl
XantPhos: 4,5-Bis(diphenylphosphino)-9,9-dimethylxanthene
Pd₂(dba)₃: Tris(dibenzylideneacetone)dipalladium(0)
DEA: Diethanolamine
SEM-Cl: (2-Chloromethoxyethyl)trimethylsilane
Pd(dppf)Cl₂:1,1′-Bis(diphenylphosphino)ferrocene]palladium (II) chloride
DMAP: 4-Dimethylaminopyridine
T₃P: Propyl phosphate tricyclic anhydride solution
STAB: Sodium triacetoxyborohydride
MTBE: Methyl tert-butyl ether

### Exemplification

### Chemical Synthesis

The representative examples that follow are intended to help illustrate the present disclosure, and are not intended to, nor should they be construed to, limit the scope of the invention. General starting materials used were obtained from commercial sources or prepared in other examples, unless otherwise noted.

### Preparation of Compounds

The compounds claimed herein were prepared following the procedures outlined in the following protocols.

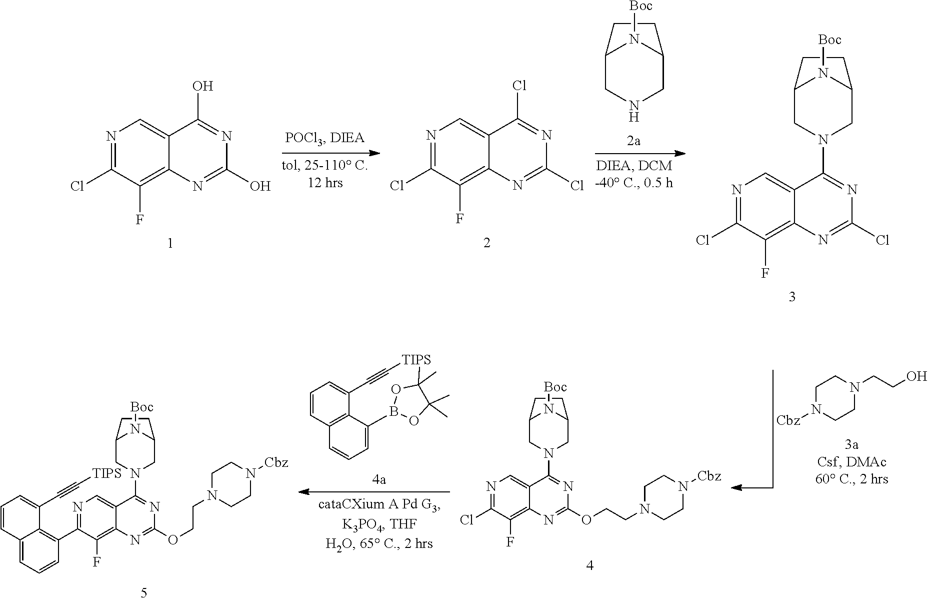

### Intermediate 2:

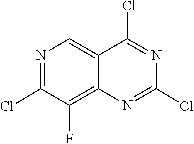 [SMILES: Fc1c(Cl)ncc2c(Cl)nc(Cl)nc12]

2,4,7-trichloro-8-fluoropyrido[4,3-d]pyrimidine. 2 batches: To a mixture of compound 1 (50.0 g, 232 mmol, 1.00 eq) in Tol. (150 mL) was added POCl₃ (178 g, 1.16 mol, 108 mL, 5.00 eq) at 25° C. Then DIEA (65.9 g, 510 mmol, 88.9 mL, 2.20 eq) was added into the mixture blow 40° C. The mixture was stirred at 110° C. for 12 hrs. LCMS showed desired MS was detected. The reaction mixture was distilled under reduced pressure to remove POCl₃ at 90° C. The residue was poured into Sat.NaHCO₃ slowly (keep pH=8). During this period, yellow precipitate was formed. It was collected by filtration and washed by H₂O. The solid was used for next step directly. Compound 2 (101 g, 400 mmol, 86.2% yield) was obtained as brown solid. HNMR (DMSO-d₆, 400 MHz): δ 8.92-8.86 (m, 1H). LC-MS: m/z 253.9 [M+H]⁺.

### Intermediate 3:

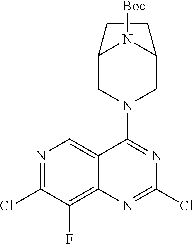 [SMILES: CC(C)(C)OC(=O)N1C2CCC1CN(c1nc(Cl)nc3c(F)c(Cl)ncc13)C2]

tert-butyl 3-(2,7-dichloro-8-fluoropyrido[4,3-d]pyrimidin-4-yl)-3,8-diazabicyclo[3.2.1]octane-8-carboxylate, 2 batches: To a mixture of compound 2 (48.5 g, 192 mmol, 1.00 eq) in DCM (485 mL) was added a solution of compound 2a (38.7 g, 183 mmol, 0.950 eq) in DCM (120 mL). Then DIEA (49.7 g, 384 mmol, 66.9 mL, 2.00 eq) was added into the mixture at −40° C. and stirred at −40° C. for 0.5 hr under N₂. LCMS showed compound 2 was consumed, and desired MS was detected. The mixture was quenched by HCl (0.5 M) and the pH of the aqueous phase was acidified to 6~7, then separated, the organic layer was dried over Na₂SO₄. The residue was purified by column chromatography (SiO₂, TLC:Petroleum ether:Ethyl acetate=3:1, R_{f}=0.4, Petroleum ether: Ethyl acetate=10:1 to 1:1, R_{f}=0.4). Compound 3 (120 g, 280 mmol, 72.9% yield) was obtained as white solid. HNMR (DMSO-d₆, 400 MHz): δ 9.13-8.98 (m, 1H), 4.67-4.36 (m, 2H), 4.35-4.21 (m, 2H), 3.87-3.50 (m, 2H), 1.85-1.71 (m, 2H), 1.66-1.56 (m, 2H), 1.46 (s, 9H). LC-MS: m/z 428.0 [M+H]⁺.

### Intermediate 4:

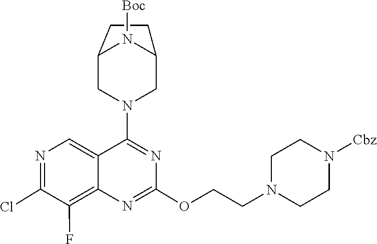 [SMILES: CC(C)(C)OC(=O)N1C2CCC1CN(c1nc(OCCN3CCN(C(=O)OCc4ccccc4)CC3)nc3c(F)c(Cl)ncc13)C2]

tert-butyl 3-(2-(2-(4-((benzyloxy) carbonyl) piperazin-1-yl) ethoxy)-7-chloro-8-fluoropyrido[4,3-d]pyrimidin-4-yl)-3,8-diazabicyclo[3.2.1]octane-8-carboxylate. To a mixture of compound 3 (10.0 g, 23.4 mmol, 1.00 eq) and compound 3a (9.26 g, 35.0 mmol, 1.50 eq) in DMAc (100 mL) was added CsF (10.6 g, 70.0 mmol, 2.58 mL, 3.00 eq) and stirred at 60° C. for 2 hrs under N₂. LCMS showed compound 3 was consumed, and desired MS was detected. The mixture was added into H₂O (200 mL) and extracted with Ethyl acetate (200 mL*2), washed with sat. NaCl (300 mL), dried over Na₂SO₄, filtered and concentrated under reduced pressure to give oil. The oil was purification by column chromatography (SiO₂, TLC:Petroleum ether:Ethyl acetate=1:1, R_{f}=0.1, Petroleum ether:Ethyl acetate=10:1 to 1:1, R_{f}=0.1). Compound 4 (7.50 g, 11.4 mmol, 49.0% yield) was obtained as white solid. HNMR (CDCl₃, 400 MHz): δ 8.74 (s, 1H), 7.30 (s, 5H), 5.13 (s, 2H), 4.62-4.58 (m, 2H), 4.49-4.45 (m, 2H), 4.41-4.29 (m, 2H), 3.71-3.60 (m, 2H), 3.54-3.50 (m, 4H), 2.87-2.83 (m, 2H), 2.60-2.53 (m, 4H), 1.99-1.93 (m, 2H), 1.74-1.69 (m, 2H), 1.53-1.51 (m, 9H). LC-MS: m/z 656.2 [M+H]⁺.

### Intermediate 5:

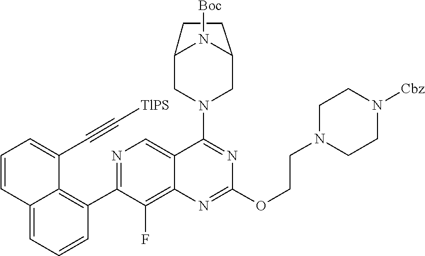

tert-butyl 3-(2-(2-(4-((benzyloxy) carbonyl) piperazin-1-yl) ethoxy)-8-fluoro-7-(8-((triisopropylsilyl)ethynyl)naphthalen-1-yl)pyrido[4,3-d]pyrimidin-4-yl)-3,8-diazabicyclo[3.2.1]octane-8-carboxylate. To a mixture of compound 4 (12.7 g, 19.4 mmol, 1.00 eq) and compound 4a (12.6 g, 29.0 mmol, 1.50 eq) in THF (127 mL) was added K₃PO₄ (1.50 M, 38.7 mL, 3.00 eq) and cataCXium A Pd G3 (2.11 g, 2.90 mmol, 0.15 eq). The mixture was stirred at 65° C. for 2 hrs under N₂. LCMS showed compound 4 was consumed, and desired MS was detected. The mixture was added into H₂O (100 mL) and extracted with Ethyl acetate (50 mL*2), washed with sat.NaCl (150 mL), dried over Na₂SO₄, filtered and concentrated under reduced pressure to give oil. The oil was purification by silica gel chromatography eluted with Petroleum ether:Ethyl acetate=100:0 to 1:1, Petroleum ether:Ethyl acetate=1:1, R_{f}=0.3. Compound 5 (10.0 g, 10.5 mmol, 54.3% yield, 97.6% purity) was obtained as brown solid. HNMR (CDCl₃, 400 MHz): δ 9.11-9.07 (m, 1H), 7.99-7.90 (m, 2H), 7.84-7.79 (m, 1H), 7.60-7.52 (m, 2H), 7.50-7.44 (m, 1H), 7.40-7.28 (m, 5H), 5.16-5.09 (m, 2H), 4.79-4.70 (m, 1H), 4.70-4.61 (m, 1H), 4.60-4.52 (m, 1H), 4.46-4.32 (m, 2H), 4.24-4.16 (m, 1H), 3.84-3.66 (m, 1H), 3.56-3.41 (m, 5H), 2.94-2.81 (m, 2H), 2.62-2.51 (m, 4H), 2.03-1.96 (m, 4H), 1.55-1.51 (m, 9H), 0.90-0.83 (m, 18H), 0.61 (s, 3H). LC-MS: m/z 928.4 [M+H]⁺.

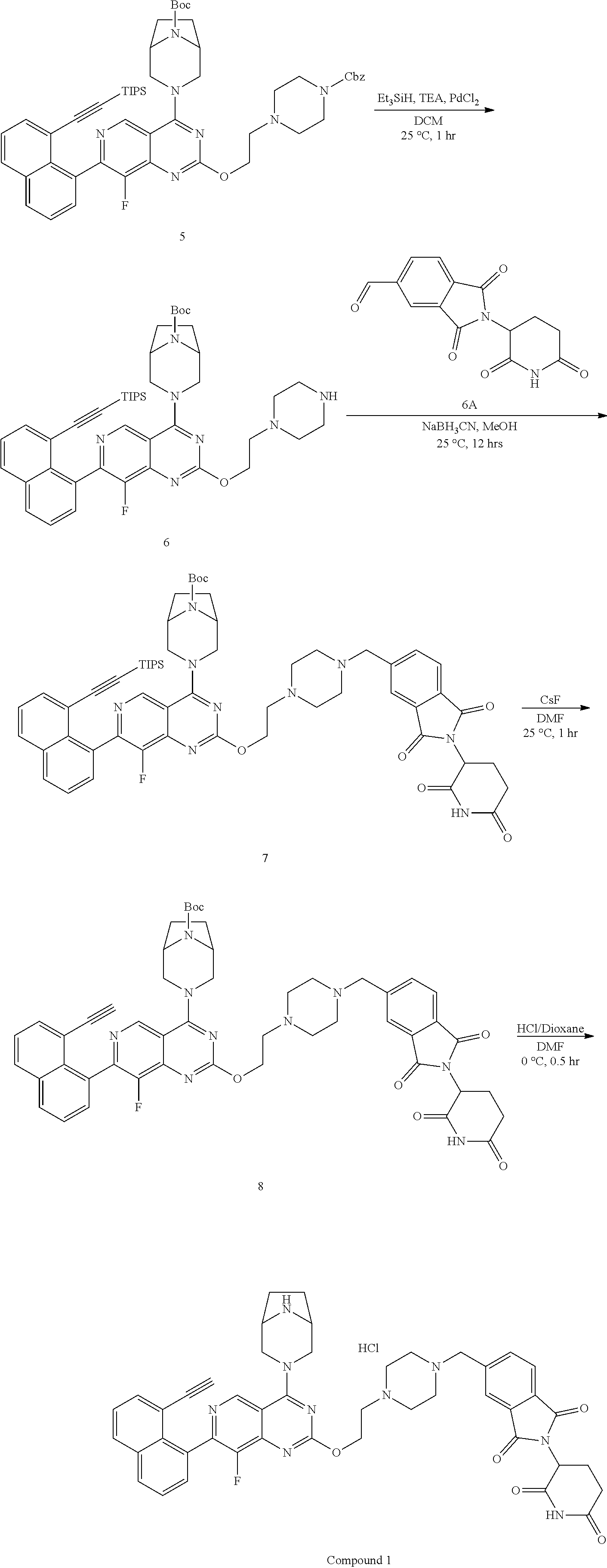

### Intermediate 6:

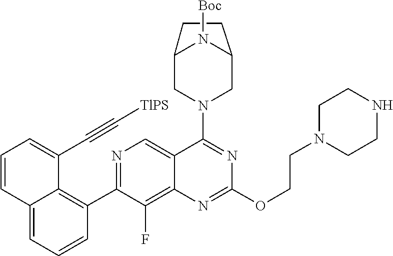

tert-butyl 3-(8-fluoro-2-(2-(piperazin-1-yl) ethoxy)-7-(8-((triisopropylsilyl)ethynyl)naphthalen-1-yl)pyrido[4,3-d]pyrimidin-4-yl)-3,8-diazabicyclo[3.2.1]octane-8-carboxylate. To a mixture of compound 5 (5.50 g, 5.86 mmol, 98.9% purity, 1.00 eq) in DCM (55.0 mL) was added TEA (4.74 g, 46.9 mmol, 6.53 mL, 8.00 eq), Et₃SiH (4.09 g, 35.2 mmol, 5.62 mL, 6.00 eq) and PdCl₂ (155 mg, 879 μmol, 0.15 eq). The mixture was stirred at 25° C. for 1 h. LCMS showed compound 5 was consumed, and desired MS was detected. The mixture was filtered. The solution was added into H₂O (50 mL) and extracted with DCM (40 mL*2), washed with sat.NaCl (100 mL), dried over Na₂SO₄, filtered and concentrated under reduced pressure to give oil. The oil was used for next step directly. Compound 6 (3.30 g, 4.16 mmol, 70.9% yield) was obtained as brown oil. LC-MS: m/z 794.4 [M+H]⁺.

### Intermediate 7:

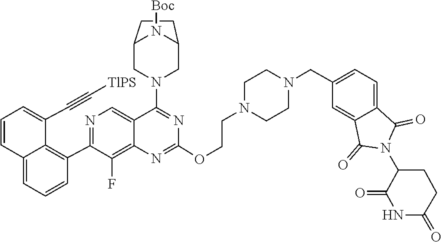

tert-butyl 3-(2-(2-(4-((2-(2,6-dioxopiperidin-3-yl)-1,3-dioxoisoindolin-5-yl)methyl) piperazin-1-yl) ethoxy)-8-fluoro-7-(8-((triisopropylsilyl)ethynyl)naphthalen-1-yl)pyrido[4,3-d]pyrimidin-4-yl)-3,8-diazabicyclo[3.2.1]octane-8-carboxylate. To a mixture of compound 6 (300 mg, 377 μmol, 1.00 eq) and compound 6A (129 mg, 453 μmol, 1.20 eq) in MeOH (3.00 mL) was added NaBH₃CN (30.8 mg, 491 μmol, 1.30 eq) and stirred at 25° C. for 12 hrs. LCMS showed compound 6 was consumed, and desired MS was detected. The mixture was added into H₂O (8 mL) and extracted with Ethyl acetate (10 mL*2), washed with sat.NaCl (10 mL), dried over Na₂SO₄, filtered and concentrated under reduced pressure to give oil. The oil was purification by reversed-phase HPLC (0.1% TFA condition) to give solid. Compound 7 (400 mg, crude) was obtained as yellow solid. LC-MS: m/z 533.0 [M/2+H]⁺.

### Intermediate 8:

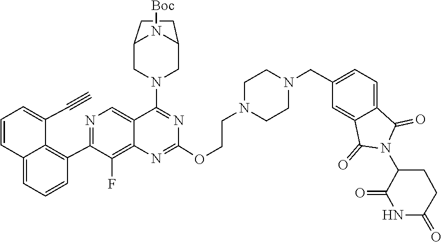 [SMILES: C#Cc1cccc2cccc(-c3ncc4c(N5CC6CCC(C5)N6C(=O)OC(C)(C)C)nc(OCCN5CCN(Cc6ccc7c(c6)C(=O)N(C6CCC(=O)NC6=O)C7=O)CC5)nc4c3F)c12]

tert-butyl 3-(2-(2-(4-((2-(2,6-dioxopiperidin-3-yl)-1,3-dioxoisoindolin-5-yl)methyl) piperazin-1-yl) ethoxy)-7-(8-ethynylnaphthalen-1-yl)-8-fluoropyrido[4,3-d]pyrimidin-4-yl)-3,8-diazabicyclo[3.2.1]octane-8-carboxylate. To a mixture of compound 7 (400 mg, 375 μmol, 1.00 eq) in DMF (4.00 mL) was added CsF (570 mg, 3.76 mmol, 138 μL, 10.0 eq) and stirred at 25° C. for 1 hr. LCMS showed compound 7 was consumed, and desired MS was detected. The mixture was added into H₂O (5 mL) and extracted with Ethyl acetate (5 mL*2), washed with sat.NaCl (5 mL), dried over Na₂SO₄, filtered and concentrated under reduced pressure to give solid. The solid was used for next step directly. Compound 8 (240 mg, 264 μmol, 70.3% yield) was obtained as yellow solid. LC-MS: m/z 908.4 [M+H]⁺.

### Compound 1:

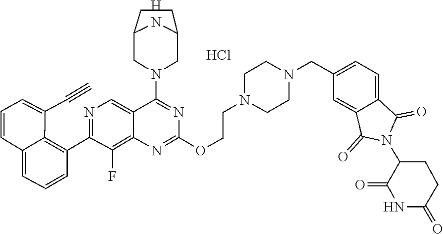

5-((4-(2-((4-(3,8-diazabicyclo[3.2.1]octan-3-yl)-7-(8-ethynylnaphthalen-1-yl)-8-fluoropyrido[4,3-d]pyrimidin-2-yl)oxy)ethyl) piperazin-1-yl)methyl)-2-(2,6-dioxopiperidin-3-yl) isoindoline-1,3-dione hydrochloride. To a mixture of compound 8 (120 mg, 132 μmol, 1.00 eq) in ACN (1.20 mL) was added HCl/dioxane (4.00 M, 500 μL, 15.1 eq) at 0° C. and stirred at 0° C. for 0.5 hrs. LCMS showed compound 8 was consumed, and desired MS was detected. The mixture was concentrated under reduced pressure at 25° C. to give oil. The oil was purification by prep-HPLC (column: Welch Xtimate C18 150*25 mm*5 μm; mobile phase: [water (FA)-ACN]; B %: 8%-38%, 10 min). Compound 9 (55.0 mg, 65.9 μmol, 49.9% yield, 96.8% purity) was obtained as yellow solid. HNMR (DMSO-d₆, 400 MHz): δ 11.15-11.07 (m, 1H), 9.07-9.02 (m, 1H), 8.17-8.10 (m, 2H), 7.89-7.85 (m, 1H), 7.84-7.81 (m, 1H), 7.81-7.77 (m, 1H), 7.73-7.66 (m, 2H), 7.60-7.53 (m, 2H), 5.18-5.09 (m, 1H), 4.51-4.41 (m, 3H), 4.35-4.28 (m, 1H), 3.72-3.54 (m, 10H), 2.90-2.83 (m, 1H), 2.70 (t, J=5.60 Hz, 2H), 2.64-2.60 (m, 1H), 2.60-2.56 (m, 1H), 2.41 (s, 5H), 2.08-2.01 (m, 1H), 1.73-1.64 (m, 4H). LC-MS: m/z 808.3 [M−HCl+H]⁺.

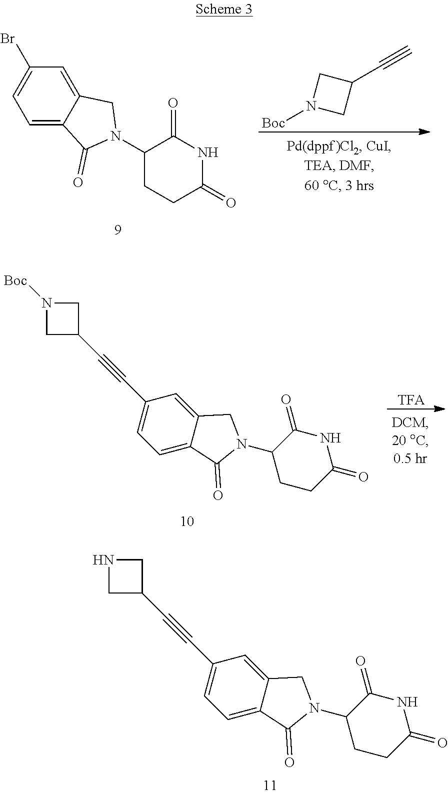 [SMILES: O=C1CCC(N2Cc3cc(Br)ccc3C2=O)C(=O)N1 ; C#CC1CN(C(=O)OC(C)(C)C)C1 ; CC(C)(C)OC(=O)N1CC(C#Cc2ccc3c(c2)CN(C2CCC(=O)NC2=O)C3=O)C1 ; "TFA" ; O=C1CCC(N2Cc3cc(C#CC4CNC4)ccc3C2=O)C(=O)N1]

### Intermediate 10:

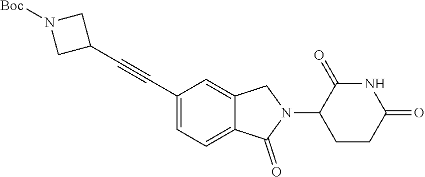 [SMILES: CC(C)(C)OC(=O)N1CC(C#Cc2ccc3c(c2)CN(C2CCC(=O)NC2=O)C3=O)C1]

tert-butyl 3-((2-(2,6-dioxopiperidin-3-yl)-1-oxoisoindolin-5-yl) ethynyl) azetidine-1-carboxylate. A mixture of compound 9 (180 mg, 557 μmol, 1.00 eq), tert-butyl 3-ethynylazetidine-1-carboxylate (100 mg, 557 μmol, 1.00 eq), Pd(dppf)Cl₂ (39.1 mg, 55.7 μmol, 0.100 eq), CuI (21.2 mg, 111 μmol, 0.200 eq) and TEA (1.35 g, 13.3 mmol, 1.86 mL, 24.0 eq) in DMF (10.0 mL) was degassed and purged with N₂ for 3 times, and then the mixture was stirred at 65° C. for 3 times under N₂ atmosphere for 3 hrs. LCMS showed desired MS was detected. The reaction mixture was quenched with NH₄Cl (aq) (300 mL) at 0° C., extracted with ethyl acetate (100 mL*3), filtrate and washed with ethyl acetate (100 mL) to give a residue. The residue was purified by column chromatography (SiO₂, petroleum ether:ethyl acetae=1:1 to 0:1, TLC:petroleum ether:ethyl acetate=0:1, P1 R_{f}=0.5) to give Intermediate 10 (430 mg, 1.02 mmol, 60.7% yield) as a gray solid. LCMS: m/z 424.2 [M+H]⁺.

### Intermediate 11:

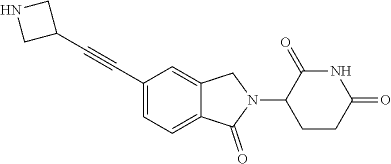 [SMILES: O=C1CCC(N2Cc3cc(C#CC4CNC4)ccc3C2=O)C(=O)N1]

3-(5-(azetidin-3-ylethynyl)-1-oxoisoindolin-2-yl) piperidine-2,6-dione. To a solution of Intermediate 10 (100 mg, 236 μmol, 1.00 eq) in DCM (1.00 mL) was added TFA (307 mg, 2.69 mmol, 0.200 mL, 11.4 eq) and stirred at 25° C. for 0.5 hr. LCMS showed desired MS was detected. The reaction mixture was concentrated to give a residue. The crude product was triturated with MTBE (4.00 mL) at 25° C. for 30 mins to give Intermediate 11 (300 mg, crude, TFA) as a yellow solid. LCMS: m/z 324.1 [M+H]⁺.

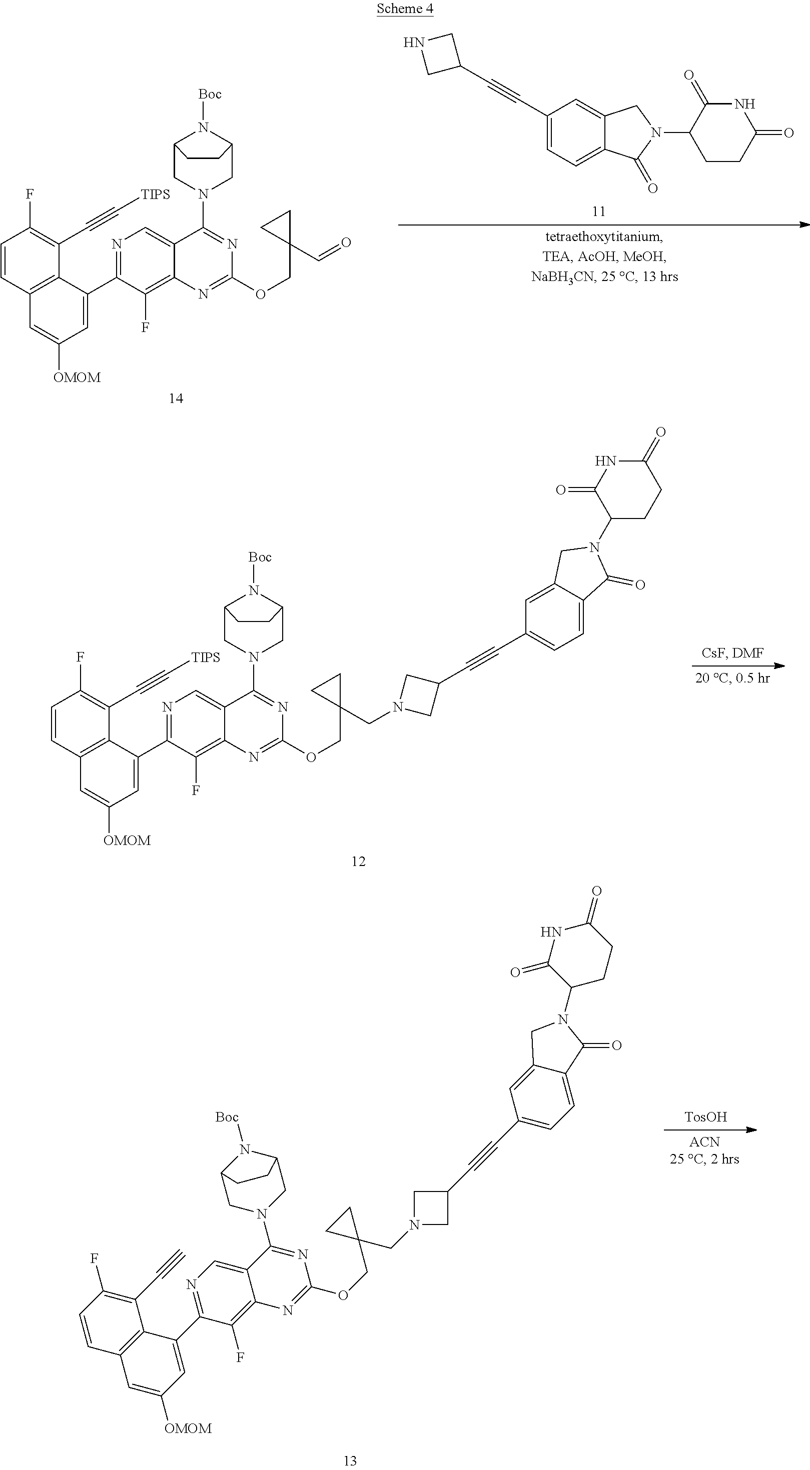

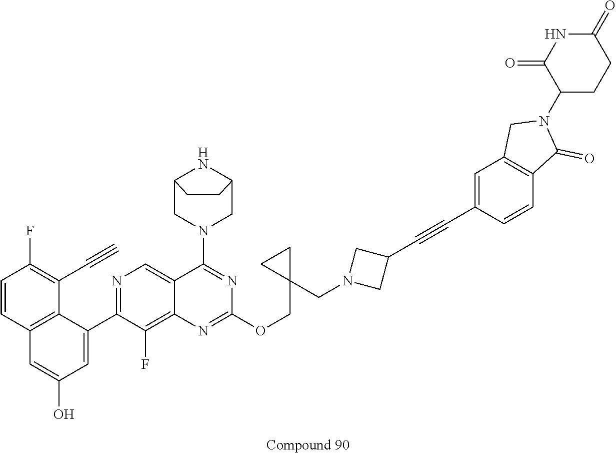 [SMILES: C#Cc1c(F)ccc2cc(O)cc(-c3ncc4c(N5CC6CCC(C5)N6)nc(OCC5(CN6CC(C#Cc7ccc8c(c7)CN(C7CCC(=O)NC7=O)C8=O)C6)CC5)nc4c3F)c12]

### Intermediate 12:

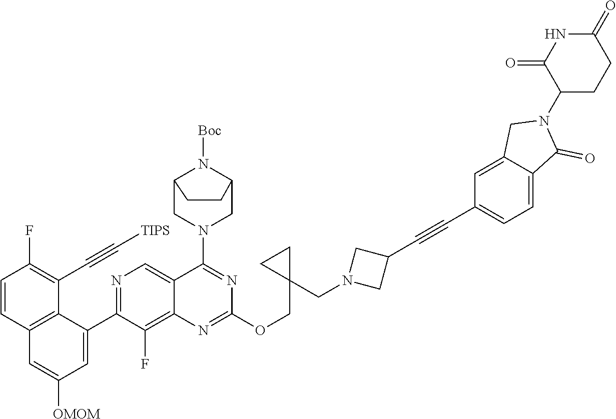

tert-butyl (1R,5S)-3-(2-((1-((3-((2-(2,6-dioxopiperidin-3-yl)-1-oxoisoindolin-5-yl) ethynyl) azetidin-1-yl)methyl)cyclopropyl)methoxy)-8-fluoro-7-(7-fluoro-3-(methoxymethoxy)-8-((triisopropylsilyl)ethynyl)naphthalen-1-yl)pyrido[4,3-d]pyrimidin-4-yl)-3,8-diazabicyclo[3.2.1]octane-8-carboxylate. To a solution of compound 14 (30. 0 mg, 68.5 μmol, 1.16 eq, TFA) in MeOH (1.00 mL) was added TEA (36.3 mg, 359 μmol, 50.0 μL, 6.05 eq), CH₃COOH (3.57 mg, 59.3 μmol, 3.40 μL, 1.00 eq), Ti(OEt)₄ (81.2 mg, 356 μmol, 73.8 μL, 6.00 eq) and stirred for 1 hr, then added Intermediate 11 (50.0 mg, 59.3 μmol, 1.00 eq) and TEA (36.3 mg, 359 μmol, 50.0 μL, 6.05 eq) and stirred 1 hr, then NaBH₃CN (11.1 mg, 178 μmol, 3.00 eq) was added into the reaction mixture and stirred at 20° C. for 12 hrs. LCMS showed desired MS was detected. The reaction mixture was concentrated reduced pressure and poured into water (10.0 mL), extracted with ethyl acetae (5.00 ml*3), dried over Na₂SO₄, filtrate was concentrated under reduced pressure to give a residue. The residue was purified by column chromatography (SiO₂, MeOH:DCM=1:30 to 1:15, TLC:MeOH:DCM=10:1, P1 R_{f}=0.3) to give Intermediate 12: (102 mg, 88.7 μmol, 37.3% yield) as a yellow solid. LCMS: m/z 1150.3 [M+H]⁺.

### Intermediate 13:

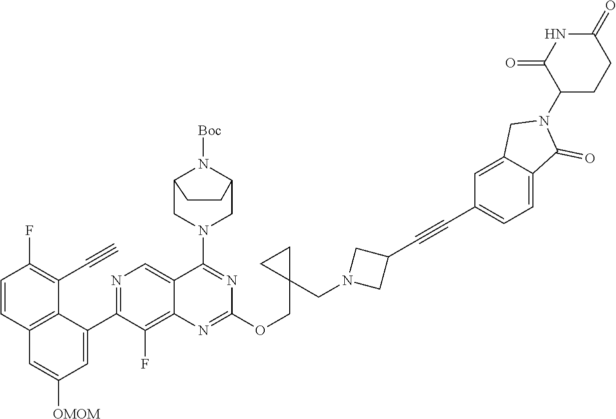

tert-butyl (1R,5S)-3-(2-((1-((3-((2-(2,6-dioxopiperidin-3-yl)-1-oxoisoindolin-5-yl) ethynyl) azetidin-1-yl)methyl)cyclopropyl)methoxy)-7-(8-ethynyl-7-fluoro-3-(methoxymethoxy)naphthalen-1-yl)-8-fluoropyrido[4,3-d]pyrimidin-4-yl)-3,8-diazabicyclo[3.2.1]octane-8-carboxylate. To a solution of Intermediate 12 (100 mg, 87.0 μmol, 1.00 eq) in DMF (1.00 mL) was added CsF (132 mg, 870 μmol, 32.1 μL, 10.0 eq), and stirred at 25° C. for 0.5 hr. LCMS showed desired MS was detected. The reaction mixture was concentrated reduced pressure and poured into water (20.0 mL), extracted with ethyl acetate (5.00 mL*3), dried over Na₂SO₄, filtrate was concentrated under reduced pressure to give Intermediate 13 (102 mg, crude) as a yellow solid. LCMS: m/z 994.0 [M+H]⁺.

### Compound 90:

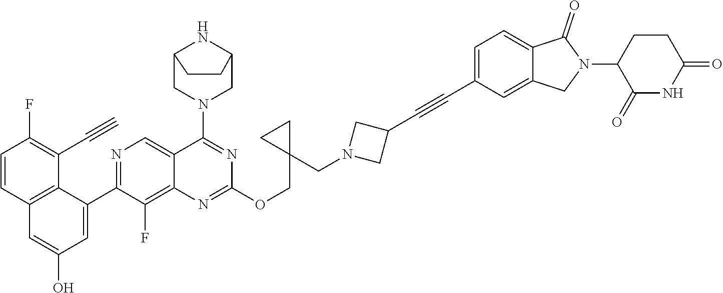 [SMILES: C#Cc1c(F)ccc2cc(O)cc(-c3ncc4c(N5CC6CCC(C5)N6)nc(OCC5(CN6CC(C#Cc7ccc8c(c7)CN(C7CCC(=O)NC7=O)C8=O)C6)CC5)nc4c3F)c12]

3-(5-((1-((1-(((4-((1R,5S)-3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)methyl)cyclopropyl)methyl) azetidin-3-yl) ethynyl)-1-oxoisoindolin-2-yl) piperidine-2,6-dione formate. To a solution of Intermediate 13 (25.0 mg, 25.1 μmol, 1.00 eq) in ACN (1 mL) was added TosOH (69.3 mg, 402 μmol, 16.0 eq), and stirred at 25° C. for 2 hrs. LCMS showed desired MS was detected. The reaction mixture was concentrated reduced pressure and poured into NaHCO₃(aq) (2.00 mL), extracted with THF (2.00 mL*3), ethyl acetae (3.00 mL*3), dried over Na₂SO₄, filtrate was concentrated under reduced pressure to give a residue. The residue was purified by prep-HPLC (column: Phenomenex luna C18 150*25 mm*10 μm; mobile phase: [water (FA)-ACN]; B %: 3%-33%, 2 min) to give compound 90 (20.5 mg, 21.5 μmol, 28.5% yield, 93.8% purity, FA) as a yellow solid. ¹H NMR (400 MHz, DMSO-d₆) δ 1.00 (s, 1H), 9.06-8.99 (m, 1H), 8.20 (s, 1H), 8.02-7.96 (m, 1H), 7.62 (s, 2H), 7.54-7.44 (m, 2H), 7.41-7.38 (m, 1H), 7.18 (d, J=2.4 Hz, 1H), 5.07 (s, 1H), 4.42 (s, 2H), 4.35-4.28 (m, 2H), 4.23-4.19 (m, 2H), 3.95 (s, 1H), 3.66-3.62 (m, 3H), 3.58 (s, 4H), 3.13-3.05 (m, 4H), 2.62-2.56 (m, 1H), 2.46-2.44 (m, 2H), 2.41-2.37 (m, 1H), 1.68 (s, 4H), 0.59-0.52 (m, 2H), 0.49-0.45 (m, 2H). LCMS: m/z 849.8 [M−HCOOH+H]⁺.

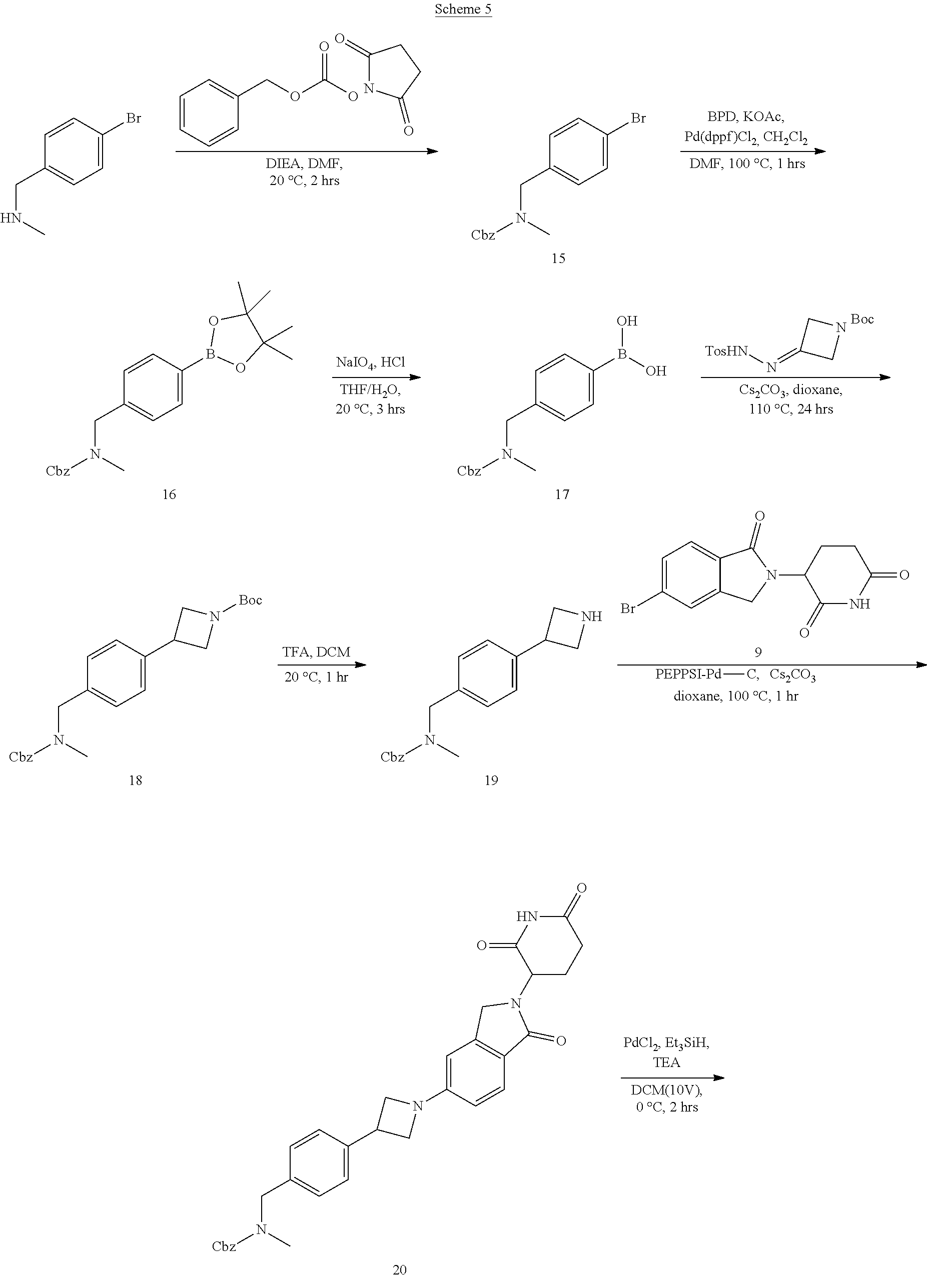

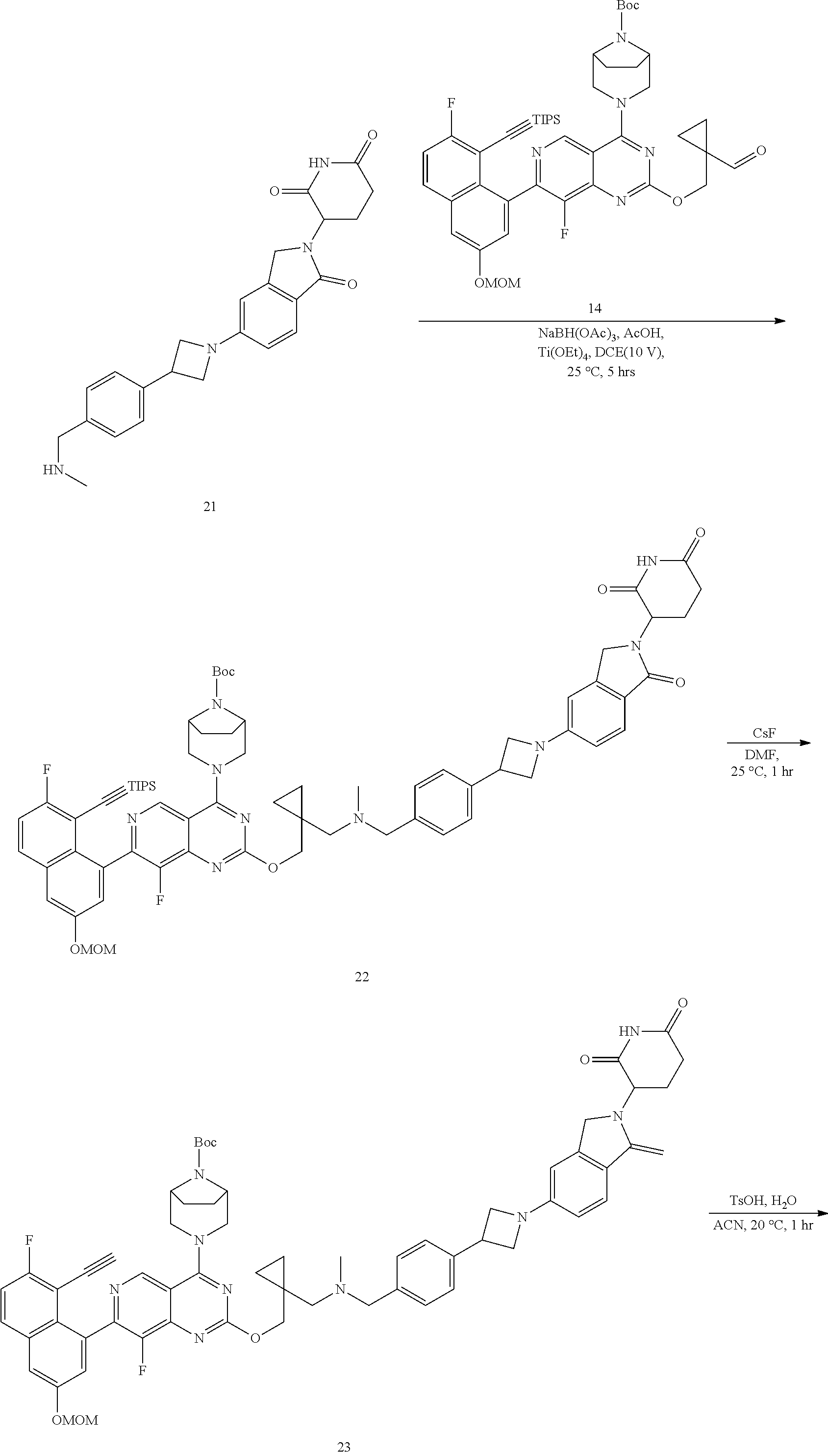

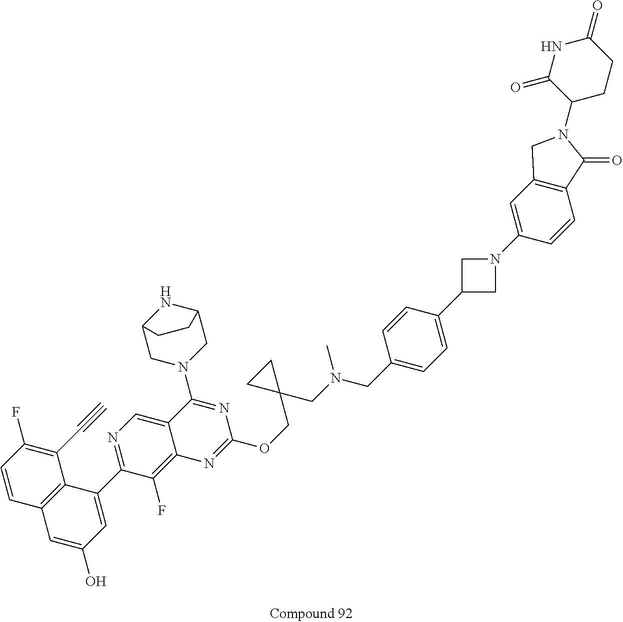 [SMILES: C#Cc1c(F)ccc2cc(O)cc(-c3ncc4c(N5CC6CCC(C5)N6)nc(OCC5(CN(C)Cc6ccc(C7CN(c8ccc9c(c8)CN(C8CCC(=O)NC8=O)C9=O)C7)cc6)CC5)nc4c3F)c12]

### Intermediate 15:

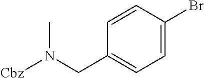 [SMILES: CN(Cc1ccc(Br)cc1)C(=O)OCc1ccccc1]

benzyl (4-bromobenzyl)(methyl) carbamate. To a solution of 1-(4-bromophenyl)-N-methylmethanamine (3.00 g, 15.0 mmol, 3.00 mL, 1.00 eq) in DMF (30.0 mL) was added DIEA (7.75 g, 56.0 mmol, 10.5 mL, 4.00 eq) and benzyl (2,5-dioxopyrrolidin-1-yl) carbonate (4.11 g, 16.5 mmol, 1.10 eq). The mixture was stirred at 20° C. for 2 hrs. LCMS showed desired MS was detected. The reaction mixture was diluted with ethyl acetate (300 mL), and then extracted with H₂O (100 mL*3). The organic layer was dried over Na₂SO₄, filtered and concentrated to give a residue. The residue was purified by column chromatography (SiO₂, Petroleum ether:Ethyl acetate=50:1 to 3:1, TLC:Petroleum ether:Ethyl acetate=5:1, P1, R_{f}=0.5) to give Intermediate 15 (4.85 g, 14.5 mmol, 96.8% yield) as a colorless liquid. LCMS: m/z 336.2 [M+H]⁺.

### Intermediate 16:

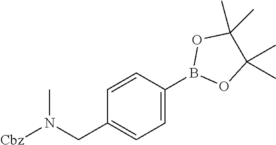 [SMILES: CN(Cc1ccc(B2OC(C)(C)C(C)(C)O2)cc1)C(=O)OCc1ccccc1]

benzyl methyl (4-(4,4,5,5-tetramethyl-1,3,2-dioxaborolan-2-yl)benzyl) carbamate. A mixture of Intermediate 15 (2.00 g, 5.98 mmol, 1.00 eq), BPD (1.82 g, 7.18 mmol, 1.20 eq), KOAc (1.76 g, 18.0 mmol, 3.00 eq) and Pd(dppf)Cl₂·CH₂Cl₂ (489 mg, 598 μmol, 0.10 eq) in DMF (40.0 mL) was degassed and purged with N₂ for 3 times, and then the mixture was stirred at 100° C. for 1 hr under N₂ atmosphere. LCMS showed desired MS was detected. The reaction mixture was poured into water (100 mL), extracted with DCM (100 mL*3), the organic layer was washed with brine (200 mL*3), dried over Na₂SO₄, filtered and concentrated to give a residue. The residue was purified by column chromatography (SiO₂, petroleum ether:ethyl acetate=1:0 to 5:1, TLC:petroleum ether:ethyl acetate=5:1, P1, R_{f}=0.5) to give Intermediate 16 (2.50 g, crude) as a yellow liquid. LCMS: m/z 382.3 [M+H]⁺.

### Intermediate 17:

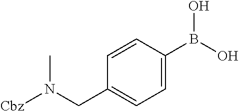 [SMILES: CN(Cc1ccc(B(O)O)cc1)C(=O)OCc1ccccc1]

(4-((((benzyloxy) carbonyl)(methyl)amino)methyl)phenyl)boronic acid. To a solution of Intermediate 16 (3.00 g, 7.87 mmol, 1.00 eq) in THF (60.0 mL) and H₂O (15.0 mL) was added NaIO₄ (5.05 g, 23.6 mmol, 1.31 mL, 3.00 eq). The mixture was stirred at 20° C. for 1 hr. Then the mixture was added HCl (1 M, 5.51 mL, 0.700 eq) and stirred at 20° C. for 2 hrs. LCMS showed desired MS was detected. The reaction mixture was quenched with Na₂SO₃ (20.0 mL), extracted with ethyl acetate (50.0 mL*3). The organic layer was dried over Na₂SO₄, filtered and concentrated to give a residue. The residue was purified by column chromatography (SiO₂, petroleum ether:ethyl acetate=50:1 to 2:1, TLC:petroleum ether:ethyl acetate=1:1, P1 R_{f}=0.2) to give Intermediate 17 (2.30 g, 7.69 mmol, 97.7% yield) as a yellow oil. LCMS: m/z 300.2 [M+H]⁺.

### Intermediate 18:

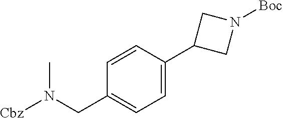 [SMILES: CN(Cc1ccc(C2CN(C(=O)OC(C)(C)C)C2)cc1)C(=O)OCc1ccccc1]

tert-butyl 3-(4-((((benzyloxy) carbonyl)(methyl)amino)methyl)phenyl) azetidine-1-carboxylate. A mixture of Intermediate 17 (1.80 g, 6.02 mmol, 1.00 eq), compound 4-1 (6.13 g, 18.1 mmol, 3.00 eq), Cs₂CO₃ (11.8 g, 36.1 mmol, 6.00 eq) in dioxane (30.0 mL) was degassed and purged with N₂ for 3 times, and then the mixture was stirred at 110° C. for 24 hrs under N₂. LCMS showed desired MS was detected. The reaction mixture was poured into water (50.0 mL), extracted with ethyl acetate (50.0 mL*3), the organic layer was dried over Na₂SO₄, filtered and concentrated to give a residue. The residue was purified by column chromatography (SiO₂, petroleum ether:ethyl acetate=1:0 to 15:1, TLC:petroleum ether:ethyl acetate=5:1, P1, R_{f}=0.2) to give Intermediate 18 (621 mg, 1.51 mmol, 25.1% yield) as a yellow oil.LCMS: m/z 433.4 [M+Na]⁺.

### Intermediate 19:

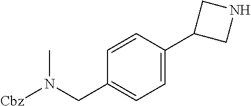 [SMILES: CN(Cc1ccc(C2CNC2)cc1)C(=O)OCc1ccccc1]

benzyl (4-(azetidin-3-yl)benzyl)(methyl) carbamate. To a solution of Intermediate 18 (300 mg, 731 μmol, 1.00 eq) in DCM (3.00 mL) and TFA (1.54 g, 13.5 mmol, 1.00 mL, 18.4 eq). The mixture was stirred at 20° C. for 1 hr. LCMS showed desired MS was detected. The reaction mixture was concentrated to give a residue. The residue was slowly dripped into ice saturated NaHCO₃ (5.00 mL), extracted with ethyl acetate (10.0 mL*3), the organic layer was dried over Na₂SO₄, filtered and concentrated to give Intermediate 19 (300 mg, crude) as a brown oil. LCMS: m/z 311.4 [M+H]⁺.

### Intermediate 20:

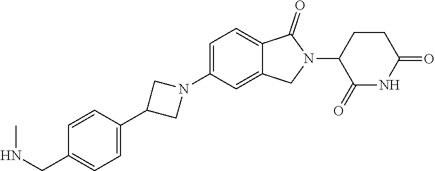 [SMILES: CNCc1ccc(C2CN(c3ccc4c(c3)CN(C3CCC(=O)NC3=O)C4=O)C2)cc1]

3-(5-(3-(4-((methylamino)methyl)phenyl) azetidin-1-yl)-1-oxoisoindolin-2-yl) piperidine-2,6-dione. To a solution of compound 9 (250 mg, 774 μmol, 1.00 eq) and Intermediate 19 (360 mg, 1.16 mmol, 1.50 eq) in dioxane (9.00 mL) was added Cs₂CO₃ (756 mg, 2.32 mmol, 3.00 eq) and PEPPSI—Pd—C(37.6 mg, 38.7 μmol, 0.0500 eq). The mixture was stirred at 100° C. for 1 hr under N₂. LCMS showed desired MS was detected. The reaction mixture was filtered and the filtered liquid was quenched by addition NH₄Cl to pH=7 at 0° C., the reaction mixture was diluted with H₂O (20.0 mL) and extracted with ethyl acetate (20.0 mL*3). The combined organic layers were washed with aqueous NaCl (20.0 mL*2), dried over Na₂SO₄, filtered and concentrated under reduced pressure to give a residue. The residue was purified by column chromatography (SiO₂, petroleum ether:ethyl acetate=10:1 to Dichloromethane:Methanol=15:1, TLC:Dichloromethane:Methanol=10:1, P1 R_{f}=0.3) to give Intermediate 20 (350 mg, 633 μmol, 81.7% yield) as a red-brown solid. LCMS: m/z 553.6 [M+H]⁺.

### Intermediate 21:

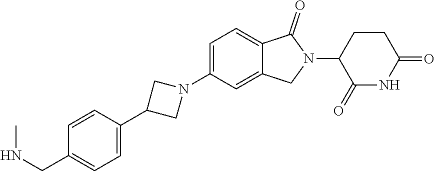 [SMILES: CNCc1ccc(C2CN(c3ccc4c(c3)CN(C3CCC(=O)NC3=O)C4=O)C2)cc1]

3-(5-(3-(4-((methylamino)methyl)phenyl) azetidin-1-yl)-1-oxoisoindolin-2-yl) piperidine-2,6-dione. To a solution of Intermediate 20 (100 mg, 181 μmol, 1.00 eq) in DCM (2.00 mL) was added TEA (183 mg, 1.81 mmol, 252 μL, 10.0 eq), PdCl₂ (4.81 mg, 27.1 μmol, 0.150 eq) and Et₃SiH (168 mg, 1.45 mmol, 231 μL, 8.00 eq), then stirred at 20° C. for 2 hrs. LCMS showed desired MS was detected. The reaction mixture was filtered, and the mother liquor concentrated under reduced pressure to give a residue. The residue was purified by prep-HPLC (FA condition) to give Intermediate 21 (40.0 mg, 95.6 μmol, 52.8% yield) as a white solid. LCMS: m/z 419.4 [M+H]⁺.

### Intermediate 22:

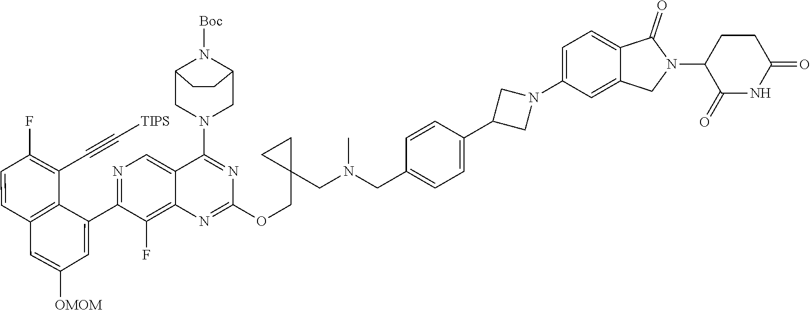

tert-butyl (1R,5S)-3-(2-((1-(((4-(1-(2-(2,6-dioxopiperidin-3-yl)-1-oxoisoindolin-5-yl) azetidin-3-yl)benzyl)(methyl)amino)methyl)cyclopropyl)methoxy)-8-fluoro-7-(7-fluoro-3-(methoxymethoxy)-8-((triisopropylsilyl)ethynyl)naphthalen-1-yl)pyrido[4,3-d]pyrimidin-4-yl)-3,8-diazabicyclo[3.2.1]octane-8-carboxylate. To a solution of Intermediate 21 (25.0 mg, 59.7 μmol, 1.00 eq) in DCE (2.00 mL) was added TEA (6.04 mg, 59.7 μmol, 8.31 μL, 1.00 eq) at 20° C. and stirred 0.1 hr. Then the compound 14 (55.3 mg, 65.7 μmol, 1.10 eq), Ti(OEt)₄ (81.8 mg, 358 μmol, 74.3 μL, 6.00 eq) and AcOH (437 mg, 7.28 mmol, 417 μL, 122 eq) was added and the mixture was stirred at 20° C. for 1 hr, then NaBH(OAc) 3 (38.0 mg, 179 μmol, 3.00 eq) was added into the reaction mixture and stirred at 20° C. for 4 hrs. LCMS showed desired MS was detected. The reaction mixture was poured into water (20.0 mL), extracted with ethyl acetate (20.0 mL*3), the organic layer was dried over Na₂SO₄, filtered and concentrated to give a residue. The residue was purified by column chromatography (SiO₂, petroleum ether:ethyl acetate=10:1 to Dichloromethane:Methanol=10:1, TLC:Dichloromethane:Methanol=10:1, P1, R_{f}=0.4) to give Intermediate 22 (58.0 mg, 46.6 μmol, 78.0% yield) as a yellow solid. LCMS: m/z 1245.6 [M+H]⁺.

### Intermediate 23:

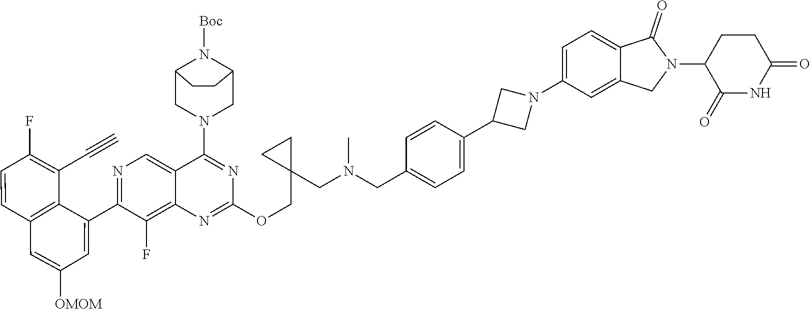

tert-butyl (1R,5S)-3-(2-((1-(((4-(1-(2-(2,6-dioxopiperidin-3-yl)-1-oxoisoindolin-5-yl) azetidin-3-yl)benzyl)(methyl)amino)methyl)cyclopropyl)methoxy)-7-(8-ethynyl-7-fluoro-3-(methoxymethoxy)naphthalen-1-yl)-8-fluoropyrido[4,3-d]pyrimidin-4-yl)-3,8-diazabicyclo[3.2.1]octane-8-carboxylate. To a solution of Intermediate 22 (58.0 mg, 46.6 μmol, 1.00 eq) in DMF (1.00 mL) was added CsF (70.8 mg, 466 μmol, 17.2 μL, 10.0 eq). The mixture was stirred at 25° C. for 1 hr. LCMS showed desired MS was detected. The reaction mixture was poured into water (10 mL), extracted with DCM (10 mL*3), the organic layer was washed with brine (10 mL*3), dried over Na₂SO₄, filtered and concentrated to give Intermediate 23 (56.0 mg, crude) as a brown oil. LCMS: m/z 1088.7 [M+H]⁺.

### Compound 92:

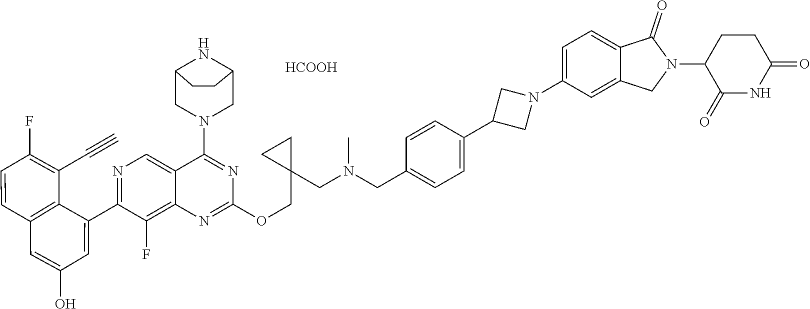 [SMILES: C#Cc1c(F)ccc2cc(O)cc(-c3ncc4c(N5CC6CCC(C5)N6)nc(OCC5(CN(C)Cc6ccc(C7CN(c8ccc9c(c8)CN(C8CCC(=O)NC8=O)C9=O)C7)cc6)CC5)nc4c3F)c12 ; "HCOOH"]

3-(5-(3-(4-((((1-(((4-((1R,5S)-3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)methyl)cyclopropyl)methyl)(methyl)amino)methyl)phenyl) azetidin-1-yl)-1-oxoisoindolin-2-yl) piperidine-2,6-dione formate. To a solution of Intermediate 23 (50.0 mg, 46.0 μmol, 1.00 eq) in ACN (1.00 mL) was added TsOH·H₂O (87.4 mg, 456 μmol, 10.0 eq). The mixture was stirred at 20° C. for 1 hr. LCMS showed desired MS was detected. The residue was slowly dripped into ice saturated NaHCO₃ (5.00 mL), extracted with ethyl acetate (10 mL*3), the organic layer was dried over Na₂SO₄, filtered and concentrated to give a residue. The residue was purified by prep-HPLC (column: Phenomenex luna C18 150*25 mm*10 μm; mobile phase: [water (FA)-ACN]; gradient: 70%-37% B over 8 min) to give compound 92 (9.00 mg, 9.00 μmol, 19.6% yield, 99% purity, FA) as a yellow solid. ¹H NMR (400 MHz, DMSO-d₆) δ 10.93 (s, 1H), 10.24-10.13 (m, 1H), 9.03 (s, 1H), 8.18 (s, 1H), 7.99 (dd, J=5.8, 9.2 Hz, 1H), 7.51-7.39 (m, 3H), 7.26-7.13 (m, 5H), 6.53-6.44 (m, 2H), 5.05 (dd, J=4.8, 13.4 Hz, 1H), 4.48-4.38 (m, 2H), 4.36-4.25 (m, 5H), 4.20-4.12 (m, 1H), 3.96-3.88 (m, 2H), 3.84-3.75 (m, 2H), 3.66-3.42 (m, 7H), 2.98-2.84 (m, 1H), 2.63-2.59 (m, 1H), 2.44-2.35 (m, 2H), 2.30-2.25 (m, 1H), 2.19 (s, 3H), 2.01-1.90 (m, 1H), 1.72-1.61 (m, 4H), 0.65 (s, 2H), 0.43 (s, 2H). LCMS: m/z 944.7 [M−HCOOH+H]⁺.

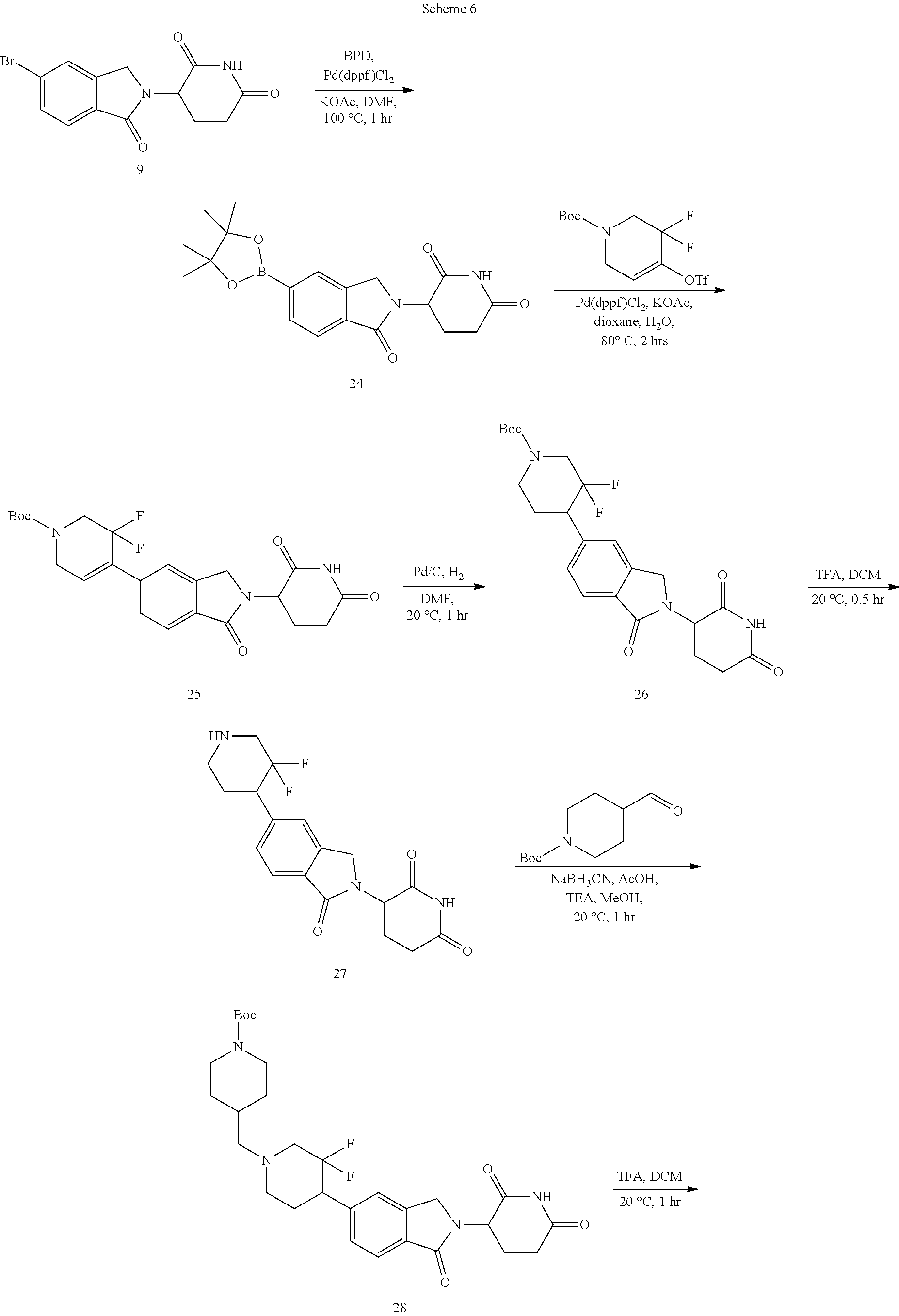

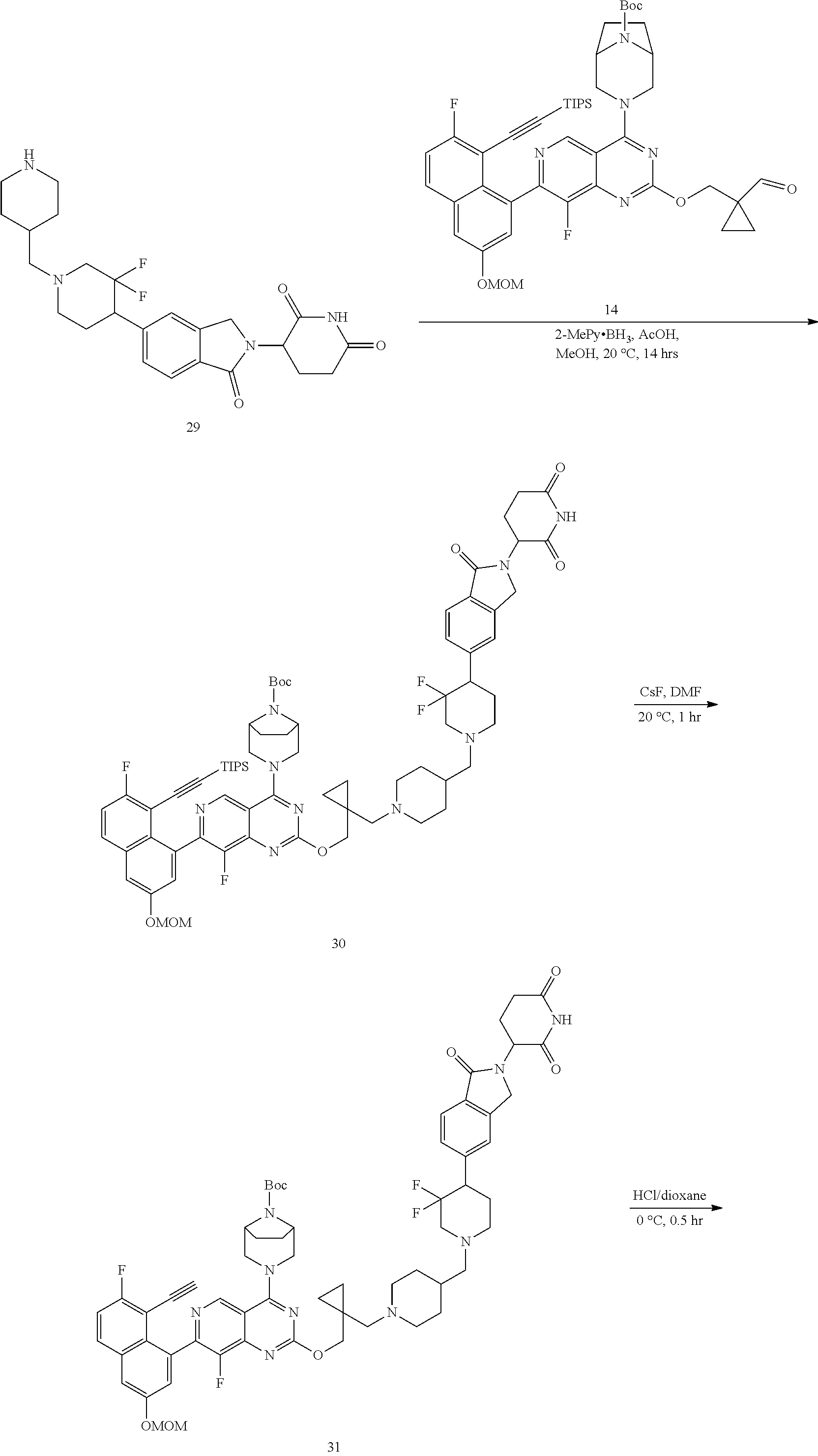

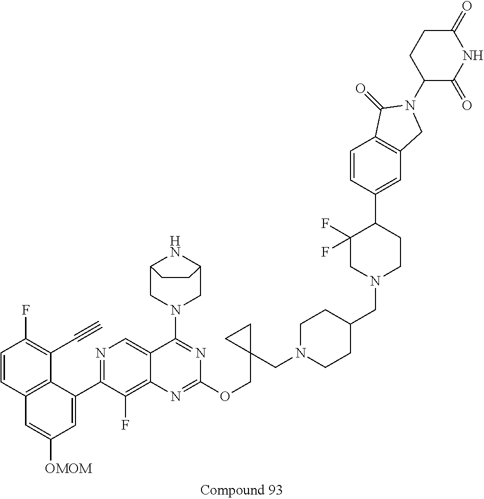

### Intermediate 24:

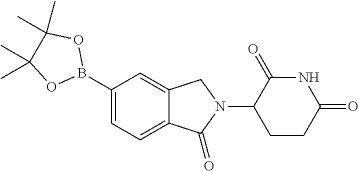 [SMILES: CC1(C)OB(c2ccc3c(c2)CN(C2CCC(=O)NC2=O)C3=O)OC1(C)C]

3-(1-oxo-5-(4,4,5,5-tetramethyl-1,3,2-dioxaborolan-2-yl) isoindolin-2-yl) piperidine-2,6-dione. A mixture of compound 9 (2.00 g, 6.19 mmol, 1.00 eq), BPD (1.89 g, 7.43 mmol, 1.20 eq), KOAc (1.82 g, 18.5 mmol, 3.00 eq) and Pd(dppf) C₁₂·CH₂Cl₂ (505 mg, 618 μmol, 0.100 eq) in DMF (40.0 mL) was degassed and purged with N₂ for 3 times, and then the mixture was stirred at 100° C. for 1 hr under N₂ atmosphere. LCMS showed 55% of desired compound was detected. The reaction mixture was diluted with H₂O 20.0 mL and extracted with Ethyl acetate 30.0 mL (10.0 mL*3), but the product was separated out. Then the mixture was filtered to produce the product. Intermediate 24 (1.60 g, crude) was obtained as a gray solid. LCMS: m/z 371.2 [M+H]⁺

### Intermediate 25:

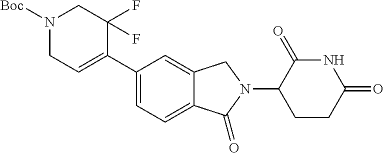 [SMILES: CC(C)(C)OC(=O)N1CC=C(c2ccc3c(c2)CN(C2CCC(=O)NC2=O)C3=O)C(F)(F)C1]

tert-butyl 4-(2-(2,6-dioxopiperidin-3-yl)-1-oxoisoindolin-5-yl)-3,3-difluoro-3,6-dihydropyridine-1 (2H)-carboxylate. A mixture of Intermediate 24 (733 mg, 1.98 mmol, 1.00 eq), tert-butyl 3,3-difluoro-4-(((trifluoromethyl) sulfonyl)oxy)-3,6-dihydropyridine-1 (2H)-carboxylate (800 mg, 2.18 mmol, 1.1 eq), Pd(dppf)Cl₂ (144 mg, 198 μmol, 0.100 eq), K₃PO₄ (840 mg, 3.96 mmol, 2.00 eq) and in dioxane (2.00 mL) was degassed and purged with N₂ for 3 times, and then the mixture was stirred at 80° C. for 2 hrs under N₂ atmosphere. LCMS showed 62% of desired compound was detected. The reaction mixture was diluted with H₂O 20.0 mL and extracted with Ethyl acetate 30.0 mL (10.0 mL*3). The combined organic layers were washed with aqueous NaCl 20.0 mL (10.0 mL*2), dried over Na₂SO₄, filtered and concentrated under reduced pressure to give a residue. The residue was purified by column chromatography (SiO₂, Dichloromethane:Methanol=1:0 to 50:1, TLC:Dichloromethane:Methanol=10:1 P1: R_{f}=0.60). Intermediate 25 (800 mg, 1.73 mmol, 87.5% yield) was obtained as a yellow solid. LCMS: m/z 462.3 [M+H]⁺.

### Intermediate 26:

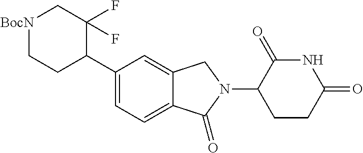 [SMILES: CC(C)(C)OC(=O)N1CCC(c2ccc3c(c2)CN(C2CCC(=O)NC2=O)C3=O)C(F)(F)C1]

tert-butyl 4-(2-(2,6-dioxopiperidin-3-yl)-1-oxoisoindolin-5-yl)-3,3-difluoropiperidine-1-carboxylate. To a solution of Intermediate 25 (400 mg, 866 μmol, 1.00 eq) in DMF (5.00 mL) was added Pd/C (400 mg, 10% purity) and H₂ (15 psi). The mixture was stirred at 20° C. for 1 hr. LCMS showed 98% of desired compound was detected. The reaction mixture was filtered and the filter liquor was diluted with H₂O 20.0 mL and extracted with Ethyl acetate 30.0 mL (10.0 mL*3). The combined organic layers were washed with aqueous NaCl 20.0 mL (10.0 mL*2), dried over Na₂SO₄, filtered and concentrated under reduced pressure to give a residue. Intermediate 26 (400 mg, crude) was obtained as a light yellow solid. LCMS: m/z 464.3 [M+H]⁺.

### Intermediate 27:

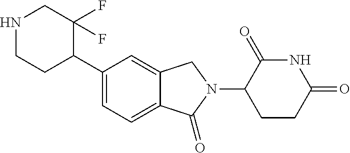 [SMILES: O=C1CCC(N2Cc3cc(C4CCNCC4(F)F)ccc3C2=O)C(=O)N1]

3-(5-(3,3-difluoropiperidin-4-yl)-1-oxoisoindolin-2-yl) piperidine-2,6-dione. To a solution of Intermediate 26 (400 mg, 863.05 μmol, 1.00 eq) in ACN (0.500 mL) was added HCl/dioxane (4 M, 10.0 mL, 46.3 eq). The mixture was stirred at 20° C. for 0.5 hr. LCMS showed desired compound was detected. The reaction mixture was concentrated under reduced pressure to give a residue. Intermediate 27 (400 mg, crude, HCl) was obtained as a white solid. LCMS: m/z 364.2 [M+H]⁺.

### Intermediate 28:

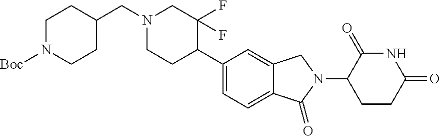 [SMILES: CC(C)(C)OC(=O)N1CCC(CN2CCC(c3ccc4c(c3)CN(C3CCC(=O)NC3=O)C4=O)C(F)(F)C2)CC1]

tert-butyl 4-((4-(2-(2,6-dioxopiperidin-3-yl)-1-oxoisoindolin-5-yl)-3,3-difluoropiperidin-1-yl)methyl) piperidine-1-carboxylate. To a solution of Intermediate 27 (200 mg, 500 μmol, 1.00 eq, HCl) and tert-butyl 4-formylpiperidine-1-carboxylate (128 mg, 600 μmol, 1.20 eq) in MeOH (2.00 mL) was added and AcOH (3.00 mg, 50 μmol, 2.86 μL, 0.100 eq). The mixture was stirred at 20° C. for 0.5 hr. Then the mixture was added NaBH₃CN (62.8 mg, 1.00 mmol, 2.00 eq) at 20° C. for 0.5 hr. LCMS showed 91% of desired compound was detected. The reaction mixture was diluted with H₂O 20.0 mL and extracted with Ethyl acetate 30.0 mL (10.0 mL*3). The combined organic layers were washed with aqueous NaCl 20.0 mL (10.0 mL*2), dried over Na₂SO₄, filtered and concentrated under reduced pressure to give a residue. The residue was purified by column chromatography (SiO₂, Petroleum ether:Ethyl acetate=1:0 to 1:1, TLC:Petroleum ether:Ethyl acetate=3:1 P1: R_{f}=0.46). Intermediate 28 (400 mg, 713 μmol, 71.3% yield) was obtained as a yellow solid. LCMS: m/z 583.5 [M+Na]⁺.

### Intermediate 29:

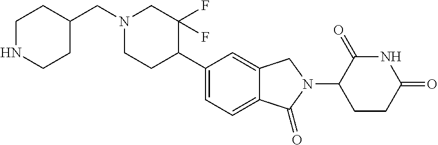 [SMILES: O=C1CCC(N2Cc3cc(C4CCN(CC5CCNCC5)CC4(F)F)ccc3C2=O)C(=O)N1]

3-(5-(3,3-difluoro-1-(piperidin-4-ylmethyl) piperidin-4-yl)-1-oxoisoindolin-2-yl) piperidine-2,6-dione. To a solution of Intermediate 28 (400 mg, 713 μmol, 1.00 eq) in ACN (2.00 mL) was added HCl/dioxane (4 M, 2.00 mL, 11.2 eq). The mixture was stirred at 20° C. for 1 hr. LCMS showed 93% of desired compound was detected. The reaction mixture was concentrated under reduced pressure to give a residue. Intermediate 29 (350 mg, crude, HCl) was obtained as colorless gum. LCMS: m/z 461.4 [M+H]⁺.

### Intermediate 30:

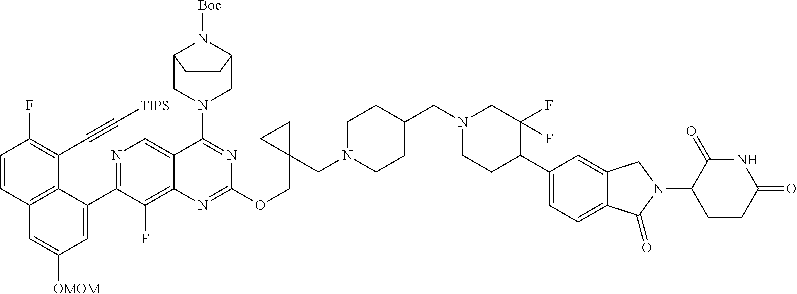

tert-butyl (1R,5S)-3-(2-((1-((4-((4-(2-(2,6-dioxopiperidin-3-yl)-1-oxoisoindolin-5-yl)-3,3-difluoropiperidin-1-yl)methyl) piperidin-1-yl)methyl)cyclopropyl)methoxy)-8-fluoro-7-(7-fluoro-3-(methoxymethoxy)-8-((triisopropylsilyl)ethynyl)naphthalen-1-yl)pyrido[4,3-d]pyrimidin-4-yl)-3,8-diazabicyclo[3.2.1]octane-8-carboxylate. To a solution of compound 14 (141 mg, 167 μmol, 1.00 eq) and Intermediate 29 (100 mg, 201 μmol, 1.20 eq, HCl) in DCE (1.00 mL) was added TEA (101 mg, 1.01 mmol, 140 μL, 6.00 eq) and Ti(OEt)₄ (229 mg, 1.01 mmol, 208 μL, 6.00 eq) and AcOH (1.01 mg, 16.7 μmol, 0.960 μL, 0.100 eq). The mixture was stirred at 20° C. for 2 hrs. Then the mixture was added NaBH(OAc) 3 (106 mg, 503 μmol, 3.00 eq) at 20° C. for 12 hrs. LCMS showed 27% of desired compound was detected. The reaction mixture was diluted with H₂O 20.0 mL and extracted with Ethyl acetate 30.0 mL (10.0 mL*3). The combined organic layers were washed with aqueous NaCl 20.0 mL (10.0 mL*2), dried over Na₂SO₄, filtered and concentrated under reduced pressure to give a residue. The residue was purified by prep-TLC (SiO₂, Dichloromethane:Methanol=10:1, P1: R_{f}=0.42). Intermediate 30 (120 mg, 93.2 μmol, 18.5% yield) was obtained as a yellow solid. LCMS: m/z 1287.5 [M+H]⁺.

### Intermediate 31:

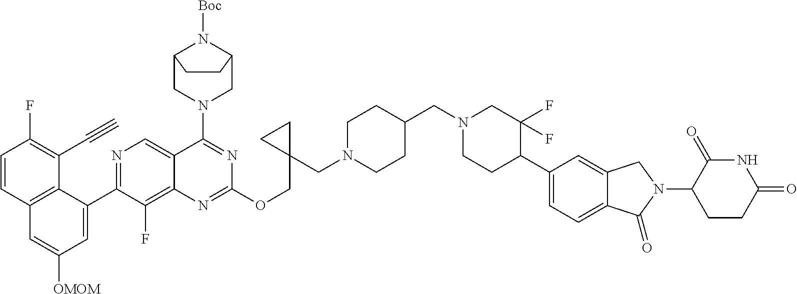

tert-butyl (1R,2S,5S)-3-(3-bromo-2,7-dichloro-8-fluoro-1,6-naphthyridin-4-yl)-2-(hydroxymethyl)-3,8-diazabicyclo[3.2.1]octane-8-carboxylate. To a solution of Intermediate 30 (60.0 mg, 46.6 μmol, 1.00 eq) in DMF (0.500 mL) was added CsF (70.8 mg, 466 μmol, 17.2 μL, 10.0 eq). The mixture was stirred at 20° C. for 1 hr. LCMS showed 83% of desired compound was detected. The reaction mixture was diluted with H₂O 20.0 mL and extracted with Ethyl acetate 30.0 mL (10.0 mL*3). The combined organic layers were washed with aqueous NaCl 20.0 mL (10.0 mL*2), dried over Na₂SO₄, filtered and concentrated under reduced pressure to give a residue. Intermediate 31 (100 mg, 88.4 μmol, 94.8% yield) was obtained as a yellow solid. LCMS: m/z 1130.8 [M+H]⁺.

### Compound 93:

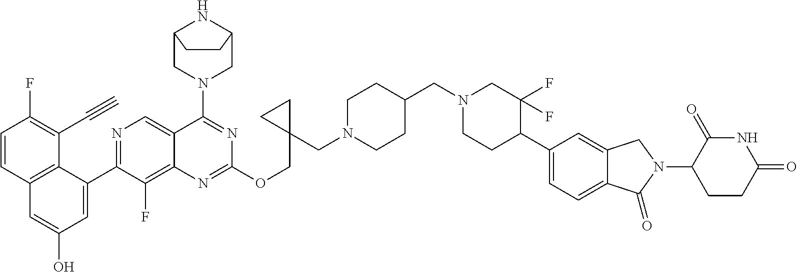 [SMILES: C#Cc1c(F)ccc2cc(O)cc(-c3ncc4c(N5CC6CCC(C5)N6)nc(OCC5(CN6CCC(CN7CCC(c8ccc9c(c8)CN(C8CCC(=O)NC8=O)C9=O)C(F)(F)C7)CC6)CC5)nc4c3F)c12]

3-(5-(1-((1-((1-(((4-((1R,5S)-3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)methyl)cyclopropyl)methyl) piperidin-4-yl)methyl)-3,3-difluoropiperidin-4-yl)-1-oxoisoindolin-2-yl) piperidine-2,6-dione. To a solution of Intermediate 31 (100 mg, 88.4 μmol, 1.00 eq) in ACN (0.200 mL) was added HCl/dioxane (4 M, 10.0 mL, 452 eq). The mixture was stirred at 0° C. for 0.5 hr. LCMS showed 71% of desired compound was detected. The reaction mixture was concentrated under reduced pressure to give a residue. The residue was purified by prep-HPLC (FA condition; column: Phenomenex luna C18 150*25 mm*10 μm; mobile phase: [water (FA)-ACN]; gradient: 7%-37% B over 10 min). Compound 93 (10.0 mg, 9.00 μmol, 10.1% yield, 92.9% purity, FA) was obtained as a white solid. 1HNMR (400 MHz, DMSO-d₆) δ 10.98 (s, 1H), 9.03 (s, 1H), 8.23 (s, 1H), 7.97 (dd, J=6.0, 9.2 Hz, 1H), 7.68 (d, J=8.0 Hz, 1H), 7.56 (s, 1H), 7.49-7.42 (m, 2H), 7.39 (d, J=2.4 Hz, 1H), 7.17 (d, J=2.4 Hz, 1H), 5.11 (dd, J=4.4, 12.6 Hz, 1H), 4.52-4.43 (m, 2H), 4.34-4.31 (m, 1H), 4.30-4.24 (m, 3H), 3.93 (s, 1H), 3.65 (d, J=12.0 Hz, 2H), 3.59 (d, J=6.8 Hz, 4H), 3.15-3.07 (m, 2H), 2.98-2.90 (m, 4H), 2.61 (d, J=2.8 Hz, 1H), 2.58 (s, 1H), 2.42-2.34 (m, 2H), 2.31 (s, 2H), 2.24 (d, J=6.0 Hz, 2H), 2.15 (d, J=9.4 Hz, 2H), 2.03-1.96 (m, 1H), 1.87 (t, J=11.0 Hz, 2H), 1.69 (s, 4H), 1.66-1.60 (m, 2H), 1.52-1.46 (m, 1H), 1.12-1.01 (m, 2H), 0.64 (s, 2H), 0.41 (s, 2H). LCMS: m/z 986.8 [M+H]⁺.

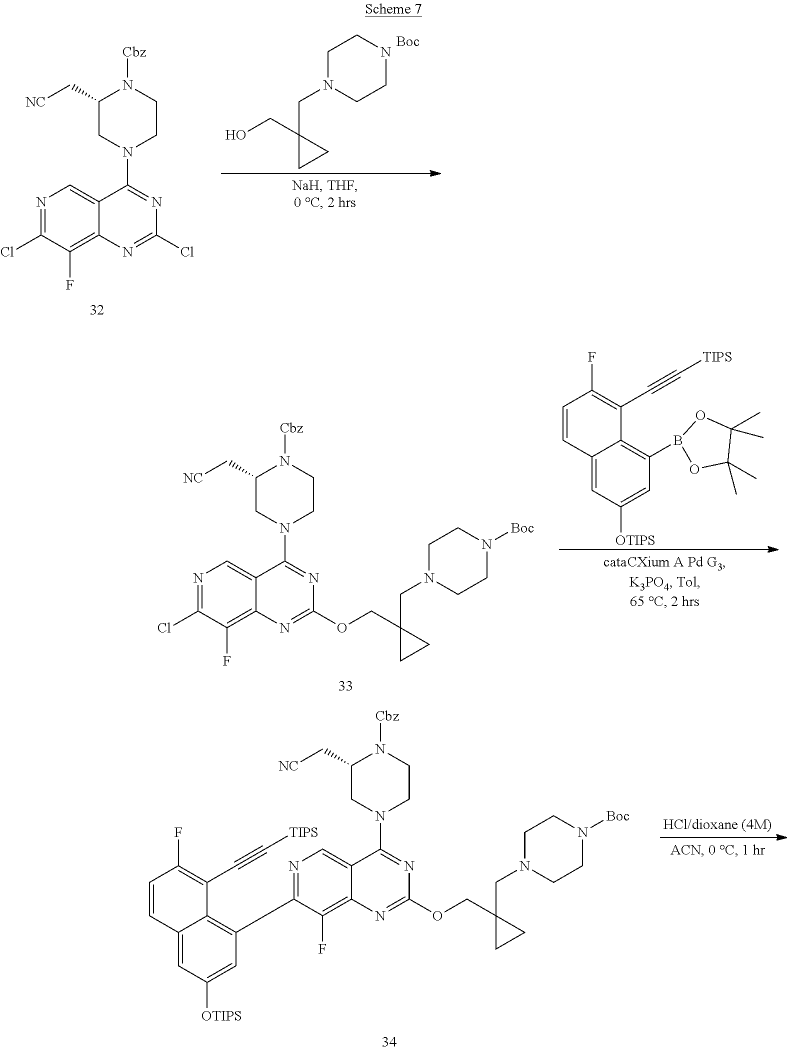

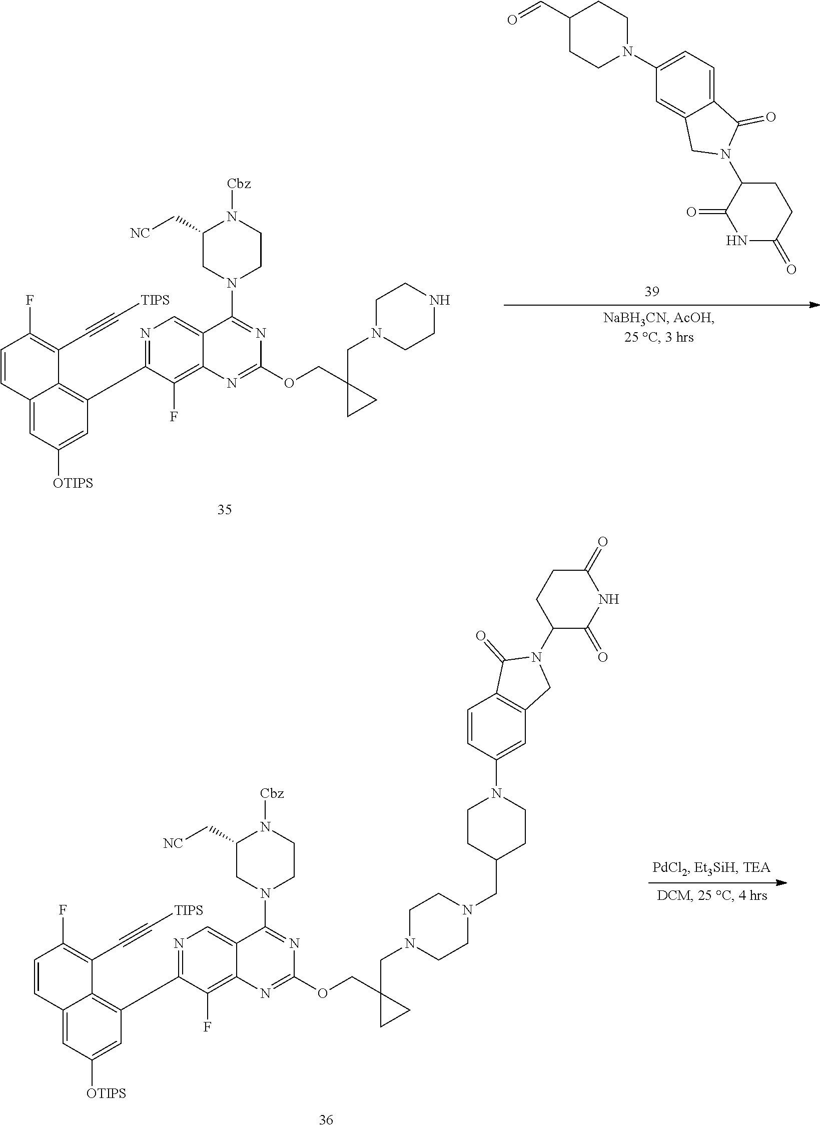

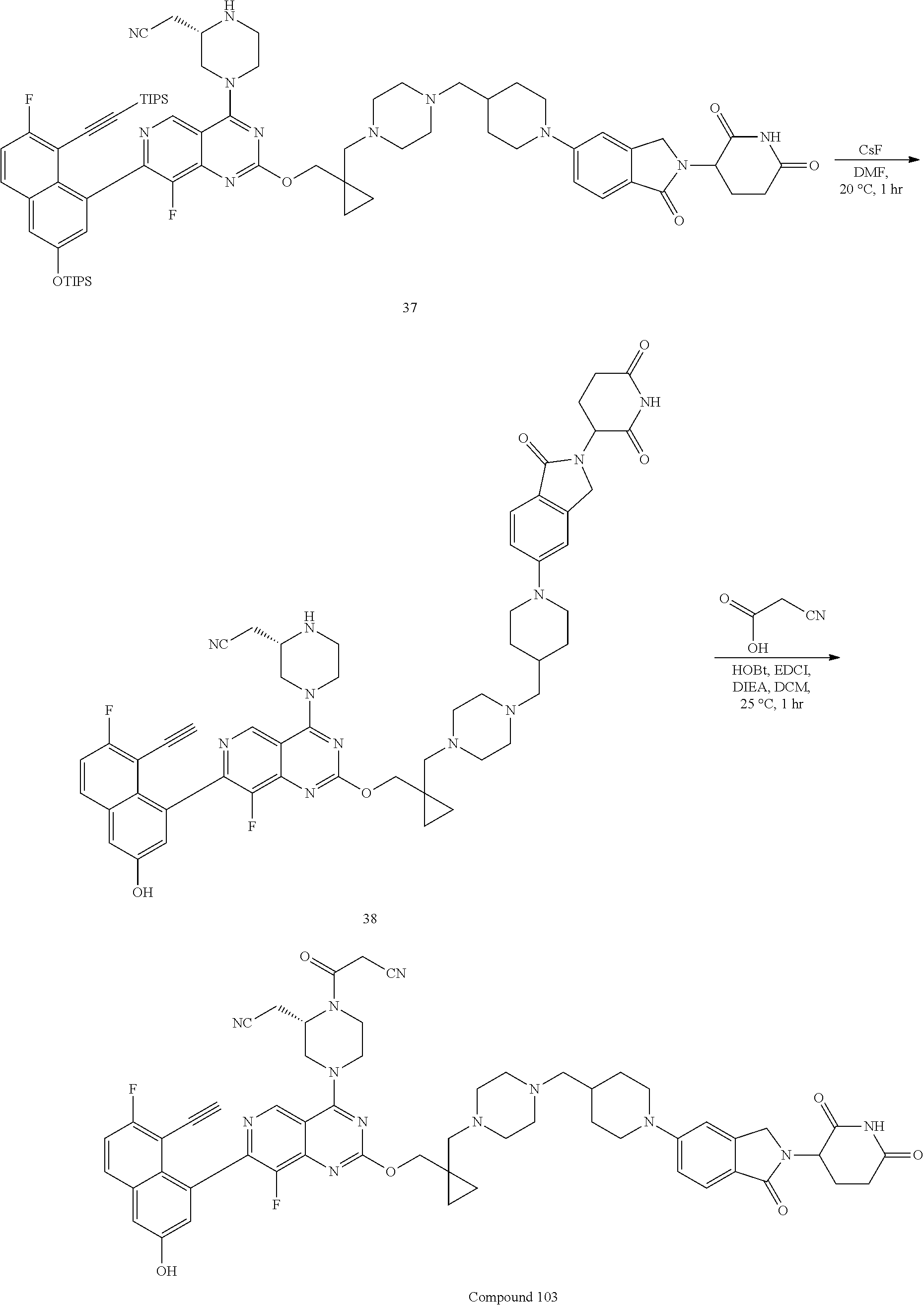

### Intermediate 33:

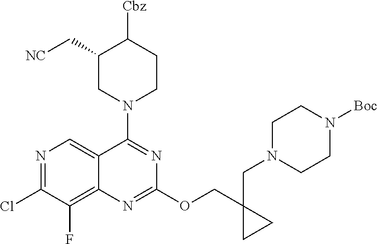 [SMILES: CC(C)(C)OC(=O)N1CCN(CC2(COc3nc(N4CCC(C(=O)OCc5ccccc5)[C@@H](CC#N)C4)c4cnc(Cl)c(F)c4n3)CC2)CC1]

benzyl(S)-4-(2-((1-((4-(tert-butoxycarbonyl) piperazin-1-yl)methyl)cyclopropyl)methoxy)-7-chloro-8-fluoropyrido[4,3-d]pyrimidin-4-yl)-2-(cyanomethyl) piperazine-1-carboxylate. To a solution of tert-butyl 4-((1-(hydroxymethyl)cyclopropyl)methyl) piperazine-1-carboxylate (1.55 g, 5.74 mmol, 1.30 eq) in THF (350 mL) was added NaH (353 mg, 8.84 mmol, 60% purity, 2.00 eq) and stirred at 0° C. for 0.5 hr, then compound 32 (2.10 g, 4.42 mmol, 1.00 eq) in THF (35.0 mL) was added and stirred at 70° C. for 2 hrs. LCMS showed desired MS was detected. The reaction mixture was poured into NH₄Cl (60.0 mL), extracted with Ethyl acetate (50 mL*3), dried over Na₂SO₄, filtrate was concentrated under reduced pressure to give a residue. The residue was purified by column chromatography (SiO₂, Petroleum ether:Ethyl acetate=20:1 to 3:1, TLC:Petroleum ether:Ethyl acetate=1:1, P1 R_{f}=0.3) to give Intermediate 33 (1.40 g, 1.97 mmol, 44.6% yield) as a yellow oil.

LCMS: m/z 709.6 [M+H]⁺.

### Intermediate 34:

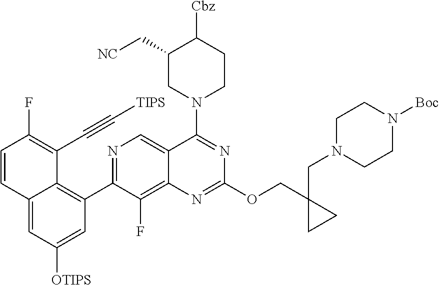

benzyl(S)-4-(2-((1-((4-(tert-butoxycarbonyl) piperazin-1-yl)methyl)cyclopropyl)methoxy)-8-fluoro-7-(7-fluoro-8-((triisopropylsilyl)ethynyl)-3-((triisopropylsilyl)oxy)naphthalen-1-yl)pyrido[4,3-d]pyrimidin-4-yl)-2-(cyanomethyl) piperazine-1-carboxylate To a solution of ((6-fluoro-4-(4,4,5,5-tetramethyl-1,3,2-dioxaborolan-2-yl)-5-((triisopropylsilyl)ethynyl)naphthalen-2-yl)oxy)triisopropylsilane (528 mg, 846 μmol, 1.20 eq) and Intermediate 33 (500 mg, 705 μmol, 1.00 eq) in THF (9.00 mL) was added cataCXium A Pd G3 (77.0 mg, 105 μmol, 0.150 eq) and K₃PO₄ (149 mg, 705 μmol, 3 mL, 1.00 eq). The mixture was stirred at 65° C. for 1 hr under N₂. LCMS showed desired MS was detected. The reaction mixture was concentrated under reduced pressure to give a residue. The residue was purified by column chromatography (SiO₂, Petroleum ether:Ethyl acetate=5:1 to 3:1, TLC:Petroleum ether:Ethyl acetate=1:1, P1 R_{f}=0.3) to give Intermediate 34 (1.40 g, 1.19 mmol, 84.7% yield) as a yellow oil. LCMS: m/z 1172.0 [M+H]⁺.

### Intermediate 35:

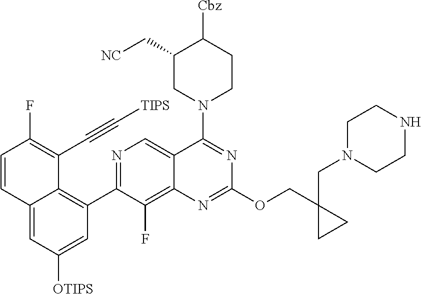

benzyl(S)-2-(cyanomethyl)-4-(8-fluoro-7-(7-fluoro-8-((triisopropylsilyl)ethynyl)-3-((triisopropylsilyl)oxy)naphthalen-1-yl)-2-((1-(piperazin-1-ylmethyl)cyclopropyl)methoxy)pyrido[4,3-d]pyrimidin-4-yl) piperazine-1-carboxylate. To a solution of Intermediate 34 (100 mg, 85.3 μmol, 1.00 eq) in ACN (1.00 mL), was added HCl/dioxane (4 M, 21.3 μL, 1.00 eq), and stirred at 0° C. for 0.5 hr. LCMS showed desired MS was detected. The reaction mixture was poured into NaHCO₃.aq (10.0 mL), extracted with Ethyl acetate (5.00 mL*3), dried over Na₂SO₄, filtrate was concentrated under reduced pressure to give Intermediate 35 (60.0 mg, 56.0 μmol, 65.6% yield) as a yellow solid. LCMS: m/z 1072.4 [M+H]⁺.

### Intermediate 36:

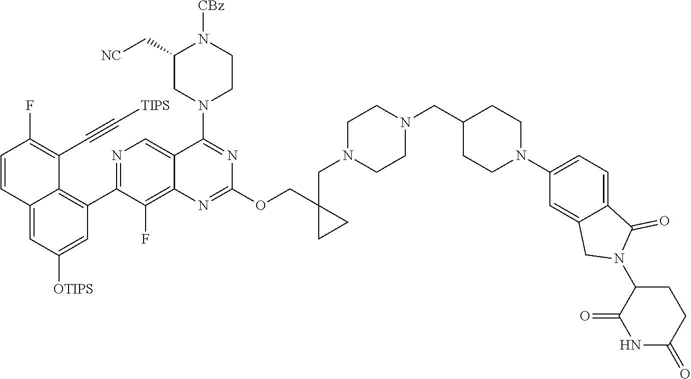

benzyl(2S)-2-(cyanomethyl)-4-(2-((1-((4-((1-(2-(2,6-dioxopiperidin-3-yl)-1-oxoisoindolin-5-yl) piperidin-4-yl)methyl) piperazin-1-yl)methyl)cyclopropyl)methoxy)-8-fluoro-7-(7-fluoro-8-((triisopropylsilyl)ethynyl)-3-((triisopropylsilyl)oxy)naphthalen-1-yl)pyrido[4,3-d]pyrimidin-4-yl) piperazine-1-carboxylate. To a solution of compound 39 (199 mg, 559 μmol, 1.20 eq) and Intermediate 35 (500 mg, 466 μmol, 1.00 eq) in Methanol (12.0 mL) was added AcOH (28.0 mg, 466 μmol, 26.7 μL, 1.00 eq), and stirred at 20° C. for 0.5 hr, then NaBH₃CN (87.9 mg, 1.40 mmol, 3.00 eq) was added. The mixture was stirred at 20° C. for 2 hrs. LCMS showed desired MS was detected. The reaction mixture was concentrated reduced pressure and poured into water (20.0 mL), extracted with Ethyl acetate (5.00 mL*3), dried over Na₂SO₄, filtrate was concentrated under reduced pressure. The residue was purified by column chromatography (SiO₂, Methanol:Dichloromethane=1:30 to 1:15, TLC:Methanol:Dichloromethane=1:10, P1 R_{f}=0.3) to give Intermediate 36 (470 mg, 333 μmol, 71.3% yield) as a yellow solid. LCMS: m/z 1411.3 [M+H]⁺.

### Intermediate 37:

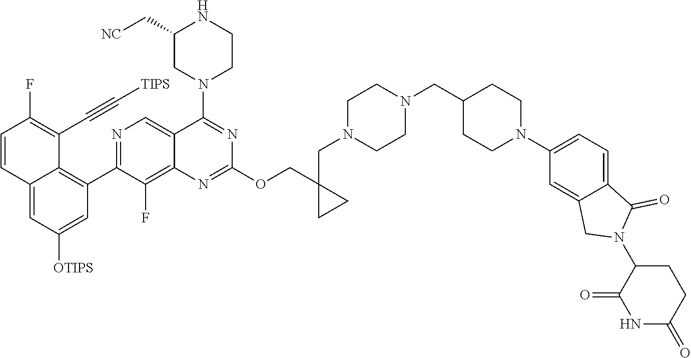

2-((2S)-4-(2-((1-((4-((1-(2-(2,6-dioxopiperidin-3-yl)-1-oxoisoindolin-5-yl) piperidin-4-yl)methyl) piperazin-1-yl)methyl)cyclopropyl)methoxy)-8-fluoro-7-(7-fluoro-8-((triisopropylsilyl)ethynyl)-3-((triisopropylsilyl)oxy)naphthalen-1-yl)pyrido[4,3-d]pyrimidin-4-yl) piperazin-2-yl) acetonitrile. To a solution of Intermediate 36 (26.3 mg, 148 μmol, 0.500 eq), Et₃SiH (207 mg, 1.79 mmol, 285 μL, 6.00 eq), TEA (240 mg, 2.38 mmol, 331 μL, 8.00 eq), PdCl₂ (0.220 mg, 1.26 mmol, 0.150 eq), then stirred at 20° C. for 2 hrs. LCMS showed desired MS was detected. The reaction mixture was concentrated reduced pressure and poured into water (20.0 mL), extracted with Dichloromethane (10.0 mL*3), dried over Na₂SO₄, filtrate was concentrated under reduced pressure to give a residue. The residue was purified by column chromatography (SiO₂, Methanol:Dichloromethane=1:30 to 1:15, TLC:Methanol:Dichloromethane=1:10, P1 R_{f}=0.1) to give Intermediate 37 (325 mg, 254 μmol, 85.5% yield) as a yellow solid. LCMS: m/z 1277.8 [M+H]⁺.

### Intermediate 38:

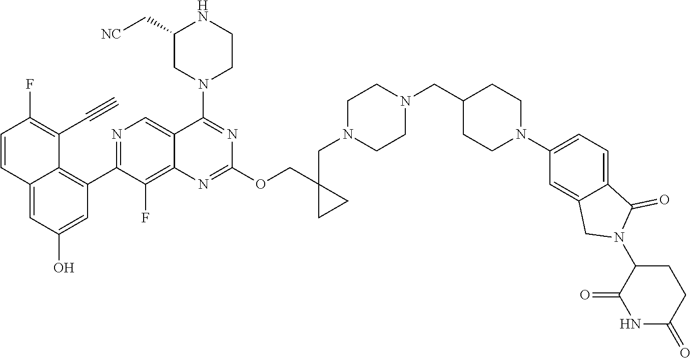 [SMILES: C#Cc1c(F)ccc2cc(O)cc(-c3ncc4c(N5CCN[C@@H](CC#N)C5)nc(OCC5(CN6CCN(CC7CCN(c8ccc9c(c8)CN(C8CCC(=O)NC8=O)C9=O)CC7)CC6)CC5)nc4c3F)c12]

2-((2S)-4-(2-((1-((4-((1-(2-(2,6-dioxopiperidin-3-yl)-1-oxoisoindolin-5-yl) piperidin-4-yl)methyl) piperazin-1-yl)methyl)cyclopropyl)methoxy)-7-(8-ethynyl-7-fluoro-3-hydroxynaphthalen-1-yl)-8-fluoropyrido[4,3-d]pyrimidin-4-yl) piperazin-2-yl) acetonitrile. To a solution of Intermediate 37 (175 mg, 137 μmol, 1.00 eq) in DMF (1.00 mL) was added CsF (624 mg, 4.11 mmol, 151 μL, 30.0 eq), and stirred at 25° C. for 12 hrs. LCMS showed desired MS was detected. The reaction mixture was poured into water (20 mL), extracted with Ethyl acetate (5.00 mL*3), washed with NaCl (aq.) (20 mL*2), dried over Na₂SO₄, filtrate was concentrated under reduced pressure to give Intermediate 38 (115 mg, 119 μmol, 87.0% yield) as a yellow solid. LCMS: m/z 965.0 [M+H]⁺.

### Compound 103:

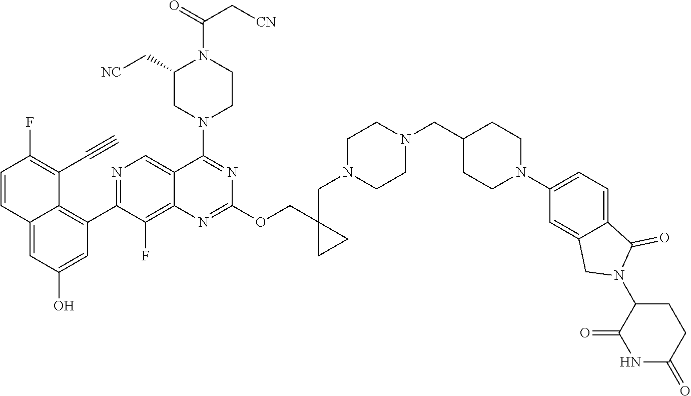 [SMILES: C#Cc1c(F)ccc2cc(O)cc(-c3ncc4c(N5CCN(C(=O)CC#N)[C@@H](CC#N)C5)nc(OCC5(CN6CCN(CC7CCN(c8ccc9c(c8)CN(C8CCC(=O)NC8=O)C9=O)CC7)CC6)CC5)nc4c3F)c12]

3-((2S)-2-(cyanomethyl)-4-(2-((1-((4-((1-(2-(2,6-dioxopiperidin-3-yl)-1-oxoisoindolin-5-yl) piperidin-4-yl)methyl) piperazin-1-yl)methyl)cyclopropyl)methoxy)-7-(8-ethynyl-7-fluoro-3-hydroxynaphthalen-1-yl)-8-fluoropyrido[4,3-d]pyrimidin-4-yl) piperazin-1-yl)-3-oxopropanenitrile. To a solution of Intermediate 38 (10.0 mg, 10.3 μmol, 1.00 eq) and 2-cyanoacetic acid (1.41 mg, 16.6 μmol, 1.60 eq) in DMF (0.600 mL) was added HOBt (1.40 mg, 10.3 μmol, 1.00 eq), EDCI (1.99 mg, 10.3 μmol, 1.00 eq), DMAP (1.27 mg, 10.3 μmol, 1.00 eq), then stirred at 25° C. for 1 hr. LCMS showed desired MS was detected. The reaction mixture was poured into water (5.00 mL), extracted with EtOAc (5.00 mL*2), dried over Na₂SO₄, filtered and concentrated to give a residue. The residue was purified by prep-HPLC (column: Phenomenex luna C18 150*25 mm*10 μm; mobile phase: [water (FA)-ACN]; B %: 10%-40%, 10 min) to give compound 103 (7.00 mg, 5.88 μmol, 28.3% yield, 90.5% purity, FA) as a yellow solid. 1HNMR (400 MHz, DMSO-d₆) δ 10.94 (s, 1H), 10.46-10.07 (m, 1H), 9.11 (d, J=6.4 Hz, 1H), 8.26-8.21 (m, 1H), 8.02-7.96 (m, 1H), 7.52-7.44 (m, 2H), 7.41 (d, J=1.9 Hz, 1H), 7.22-7.16 (m, 1H), 7.06-7.00 (m, 2H), 5.04 (dd, J=5.0, 13.5 Hz, 1H), 4.89-4.81 (m, 1H), 4.54-4.42 (m, 1H), 4.37-4.26 (m, 5H), 4.23-4.13 (m, 3H), 3.95-3.76 (m, 6H), 3.10-3.03 (m, 2H), 2.97-2.74 (m, 5H), 2.63-2.55 (m, 4H), 2.32-2.24 (m, 4H), 2.13-2.06 (m, 2H), 2.01-1.92 (m, 1H), 1.78-1.69 (m, 3H), 1.25-1.08 (m, 3H), 0.71-0.60 (m, 2H), 0.43 (s, 2H). LCMS: m/z 1031.8 [M-COOH+H]⁺.

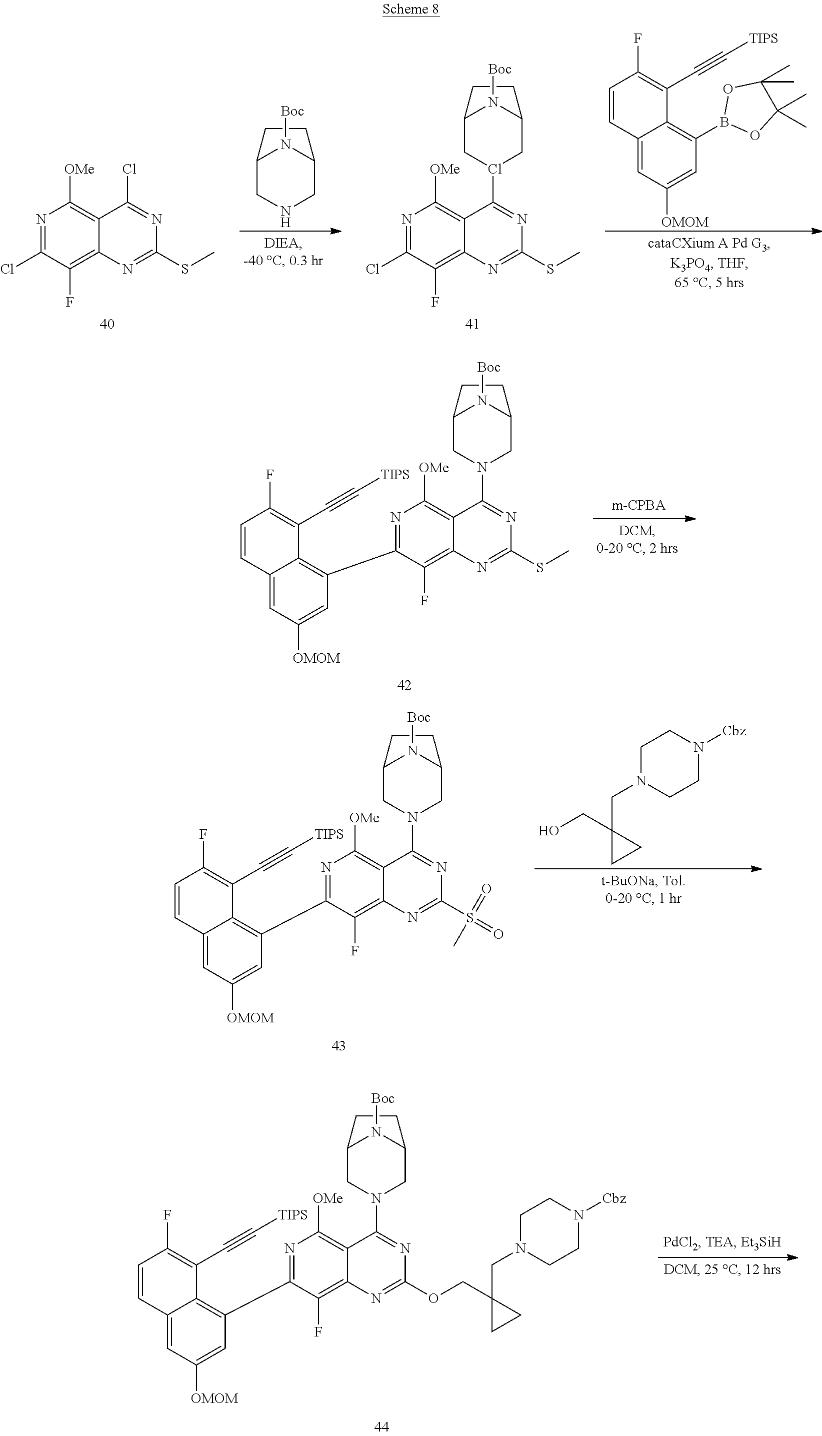

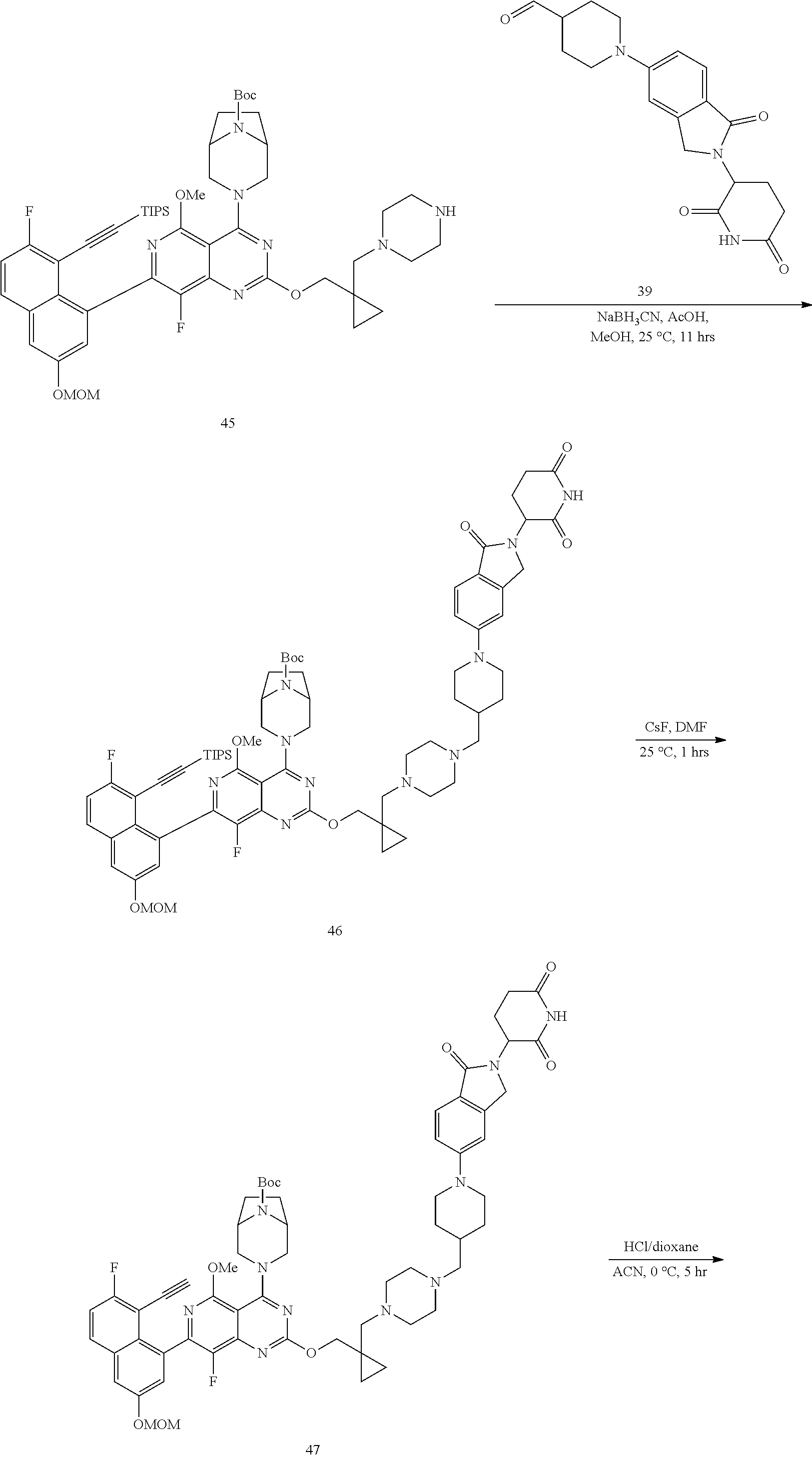

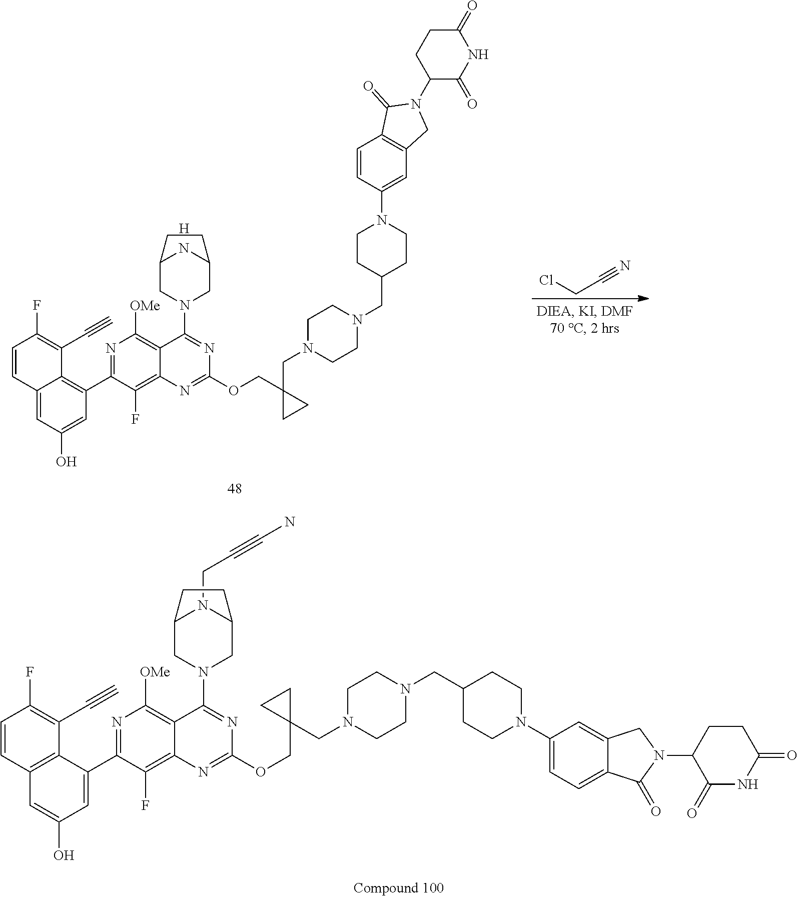

### Intermediate 41:

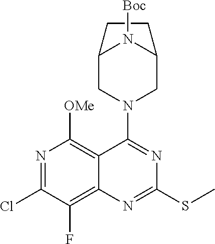 [SMILES: COc1nc(Cl)c(F)c2nc(SC)nc(N3CC4CCC(C3)N4C(=O)OC(C)(C)C)c12]

tert-butyl 3-(7-chloro-8-fluoro-5-methoxy-2-(methylthio)pyrido[4,3-d]pyrimidin-4-yl)-3,8-diazabicyclo[3.2.1]octane-8-carboxylate. To a solution of compound 40 (73.0 g, 248 mmol, 1.00 eq) in DCM (730 mL) was added tert-butyl 3,8-diazabicyclo[3.2.1]octane-8-carboxylate (47.4 g, 223 mmol, 0.900 eq) and DIEA (32.0 g, 248 mmol, 43.2 mL, 1.00 eq) at −40° C. The mixture was stirred at −40° C. for 0.3 hr. LCMS showed desired MS was detected. The reaction mixture was concentrated reduced pressure and poured into water (2000 mL), extracted with DCM (800 mL*3), dried over Na₂SO₄, filtrate was concentrated under reduced pressure to give a residue. The crude product was triturated with ethyl acetate (250 mL) at 25° C. for 10 mins to give Intermediate 41 (253 g, 538 mmol, 72.3% yield) as a yellow solid. LCMS: m/z 470.0 [M+H]⁺.

### Intermediate 42:

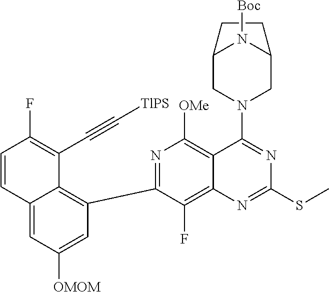

tert-butyl 3-(8-fluoro-5-methoxy-7-(3-(methoxymethoxy)-8-((triisopropylsilyl)ethynyl)naphthalen-1-yl)-2-(methylthio)pyrido[4,3-d]pyrimidin-4-yl)-3,8-diazabicyclo[3.2.1]octane-8-carboxylate. A mixture of Intermediate 41 (300 mg, 638 μmol, 1.00 eq), ((2-fluoro-6-(methoxymethoxy)-8-(4,4,5,5-tetramethyl-1,3,2-dioxaborolan-2-yl) naphthalen-1-yl) ethynyl)triisopropylsilane (473 mg, 957 μmol, 1.50 eq), cataCXium A Pd G3 (46.4 mg, 63.8 μmol, 0.100 eq), K₃PO₄ (1.50 M, 1.28 mL, 3.00 eq) in THF (2.00 mL) was degassed and purged with N₂ for 3 times, and then the mixture was stirred at 65° C. for 12 hrs under N₂ atmosphere. LCMS showed desired compound was detected. The reaction mixture was diluted with H₂O 20.0 mL and extracted with ethyl acetate 30.0 mL (10.0 mL*3). The combined organic layers were washed with aqueous NaCl 20.0 mL (10.0 mL*2), dried over Na₂SO₄, filtered and concentrated under reduced pressure to give a residue. The residue was purified by column chromatography (SiO₂, Dichloromethane:Methanol=1:0 to 20:1, TLC:Dichloromethane:Methanol=10:1 P1: R_{f}=0.56) Intermediate 42 (500 mg, crude) was obtained as a yellow gum. LCMS: m/z 802.6 [M+H]⁺

### Intermediate 43:

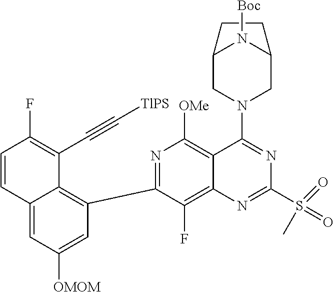

tert-butyl 3-(8-fluoro-7-(7-fluoro-3-(methoxymethoxy)-8-((triisopropylsilyl)ethynyl)naphthalen-1-yl)-5-methoxy-2-(methylsulfonyl)pyrido[4,3-d]pyrimidin-4-yl)-3,8-diazabicyclo[3.2.1]octane-8-carboxylate. To a solution of Intermediate 42 (6.00 g, 7.32 mmol, 1.00 eq) in DCM (60.0 mL) was added m-CPBA (2.97 g, 14.6 mmol, 85% purity, 2.00 eq) at 0° C. and stirred 25° C. for 2 hrs. LCMS showed desired MS was detected. The reaction mixture was quenched with Na₂SO₃ (200 mL), extracted with DCM (100 mL*3), the organic layer was dried over Na₂SO₄, filtered and concentrated to give Intermediate 43 (6.10 g, 7.16 mmol, 97.9% yield) as a brown solid. LCMS: m/z 852.6 [M+H]⁺.

### Intermediate 44:

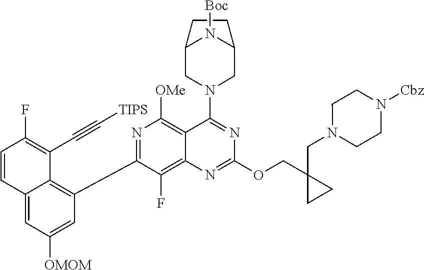

tert-butyl 3-(2-((1-((4-((benzyloxy) carbonyl) piperazin-1-yl)methyl)cyclopropyl)methoxy)-8-fluoro-7-(7-fluoro-3-(methoxymethoxy)-8-((triisopropylsilyl)ethynyl)naphthalen-1-yl)-5-methoxypyrido[4,3-d]pyrimidin-4-yl)-3,8-diazabicyclo[3.2.1]octane-8-carboxylate. To a solution of Intermediate 43 (3.00 g, 3.52 mmol, 1.00 eq) and compound 2-1 (1.07 g, 3.52 mmol, 1.00 eq) in Tol. (30.0 mL) was added t-BuONa (2 M, 3.52 mL, 2.00 eq) at 0° C., then stirred at 25° C. for 1 hr. LCMS showed desired MS was detected. The reaction mixture was poured into water (100 mL), extracted with ethyl acetate (200 mL*3), the organic layer was dried over Na₂SO₄, filtered and concentrated to give a residue. The residue was purified by column chromatography (SiO₂, Petroleum ether:Ethyl acetate=20:1 to 3:1, TLC:Petroleum ether:Ethyl acetate=1:1, P1 R_{f}=0.5) to give Intermediate 44 (2.70 g, 5.03 mmol, 71.4% yield) as a yellow solid. LCMS: m/z 1076.8 [M+H]⁺.

### Intermediate 45:

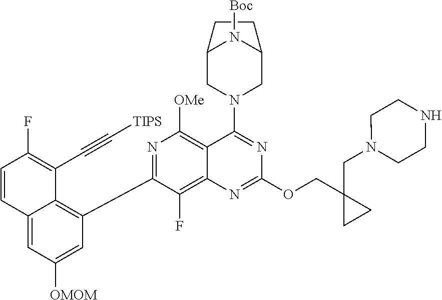

tert-butyl 3-(8-fluoro-7-(7-fluoro-3-(methoxymethoxy)-8-((triisopropylsilyl)ethynyl)naphthalen-1-yl)-5-methoxy-2-((1-(piperazin-1-ylmethyl)cyclopropyl)methoxy)pyrido[4,3-d]pyrimidin-4-yl)-3,8-diazabicyclo[3.2.1]octane-8-carboxylate. To a solution of Intermediate 44 (2.00 g, 1.86 mmol, 1.00 eq) in DCM (30.0 mL) was added TEA (1.88 g, 18.6 mmol, 2.59 mL, 10.0 eq), PdCl₂ (49.4 mg, 279 μmol, 0.150 eq) and Et₃SiH (1.73 g, 14.9 mmol, 2.37 mL, 8.00 eq), then stirred at 25° C. for 12 hrs. LCMS showed desired MS was detected. The reaction mixture was filtered and concentrated to give a residue. The residue was purified by column chromatography (SiO₂, Petroleum Ether:Ethyl acetate=20:1 to Dichloromethane:Methanol=15:1, TLC:Dichloromethane:Methanol=10:1, P1 R_{f}=0.2) to give Intermediate 45 (1.70 g, 1.60 mmol, 97.1% yield) as a brown solid. LCMS: m/z 942.6 [M+H]⁺.

### Intermediate 46:

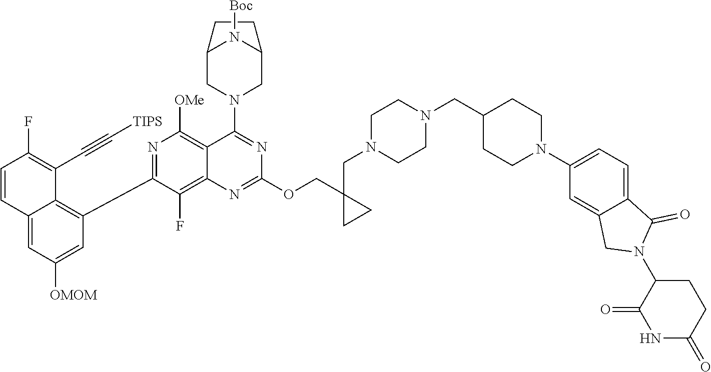

tert-butyl 3-(2-((1-((4-((1-(2-(2,6-dioxopiperidin-3-yl)-1-oxoisoindolin-5-yl) piperidin-4-yl)methyl) piperazin-1-yl)methyl)cyclopropyl)methoxy)-8-fluoro-7-(7-fluoro-3-(methoxymethoxy)-8-((triisopropylsilyl)ethynyl)naphthalen-1-yl)-5-methoxypyrido[4,3-d]pyrimidin-4-yl)-3,8-diazabicyclo[3.2.1]octane-8-carboxylate. To a solution of Intermediate 45 (500 mg, 531 μmol, 1.00 eq) and compound 39 (283 mg, 796 μmol, 1.50 eq) in MeOH (10 mL) was added AcOH (3.19 mg, 53.1 μmol, 3.03 μL, 0.100 eq) at 25° C. for 1 hr, then NaBH₃CN (100 mg, 1.59 mmol, 3.00 eq) was added into the reaction mixture and stirred at 25° C. for 10 hrs. LCMS showed desired MS was detected. The reaction mixture was poured into water (30.0 mL), extracted with ethyl acetate (30.0 mL*2), the organic layer was dried over Na₂SO₄, filtered and concentrated to give a residue. The residue was purified by column chromatography (SiO₂, Petroleum ether:Ethyl acetate=10:1 to Dichloromethane:Methanol=10:1, TLC:Dichloromethane:Methanol=10:1, P1 R_{f}=0.3) to give Intermediate 46 (375 mg, 292 μmol, 55.1% yield) as a brown solid. LCMS: m/z 1282.5 [M+H]⁺.

### Intermediate 47:

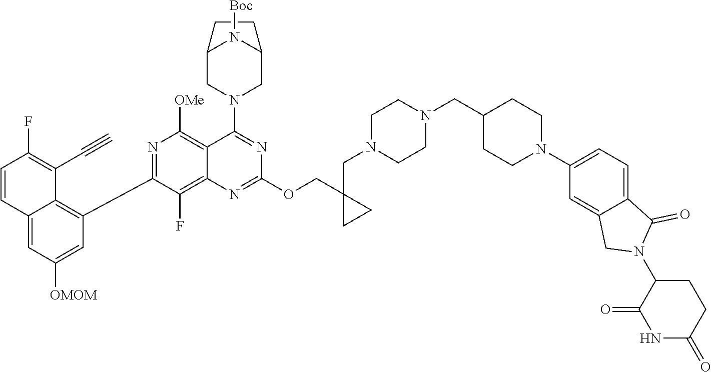

tert-butyl 3-(2-((1-((4-((1-(2-(2,6-dioxopiperidin-3-yl)-1-oxoisoindolin-5-yl) piperidin-4-yl)methyl) piperazin-1-yl)methyl)cyclopropyl)methoxy)-7-(8-ethynyl-7-fluoro-3-(methoxymethoxy)naphthalen-1-yl)-8-fluoro-5-methoxypyrido[4,3-d]pyrimidin-4-yl)-3,8-diazabicyclo[3.2.1]octane-8-carboxylate. To a solution of Intermediate 46 (350 mg, 273 μmol, 1.00 eq) in DMF (3.00 mL) was added CsF (415 mg, 2.73 mmol, 10.0 eq). The mixture was stirred at 25° C. for 1 hr. LCMS showed desired MS was detected. The reaction mixture was poured into water (10.0 mL), extracted with ethyl acetate (10.0 mL*3), the organic layer was washed with brine (20.0 mL*3), dried over Na₂SO₄ filtered and concentrated to give Intermediate 47 (350 mg, crude) as a brown oil. LCMS: m/z 1125.7 [M+H]⁺.

### Intermediate 48:

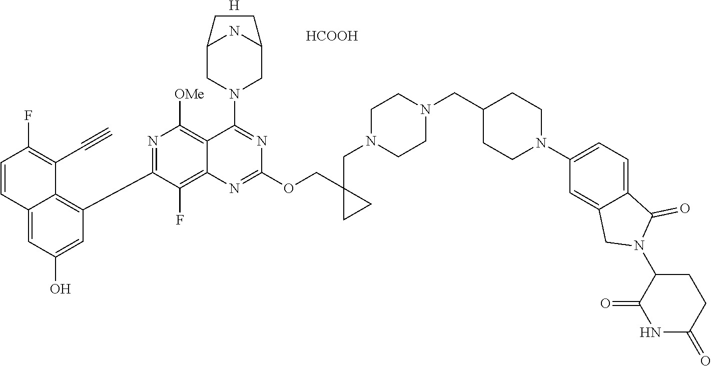

3-(5-(4-((4-((1-(((4-(3,8-diazabicyclo[3.2.1]octan-3-yl)-7-(8-ethynyl-7-fluoro-3-hydroxynaphthalen-1-yl)-8-fluoro-5-methoxypyrido[4,3-d]pyrimidin-2-yl)oxy)methyl)cyclopropyl)methyl) piperazin-1-yl)methyl) piperidin-1-yl)-1-oxoisoindolin-2-yl) piperidine-2,6-dione formate. To a solution of Intermediate 47 (150 mg, 133 μmol, 1.00 eq) in MeCN (2.00 mL) was added HCl/dioxane (4 M, 1.00 mL, 30.0 eq). The mixture was stirred at 0° C. for 0.5 hr. LCMS showed desired MS was detected. The reaction mixture concentrated to give a residue. The residue was dissolved in water (5.00 mL) and slowly dripped into ice saturated NaHCO₃ (10.0 mL), extracted with ethyl acetate (10.0 mL*3), the organic layer was dried over Na₂SO₄, filtered and concentrated to give a residue. The residue was purified by prep-HPLC (column: Phenomenex luna C18 150*25 mm*10 μm; mobile phase: [water (FA)-ACN]; B %: 1%-30%, 8 min) to give Intermediate 48 (20.0 mg, 19.9 μmol, 21.0% yield, 95.1% purity, FA) as a yellow solid. ¹H NMR (DMSO-d₆, 400 MHz) δ 10.98-10.90 (m, 1H), 10.94 (s, 1H), 8.22 (s, 1H), 7.98 (dd, J=6.0, 9.4 Hz, 1H), 7.52-7.43 (m, 2H), 7.39 (d, J=2.6 Hz, 1H), 7.22 (d, J=2.4 Hz, 1H), 7.06-6.99 (m, 2H), 5.04 (dd, J=5.2, 13.3 Hz, 1H), 4.35-4.15 (m, 5H), 3.94-3.81 (m, 8H), 3.59 (s, 4H), 2.94-2.85 (m, 1H), 2.80 (t, J=11.8 Hz, 2H), 2.63-2.53 (m, 2H), 2.38-2.24 (m, 8H), 2.13-2.06 (m, 2H), 2.00-1.92 (m, 1H), 1.77-1.59 (m, 7H), 1.21-1.09 (m, 2H), 0.63 (s, 2H), 0.41 (s, 2H). LCMS: m/z 981.6 [M−HCOOH+H]⁺.

### Compound 100:

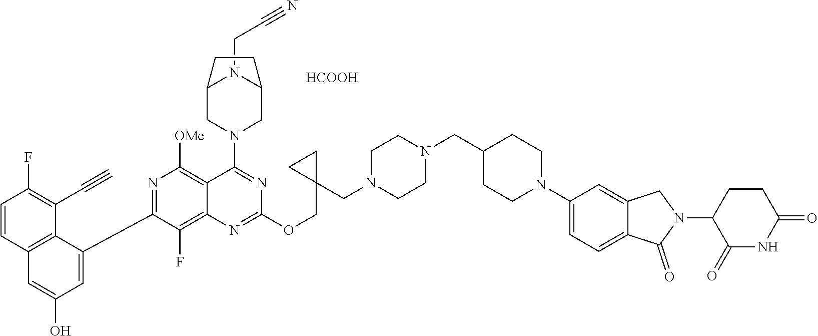

2-(3-(2-((1-((4-((1-(2-(2,6-dioxopiperidin-3-yl)-1-oxoisoindolin-5-yl) piperidin-4-yl)methyl) piperazin-1-yl)methyl)cyclopropyl)methoxy)-7-(8-ethynyl-7-fluoro-3-hydroxynaphthalen-1-yl)-8-fluoro-5-methoxypyrido[4,3-d]pyrimidin-4-yl)-3,8-diazabicyclo[3.2.1]octan-8-yl) acetonitrile formate To a solution of 2-chloroacetonitrile (21.6 mg, 286 μmol, 18.1 μL, 1.20 eq) and Intermediate 48 (234 mg, 238 μmol, 1.00 eq) in DMF (1.00 mL) was added DIEA (61.6 mg, 477 μmol, 83.0 μL, 2.00 eq) and KI (39.5 mg, 238 μmol, 1.00 eq). The mixture was stirred at 80° C. for 2 hrs. LCMS showed desired MS was detected. The reaction mixture was filtrated to give a residue. The residue was purified by prep-HPLC (column: Phenomenex luna C18 150*25 mm*10 μm; mobile phase: [water (FA)-ACN]; gradient: 15%-45% B over 8 min) to give compound 100 (48.0 mg, 44.9 μmol, 18.8% yield, 99.9% purity, FA) as a white solid. ¹H NMR (400 MHz, DMSO-d₆) δ 10.98-10.83 (m, 1H), 10.24-10.07 (m, 1H), 8.02 (d, J=3.0 Hz, 1H), 7.53-7.41 (m, 2H), 7.40-7.34 (m, 1H), 7.22 (d, J=2.2 Hz, 1H), 7.09-6.95 (m, 2H), 5.10-4.97 (m, 1H), 4.37-4.08 (m, 5H), 3.94-3.81 (m, 6H), 3.57 (s, 2H), 3.40 (s, 3H), 2.98-2.75 (m, 4H), 2.59-2.55 (m, 1H), 2.38 (d, J=4.0 Hz, 2H), 2.31-2.29 (m, 1H), 2.12-2.05 (m, 2H), 2.00-1.90 (m, 2H), 1.89-1.82 (m, 2H), 1.79-1.52 (m, 6H), 1.24-1.09 (m, 3H), 0.62 (s, 2H), 0.43-0.34 (m, 2H). LCMS: m/z 1020.4 [M+H]⁺.

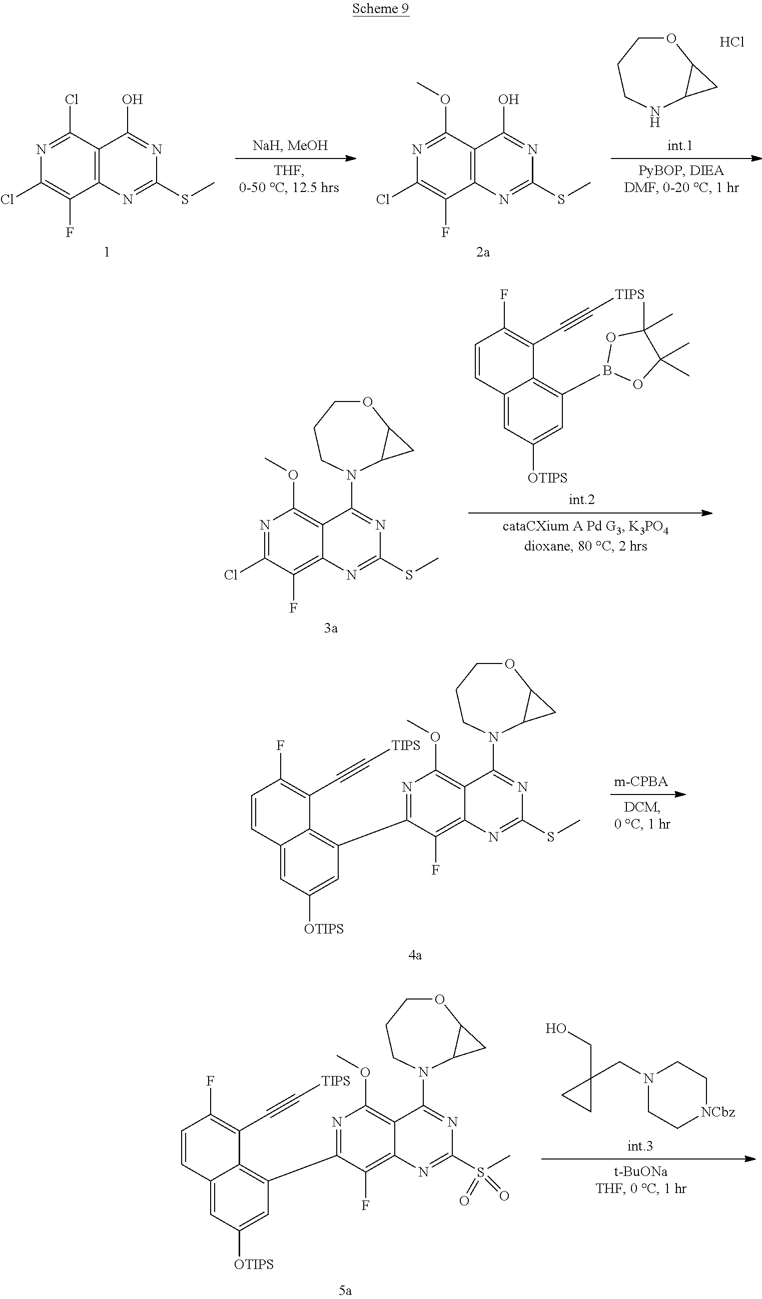

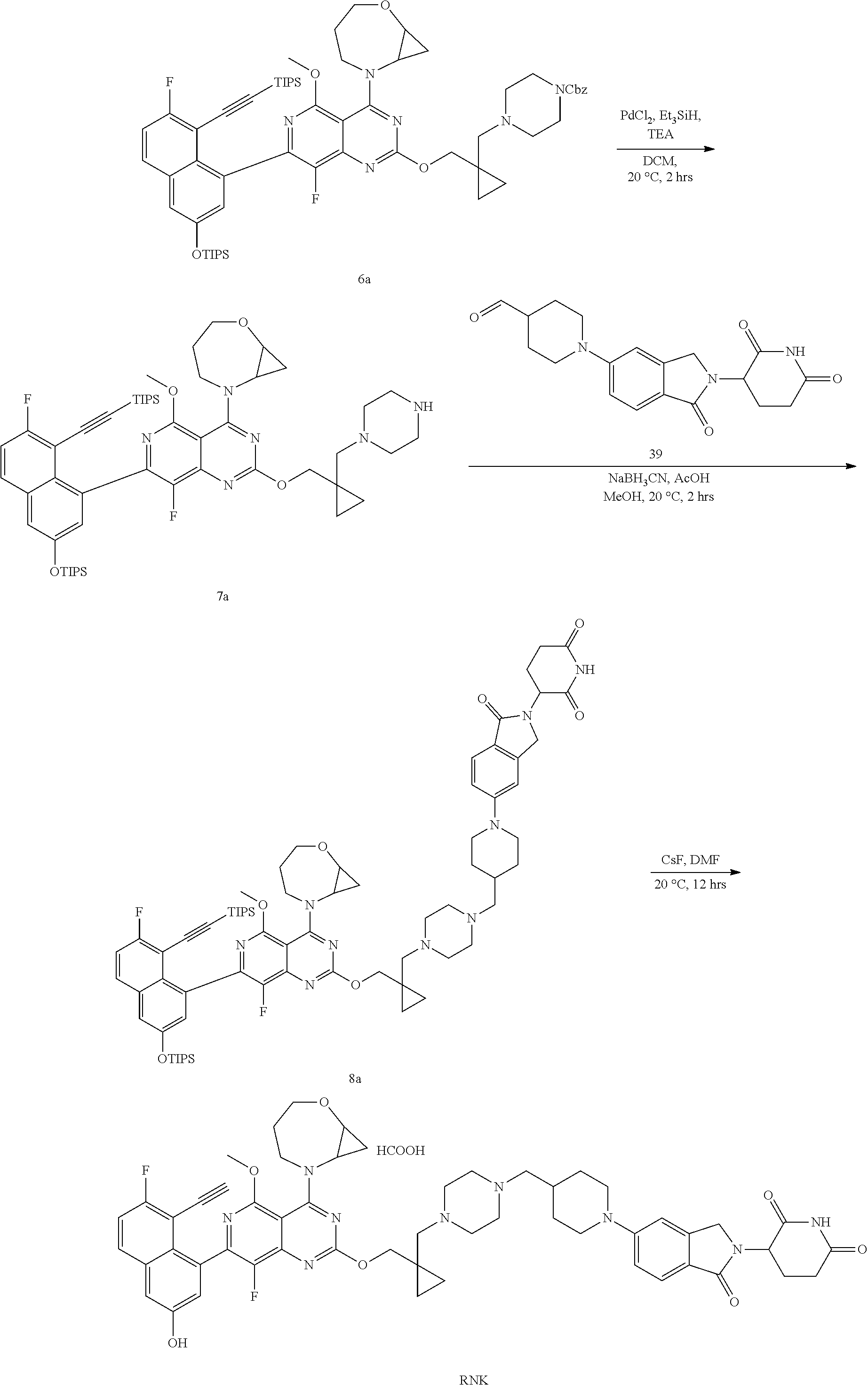

### Compound 2a:

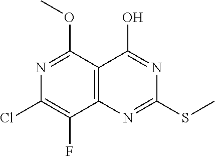 [SMILES: COc1nc(Cl)c(F)c2nc(SC)nc(O)c12]

### 7-chloro-8-fluoro-5-methoxy-2-(methylthio)pyrido[4,3-d]pyrimidin-4-ol

To a solution of THF (50 mL) and NaH (1.43 g, 35.7 mmol, 60.0% purity, 2.00 eq) was added MeOH (5.72 g, 178 mmol, 7.22 mL, 10.0 eq) at 0° C. and stirred at 25° C. for 0.5 hrs, then a solution of compound 1 (5.00 g, 17.8 mmol, 1.00 eq) was added at 0° C. The mixture was stirred at 50° C. for 12 hrs. LCMS showed the reaction was complete. The reaction mixture was quenched by water (1 L), added 1 M HCl to adjust pH to 3-4 at 0° C., filtered and concentrated the cake to give compound 2a (5.00 g, crude) as a yellow solid.

LCMS: m/z 275.9 [M+H]⁺.

### Compound 3a:

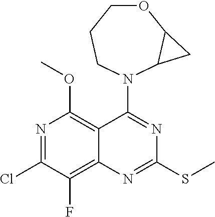 [SMILES: COc1nc(Cl)c(F)c2nc(SC)nc(N3CCCOC4CC43)c12]

### 6-(7-chloro-8-fluoro-5-methoxy-2-(methylthio)pyrido[4,3-d]pyrimidin-4-yl)-2-oxa-6-azabicyclo[5.1.0]octane

To a solution of compound 2a (700 mg, 2.54 mmol, 1.00 eq) in DMF (7 mL) was added PyBOP (2.25 g, 4.32 mmol, 1.70 eq) and DIEA (984 mg, 7.62 mmol, 1.33 mL, 3.00 eq) at 0° C. and stirred for 0.5 hr, then compound int.1 (494 mg, 3.30 mmol, 1.30 eq, HCl) was added. The mixture was stirred at 20° C. for 0.5 hr. LCMS showed the reaction was complete. The reaction solution was poured into H₂O (50 mL) and extracted with ethyl acetate 50 mL (25 mL*2). The combine organic layers were washed with brine, dried over Na₂SO₄ and concentrated to give compound 3a (1.20 g, crude) as a yellow solid.

LCMS: m/z 371.0 [M+H]⁺.

### Compound 4a:

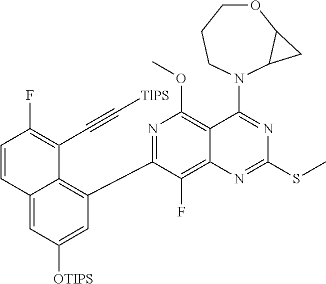

### 6-(8-fluoro-7-(7-fluoro-8-((triisopropylsilyl)ethynyl)-3-((triisopropylsilyl)oxy)naphthalen-1-yl)-5-methoxy-2-(methylthio)pyrido[4,3-d]pyrimidin-4-yl)-2-oxa-6-azabicyclo[5.1.0]octane

To a mixture of compound 3a (700 mg, 1.89 mmol, 1.00 eq), compound int.2 (1.42 g, 2.27 mmol, 1.20 eq), cataCXium A Pd G3 (206 mg, 283 μmol, 0.15 eq), K₃PO₄ (1.5 M, 3 mL, 2.38 eq) in dioxane (9 mL) was degassed and purged with N₂ for 3 times, and then the mixture was stirred at 80° C. for 2 hrs under N₂ atmosphere. LCMS showed the reaction was complete. The reaction solution was poured into H₂O (50 mL) and extracted with ethyl acetate 50 mL (25 mL*2). The combine organic layers were washed with brine, dried over Na₂SO₄ and concentrated to give the crude product. The crude product was purified by column chromatography (SiO₂, Petroleum ether:Ethyl acetate=50:1 to 5:1, SiO₂, Petroleum ether:Ethyl acetate=5:1, P1 R_{f}=0.4) to give compound 4 (1.27 g, 1.52 mmol, 80.7% yield) as a yellow solid.

LCMS: m/z 833.2 [M+H]⁺.

### Compound 5a:

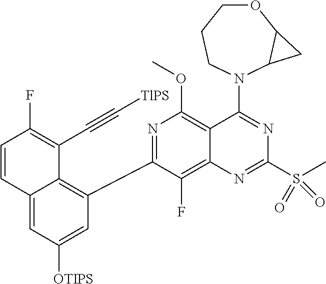

### 6-(8-fluoro-7-(7-fluoro-8-((triisopropylsilyl)ethynyl)-3-((triisopropylsilyl)oxy)naphthalen-1-yl)-5-methoxy-2-(methylsulfonyl)pyrido[4,3-d]pyrimidin-4-yl)-2-oxa-6-azabicyclo[5.1.0]octane

To a solution of compound 4a (1.20 g, 1.44 mmol, 1.00 eq) in DCM (12 mL) was added m-CPBA (731 mg, 3.60 mmol, 85% purity, 2.50 eq) at 0° C. for 1 hr. LCMS showed the reaction was complete. The reaction mixture was quenched with Na₂SO₃ (50 mL), extracted with dichloromethane 50 mL (25 mL*2), the organic layer was washed with NaHCO₃50 mL (25 mL*2), dried over Na₂SO₄, filtered and concentrated to give the crude product. The crude product was purified by column chromatography (SiO₂, Petroleum ether:Ethyl acetate=20:1 to 1:1, SiO₂, Petroleum ether:Ethyl acetate=1:1, P1 R_{f}=0.4) to give compound 5a (1.00 g, 1.11 mmol, 77.3% yield, 96.3% purity) as a yellow solid. LCMS: m/z 865.2 [M+H]⁺.

### Compound 6a:

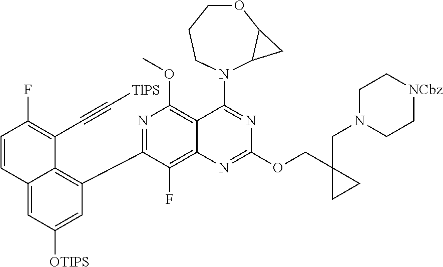

### benzyl 4-((1-(((4-(2-oxa-6-azabicyclo[5.1.0]octan-6-yl)-8-fluoro-7-(7-fluoro-8-((triisopropylsilyl)ethynyl)-3-((triisopropylsilyl)oxy)naphthalen-1-yl)-5-methoxypyrido[4,3-d]pyrimidin-2-yl)oxy)methyl)cyclopropyl)methyl) piperazine-1-carboxylate

To a solution of compound 5a (500 mg, 578 μmol, 1.00 eq) and compound int.3 (299 mg, 982 μmol, 1.70 eq) in Tol. (5 mL) was added t-BuONa (2 M, 578 μL, 2.00 eq). The mixture was stirred at 0° C. for 1 hr. LCMS showed the reaction was complete. The reaction solution was poured into H₂O (50 mL) and extracted with ethyl acetate 50 mL (25 mL*2). The combine organic layers were washed with brine, dried over Na₂SO₄ and concentrated to give the crude product. The crude product was purified by column chromatography (SiO₂, Petroleum ether:Ethyl acetate=20:1 to 1:1, SiO₂, Petroleum ether:Ethyl acetate=1:1, P1 R_{f}=0.4) to give compound 6a (600 mg, 551 μmol, 95.3% yield) as a yellow solid.

LCMS: m/z 1089.5 [M+H]⁺.

### Compound 7a:

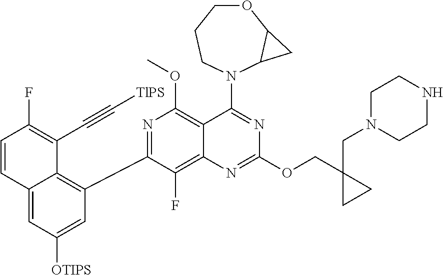

### 6-(8-fluoro-7-(7-fluoro-8-((triisopropylsilyl)ethynyl)-3-((triisopropylsilyl)oxy)naphthalen-1-yl)-5-methoxy-2-((1-(piperazin-1-ylmethyl)cyclopropyl)methoxy)pyrido[4,3-d]pyrimidin-4-yl)-2-oxa-6-azabicyclo[5.1.0]octane

To a solution of compound 6a (550 mg, 505 μmol, 1.00 eq) in DCM (1 mL) was added TEA (408 mg, 4.04 mmol, 562 μL, 8.00 eq), PdCl₂ (17.9 mg, 101 μmol, 0.20 eq) and Et₃SiH (352 mg, 3.03 mmol, 484 μL, 6.00 eq), then stirred at 20° C. for 2 hrs. LCMS showed the reaction was complete. The reaction solution was poured into H₂O (50 mL) and extracted with dichloromethane 50 mL (25 mL*2). The combine organic layers were washed with brine, dried over Na₂SO₄ and concentrated to give the crude product. The crude product was purified by column chromatography (SiO₂, Dichloromethane:Methanol=100:1 to 10:1, SiO₂, Dichloromethane:Methanol=10:1, P1 R_{f}=0.3) to give compound 7a (500 mg, crude) as a yellow solid.

LCMS: m/z 955.5 [M+H]⁺.

### Compound 8a:

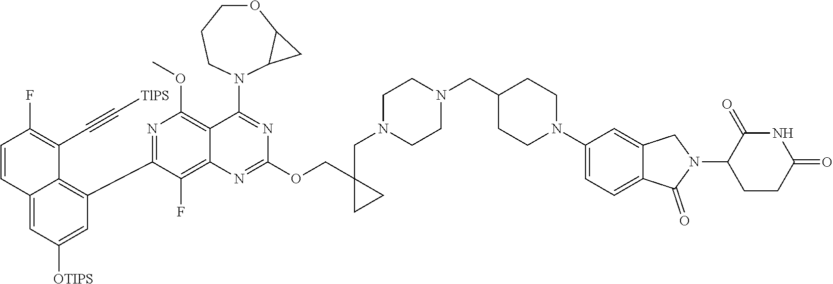

3-(5-(4-((4-((1-(((4-(2-oxa-6-azabicyclo[5.1.0]octan-6-yl)-8-fluoro-7-(7-fluoro-8-((triisopropylsilyl)ethynyl)-3-((triisopropylsilyl)oxy)naphthalen-1-yl)-5-methoxypyrido[4,3-d]pyrimidin-2-yl)oxy)methyl)cyclopropyl)methyl) piperazin-1-yl)methyl) piperidin-1-yl)-1-oxoisoindolin-2-yl) piperidine-2,6-dione.

To a solution of compound 7 (350 mg, 366 μmol, 1.00 eq) and compound CRBN11 (195 mg, 549 μmol, 1.50 eq) in MeOH (3 mL) was added AcOH (2.20 mg, 36.6 μmol, 2.10 μL, 0.10 eq) and stirred at 20° C. for 1 hr, then NaBH₃CN (69.1 mg, 1.10 mmol, 3.00 eq) was added, and the mixture was stirred at 20° C. for 1 hr. LCMS showed the reaction was complete. The reaction solution was poured into H₂O (50 mL) and extracted with ethyl acetate 50 mL (25 mL*2). The combine organic layers were washed with brine, dried over Na₂SO₄ and concentrated to give the crude product. The crude product was purified by column chromatography (SiO₂, Dichloromethane:Methanol=100:1 to 10:1, SiO₂, Dichloromethane:Methanol=10:1, P1 R_{f}=0.2) to give compound 8 (500 mg, 316 μmol, 86.4% yield, 82.0% purity) as a yellow solid.

LCMS: m/z 1294.5 [M+H]⁺.

### Compound RNK

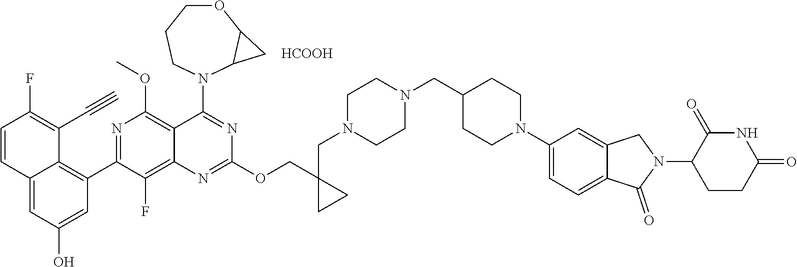 [SMILES: C#Cc1c(F)ccc2cc(O)cc(-c3nc(OC)c4c(N5CCCOC6CC65)nc(OCC5(CN6CCN(CC7CCN(c8ccc9c(c8)CN(C8CCC(=O)NC8=O)C9=O)CC7)CC6)CC5)nc4c3F)c12 ; "HCOOH"]

### 3-(5-(4-((4-((1-(((4-(2-oxa-6-azabicyclo[5.1.0]octan-6-yl)-7-(8-ethynyl-7-fluoro-3-hydroxynaphthalen-1-yl)-8-fluoro-5-methoxypyrido[4,3-d]pyrimidin-2-yl)oxy)methyl)cyclopropyl)methyl) piperazin-1-yl)methyl) piperidin-1-yl)-1-oxoisoindolin-2-yl) piperidine-2,6-dione formate

To a solution of compound 8 (200 mg, 154 μmol, 1.00 eq) in DMF (4 mL) was added CsF (2.35 g, 15.5 mmol, 100 eq) and the mixture was stirred at 20° C. for 12 hrs. LCMS showed the reaction was complete. The reaction mixture was diluted with H₂O 20 mL and extracted with ethyl acetate 50 mL (25 mL*2). The combined organic layers were washed with aqueous NaCl 50 mL (25 mL*2), dried over Na₂SO₄, filtered and concentrated under reduced pressure to give crude product. The crude product was purification by prep-HPLC (column: CD01-Phenomenex luna C18 150*25*10 μm; mobile phase: [water (FA)-ACN]; gradient: 15%-45% B over 11 min) to afford the compound (45.6 mg, 43.5 μmol, 28.1% yield, 98.0% purity, FA) as a white solid.

¹H NMR (400 MHz, DMSO-d₆) δ 10.94 (s, 1H), 10.43-9.93 (m, 1H), 8.16 (s, 1H), 7.97 (d, J=3.4 Hz, 1H), 7.50-7.44 (m, 2H), 7.38 (d, J=2.2 Hz, 1H), 7.26-7.23 (m, 1H), 7.17-7.14 (m, 1H), 7.04-7.00 (m, 2H), 5.03 (d, J=5.2 Hz, 1H), 4.52-4.43 (m, 1H), 4.34-4.18 (m, 4H), 4.14-4.02 (m, 1H), 3.90-3.82 (m, 6H), 3.71 (d, J=11.8 Hz, 1H), 3.56-3.52 (m, 2H), 3.17 (d, J=5.2 Hz, 2H), 2.95-2.82 (m, 2H), 2.81-2.73 (m, 2H), 2.63-2.55 (m, 2H), 2.42-2.27 (m, 9H), 2.09 (d, J=10.8 Hz, 2H), 1.99-1.93 (m, 1H), 1.74 (d, J=12.2 Hz, 4H), 1.19-1.09 (m, 2H), 0.97-0.88 (m, 1H), 0.62 (d, J=4.2 Hz, 2H), 0.40 (s, 2H).

LCMS: m/z 982.4 [M−HCOOH+H]⁺.

The following compounds in Table 1 were prepared according to the methods described ab ove using the appropriate starting materials.

TABLE 1

| | | LCMS: | |
|---|---|---|---|
| Com- | | m/z | ¹H NMR |
| pound | Structure | [M + H]⁺ | (DMSO-d₆, 400 MHz) |
| 1 | 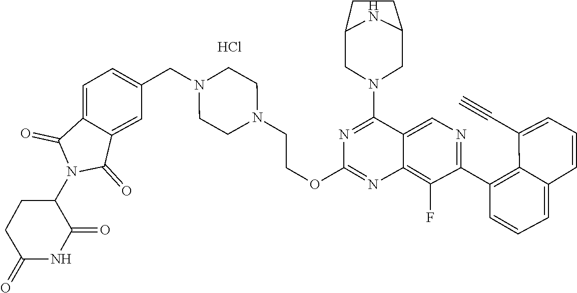 [SMILES: C#Cc1cccc2cccc(-c3ncc4c(N5CC6CCC(C5)N6)nc(OCCN5CCN(Cc6ccc7c(c6)C(=O)N(C6CCC(=O)NC6=O)C7=O)CC5)nc4c3F)c12 ; Cl] | 808.3 | 11.15 (s, 1H), 9.07-9.02 (m, 1H), 8.17- 8.10 (m, 2H), 7.89-7.85 (m, 1H), 7.84- 7.81 (m, 1H), 7.81-7.77 (m, 1H), 7.73- 7.66 (m, 2H), 7.60-7.53 (m, 2H), 5.18- 5.09 (m, 1H), 4.51-4.41 (m, 3H), 4.35- 4.28 (m, 1H), 3.72-3.54 (m, 10H), 2.90- 2.83 (m, 1H), 2.70 (t, J = 5.6 Hz, 2H), 2.64-2.60 (m, 1H), 2.60-2.56 (m, 1H), 2.41 (s, 5H), 2.08-2.01 (m, 1H), 1.73-1.64 (m, 4H). |
| 2 | 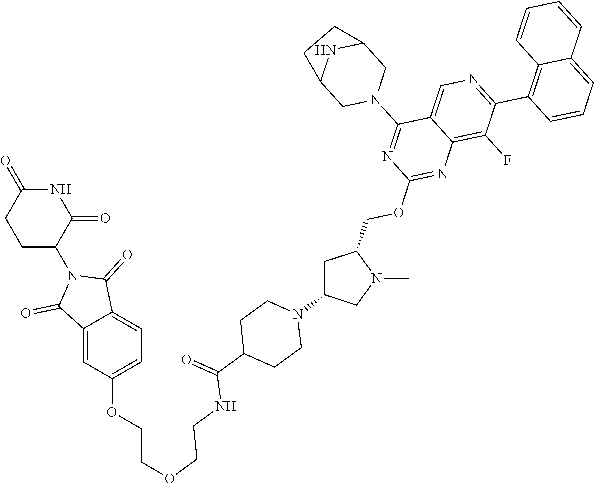 [SMILES: CN1C[C@H](N2CCC(C(=O)NCCOCCOc3ccc4c(c3)C(=O)N(C3CCC(=O)NC3=O)C4=O)CC2)C[C@@H]1COc1nc(N2CC3CCC(C2)N3)c2cnc(-c3cccc4ccccc34)c(F)c2n1] | 952.4 | 9.14 (s, 1H), 8.06 (dd, J = 3.6 Hz, 1H), 8.01 (d, J = 8.4 Hz, 1H), 7.81 (d, J = 8.4 Hz, 1H), 7.72 (d, J = 7.8 Hz, 1H), 7.68- 7.63 (m, 2H), 7.56 (dt, J = 1.2 Hz, 1H), 7.52-7.47 (m, 1H), 7.43 (s, 1H), 7.36-7.32 (m, 1H), 5.11 (dd, J = 5.6 Hz, 1H), 4.70- 4.64 (m, 3H), 4.62-4.53 (m, 3H), 4.31- 4.27 (m, 2H), 3.87-3.84 (m, 2H), 3.78- 3.66 (m, 5H), 3.61 (t, J = 5.4 Hz, 2H), 3.38 (t, J = 5.4 Hz, 2H), 3.17-3.13 (m, 1H), 3.05-2.85 (m, 5H), 2.77-2.69 (m, 2H), 2.62-2.50 (m, 2H), 2.48 (s, 3H), 2.31-2.24 (m, 1H), 2.17-2.10 (m, 3H), 1.89-1.81 (m, 4H), 1.77-1.72 (m, 4H). |
| 3 | 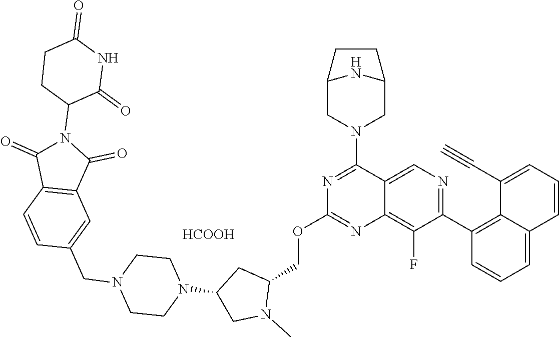 [SMILES: C#Cc1cccc2cccc(-c3ncc4c(N5CC6CCC(C5)N6)nc(OC[C@H]5C[C@@H](N6CCN(Cc7ccc8c(c7)C(=O)N(C7CCC(=O)NC7=O)C8=O)CC6)CN5C)nc4c3F)c12 ; "HCOOH"] | 863.5 | 11.11-10.89 (m, 1H), 9.06 (s, 1H), 8.23 (s, 2H), 8.15 (t, J = 8.0 Hz, 2H), 7.75-7.71 (m, 1H), 7.67 (d, J = 7.0 Hz, 1H), 7.62- 7.55 (m, 2H), 7.52 (s, 1H), 7.43 (d, J = 7.8 Hz, 1H), 5.11 (dd, J = 5.2, 13.3 Hz, 1H), 4.52 (d, J = 13.0 Hz, 1H), 4.47-4.41 (m, 2H), 4.39-4.27 (m, 3H), 3.70 (s, 4H), 3.68-3.65 (m, 2H), 3.64-3.61 (m, 1H), 3.56 (s, 3H), 3.05-2.98 (m, 1H), 2.97-2.85 (m, 2H), 2.68-2.59 (m, 2H), 2.38 (dd, J = 4.6, 13.1 Hz, 6H), 2.36-2.32 (m, 2H), 2.31 (s, 2H), 2.14-2.06 (m, 1H), 2.04-1.94 (m, 1H), 1.73 (s, 3H), 1.64-1.56 (m, 1H). |
| 4 | 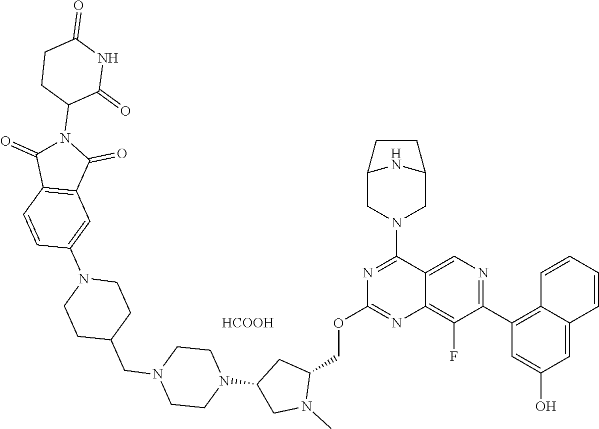 [SMILES: CN1C[C@H](N2CCN(CC3CCN(c4ccc5c(c4)C(=O)N(C4CCC(=O)NC4=O)C5=O)CC3)CC2)C[C@@H]1COc1nc(N2CC3CCC(C2)N3)c2cnc(-c3cc(O)cc4ccccc34)c(F)c2n1 ; "HCOOH"] | 952.3 | 11.15-11.01 (m, 1H), 9.18 (s, 1H), 8.21 (s, 1H), 7.80 (d, J = 8.0 Hz, 1H), 7.65 (d, J = 8.6 Hz, 1H), 7.59-7.53 (m, 1H), 7.47-7.40 (m, 1H), 7.33-7.26 (m, 3H), 7.25-7.19 (m, 3H), 5.10-5.02 (m, 1H), 4.52-4.33 (m, 5H), 4.02 (d, J = 11.8 Hz, 3H), 3.62 (s, 4H), 3.03-2.85 (m, 7H), 2.69-2.59 (m, 3H), 2.35 (s, 2H), 2.31 (s, 5H), 2.25 (s, 1H), 2.11 (d, J = 6.4 Hz, 3H), 2.05-1.96 (m, 2H), 1.76 (d, J = 11.0 Hz, 3H), 1.67 (s, 3H), 1.20-0.96 (m, 3H). |
| 5 | 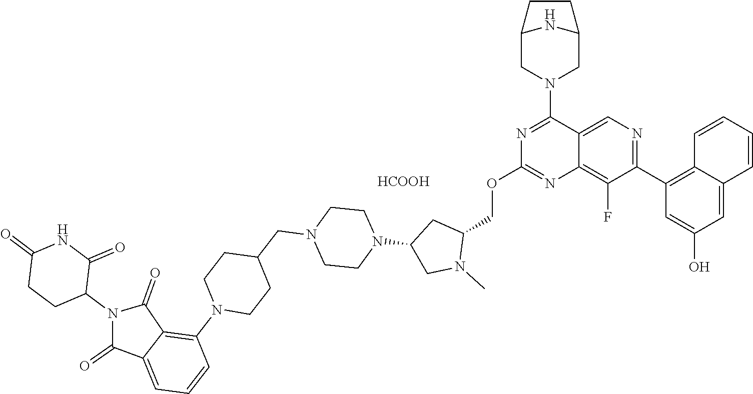 [SMILES: CN1C[C@H](N2CCN(CC3CCN(c4cccc5c4C(=O)N(C4CCC(=O)NC4=O)C5=O)CC3)CC2)C[C@@H]1COc1nc(N2CC3CCC(C2)N3)c2cnc(-c3cc(O)cc4ccccc34)c(F)c2n1 ; "HCOOH"] | 952.8 | 11.12-11.05 (m, 1H), 9.18 (s, 1H), 8.20 (s, 2H), 7.80 (d, J = 8.1 Hz, 1H), 7.70-7.64 (m, 1H), 7.56 (d, J = 8.8 Hz, 1H), 7.44 (t, J = 7.6 Hz, 1H), 7.34-7.22 (m, 5H), 5.08 (dd, J = 5.6, 12.9 Hz, 1H), 4.52-4.42 (m, 3H), 4.41-4.34 (m, 1H), 3.70-3.57 (m, 11H), 3.05-2.98 (m, 2H), 2.93-2.81 (m, 5H), 2.66-2.59 (m, 3H), 2.42-2.38 (m, 3H), 2.32 (s, 3H), 2.16 (d, J = 6.6 Hz, 2H), 2.09-2.01 (m, 2H), 1.97-1.86 (m, 1H), 1.82-1.76 (m, 2H), 1.67 (s, 3H), 1.64-1.54 (m, 2H), 1.39-1.21 (m, 3H). |
| 6 | 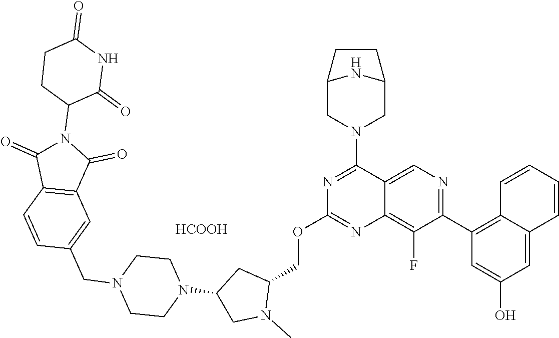 [SMILES: CN1C[C@H](N2CCN(Cc3ccc4c(c3)C(=O)N(C3CCC(=O)NC3=O)C4=O)CC2)C[C@@H]1COc1nc(N2CC3CCC(C2)N3)c2cnc(-c3cc(O)cc4ccccc34)c(F)c2n1 ; "HCOOH"] | 869.3 | 11.20-11.04 (m, 1H), 9.17 (s, 1H), 8.20 (s, 2H), 7.90-7.85 (m, 1H), 7.81 (dd, J = 5.8, 9.0 Hz, 3H), 7.56 (d, J = 8.0 Hz, 1H), 7.44 (t, J = 7.6 Hz, 1H), 7.30-7.21 (m, 3H), 5.18-5.10 (m, 1H), 4.51-4.33 (m, 5H), 3.64 (s, 3H), 3.05-2.98 (m, 3H), 2.90 (ddd, J = 3.8, 11.9, 17.3 Hz, 4H), 2.62 (d, J = 2.4 Hz, 3H), 2.59-2.56 (m, 3H), 2.35-2.32 (m, 3H), 2.31 (s, 3H), 2.12-2.02 (m, 3H), 1.70-1.60 (m, 5H). |
| 7 | 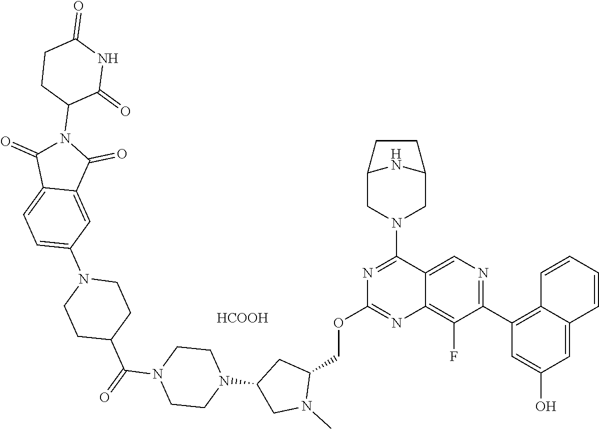 [SMILES: CN1C[C@H](N2CCN(C(=O)C3CCN(c4ccc5c(c4)C(=O)N(C4CCC(=O)NC4=O)C5=O)CC3)CC2)C[C@@H]1COc1nc(N2CC3CCC(C2)N3)c2cnc(-c3cc(O)cc4ccccc34)c(F)c2n1 ; "HCOOH"] | 966.9 | 11.07 (s, 1H), 9.18 (s, 1H), 8.16 (s, 1H), 7.79 (d, J = 8.4 Hz, 1H), 7.65 (d, J = 8.6 Hz, 1H), 7.55 (d, J = 7.8 Hz, 1H), 7.44 (t, J = 7.4 Hz, 1H), 7.34-7.20 (m, 5H), 5.12- 5.02 (m, 1H), 4.53-4.43 (m, 3H), 4.38 (dd, J = 5.0, 10.8 Hz, 1H), 4.06-3.99 (m, 2H), 3.64 (s, 7H), 3.09-2.98 (m, 7H), 2.69-2.58 (m, 4H), 2.42 (d, J = 3.4 Hz, 2H), 2.37- 2.28 (m, 8H), 2.19-2.05 (m, 2H), 2.03- 1.98 (m, 1H), 1.74-1.63 (m, 7H). |
| 8 | 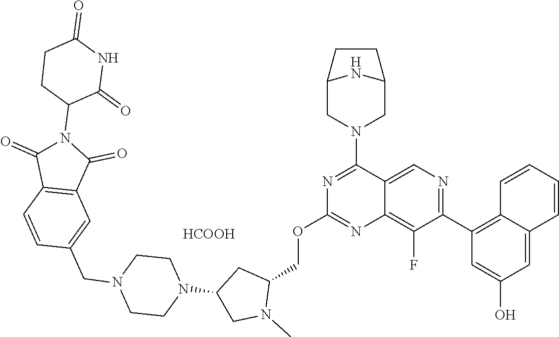 [SMILES: CN1C[C@H](N2CCN(Cc3ccc4c(c3)C(=O)N(C3CCC(=O)NC3=O)C4=O)CC2)C[C@@H]1COc1nc(N2CC3CCC(C2)N3)c2cnc(-c3cc(O)cc4ccccc34)c(F)c2n1 ; "HCOOH"] | 855.3 | 10.99 (s, 1H), 9.18 (s, 1H), 8.22 (s, 2H), 7.80 (d, J = 8.3 Hz, 1H), 7.67 (d, J = 7.8 Hz, 1H), 7.58-7.50 (m, 2H), 7.48-7.38 (m, 2H), 7.31-7.20 (m, 3H), 5.11 (dd, J = 5.0, 13.3 Hz, 1H), 4.51 (s, 1H), 4.49-4.27 (m, 6H), 3.56 (s, 6H), 3.02 (dd, J = 3.4, 10.1 Hz, 1H), 2.97-2.85 (m, 2H), 2.70-2.61 (m, 2H), 2.60-2.56 (m, 1H), 2.53 (d, J = 1.8 Hz, 2H), 2.44-2.37 (m, 6H), 2.35 (s, 1H), 2.31 (s, 2H), 2.12-2.06 (m, 1H), 2.02-1.95 (m, 1H), 1.69 (s, 4H), 1.62 (s, 1H). |
| 9 | 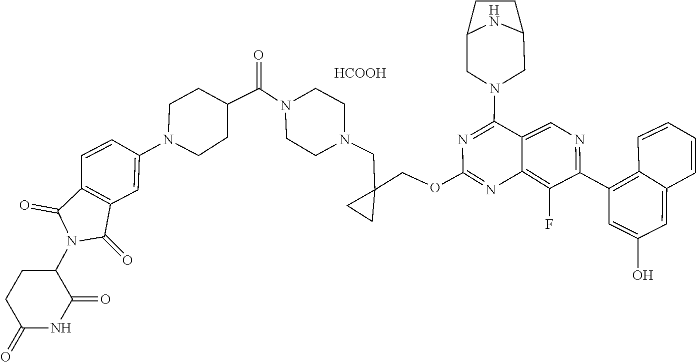 [SMILES: O=C1CCC(N2C(=O)c3ccc(N4CCC(C(=O)N5CCN(CC6(COc7nc(N8CC9CCC(C8)N9)c8cnc(-c9cc(O)cc%10ccccc9%10)c(F)c8n7)CC6)CC5)CC4)cc3C2=O)C(=O)N1 ; "HCOOH"] | 937.2 | 11.07 (s, 1H), 9.17 (s, 1H), 8.17 (s, 1H), 7.80 (d, J = 8.2 Hz, 1H), 7.68-7.63 (m, 1H), 7.54 (d, J = 8.6 Hz, 1H), 7.44 (t, J = 7.4 Hz, 1H), 7.30 (dd, J = 2.0, 8.8 Hz, 2H), 7.26-7.20 (m, 3H), 5.06 (dd, J = 5.4, 12.8 Hz, 1H), 4.50 (d, J = 11.8 Hz, 2H), 4.34 (s, 2H), 4.04 (d, J = 12.6 Hz, 2H), 3.74-3.63 (m, 5H), 3.54-3.46 (m, 4H), 3.04 (t, J = 11.4 Hz, 3H), 2.98-2.88 (m, 2H), 2.57-2.53 (m, 2H), 2.43 (d, J = 5.4 Hz, 2H), 2.37-2.32 (m, 4H), 2.04-1.98 (m, 1H), 1.74-1.54 (m, 8H), 0.67 (s, 2H), 0.43 (s, 2H). |
| 10 | 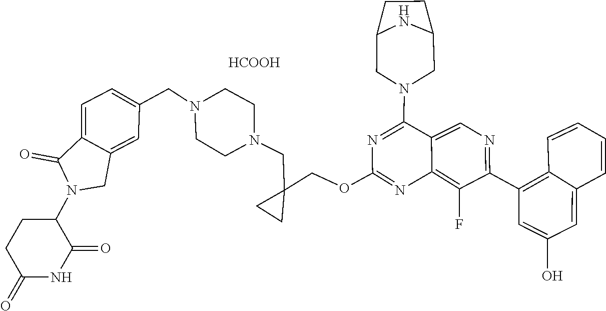 [SMILES: O=C1CCC(N2Cc3cc(CN4CCN(CC5(COc6nc(N7CC8CCC(C7)N8)c7cnc(-c8cc(O)cc9ccccc89)c(F)c7n6)CC5)CC4)ccc3C2=O)C(=O)N1 ; "HCOOH"] | 826.3 | 11.04 (s, 1H), 9.22 (s, 1H), 8.29 (s, 1H), 7.86 (d, J = 8.4 Hz, 1H), 7.72 (d, J = 7.8 Hz, 1H), 7.62 (d, J = 8.4 Hz, 1H), 7.54 (s, 1H), 7.52-7.43 (m, 2H), 7.35 (d, J = 2.3 Hz, 1H), 7.32-7.26 (m, 2H), 5.16 (dd, J = 5.0, 13.3 Hz, 1H), 4.57-4.45 (m, 3H), 4.39-4.32 (m, 3H), 3.73-3.71 (m, 3H), 3.68 (s, 5H), 3.02-2.94 (m, 1H), 2.69-2.63 (m, 1H), 2.60-2.58 (m, 2H), 2.45 (d, J = 4.4 Hz, 2H), 2.41 (d, J = 4.4 Hz, 2H), 2.40-2.33 (m, 4H), 2.08-2.02 (m, 1H), 1.74 (s, 4H), 0.69 (s, 2H), 0.46 (s, 2H). |
| 11 | 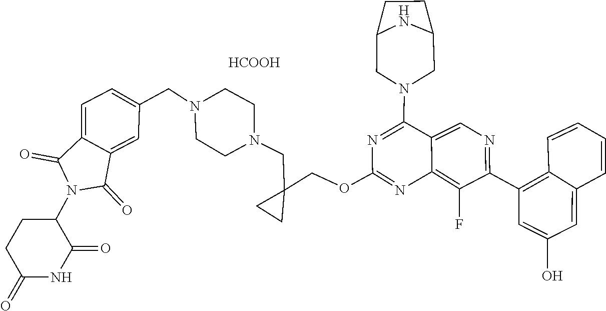 [SMILES: O=C1CCC(N2C(=O)c3ccc(CN4CCN(CC5(COc6nc(N7CC8CCC(C7)N8)c7cnc(-c8cc(O)cc9ccccc89)c(F)c7n6)CC5)CC4)cc3C2=O)C(=O)N1 ; "HCOOH"] | 840.2 | 11.23-11.04 (m, 1H), 9.17 (s, 1H), 8.32- 8.11 (m, 2H), 7.90-7.85 (m, 1H), 7.83- 7.72 (m, 3H), 7.60-7.52 (m, 1H), 7.47- 7.40 (m, 1H), 7.29 (s, 1H), 7.27-7.19 (m, 2H), 5.21-5.05 (m, 1H), 4.48 (d, J = 11.0 Hz, 2H), 4.34-4.26 (m, 2H), 3.61 (s, 4H), 2.95-2.84 (m, 2H), 2.68 (s, 1H), 2.62 (d, J = 1.0 Hz, 1H), 2.57 (d, J = 4.0 Hz, 1H), 2.55 (s, 2H), 2.42-2.36 (m, 4H), 2.32 (s, 4H), 2.09-2.02 (m, 1H), 1.68 (s, 4H), 0.64 (s, 2H), 0.46-0.37 (m, 2H). |
| 12 | 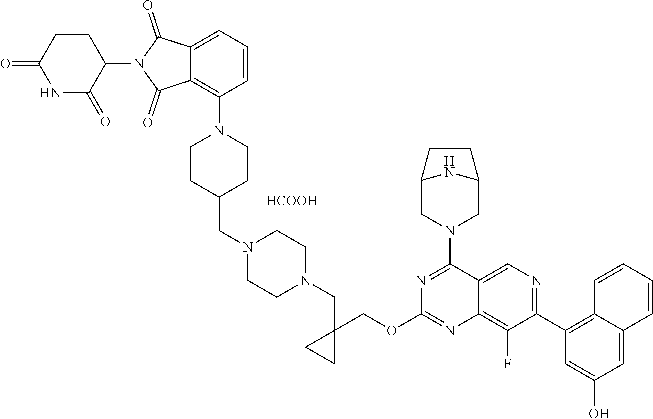 [SMILES: O=C1CCC(N2C(=O)c3cccc(N4CCC(CN5CCN(CC6(COc7nc(N8CC9CCC(C8)N9)c8cnc(-c9cc(O)cc%10ccccc9%10)c(F)c8n7)CC6)CC5)CC4)c3C2=O)C(=O)N1 ; "HCOOH"] | 923.5 | — |
| 13 | 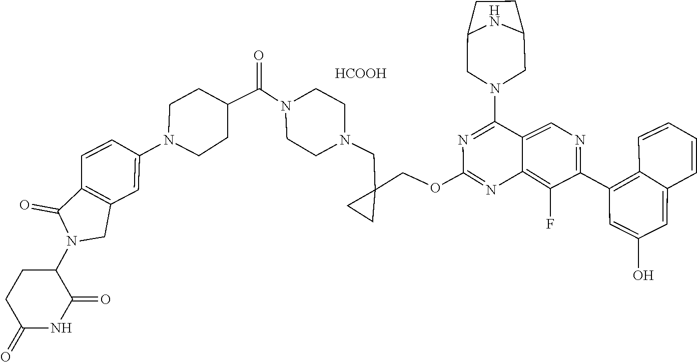 [SMILES: O=C1CCC(N2Cc3cc(N4CCC(C(=O)N5CCN(CC6(COc7nc(N8CC9CCC(C8)N9)c8cnc(-c9cc(O)cc%10ccccc9%10)c(F)c8n7)CC6)CC5)CC4)ccc3C2=O)C(=O)N1 ; "HCOOH"] | 925.3 | 10.94 (s, 1H), 9.17 (s, 1H), 8.19 (s, 1H), 7.81 (d, J = 8.4 Hz, 1H), 7.57-7.42 (m, 3H), 7.30-7.22 (m, 3H), 7.07-7.02 (m, 2H), 5.04 (dd, J = 5.0, 13.3 Hz, 1H), 4.48 (d, J = 11.4 Hz, 2H), 4.36-4.30 (m, 3H), 4.23-4.17 (m, 1H), 3.88 (d, J = 12.6 Hz, 2H), 3.67-3.59 (m, 5H), 3.50 (d, J = 2.0 Hz, 4H), 2.96-2.82 (m, 5H), 2.61 (d, J = 3.0 Hz, 1H), 2.37-2.32 (m, 5H), 1.99-1.93 (m, 1H), 1.68 (s, 4H), 1.66-1.59 (m, 4H), 0.67 (s, 2H), 0.44 (s, 2H). |
| 14 | 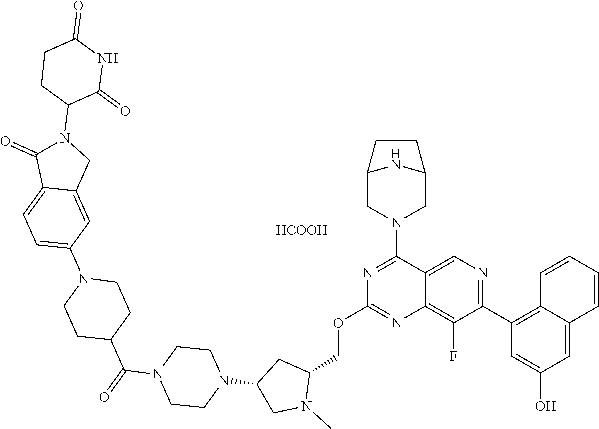 [SMILES: CN1C[C@H](N2CCN(C(=O)C3CCN(c4ccc5c(c4)CN(C4CCC(=O)NC4=O)C5=O)CC3)CC2)C[C@@H]1COc1nc(N2CC3CCC(C2)N3)c2cnc(-c3cc(O)cc4ccccc34)c(F)c2n1 ; "HCOOH"] | 952.4 | 10.94 (s, 1H), 9.18 (s, 1H), 8.18 (s, 1H), 7.80(d, J = 8.2 Hz, 1H), 7.58-7.42 (m, 3H), 7.30-7.22 (m, 3H), 7.06-7.01 (m,2H), 5.05 (dd, J = 5.0, 13.3 Hz, 1H), 4.54-4.44 (m, 3H), 4.42-4.17 (m, 4H), 3.93-3.81 (m, 3H), 3.65 (s, 5H), 3.51 (s, 4H), 3.06-2.99 (m, 2H), 2.93-2.85 (m, 4H), 2.69-2.56 (m, 2H), 2.37-2.29 (m, 8H), 2.14-2.08 (m, 1H), 1.98-1.93 (m, 1H), 1.69 (s, 3H), 1.66 (s, 4H). |
| 15 | 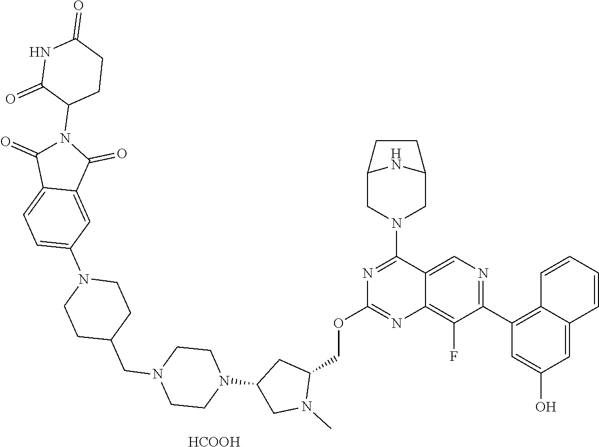 [SMILES: CN1C[C@H](N2CCN(CC3CCN(c4ccc5c(c4)C(=O)N(C4CCC(=O)NC4=O)C5=O)CC3)CC2)C[C@@H]1COc1nc(N2CC3CCC(C2)N3)c2cnc(-c3cc(O)cc4ccccc34)c(F)c2n1 ; "HCOOH"] | 938.3 | 10.95 (s, 1H), 9.18 (s, 1H), 8.21 (s, 2H), 7.80 (d, J = 8.4 Hz, 1H), 7.56 (d, J = 8.4 Hz, 1H), 7.49 (d, J = 9.0 Hz, 1H), 7.44 (t, J = 7.2 Hz, 1H), 7.29 (d, J = 2.0 Hz, 1H), 7.27-7.22 (m, 2H), 7.05-7.00 (m, 2H), 5.04 (dd, J = 5.0, 13.3 Hz, 1H), 4.51- 4.43 (m, 3H), 4.39-4.29 (m, 2H), 4.22- 4.16 (m, 1H), 3.85 (d, J = 12.0 Hz, 3H), 3.66 (s, 2H),3.63 (s, 4H), 3.01 (dd, J = 3.2, 10.1 Hz, 1H), 2.95-2.85 (m, 3H), 2.83-2.76 (m, 2H), 2.69-2.60 (m, 3H), 2.53 (s, 2H), 2.52 (s, 2H), 2.36-2.33 (m, 3H), 2.31 (s, 3H), 2.11 (d, J = 6.6 Hz, 2H), 1.99-1.92 (m, 1H), 1.79-1.72 (m, 3H), 1.68 (s, 3H), 1.63-1.56 (m, 1H), 1.20-1.10 (m, 2H). |
| 16 | 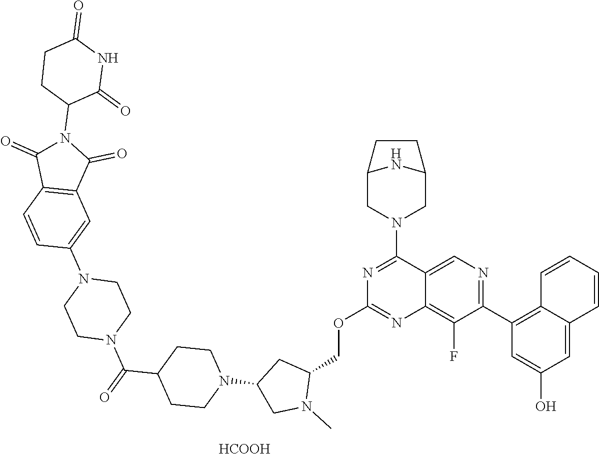 [SMILES: CN1C[C@H](N2CCC(C(=O)N3CCN(c4ccc5c(c4)C(=O)N(C4CCC(=O)NC4=O)C5=O)CC3)CC2)C[C@@H]1COc1nc(N2CC3CCC(C2)N3)c2cnc(-c3cc(O)cc4ccccc34)c(F)c2n1 ; "HCOOH"] | 966.3 | 11.19-11.00 (m, 1H), 9.18 (s, 1H), 8.26 (s, 3H), 7.80 (d, J = 8.4 Hz, 1H), 7.71 (d, J = 8.4 Hz, 1H), 7.56 (br d, J = 8.2 Hz, 1H), 7.44 (t, J = 7.2 Hz, 1H), 7.35 (d, J = 1.9 Hz, 1H), 7.29 (d, J = 2.0 Hz, 1H), 7.25- 7.22 (m, 2H), 5.08 (dd, J = 5.3, 12.9 Hz, 1H), 4.47 (dd, J = 4.8, 10.5 Hz, 3H), 4.37 (dd, J = 5.8, 11.0 Hz, 1H), 3.67-3.58 (m, 9H), 3.06-2.81 (m, 6H), 2.65-2.55 (m, 3H), 2.32 (s, 3H), 2.14-1.98 (m, 5H), 1.70-1.51 (m, 10H). |
| 17 | 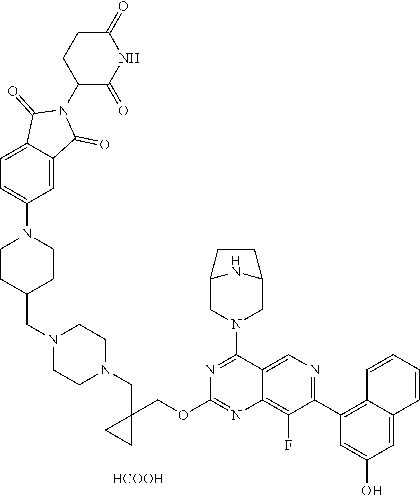 [SMILES: O=C1CCC(N2C(=O)c3ccc(N4CCC(CN5CCN(CC6(COc7nc(N8CC9CCC(C8)N9)c8cnc(-c9cc(O)cc%10ccccc9%10)c(F)c8n7)CC6)CC5)CC4)cc3C2=O)C(=O)N1 ; "HCOOH"] | 923.3 | 11.19 (s, 1H), 9.17 (s, 1H), 8.20 (s, 1H), 7.83-7.78 (m, 1H), 7.67-7.40 (m, 3H), 7.33-7.12 (m, 4H), 5.12-4.93 (m, 1H), 4.54-4.39 (m, 2H), 4.31 (s, 1H), 4.01 (d, J = 12.4 Hz, 2H), 3.69-3.59 (m, 6H), 3.34- 3.24 (m, 3H), 3.00-2.84 (m, 3H), 2.60 (d, J = 2.8 Hz, 2H), 2.35-2.23 (m, 7H), 2.18- 1.97 (m, 4H), 1.95-1.86 (m, 1H), 1.82- 1.65 (m, 5H), 1.60-1.53 (m, 1H), 1.16- 1.03 (m, 2H), 0.64 (s, 1H), 0.41 (s, 1H). |
| 18 | 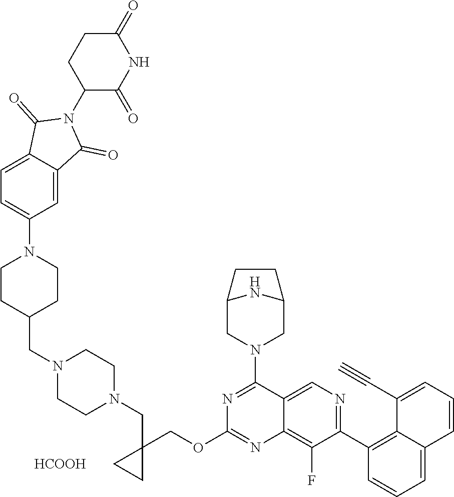 [SMILES: C#Cc1cccc2cccc(-c3ncc4c(N5CC6CCC(C5)N6)nc(OCC5(CN6CCN(CC7CCN(c8ccc9c(c8)C(=O)N(C8CCC(=O)NC8=O)C9=O)CC7)CC6)CC5)nc4c3F)c12 ; "HCOOH"] | 931.5 | 11.08 (s, 1H), 9.04 (s, 1H), 8.25 (s, 1H), 8.18-8.12 (m, 2H), 7.75-7.62 (m, 3H), 7.60-7.54 (m, 2H), 7.31-7.28 (m, 1H), 7.25-7.20 (m, 1H), 5.06 (dd, J = 5.4, 12.6 Hz, 1H), 4.50 (d, J = 10.4 Hz, 1H), 4.33- 4.25 (m, 3H), 4.07-3.98 (m, 2H), 3.69- 3.63 (m, 2H), 3.59-3.52 (m, 4H), 2.99- 2.83 (m, 4H), 2.60 (d, J = 1.9 Hz, 2H), 2.57-2.55 (m, 2H), 2.32-2.24 (m, 4H), 2.13-1.97 (m, 3H), 1.81-1.63 (m, 7H), 1.28-1.04 (m, 3H), 0.64 (s, 2H), 0.41 (s, 2H). |
| 19 | 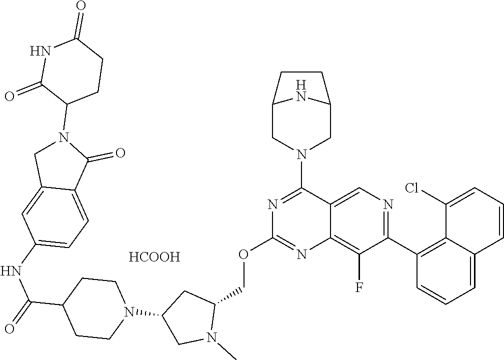 [SMILES: CN1C[C@H](N2CCC(C(=O)Nc3ccc4c(c3)CN(C3CCC(=O)NC3=O)C4=O)CC2)C[C@@H]1COc1nc(N2CC3CCC(C2)N3)c2cnc(-c3cccc4cccc(Cl)c34)c(F)c2n1 ; "HCOOH"] | 901.3 | 11.06-10.88 (m, 1H), 10.23-10.16 (m, 1H), 9.09 (s, 1H), 8.38-8.31 (m, 3H), 8.24-8.17 (m, 1H), 8.13-8.06 (m, 1H), 8.03-7.98 (m, 1H), 7.76-7.69 (m, 1H), 7.67-7.63 (m, 2H), 7.27-7.18 (m, 1H), 5.16-5.01 (m, 1H), 4.47-4.37 (m, 4H), 4.36-4.26 (m, 2H), 3.05-2.85 (m, 8H), 2.61 (d, J = 4.0 Hz, 2H), 2.32 (d, J = 7.8 Hz, 6H), 2.16-2.05 (m, 2H), 2.04-1.93 (m, 4H), 1.81-1.75 (m, 2H), 1.64 (d, J = 4.0 Hz, 5H), 1.27-1.20 (m, 2H). |
| 20 | 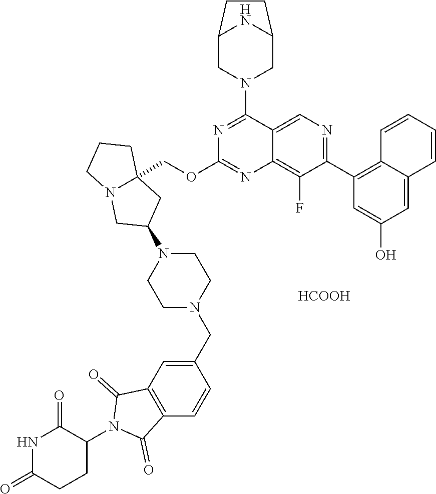 | 895.7 | 11.13 (s, 1H), 9.18 (s, 1H), 8.22 (s, 2H), 7.90-7.85 (m, 1H), 7.80 (d, J = 9.8 Hz, 2H), 7.55 (d, J = 8.4 Hz, 1H), 7.44 (t, J = 7.4 Hz, 1H), 7.29 (d, J = 2.0 Hz, 1H), 7.27-7.21 (m, 2H), 5.15 (dd, J = 5.4, 12.9 Hz, 1H), 4.50 (d, J = 10.6 Hz, 2H), 4.17- 4.00 (m, 5H), 3.65-3.62 (m, 3H), 3.27- 3.21 (m, 1H), 2.95-2.79 (m, 3H), 2.62 (d, J = 2.4 Hz, 1H), 2.58 (s, 1H), 2.55 (s, 1H), 2.53 (d, J = 1.8 Hz, 2H), 2.39 (d, J = 8.3 Hz, 4H), 2.34 (dd, J = 1.8, 3.7 Hz, 2H), 2.31-2.20 (m, 2H), 2.10-1.96 (m, 2H), 1.92-1.64 (m, 8H), 1.42 (t, J = 11.2 Hz, 1H). |
| 21 | 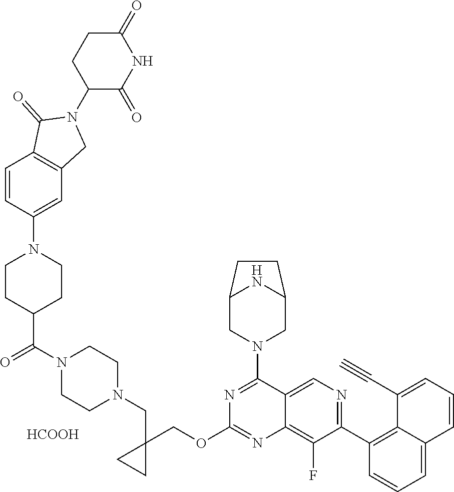 [SMILES: "HCOOH" ; C#Cc1cccc2cccc(-c3ncc4c(N5CC6CCC(C5)N6)nc(OCC5(CN6CCN(C(=O)C7CCN(c8ccc9c(c8)CN(C8CCC(=O)NC8=O)C9=O)CC7)CC6)CC5)nc4c3F)c12] | 931.7 | 10.95 (s, 1H), 9.05 (s, 1H), 8.24 (s, 1H), 8.14 (t, J = 7.8 Hz, 2H), 7.76-7.67 (m, 2H), 7.61-7.54 (m, 2H), 7.54-7.48 (m, 1H), 7.08-7.01 (m, 2H), 5.05 (dd, J = 5.0, 13.7 Hz, 1H), 4.53-4.46 (m, 1H), 4.35- 4.29 (m, 4H), 4.23-4.17 (m, 1H), 3.91- 3.85 (m, 2H), 3.69-3.65 (m, 2H), 3.63 (s, 1H), 3.57 (s, 3H), 3.55-3.50 (m, 4H), 2.94-2.85 (m, 4H), 2.69-2.66 (m, 3H), 2.46-2.42 (m, 2H), 2.34 (dd, J = 1.8, 3.9 Hz, 5H), 2.00-1.91 (m, 1H), 1.67 (s, 5H), 0.66 (s, 2H), 0.46-0.40 (m, 2H). |
| 22 | 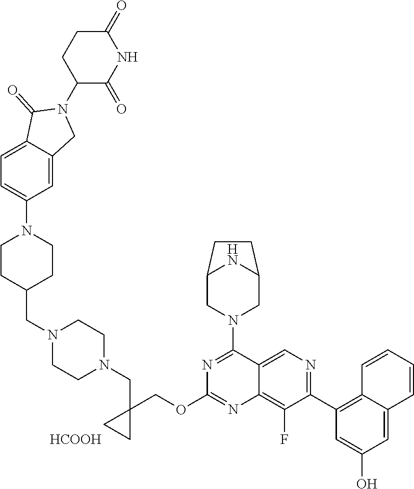 [SMILES: O=C1CCC(N2Cc3cc(N4CCC(CN5CCN(CC6(COc7nc(N8CC9CCC(C8)N9)c8cnc(-c9cc(O)cc%10ccccc9%10)c(F)c8n7)CC6)CC5)CC4)ccc3C2=O)C(=O)N1 ; "HCOOH"] | 909.6 | 10.97-10.92 (m, 1H), 9.16 (s, 1H), 8.27 (s, 1H), 7.83-7.77 (m, 1H), 7.56 (d, J = 7.8 Hz, 1H), 7.51-7.42 (m, 2H), 7.29 (d, J = 2.4 Hz, 1H), 7.25 (s, 1H), 7.23 (d, J = 1.8 Hz, 1H), 7.05-7.00 (m, 2H), 5.04 (dd, J = 4.8, 13.1 Hz, 1H), 4.46 (d, J = 11.6 Hz, 2H), 4.35-4.28 (m, 3H), 4.22-4.16 (m, 1H), 3.84 (d, J = 11.4 Hz, 2H), 3.63 (s, 1H), 3.59 (s, 1H), 3.56 (s, 2H), 2.96-2.75 (m, 4H), 2.71-2.64 (m, 1H), 2.62-2.59 (m, 1H), 2.55 (d, J = 5.4 Hz, 1H), 2.43-2.36 (m, 3H), 2.34 (s, 1H), 2.30 (s, 3H), 2.26 (s, 1H), 2.07 (d, J = 6.4 Hz, 2H), 1.98- 1.92 (m, 1H), 1.73 (d, J = 10.0 Hz, 3H), 1.65 (s, 4H), 1.19-1.08 (m, 2H), 0.64 (s, 2H), 0.41 (s, 2H). |
| 23 | 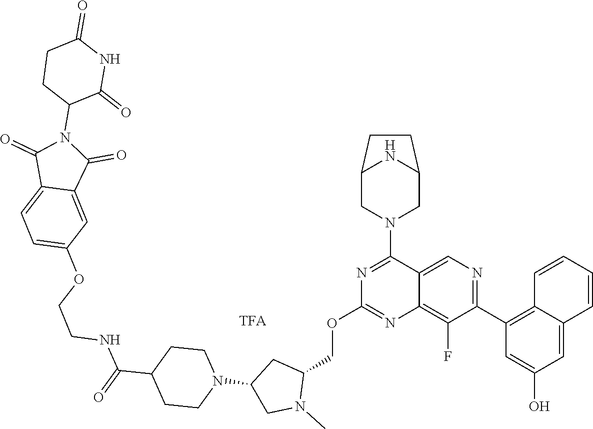 [SMILES: CN1C[C@H](N2CCC(C(=O)NCCOc3ccc4c(c3)C(=O)N(C3CCC(=O)NC3=O)C4=O)CC2)C[C@@H]1COc1nc(N2CC3CCC(C2)N3)c2cnc(-c3cc(O)cc4ccccc34)c(F)c2n1] | 941.3 | 11.12 (s, 1H), 10.15-9.98 (m, 1H), 9.44- 9.36 (m, 1H), 9.30-9.26 (m, 1H), 9.22- 9.11 (m, 1H), 8.30-8.19 (m, 1H), 7.88- 7.80 (m, 2H), 7.54 (d, J = 8.4 Hz, 1H), 7.49-7.42 (m, 2H), 7.37-7.30 (m, 2H), 7.28-7.23 (m, 2H), 5.17-5.06 (m, 1H), 4.74-4.62 (m, 4H), 4.21 (s, 5H), 3.94-3.84 (m, 4H), 2.93-2.86 (m, 4H), 2.74 (s, 2H), 2.68 (d, J = 3.2 Hz, 1H), 2.60-2.53 (m, 4H), 2.10-2.06 (m, 2H), 1.94 (s, 4H), 1.82-1.74 (m, 2H), 1.06 (t, J = 7.0 Hz, 3H). |
| 24 | 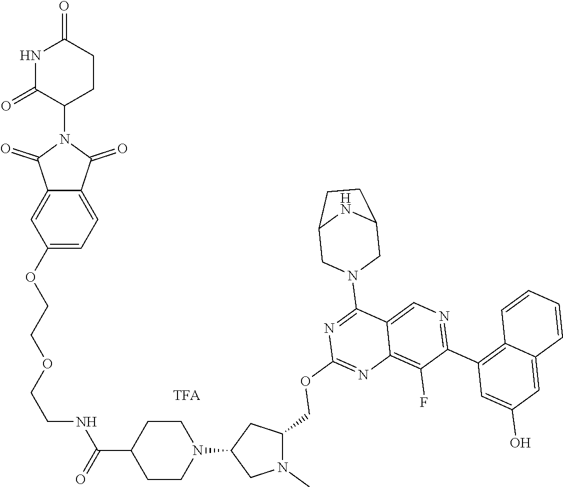 [SMILES: CN1C[C@H](N2CCC(C(=O)NCCOCCOc3ccc4c(c3)C(=O)N(C3CCC(=O)NC3=O)C4=O)CC2)C[C@@H]1COc1nc(N2CC3CCC(C2)N3)c2cnc(-c3cc(O)cc4ccccc34)c(F)c2n1 ; "TFA"] | 985.3 | 11.28 (s, 1H), 9.19-9.15 (m, 1H), 8.30 (s, 2H), 7.86-7.76 (m, 3H), 7.56 (d, J = 8.1 Hz, 1H), 7.46-7.41 (m, 2H), 7.39-7.34 (m, 1H), 7.31-7.20 (m, 4H), 5.18-5.07 (m, 1H), 4.50-4.43 (m, 3H), 4.35-4.28 (m, 3H), 3.76 (s, 3H), 3.22-3.19 (m, 3H), 3.01-2.76 (m, 9H), 2.64-2.56 (m, 3H), 2.31 (s, 4H), 2.10-1.99 (m, 4H), 1.95 (s, 3H), 1.66 (s, 4H). |
| 25 | 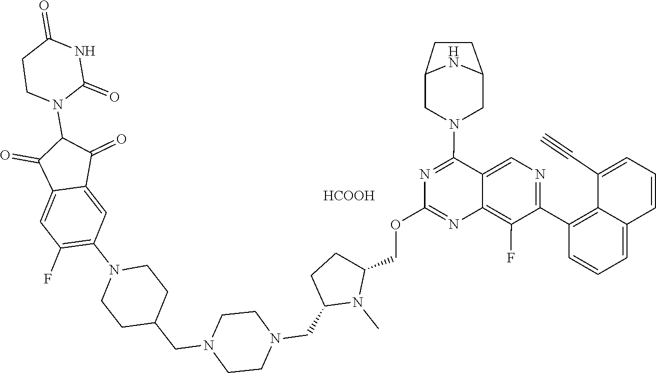 [SMILES: C#Cc1cccc2cccc(-c3ncc4c(N5CC6CCC(C5)N6)nc(OC[C@H]5CC[C@@H](CN6CCN(CC7CCN(c8cc9c(cc8F)C(=O)C(N8CCC(=O)NC8=O)C9=O)CC7)CC6)N5C)nc4c3F)c12 ; "HCOOH"] | 992.3 | 11.19-11.04 (m, 1H), 9.05 (s, 1H), 8.25 (s, 3H), 8.14 (t, J = 8.2 Hz, 2H), 7.75-7.67 (m, 3H), 7.62-7.54 (m, 2H), 7.45-7.40 (m, 1H), 5.20-5.02 (m, 1H), 4.54-4.45 (m, 1H), 4.42-4.30 (m, 3H), 4.18-4.10 (m, 2H), 3.67 (d, J = 2.5 Hz, 2H), 3.60-3.59 (m, 2H), 2.92-2.82 (m, 4H), 2.79-2.73 (m, 1H), 2.63-2.56 (m, 2H), 2.42-2.40 (m, 4H), 2.23-2.12 (m, 5H), 2.09-1.98 (m, 2H), 1.93-1.83 (m, 3H), 1.79 (d, J = 13.3 Hz, 3H), 1.68 (s, 5H), 1.64-1.43 (m, 4H), 1.32-1.16 (m, 5H). |
| 26 | 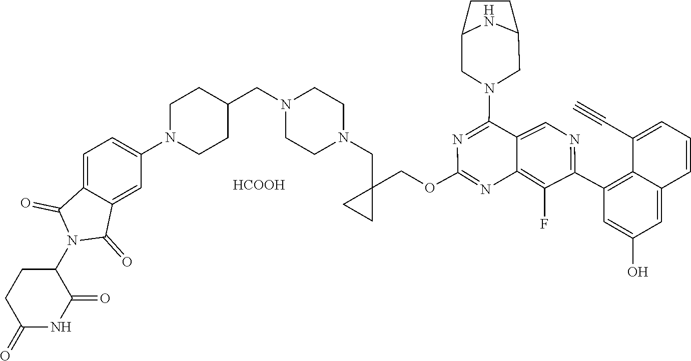 [SMILES: C#Cc1cccc2cc(O)cc(-c3ncc4c(N5CC6CCC(C5)N6)nc(OCC5(CN6CCN(CC7CCN(c8ccc9c(c8)C(=O)N(C8CCC(=O)NC8=O)C9=O)CC7)CC6)CC5)nc4c3F)c12 ; "HCOOH"] | 947.5 | 11.07 (s, 1H), 9.06-9.01 (m, 1H), 8.20 (s, 1H), 7.89 (d, J = 7.8 Hz, 1H), 7.65 (d, J = 8.4 Hz, 1H), 7.49-7.39 (m, 2H), 7.34 (d, J = 2.5 Hz, 1H), 7.29 (s, 1H), 7.22 (d, J = 8.4 Hz, 1H), 7.12 (d, J = 2.4 Hz, 1H), 5.06 (dd, J = 5.2, 13.3 Hz, 1H), 4.57-4.43 (m, 1H), 4.35-4.21 (m, 3H), 4.05-3.98 (m, 2H), 3.74-3.63 (m, 2H), 3.62-3.55 (m, 4H), 3.00-2.83 (m, 4H), 2.62-2.59 (m, 1H), 2.58-2.54 (m, 3H), 2.31-2.25 (m, 4H), 2.12-2.06 (m, 2H), 2.04-1.92 (m, 2H), 1.81-1.66 (m, 6H), 1.26-1.23 (m, 1H), 1.18-1.03 (m, 3H), 0.64 (s, 2H), 0.41 (s, 2H). |
| 27 | 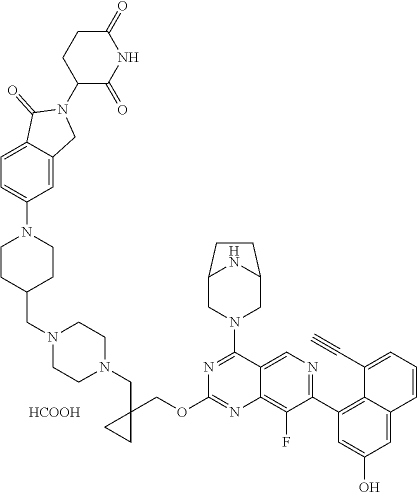 [SMILES: C#Cc1cccc2cc(O)cc(-c3ncc4c(N5CC6CCC(C5)N6)nc(OCC5(CN6CCN(CC7CCN(c8ccc9c(c8)CN(C8CCC(=O)NC8=O)C9=O)CC7)CC6)CC5)nc4c3F)c12 ; "HCOOH"] | 933.6 | 10.94 (s, 1H), 9.02 (s, 1H), 8.19 (s, 1H), 7.90-7.86 (m, 1H), 7.50-7.39 (m, 3H), 7.33 (d, J = 2.4 Hz, 1H), 7.10 (d, J = 2.4 Hz, 1H), 7.05-6.99 (m, 2H), 5.03 (dd, J = 5.0, 13.2 Hz, 1H), 4.56-4.47 (m, 1H), 4.34-4.23 (m, 4H), 4.20-4.15 (m, 1H), 3.85 (s, 1H), 3.83 (s, 1H), 3.68 (s, 1H), 3.66-3.60 (m, 3H), 3.59 (s, 1H), 3.54 (s, 1H), 2.94-2.87 (m, 1H), 2.87-2.74 (m, 3H), 2.59 (s, 1H), 2.56 (d, J = 3.4 Hz, 1H), 2.36 (d, J = 4.6 Hz, 2H), 2.34-2.24 (m, 6H), 2.09 (d, J = 6.4 Hz, 2H), 1.94 (dd, J = 4.8, 9.8 Hz, 1H), 1.76-1.65 (m, 7H), 1.13 (d, J = 11.4 Hz, 2H), 0.68-0.58 (m, 2H), 0.43-0.36 (m, 2H). |
| 28 | 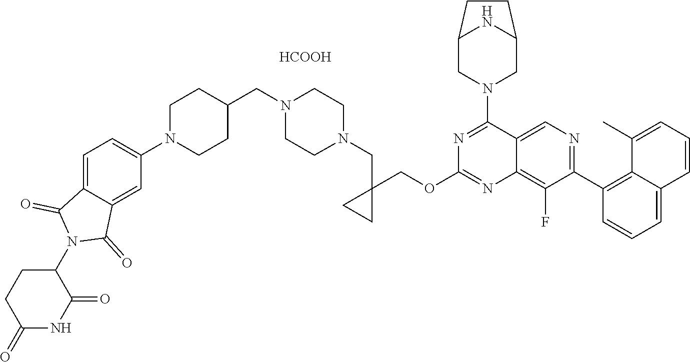 [SMILES: Cc1cccc2cccc(-c3ncc4c(N5CC6CCC(C5)N6)nc(OCC5(CN6CCN(CC7CCN(c8ccc9c(c8)C(=O)N(C8CCC(=O)NC8=O)C9=O)CC7)CC6)CC5)nc4c3F)c12 ; "HCOOH"] | 921.5 | 9.09 (s, 1H), 8.56-8.46 (m, 1H), 8.10-8.02 (m, 1H), 7.89 (d, J = 8.1 Hz, 1H), 7.70- 7.64 (m, 1H), 7.61-7.56 (m, 1H), 7.49- 7.42 (m, 2H), 7.35 (s, 2H), 7.21 (dd, J = 1.8, 8.7 Hz, 1H), 5.13-5.04 (m, 2H), 4.77- 4.64 (m, 3H), 4.52-4.41 (m, 2H), 4.03 (d, J = 12.4 Hz, 2H), 3.88-3.73 (m, 5H), 3.55-3.49 (m, 1H), 3.24-3.22 (m, 1H), 3.03-2.94 (m, 3H), 2.90-2.82 (m, 3H), 2.78 (d, J = 2.6 Hz, 2H), 2.72-2.67 (m, 3H), 2.42-2.34 (m, 2H), 2.08-2.05 (m, 3H), 1.99-1.95 (m, 2H), 1.92-1.86 (m, 3H), 1.39-1.27 (m, 5H), 0.83-0.77 (m, 2H), 0.65-0.57 (m, 2H). |
| 29 | 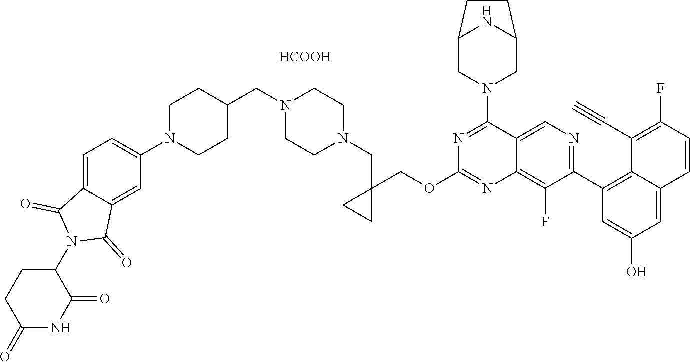 [SMILES: C#Cc1c(F)ccc2cc(O)cc(-c3ncc4c(N5CC6CCC(C5)N6)nc(OCC5(CN6CCN(CC7CCN(c8ccc9c(c8)C(=O)N(C8CCC(=O)NC8=O)C9=O)CC7)CC6)CC5)nc4c3F)c12 ; "HCOOH"] | 965.5 | 11.14-11.04 (m, 1H), 9.04 (s, 1H), 8.18 (s, 1H), 7.98 (dd, J = 6.0, 8.9 Hz, 1H), 7.65 (d, J = 8.6 Hz, 1H), 7.47 (t, J = 9.0 Hz, 1H), 7.40 (d, J = 2.0 Hz, 1H), 7.32-7.27 (m, 1H), 7.26-7.15 (m, 2H), 5.11-5.02 (m, 1H), 4.59-4.44 (m, 1H), 4.36-4.20(m, 3H), 4.06-3.90 (m, 3H), 3.69-3.54 (m, 5H), 3.02-2.81 (m, 4H), 2.69-2.59 (m, 2H), 2.36-2.24 (m, 7H), 2.11-2.07 (m, 2H), 2.05-1.96 (m, 2H), 1.79-1.67 (m, 7H), 1.30-1.21 (m, 2H), 1.15-1.05 (m, 2H), 0.64 (s, 2H), 0.41 (s, 2H). |
| 30 | 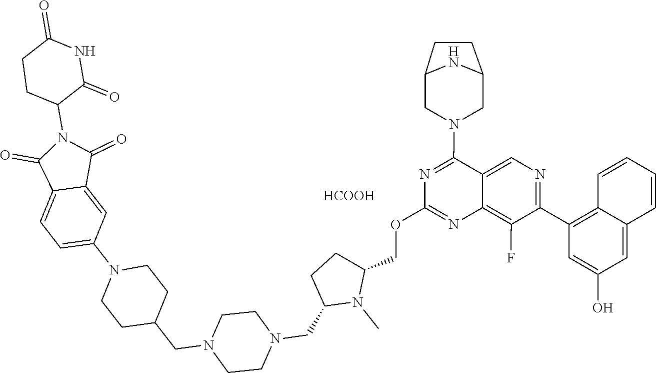 [SMILES: CN1[C@@H](COc2nc(N3CC4CCC(C3)N4)c3cnc(-c4cc(O)cc5ccccc45)c(F)c3n2)CC[C@H]1CN1CCN(CC2CCN(c3ccc4c(c3)C(=O)N(C3CCC(=O)NC3=O)C4=O)CC2)CC1 ; "HCOOH"] | 966.8 | 11.09 (s, 1H), 9.18 (s, 1H), 8.20 (s, 2H), 7.81 (d, J = 8.4 Hz, 1H), 7.65 (d, J = 8.4 Hz, 1H), 7.56 (d, J = 8.0 Hz, 1H), 7.44 (t, J = 7.4 Hz, 1H), 7.32-7.18 (m, 5H), 5.07 (dd, J = 5.6, 12.9 Hz, 1H), 4.54-4.35 (m, 4H), 4.20 (dd, J = 6.4, 10.3 Hz, 1H), 4.02 (d, J = 12.10Hz, 2H), 3.68-3.61 (m, 5H), 2.99-2.84 (m, 4H), 2.79-2.70 (m, 1H), 2.69-2.60 (m, 3H), 2.43-2.39 (m, 5H), 2.26 (s, 2H), 2.18 (dd, J = 6.4, 12.1 Hz, 2H), 2.12-2.08 (m, 2H), 2.06-1.97 (m, 2H), 1.93-1.82 (m, 3H), 1.75 (d, J = 12.4 Hz, 3H), 1.68 (s, 3H), 1.63-1.56 (m, 1H), 1.53-1.44 (m, 1H), 1.17-1.01 (m, 3H). |
| 31 | 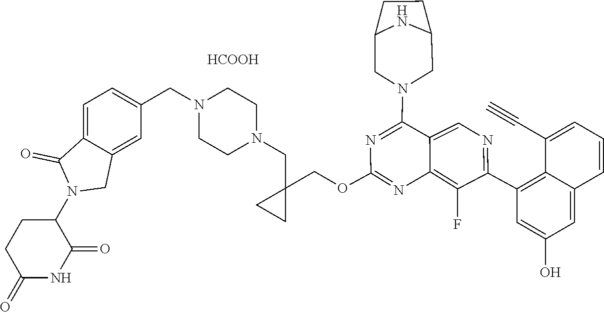 [SMILES: C#Cc1cccc2cc(O)cc(-c3ncc4c(N5CC6CCC(C5)N6)nc(OCC5(CN6CCN(Cc7ccc8c(c7)CN(C7CCC(=O)NC7=O)C8=O)CC6)CC5)nc4c3F)c12 ; "HCOOH"] | 850.3 | 11.16-11.08 (m, 1H), 8.98 (s, 1H), 8.24 (s, 3H), 7.98-7.90 (m, 1H), 7.88-7.86 (m, 1H), 7.82 (s, 1H), 7.77 (d, J = 7.8 Hz, 1H), 7.58 (d, J = 6.4 Hz, 1H), 7.52-7.46 (m, 1H), 7.36 (d, J = 2.4 Hz, 1H), 7.30- 7.26 (m, 1H), 5.16-5.12 (m, 1H), 4.42- 4.34 (m, 2H), 4.30 (s, 2H), 3.60 (d, J = 14.6 Hz, 7H), 3.44 (q, J = 7.0 Hz, 4H), 2.94-2.88 (m, 1H), 2.69-2.59 (m, 1H), 2.34 (s, 3H), 2.28 (s, 3H), 2.08 (s, 2H), 1.66 (s, 1H), 1.62 (s, 1H), 1.06 (t, J = 7.0 Hz, 3H), 0.66 (s, 2H), 0.46-0.37 (m, 2H). |
| 32 | 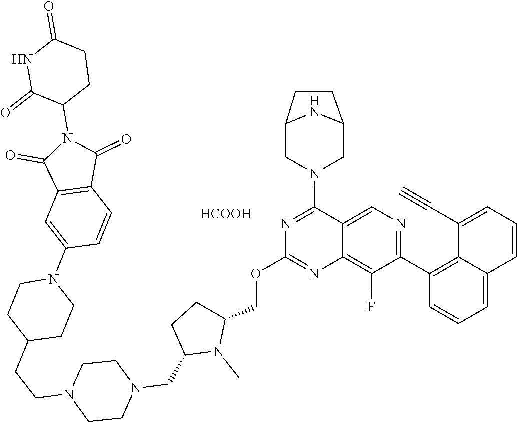 [SMILES: C#Cc1cccc2cccc(-c3ncc4c(N5CC6CCC(C5)N6)nc(OC[C@H]5CC[C@@H](CN6CCN(CCC7CCN(c8ccc9c(c8)C(=O)N(C8CCC(=O)NC8=O)C9=O)CC7)CC6)N5C)nc4c3F)c12 ; "HCOOH"] | 988.50 | 11.19-10.98 (m, 1H), 9.11 8.99 (m, 1H), 8.32-8.28 (m, 3H), 8.19-8.11 (m, 2H), 7.75-7.63 (m, 3H), 7.61-7.54 (m, 2H), 7.32-7.28 (m, 1H), 7.22 (d, J = 8.6 Hz, 1H), 5.07 (dd, J = 5.6, 12.9 Hz, 1H), 4.53- 4.44 (m, 1H), 4.40-4.30 (m, 2H), 4.17- 4.09 (m, 1H), 4.02 (d, J = 13.1 Hz, 3H), 3.69-3.66 (m, 2H), 3.57 (s, 5H), 2.99-2.83 (m, 4H), 2.78-2.70 (m, 1H), 2.62-2.58 (m, 3H), 2.41 (s, 3H), 2.31-2.25 (m, 4H), 2.17 (dd, J = 7.4, 12.1 Hz, 2H), 2.07-1.97 (m, 2H), 1.91-1.82 (m, 2H), 1.77-1.65 (m, 6H), 1.62-1.44 (m, 4H), 1.40-1.32 (m, 2H), 1.26-1.11 (m, 3H). |
| 33 | 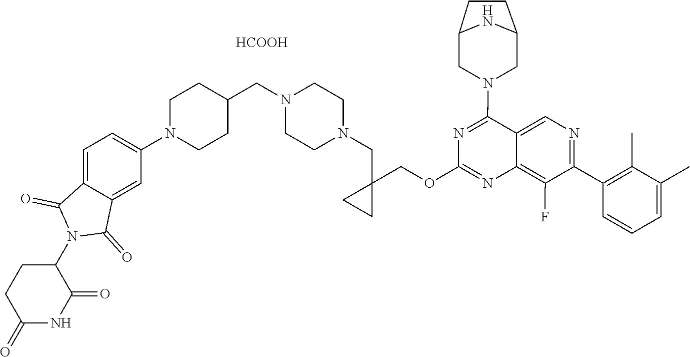 [SMILES: Cc1cccc(-c2ncc3c(N4CC5CCC(C4)N5)nc(OCC4(CN5CCN(CC6CCN(c7ccc8c(c7)C(=O)N(C7CCC(=O)NC7=O)C8=O)CC6)CC5)CC4)nc3c2F)c1C ; "HCOOH"] | 885.9 | 11.07 (s, 1H), 9.09 (s, 1H), 8.21 (s, 1H), 7.64 (d, J = 8.4 Hz, 1H), 7.34-7.15 (m, 5H), 5.06 (dd, J = 5.2, 12.8 Hz, 1H), 4.43 (d, J = 11.8 Hz, 2H), 4.29 (s, 2H), 4.01 (d, J = 12.1 Hz, 3H), 3.60 (d, J = 10.4 Hz, 6H), 3.03-2.81 (m, 5H), 2.34-2.25 (m, 8H), 2.12-1.95 (m, 7H), 1.84-1.60 (m, 8H), 1.18-1.05 (m, 2H), 0.63 (s, 2H), 0.40 (s, 2H). |
| 34 | 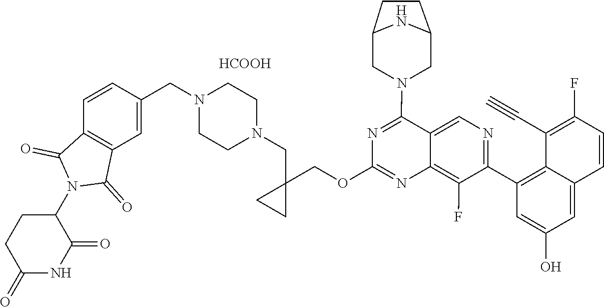 [SMILES: C#Cc1c(F)ccc2cc(O)cc(-c3ncc4c(N5CC6CCC(C5)N6)nc(OCC5(CN6CCN(Cc7ccc8c(c7)C(=O)N(C7CCC(=O)NC7=O)C8=O)CC6)CC5)nc4c3F)c12 ; "HCOOH"] | 882.4 | 11.13 (s, 1H), 9.04 (s, 1H), 8.21 (s, 2H), 7.98 (dd, J = 6.0, 9.0 Hz, 1H), 7.87 (d, J = 7.6 Hz, 1H), 7.82-7.75 (m, 2H), 7.47 (t, J = 9.0 Hz, 1H), 7.40 (d, J = 2.4 Hz, 1H), 7.18 (d, J = 2.4 Hz,1H), 5.15 (dd, J = 5.2, 12.8 Hz, 1H), 4.51 (d, J = 12.6 Hz, 1H), 4.34-4.24(m, 3H), 3.95 (s, 1H), 3.63 (s, 6H), 2.95-2.81 (m, 2H), 2.69-2.59 (m, 3H), 2.41-2.27 (m, 8H), 2.11-1.99 (m, 2H), 1.70 (s, 4H), 0.63 (s, 2H), 0.41 (s, 2H). |
| 35 | 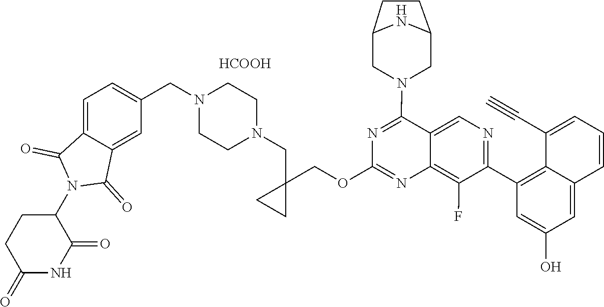 [SMILES: C#Cc1cccc2cc(O)cc(-c3ncc4c(N5CC6CCC(C5)N6)nc(OCC5(CN6CCN(Cc7ccc8c(c7)C(=O)N(C7CCC(=O)NC7=O)C8=O)CC6)CC5)nc4c3F)c12 ; "HCOOH"] | 864.5 | 11.25-11.00 (m, 1H), 9.00 (s, 1H), 8.27 (s, 1H), 7.91-7.85 (m, 2H), 7.82-7.77 (m, 2H), 7.48-7.39 (m, 2H), 7.34 (d, J = 2.4 Hz, 1H), 7.12 (d, J = 2.4 Hz, 1H), 5.15 (dd, J = 5.4, 13.0 Hz, 1H), 4.53-4.46 (m, 1H), 4.26 (s, 2H), 3.63 (s, 3H), 3.59 (s, 1H), 3.57 (s, 3H), 3.53 (s, 1H), 2.95-2.82 (m, 2H), 2.69-2.67 (m, 1H), 2.62 (d, J = 2.0 Hz, 1H), 2.58 (s, 1H), 2.43-2.36 (m, 5H), 2.35-2.33 (m, 2H), 2.32 (s, 2H), 1.72-1.61 (m, 4H), 0.63 (s, 2H), 0.41 (s, 2H). |
| 36 | 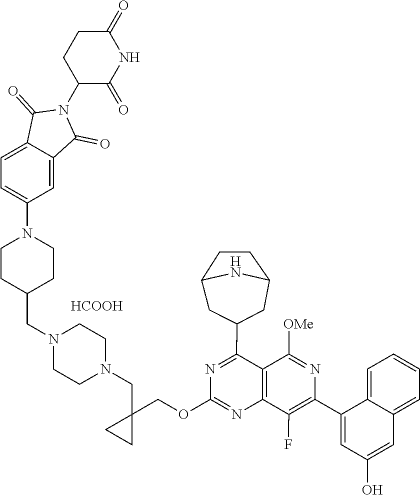 [SMILES: COc1nc(-c2cc(O)cc3ccccc23)c(F)c2nc(OCC3(CN4CCN(CC5CCN(c6ccc7c(c6)C(=O)N(C6CCC(=O)NC6=O)C7=O)CC5)CC4)CC3)nc(C3CC4CCC(C3)N4)c12 ; "HCOOH"] | 953.6 | 11.04 (s, 1H), 8.32-8.13 (m, 1H), 7.75 (d, J = 8.2 Hz, 1H), 7.68 (d, J = 8.0 Hz, 1H), 7.60 (d, J = 8.6 Hz, 1H), 7.42-7.37 (m, 1H), 7.25-7.21 (m, 4H), 7.18-7.15 (m, 1H), 5.02 (dd, J = 5.2, 12.8 Hz, 1H), 4.25 (s, 2H), 3.98 (s, 1H), 3.95 (s, 1H), 3.91 (s, 3H), 3.46 (s, 4H), 3.37 (s, 2H), 3.34 (s, 2H), 2.94-2.78 (m, 4H), 2.63 (dd, J = 1.8, 3.6 Hz, 1H), 2.29 (td, J = 1.8, 3.6 Hz, 2H), 2.27-2.22 (m, 4H), 2.05-1.95 (m, 4H), 1.69 (d, J = 10.8 Hz, 4H), 1.55 (s, 4H), 1.10-1.02 (m, 2H), 0.58 (s, 2H), 0.35 (s, 2H). |
| 37 | 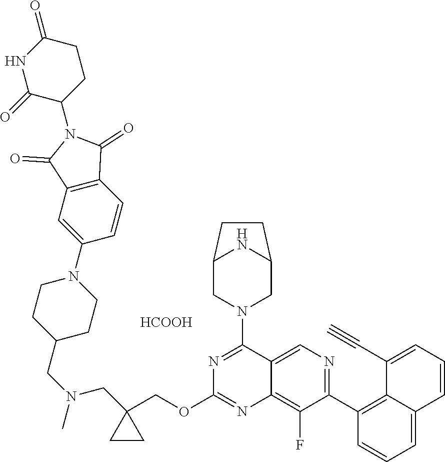 [SMILES: C#Cc1cccc2cccc(-c3ncc4c(N5CC6CCC(C5)N6)nc(OCC5(CN(C)CC6CCN(c7ccc8c(c7)C(=O)N(C7CCC(=O)NC7=O)C8=O)CC6)CC5)nc4c3F)c12 ; "HCOOH"] | 876.7 | 11.08 (s, 1H), 9.05 (s, 1H), 8.22 (s, 1H), 8.13 (t, J = 7.4 Hz, 2H), 7.76-7.64 (m, 2H), 7.59-7.49 (m, 3H), 7.17 (d, J = 1.8 Hz, 1H), 7.03 (d, J = 8.6 Hz, 1H), 5.09- 5.02 (m, 1H), 4.46 (d, J = 11.6 Hz, 1H), 4.37-4.29 (m, 2H), 4.18 (d, J = 10.4 Hz, 1H), 3.83 (t, J = 11.8 Hz, 3H), 3.57 (d, J = 11.0 Hz, 6H), 2.86-2.74 (m, 3H), 2.57- 2.53 (m, 2H), 2.23-2.18 (m, 4H), 2.15 (d, J = 6.4 Hz, 2H), 2.03-1.96 (m, 1H), 1.72 (d, J = 11.4 Hz, 3H), 1.63 (s, 3H), 1.09- 0.98 (m, 2H), 0.66-0.58 (m, 2H), 0.44- 0.36 (m, 2H). |
| 38 | 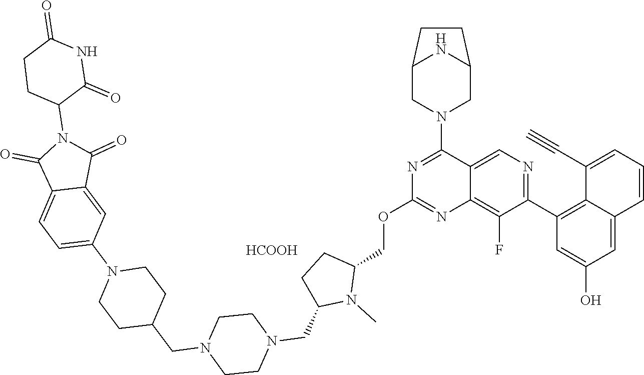 [SMILES: C#Cc1cccc2cc(O)cc(-c3ncc4c(N5CC6CCC(C5)N6)nc(OC[C@H]5CC[C@@H](CN6CCN(CC7CCN(c8ccc9c(c8)C(=O)N(C8CCC(=O)NC8=O)C9=O)CC7)CC6)N5C)nc4c3F)c12 ; "HCOOH"] | 991.7 | 11.19-0.95 (m, 1H), 9.09-8.97 (m, 1H), 8.19 (s, 2H), 7.89 (d, J = 8.5 Hz, 1H), 7.68-7.62 (m, 1H), 7.48-7.40 (m, 2H), 7.35 (d, J = 2.4 Hz, 1H), 7.30 (s, 1H), 7.22 (dd, J = 2.2, 8.8 Hz, 1H), 7.12 (d, J = 2.4 Hz, 1H), 5.10-5.02 (m, 1H), 4.56-4.44 (m, 1H), 4.41-4.25 (m, 3H), 4.19-4.11 (m, 1H), 4.02 (d, J = 13.4 Hz, 2H), 3.65 (t, J = 14.8 Hz, 5H), 3.59 (d, J = 2.6 Hz, 2H), 2.99-2.84 (m, 4H), 2.80-2.72 (m, 1H), 2.63-2.55 (m, 2H), 2.42 (s, 5H), 2.26 (s, 1H), 2.23-2.16 (m, 2H), 2.11 (d, J = 7.0 Hz, 2H), 2.02 (dt, J = 5.0, 10.0 Hz, 2H), 1.96-1.83 (m, 3H), 1.80-1.69 (m, 6H), 1.64-1.54 (m, 2H), 1.52-1.42 (m, 1H), 1.17-1.07 (m, 2H). |
| 39 | 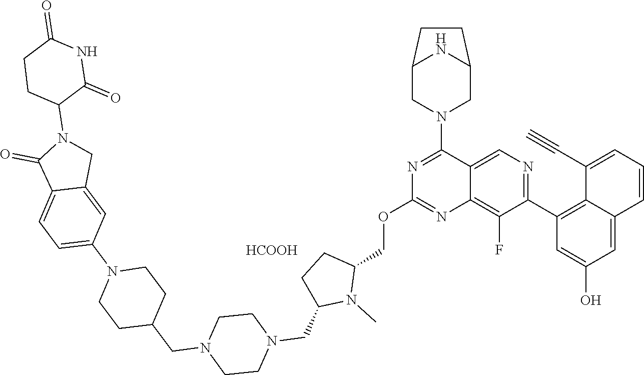 [SMILES: C#Cc1cccc2cc(O)cc(-c3ncc4c(N5CC6CCC(C5)N6)nc(OC[C@H]5CC[C@@H](CN6CCN(CC7CCN(c8ccc9c(c8)CN(C8CCC(=O)NC8=O)C9=O)CC7)CC6)N5C)nc4c3F)c12 ; "HCOOH"] | 976.8 | 10.93 (s, 1H), 9.04 (s, 1H), 8.19 (s, 2H), 7.88 (d, J = 7.8 Hz, 1H), 7.50-7.39 (m, 2H), 7.34 (d, J = 2.4 Hz, 1H), 7.12 (d, J = 2.4 Hz, 1H), 7.06-6.98 (m, 2H), 5.03 (dd, J = 5.0, 13.2 Hz, 1H), 4.64-4.42 (m, 2H), 4.40-4.28 (m, 3H), 4.23-4.08 (m, 3H), 3.84 (d, J = 12.6 Hz, 2H), 3.76-3.54 (m, 5H), 3.27 (s, 1H), 2.97-2.70 (m, 4H), 2.62-2.51 (m, 3H), 2.45-2.29 (m, 10H), 2.26 (s, 1H), 2.23-2.09 (m, 3H), 1.99-1.82 (m, 3H), 1.74 (s, 5H), 1.62-1.45 (m, 2H), 1.25-1.06 (m, 2H). |
| 40 | 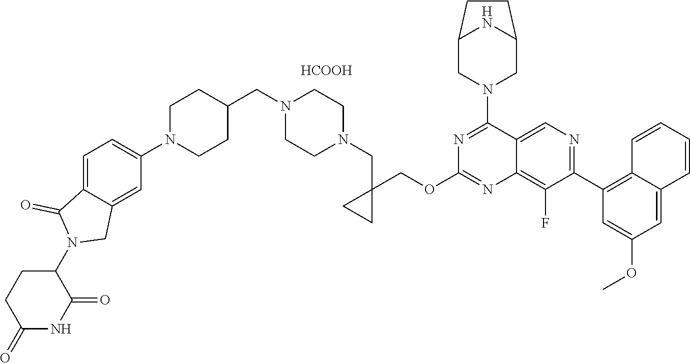 [SMILES: COc1cc(-c2ncc3c(N4CC5CCC(C4)N5)nc(OCC4(CN5CCN(CC6CCN(c7ccc8c(c7)CN(C7CCC(=O)NC7=O)C8=O)CC6)CC5)CC4)nc3c2F)c2ccccc2c1 ; "HCOOH"] | 923.8 | 10.93 (s, 1H), 9.17 (s, 1H), 8.19 (s, 1H), 7.94 (d, J = 8.3 Hz, 1H), 7.61 (d, J = 8.6 Hz, 1H), 7.55-7.46 (m, 3H), 7.39-7.26 (m, 2H), 7.06-6.98 (m, 2H), 5.03 (dd, J = 5.0 Hz, 1H), 4.47 (d, J = 10.8 Hz, 2H), 4.34- 4.27 (m, 3H), 4.21-4.16 (m, 1H), 3.94 (s, 3H), 3.83 (d, J = 12.4 Hz, 3H), 3.69-3.58 (m, 6H), 2.94-2.84 (m, 1H), 2.78 (t, J = 11.6 Hz, 2H), 2.62-2.52 (m, 2H), 2.42- 2.27 (m, 8H), 2.06 (d, J = 6.4 Hz, 2H), 1.98-1.91 (m, 1H), 1.77-1.63 (m, 7H), 1.20-1.06 (m, 2H), 0.64 (s, 2H), 0.41 (s, 2H). |
| 41 | 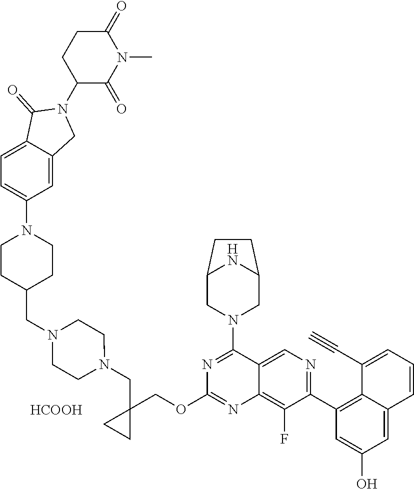 [SMILES: C#Cc1cccc2cc(O)cc(-c3ncc4c(N5CC6CCC(C5)N6)nc(OCC5(CN6CCN(CC7CCN(c8ccc9c(c8)CN(C8CCC(=O)N(C)C8=O)C9=O)CC7)CC6)CC5)nc4c3F)c12 ; "HCOOH"] | 948.6 | 10.23-10.11 (m, 1H), 9.11-9.05 (m, 1H), 8.14 (s, 1H), 7.89 (dd, J = 1.8, 7.9 Hz, 1H), 7.50 (d, J = 9.2 Hz, 1H), 7.47-7.40 (m, 2H), 7.35 (d, J = 2.4 Hz, 1H), 7.13 (d, J = 2.4 Hz, 1H), 7.05-7.01 (m, 2H), 5.10 (dd, J = 5.0, 13.4 Hz, 1H), 4.65 (d, J = 12.9 Hz, 1H), 4.44 (d, J = 13.4 Hz, 1H), 4.34-4.27 (m, 3H), 4.20 (s, 1H), 4.12 (s, 2H), 3.89-3.78 (m, 4H), 3.62-3.58 (m, 1H), 3.32 (s, 8H), 2.99 (s, 3H), 2.83-2.75 (m, 3H), 2.72 (d, J = 2.4 Hz, 1H), 2.68- 2.65 (m, 1H), 2.38 (d, J = 5.0 Hz, 2H), 2.35-2.30 (m, 2H), 2.21 (dd, J = 3.9, 5.6 Hz, 1H), 1.98-1.91 (m, 4H), 1.75 (d, J = 10.6 Hz, 3H), 1.16 (q, J = 11.0 Hz, 2H), 0.66 (s, 2H), 0.45 (s, 2H). |
| 42 | 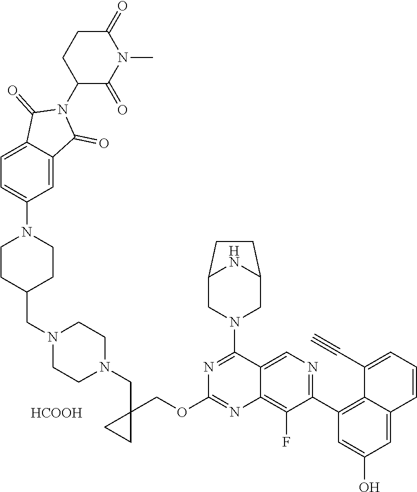 [SMILES: C#Cc1cccc2cc(O)cc(-c3ncc4c(N5CC6CCC(C5)N6)nc(OCC5(CN6CCN(CC7CCN(c8ccc9c(c8)C(=O)N(C8CCC(=O)N(C)C8=O)C9=O)CC7)CC6)CC5)nc4c3F)c12 ; "HCOOH"] | 961.4 | 9.03 (s, 1H), 8.20 (s,2H), 7.89 (dd, J = 1.2, 7.8 Hz, 1H), 7.65 (d, J = 8.4 Hz, 1H), 7.49-7.39 (m, 2H), 7.36-7.29 (m, 2H), 7.25-7.20 (m, 1H), 7.12 (d, J = 2.4 Hz, 1H), 5.13 (dd, J = 5.0, 12.8 Hz, 1H), 4.51 (d, J = 11.2 Hz, 1H), 4.33-4.22 (m, 3H), 4.08-3.94 (m, 3H), 3.70-3.64 (m, 2H), 3.61-3.58 (m, 3H), 3.02 (s, 3H), 2.98 (s, 4H), 2.81-2.71 (m, 2H), 2.56 (d, J = 5.4 Hz, 2H), 2.45-2.36 (m, 4H), 2.32-2.26 (m, 4H), 2.14-1.99 (m, 4H), 1.80-1.74 (m, 3H), 1.69 (s, 2H), 1.18-1.07 (m, 2H), 0.65 (s, 2H), 0.41 (s, 2H). |
| 43 | 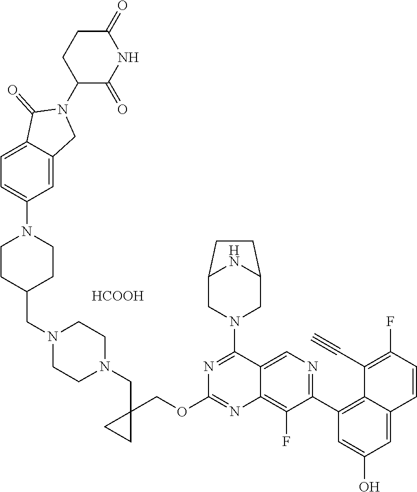 [SMILES: C#Cc1c(F)ccc2cc(O)cc(-c3ncc4c(N5CC6CCC(C5)N6)nc(OCC5(CN6CCN(CC7CCN(c8ccc9c(c8)CN(C8CCC(=O)NC8=O)C9=O)CC7)CC6)CC5)nc4c3F)c12 ; "HCOOH"] | 951.7 | 9.04 (s, 1H), 8.45 (s, 1H), 7.86 (dd, J = 5.9, 9.3 Hz, 1H), 7.61 (d, J = 8.2 Hz, 1H), 7.38-7.27 (m, 2H), 7.22 (d, J = 2.4 Hz, 1H), 7.08-7.01 (m, 2H), 5.10 (dd, J = 5.2, 13.4 Hz, 1H), 4.69 (t, J = 14.0 Hz, 2H), 4.54 (d, J = 11.2 Hz, 1H), 4.41-4.37 (m, 2H), 3.97-3.84 (m, 4H), 3.81 (d, J = 14.0 Hz, 2H), 3.43-3.34 (m, 3H), 2.96-2.70 (m, 13H), 2.58-2.39 (m, 4H), 2.18-2.12 (m, 1H), 2.03-1.92 (m, 3H), 1.83 (d, J = 11.9 Hz, 3H), 1.35-1.26 (m, 2H), 0.82 (s, 2H), 0.63 (s, 2H). |
| 44 | 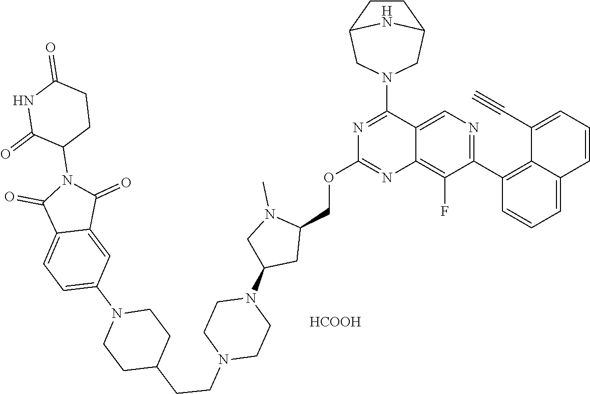 [SMILES: C#Cc1cccc2cccc(-c3ncc4c(N5CC6CCC(C5)N6)nc(OC[C@H]5C[C@@H](N6CCN(CCC7CCN(c8ccc9c(c8)C(=O)N(C8CCC(=O)NC8=O)C9=O)CC7)CC6)CN5C)nc4c3F)c12 ; "HCOOH"] | 974.4 | 11.07 (s, 1H), 9.05 (s, 1H), 8.21 (s, 2H), 8.14 (t, J = 7.6 Hz, 2H), 7.74-7.68 (m, 2H), 7.64 (d, J = 8.6 Hz, 1H), 7.61- 7.53 (m, 2H), 7.30-7.17 (m, 2H), 5.06 (dd, J = 5.3, 12.8 Hz, 1H), 4.52-4.40 (m,2H), 4.37-4.29 (m, 2H), 4.02 (d, J = 11.6 Hz, 3H), 3.69-3.63 (m, 5H), 3.60 (s, 5H), 3.03-2.96 (m, 2H), 2.95-2.81 (m, 5H), 2.69-2.66 (m, 1H), 2.66-2.54 (m, 3H), 2.33 (td, J = 2.0, 3.8 Hz, 3H), 2.31 (s, 3H), 2.12-1.97 (m, 3H), 1.69 (s, 3H), 1.63-1.55 (m, 2H), 1.39-1.31 (m, 2H), 1.26-1.10 (m, 3H). |
| 45 | 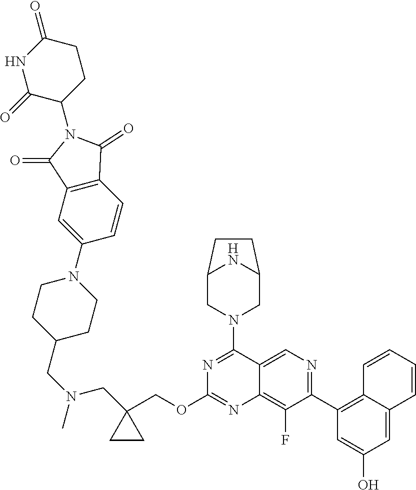 [SMILES: CN(CC1CCN(c2ccc3c(c2)C(=O)N(C2CCC(=O)NC2=O)C3=O)CC1)CC1(COc2nc(N3CC4CCC(C3)N4)c3cnc(-c4cc(O)cc5ccccc45)c(F)c3n2)CC1] | 868.3 | 11.07 (s, 1H), 9.18 (s, 1H), 8.20 (s, 1H), 7.79 (d, J = 8.4 Hz, 1H), 7.58-7.48 (m, 2H), 7.42 (t, J = 7.4 Hz, 1H), 7.28 (d, J = 2.1 Hz, 1H), 7.23-7.13 (m, 3H), 7.00 (dd, J = 2.1, 8.6 Hz, 1H), 5.04 (dd, J = 5.4, 12.8 Hz, 1H), 4.46 (d, J = 11.6 Hz, 2H), 4.29 (s, 2H), 3.84-3.78 (m, 2H), 3.60 (d, J = 11.8 Hz, 3H), 3.55 (s, 2H), 2.92-2.72 (m, 4H), 2.54 (s, 2H), 2.31 (s, 1H), 2.21 (s, 3H), 2.15 (d, J = 6.4 Hz, 2H), 2.02- 1.94 (m, 1H), 1.71 (d, J = 12.0 Hz, 3H), 1.60 (s, 3H), 1.07-0.95 (m, 2H), 0.63 (s, 2H), 0.41 (s, 2H). |
| 46 | 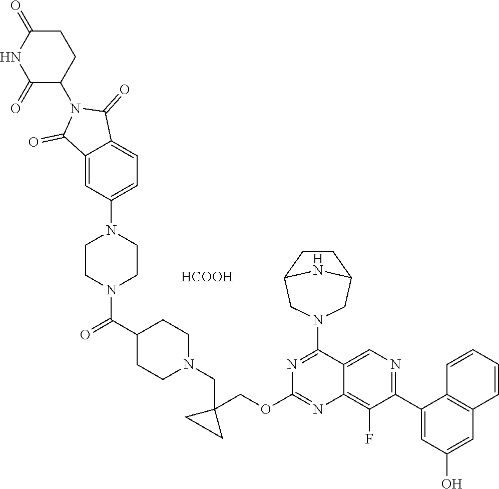 [SMILES: O=C1CCC(N2C(=O)c3ccc(N4CCN(C(=O)C5CCN(CC6(COc7nc(N8CC9CCC(C8)N9)c8cnc(-c9cc(O)cc%10ccccc9%10)c(F)c8n7)CC6)CC5)CC4)cc3C2=O)C(=O)N1 ; "HCOOH"] | 937.4 | 11.08 (s, 1H), 9.98 (s, 1H), 9.19 (s, 1H), 8.15 (s, 1H), 7.81 (d, J = 7.8 Hz, 1H), 7.74-7.69 (m, 1H), 7.56 (s, 1H), 7.47-7.42 (m, 1H), 7.35 (d, J = 1.8 Hz, 1H), 7.29 (d, J = 2.0 Hz, 1H), 7.27-7.22 (m, 3H), 5.08 (dd, J = 5.4, 12.9 Hz, 1H), 4.57-4.50 (m, 2H), 4.36-4.29 (m, 2H), 3.82-3.74 (m, 2H), 3.71 (s, 1H), 3.67 (d, J = 6.8 Hz, 2H), 3.62-3.58 (m, 2H), 3.52-3.48 (m, 3H), 3.44 (d, J = 4.8 Hz, 5H), 3.08-2.96 (m, 3H), 2.95-2.83 (m, 2H), 2.70-2.66 (m, 2H), 2.34 (dd, J = 1.8, 3.6 Hz, 2H), 2.06- 1.92 (m, 4H), 1.77 (s, 4H), 1.63-1.58 (m, 3H), 1.29-1.22 (m, 2H), 0.72-0.63 (m, 2H), 0.49-0.38 (m, 2H). |
| 47 | 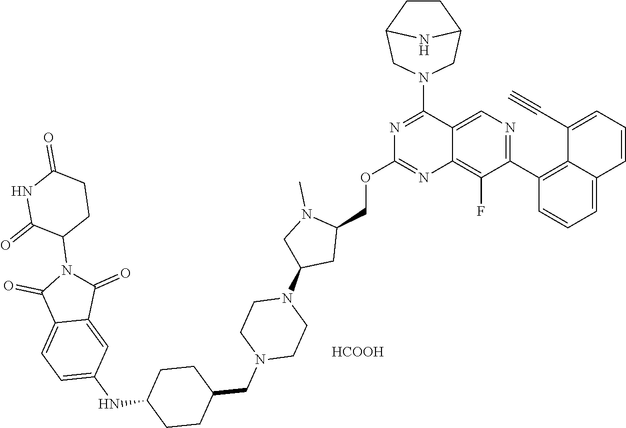 | 974.4 | 11.09-11.02 (m, 1H), 9.05 (s, 1H), 8.20 (s, 1H), 8.17-8.12 (m, 2H), 7.76-7.66 (m, 2H), 7.63-7.50 (m, 3H), 7.02-6.93 (m, 2H), 6.88-6.81 (m, 1H), 5.02 (dd, J = 5.4, 12.4 Hz, 1H), 4.53-4.40 (m, 2H), 4.38- 4.27 (m, 2H), 3.69-3.63(m, 2H), 3.59 (s, 3H), 3.06-2.97 (m, 1H), 2.94-2.81 (m, 2H), 2.69-2.65 (m, 2H), 2.47 (td, J = 2.0, 4.0 Hz, 3H), 2.34 (dd, J = 1.8, 3.6 Hz, 4H), 2.31 (s, 3H), 2.07 (d, J = 6.8 Hz, 3H), 2.03-1.94 (m, 4H), 1.85-1.76 (m, 3H), 1.68 (s, 4H), 1.62-1.42 (m, 3H), 1.28-1.12 (m, 3H), 1.09-0.92 (m, 3H). |
| 48 | 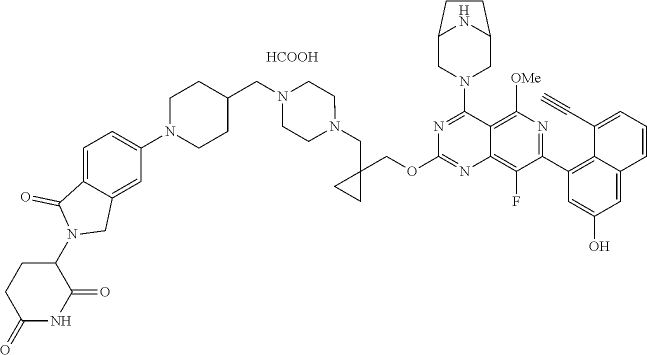 [SMILES: C#Cc1cccc2cc(O)cc(-c3nc(OC)c4c(N5CC6CCC(C5)N6)nc(OCC5(CN6CCN(CC7CCN(c8ccc9c(c8)CN(C8CCC(=O)NC8=O)C9=O)CC7)CC6)CC5)nc4c3F)c12 ; "HCOOH"] | 963.8 | 11.01 (s, 1H), 8.26-8.18 (m, 1H), 7.88 (d, J = 8.2 Hz, 1H), 7.52-7.46 (m, 2H), 7.45- 7.39 (m, 1H), 7.36-7.30 (m, 1H), 7.17 (d, J = 2.2 Hz, 1H), 7.07-7.00 (m, 2H), 5.04 (dd, J = 4.8, 13.3 Hz, 1H), 4.35-4.24 (m, 3H), 4.19 (d, J = 16.8 Hz, 2H), 3.88 (s, 3H), 3.84 (s, 2H), 3.55 (d, J = 9.2 Hz, 5H), 3.20 (d, J = 2.0 Hz, 2H), 2.86-2.75 (m, 3H), 2.55 (s, 3H), 2.32 (d, J = 12.4 Hz, 6H), 2.23 (s, 2H), 2.13-2.06 (m, 3H), 2.00-1.92 (m, 2H), 1.91-1.85 (m, 1H), 1.77-1.71 (m, 3H), 1.68-1.63 (m, 2H), 1.28-1.21 (m, 1H), 1.18-1.10 (m, 2H), 0.63 (s, 2H), 0.40 (s, 2H). |
| 49 | 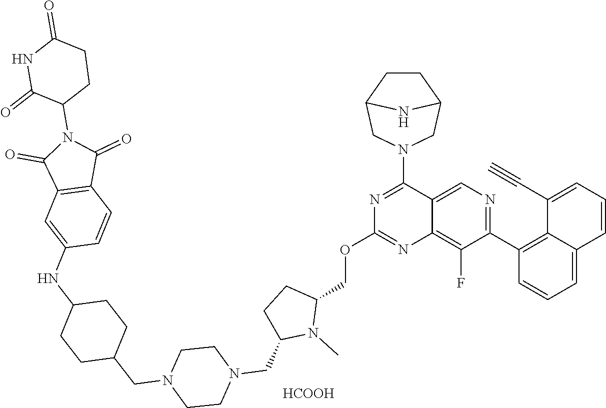 [SMILES: C#Cc1cccc2cccc(-c3ncc4c(N5CC6CCC(C5)N6)nc(OC[C@H]5CC[C@@H](CN6CCN(CC7CCC(Nc8ccc9c(c8)C(=O)N(C8CCC(=O)NC8=O)C9=O)CC7)CC6)N5C)nc4c3F)c12 ; "HCOOH"] | 988.6 | 11.06 (s, 1H), 9.10-9.00 (m, 1H), 8.20 (s, 1H), 8.15 (t, J = 8.0 Hz, 2H), 7.75-7.66 (m, 2H), 7.62-7.50 (m, 3H), 6.99 (J = 7.8 Hz, 1H), 6.94 (s, 1H), 6.87-6.82 (m, 1H), 5.09-4.97 (m, 1H), 4.49 (J = 11.8 Hz, 1H), 4.40-4.30 (m, 2H), 4.19-4.09 (m, 1H), 3.67 (d, J = 3.0 Hz, 1H), 3.65-3.56 (m, 6H), 3.47 (1H), 2.96-2.79 (m, 2H), 2.78-2.70 (m, 1H), 2.62-2.55 (m, 2H), 2.41 (s, 6H), 2.18 (J = 6.6, 11.9 Hz, 2H), 2.10-2.06 (m, 2H), 1.98 (J = 3.4, 10.8 Hz, 3H), 1.93-1.75 (m, 5H), 1.68 (4H), 1.63- 1.53 (m, 2H), 1.52-1.41 (m, 2H), 1.24 (m, 2H), 1.09-0.96 (m, 3H). |
| 50 | 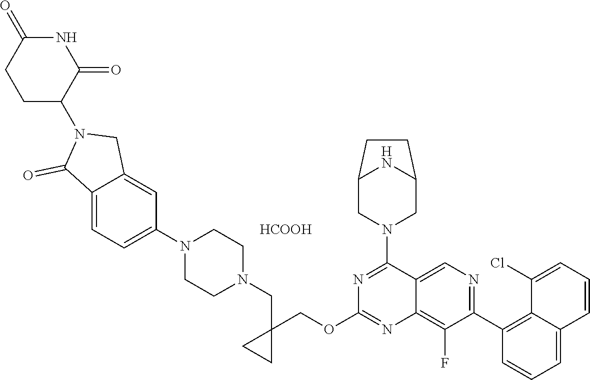 [SMILES: O=C1CCC(N2Cc3cc(N4CCN(CC5(COc6nc(N7CC8CCC(C7)N8)c7cnc(-c8cccc9cccc(Cl)c89)c(F)c7n6)CC5)CC4)ccc3C2=O)C(=O)N1 ; "HCOOH"] | 830.4 | 11.00-10.91 (m, 1H), 9.11-9.04 (m, 1H), 8.24 (s, 1H), 8.20-8.16 (m, 1H), 8.09 (d, J = 7.6 Hz, 1H), 7.72-7.65 (m, 1H), 7.63- 7.49 (m, 4H), 7.05-6.98 (m, 2H), 5.14- 4.97 (m, 1H), 4.49-4.16 (m, 7H), 3.64- 3.54 (m, 5H), 3.22 (s, 5H), 2.98-2.85 (m, 1H), 2.61 (s, 2H), 2.43-2.35 (m, 3H), 2.01-1.91 (m, 1H), 1.67-1.60 (m, 4H), 0.68 (s, 2H), 0.48 (s, 2H). |
| 51 | 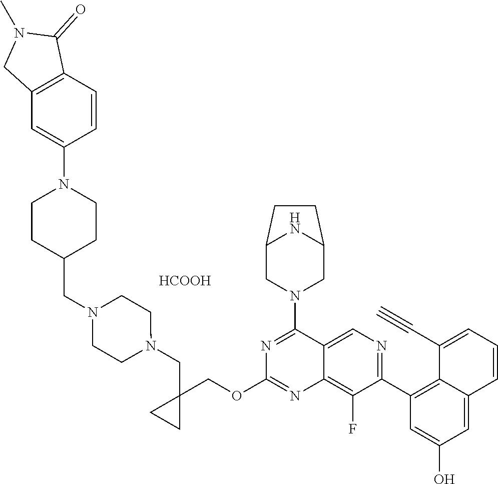 [SMILES: C#Cc1cccc2cc(O)cc(-c3ncc4c(N5CC6CCC(C5)N6)nc(OCC5(CN6CCN(CC7CCN(c8ccc9c(c8)CN(C)C9=O)CC7)CC6)CC5)nc4c3F)c12 ; "HCOOH"] | 836.4 | 9.03 (s, 1H), 8.20 (s, 1H), 7.89 (dd, J = 1.6, 8.0 Hz, 1H), 7.49-7.39 (m, 3H), 7.34 (d, J = 2.6 Hz, 1H), 7.12 (d, J = 2.6 Hz, 1H), 7.04-6.96 (m, 2H), 4.55-4.48 (m, 1H), 4.34-4.21 (m, 6H), 3.82 (d, J = 12.0 Hz, 3H), 3.67 (d, J = 12.8 Hz, 2H), 3.63 (s, 2H), 3.60-3.58 (m, 2H), 3.00 (s, 3H), 2.82-2.64 (m, 3H), 2.35-2.26 (m, 9H), 2.10 (d, J = 7.0 Hz, 2H), 1.77-1.68 (m, 6H), 1.18-1.10 (m, 2H), 0.64 (s, 2H), 0.41 (s, 2H). |
| 52 | 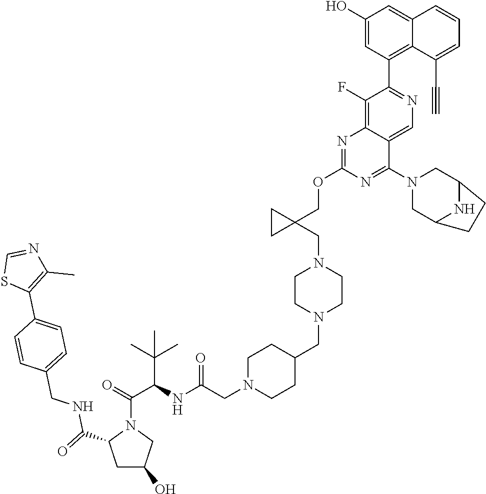 [SMILES: C#Cc1cccc2cc(O)cc(-c3ncc4c(N5CC6CCC(C5)N6)nc(OCC5(CN6CCN(CC7CCN(CC(=O)N[C@@H](C(=O)N8C[C@@H](O)C[C@@H]8C(=O)NCc8ccc(-c9scnc9C)cc8)C(C)(C)C)CC7)CC6)CC5)nc4c3F)c12] | 1162.3 | 10.16 (s, 1H), 9.02 (s, 1H), 8.99-8.96 (m, 1H), 8.60 (t, J = 6.0 Hz, 1H), 7.90-7.87 (m, 1H), 7.79 (d, J = 9.4 Hz, 1H), 7.47- 7.43 (m, 2H), 7.41 (d, J = 4.6 Hz, 4H), 7.34 (d, J = 2.4 Hz, 1H), 7.12 (d, J = 2.4 Hz, 1H), 5.17-5.09 (m, 1H), 4.50 (d, J = 9.6 Hz, 2H), 4.46-4.39 (m, 2H), 4.39-4.34 (m, 2H), 4.30-4.24 (m, 4H), 3.69-3.63 (m, 2H), 3.61 (s, 1H), 3.58 (s, 1H), 3.55 (s, 2H), 3.52 (s, 1H), 3.03-2.96 (m, 1H), 2.89 (s, 1H), 2.82-2.75 (m, 2H), 2.69-2.66 (m, 1H), 2.44 (s, 3H), 2.35-2.32 (m, 2H), 2.26 (dd, J = 9.2, 12.6 Hz, 7H), 2.11-2.01 (m, 6H), 1.67 (s, 6H), 1.13-1.06 (m, 2H), 0.94 (s, 9H), 0.92 (s, 1H), 0.66-0.61 (m, 2H), 0.40 (s, 2H). |
| 53 | 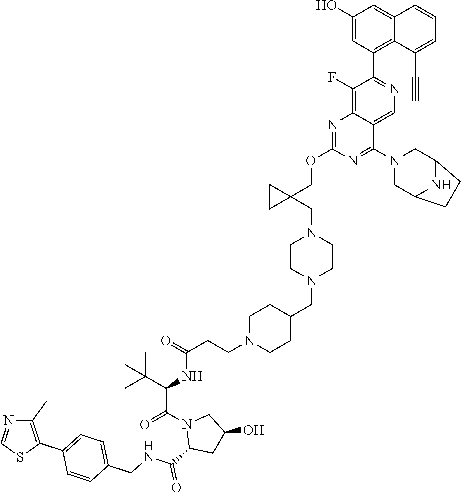 [SMILES: C#Cc1cccc2cc(O)cc(-c3ncc4c(N5CC6CCC(C5)N6)nc(OCC5(CN6CCN(CC7CCN(CCC(=O)N[C@@H](C(=O)N8C[C@@H](O)C[C@@H]8C(=O)NCc8ccc(-c9scnc9C)cc8)C(C)(C)C)CC7)CC6)CC5)nc4c3F)c12] | 1176.3 | 10.22-10.08 (m, 1H), 9.02 (s, 1H), 8.97 (s, 1H), 8.85 (d, J = 9.8 Hz, 1H), 8.63 (t, J = 5.9 Hz, 1H), 7.89 (d, J = 8.0 Hz, 1H), 7.45 (d, J = 6.6 Hz, 3H), 7.43-7.40 (m, 1H), 7.38 (s, 1H), 7.36 (s, 1H), 7.34 (d, J = 2.4 Hz, 1H), 7.12 (d, J = 2.4 Hz, 1H), 5.16-5.10 (m, 1H), 4.55 (d, J = 9.8 Hz, 1H), 4.48 (s, 1H), 4.47-4.41 (m, 2H), 4.36 (s, 1H), 4.29 (s, 1H), 4.24 (s, 2H), 4.20 (d, J = 4.3 Hz, 1H), 3.66 (s, 1H), 3.63 (s, 1H), 3.58 (s, 1H), 3.54 (s, 2H), 2.96-2.84 (m, 2H), 2.70-2.66 (m, 1H), 2.44 (s, 3H), 2.40 (s, 2H), 2.35-2.33 (m, 2H), 2.29-2.21 (m, 6H), 2.20-2.16 (m, 2H), 2.08-1.98 (m, 4H), 1.96-1.87 (m, 3H), 1.82 (d, J = 11.4 Hz, 1H), 1.66 (s, 6H), 1.51-1.35 (m, 2H), 1.26-1.06 (m, 3H), 0.95 (s, 9H), 0.65-0.60 (m, 2H), 0.39 (s, 2H). |
| 54 | 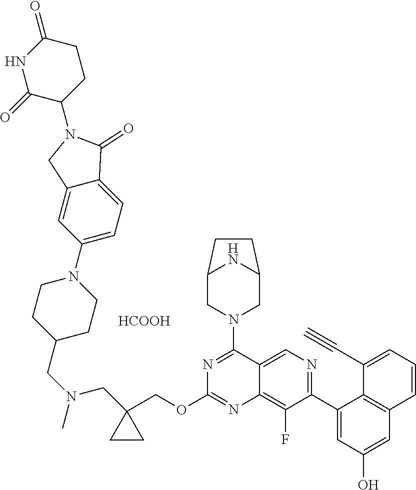 [SMILES: C#Cc1cccc2cc(O)cc(-c3ncc4c(N5CC6CCC(C5)N6)nc(OCC5(CN(C)CC6CCN(c7ccc8c(c7)CN(C7CCC(=O)NC7=O)C8=O)CC6)CC5)nc4c3F)c12 ; "HCOOH"] | 878.7 | 10.94 (s, 1H), 9.02 (d, J = 4.0 Hz, 1H), 8.21 (s, 1H), 7.88 (d, J = 8.0 Hz, 1H), 7.47-7.38 (m, 3H), 7.33 (s, 1H), 7.14-7.09 (m, 1H), 6.88-6.79 (m, 2H), 5.05-4.97 (m, 1H), 4.43 (dd, J = 3.8, 10.0 Hz, 2H), 4.32 (d, J = 14.4 Hz, 1H), 4.20-3.98 (m, 4H), 3.67-3.62 (m, 3H), 3.59 (d, J = 2.8 Hz, 3H), 3.52 (s, 1H), 2.98-2.80 (m, 2H), 2.69-2.62 (m, 2H), 2.54 (s, 2H), 2.25-2.13 (m, 8H), 1.90 (dd, J = 5.4, 10.2 Hz, 1H), 1.72-1.66 (m, 3H), 1.63 (s, 3H), 1.10-0.98 (m, 2H), 0.63 (s, 2H), 0.40 (d, J = 4.4 Hz, 2H). |
| 55 | 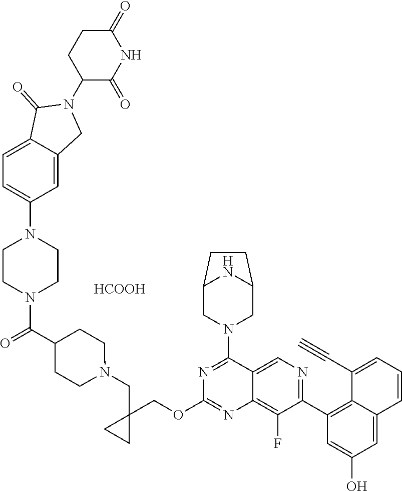 [SMILES: C#Cc1cccc2cc(O)cc(-c3ncc4c(N5CC6CCC(C5)N6)nc(OCC5(CN6CCC(C(=O)N7CCN(c8ccc9c(c8)CN(C8CCC(=O)NC8=O)C9=O)CC7)CC6)CC5)nc4c3F)c12 ; "HCOOH"] | 947.5 | 11.05-10.80 (m, 1H), 9.02 (s, 1H), 8.22 (s, 1H), 7.91-7.84 (m, 1H), 7.57-7.52 (m, 1H), 7.48-7.39 (m, 2H), 7.34 (d, J = 2.4 Hz, 1H), 7.13 (d, J = 2.6 Hz, 1H), 7.10- 7.05 (m, 2H), 5.09-4.99 (m, 1H), 4.54- 4.47 (m, 1H), 4.39-4.17 (m, 6H), 3.67- 3.55 (m, 10H), 3.01-2.99 (m, 1H), 2.37 (dd, J = 3.4, 13.2 Hz, 3H), 2.30-2.23 (m, 2H), 2.01-1.92 (m, 4H), 1.72-1.64 (m, 5H), 1.62-1.55 (m, 4H), 1.29-1.20 (m, 3H), 0.71-0.61 (m, 2H), 0.45-0.36 (m, 2H). |
| 56 | 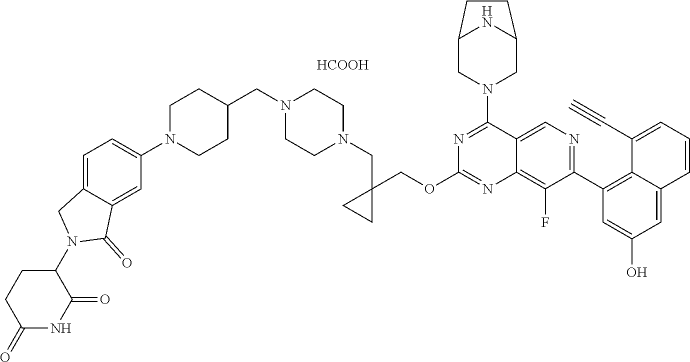 [SMILES: C#Cc1cccc2cc(O)cc(-c3ncc4c(N5CC6CCC(C5)N6)nc(OCC5(CN6CCN(CC7CCN(c8ccc9c(c8)C(=O)N(C8CCC(=O)NC8=O)C9)CC7)CC6)CC5)nc4c3F)c12 ; "HCOOH"] | 933.6 | 11.02-10.88 (m, 1H), 9.06-9.01 (m, 1H), 8.20 (s, 1H), 7.92-7.86 (m, 1H), 7.49-7.38 (m, 3H), 7.34 (d, J = 2.6 Hz, 1H), 7.27- 7.21 (m, 1H), 7.15-7.10 (m, 2H), 5.14- 5.03 (m, 1H), 4.57-4.44 (m, 1H), 4.39- 4.14 (m, 6H), 3.76-3.63 (m, 6H), 3.61- 3.56 (m, 5H), 2.97-2.85 (m, 2H), 2.70- 2.63 (m, 4H), 2.30 (d, J = 9.6 Hz, 3H), 2.14-2.07 (m, 2H), 2.04-1.93 (m, 2H), 1.69 (s, 7H), 1.27-1.12 (m, 3H), 0.68-0.61 (m, 2H), 0.48-0.37 (m, 2H). |
| 57 | 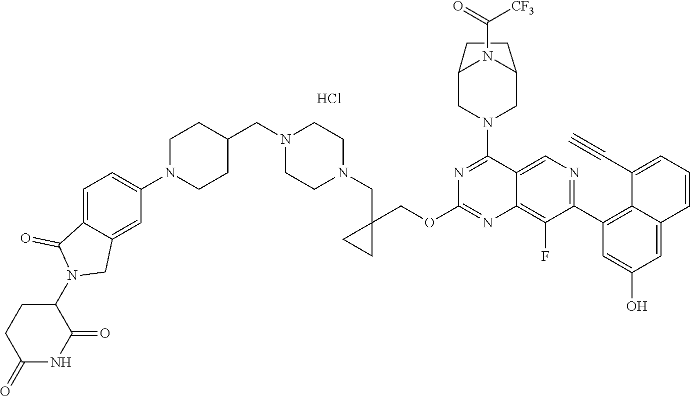 [SMILES: C#Cc1cccc2cc(O)cc(-c3ncc4c(N5CC6CCC(C5)N6C(=O)C(F)(F)F)nc(OCC5(CN6CCN(CC7CCN(c8ccc9c(c8)CN(C8CCC(=O)NC8=O)C9=O)CC7)CC6)CC5)nc4c3F)c12 ; Cl] | 1029.9 | 10.94 (s, 1H), 10.18 (s, 1H), 9.06 (s, 1H), 8.13 (s, 1H), 7.89 (d, J = 7.0 Hz, 1H), 7.55-7.38 (m, 3H), 7.35 (d, J = 2.6 Hz, 1H), 7.12 (t, J = 2.4 Hz, 1H), 7.06-7.00 (m, 2H), 5.04 (dd, J = 5.0, 13.2 Hz, 1H), 4.80 (s, 1H), 4.73 (d, J = 12.4 Hz, 1H), 4.64 (d, J = 5.9 Hz, 1H), 4.56-4.47 (m, 1H), 4.35-4.26 (m, 3H), 4.23-4.14 (m, 1H), 3.85 (d, J = 11.4 Hz, 2H), 3.76 (d, J = 13.4 Hz, 1H), 3.71-3.65 (m, 1H), 3.59 (d, J = 6.4 Hz, 1H), 2.95-2.76 (m, 5H), 2.56 (s, 3H), 2.47-2.29 (m, 8H), 2.05-1.85 (m, 6H), 1.74 (d, J = 11.8 Hz, 3H), 1.17 (d, J = 10.8 Hz, 2H), 0.69 (s, 2H), 0.47 (s, 2H). |
| 58 | 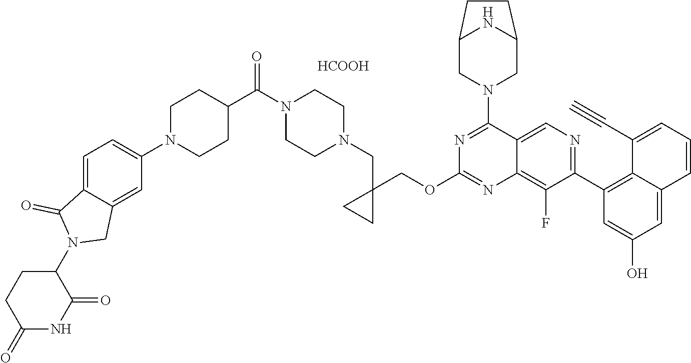 [SMILES: C#Cc1cccc2cc(O)cc(-c3ncc4c(N5CC6CCC(C5)N6)nc(OCC5(CN6CCN(C(=O)C7CCN(c8ccc9c(c8)CN(C8CCC(=O)NC8=O)C9=O)CC7)CC6)CC5)nc4c3F)c12 ; "HCOOH"] | 948.6 | 11.28 (s, 1H), 9.09-8.94 (m, 1H), 8.25 (s, 1H), 7.92-7.83 (m, 1H), 7.56-7.52 (m, 1H), 7.48-7.39 (m, 2H), 7.35-7.32 (m, 1H), 7.13-7.04 (m, 3H), 5.15-4.93 (m, 1H), 4.52-4.47 (m, 1H), 4.38-4.07 (m, 7H), 3.67-3.62 (m, 2H), 3.59-3.52 (m, 7H), 3.20-3.15 (m, 6H), 2.64 (s, 3H), 2.37-2.27 (m, 6H), 1.75-1.59 (m, 5H), 1.29-1.19 (m, 1H), 0.66 (s, 2H), 0.43 (s, 2H). |
| 59 | 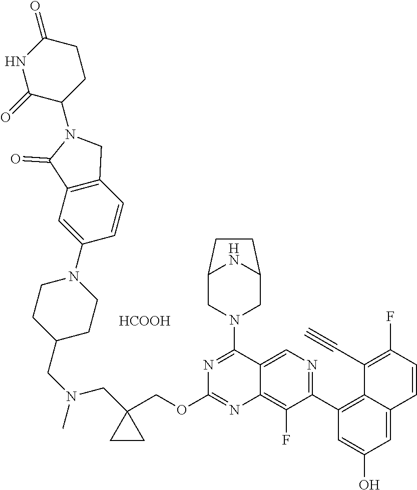 [SMILES: C#Cc1c(F)ccc2cc(O)cc(-c3ncc4c(N5CC6CCC(C5)N6)nc(OCC5(CN(C)CC6CCN(c7ccc8c(c7)C(=O)N(C7CCC(=O)NC7=O)C8)CC6)CC5)nc4c3F)c12 ; "HCOOH"] | 896.7 | 11.00 (s, 1H), 9.04 (d, J = 3.8 Hz, 1H), 8.20 (s, 1H), 8.02-7.93 (m, 1H), 7.46 (t, J = 9.0 Hz, 1H), 7.43-7.38 (m, 2H), 7.18 (t, J = 3.0 Hz, 1H), 6.89-6.79 (m, 2H), 5.06- 4.97 (m, 1H), 4.47-4.39 (m, 2H), 4.37- 4.31 (m, 1H), 4.20-4.04 (m, 3H), 3.90 (d, J = 1.8 Hz, 1H), 3.67 (dd, J = 7.0, 8.6 Hz, 3H), 3.58 (d, J = 11.8 Hz, 6H), 2.72-2.64 (m, 2H), 2.60 (s, 1H), 2.55 (d, J = 3.4 Hz, 1H), 2.35-2.29 (m, 1H), 2.28-2.24 (m, 1H), 2.22-2.18 (m, 3H), 2.15 (d, J = 4.9 Hz, 2H), 1.96-1.89 (m, 1H), 1.70 (dd, J = 1.4, 10.7 Hz, 3H), 1.64 (s, 4H), 1.12-0.97 (m, 2H), 0.66-0.60 (m, 2H), 0.45-0.36 (m, 2H). |
| 60 | 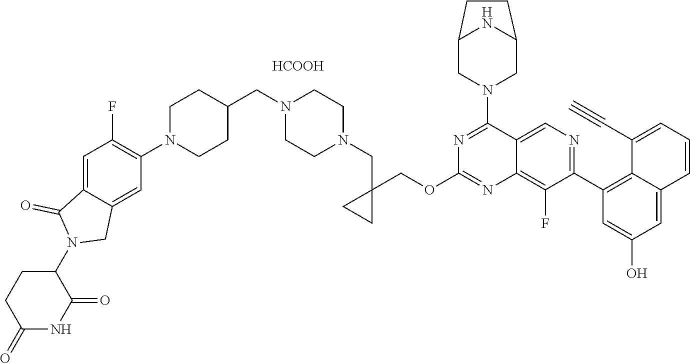 [SMILES: C#Cc1cccc2cc(O)cc(-c3ncc4c(N5CC6CCC(C5)N6)nc(OCC5(CN6CCN(CC7CCN(c8cc9c(cc8F)C(=O)N(C8CCC(=O)NC8=O)C9)CC7)CC6)CC5)nc4c3F)c12 ; "HCOOH"] | 951.7 | 10.97 (s, 1H), 9.02 (s, 1H), 8.19 (s, 2H), 7.88 (dd, J = 1.6, 8.1 Hz, 1H), 7.47-7.38 (m, 2H), 7.33 (d, J = 2.6 Hz, 1H), 7.25- 7.18 (m, 1H), 7.11 (d, J = 2.6 Hz, 1H), 5.06 (dd, J = 4.8, 13.3 Hz, 1H), 4.51 (d, J = 13.6 Hz, 1H), 4.38-4.19 (m, 5H), 3.69- 3.63 (m, 3H), 3.61-3.58 (m, 3H), 3.54 (s, 2H), 2.94-2.86 (m, 2H), 2.75-2.66 (m, 3H), 2.62-2.56 (m, 2H), 2.38-2.28 (m, 8H), 2.13 (d, J = 6.8 Hz, 2H), 2.00-1.94 (m, 1H), 1.81-1.73 (m, 2H), 1.69 (s, 4H), 1.29-1.20 (m, 2H), 0.63 (s, 2H), 0.40 (s, 2H). |
| 61 | 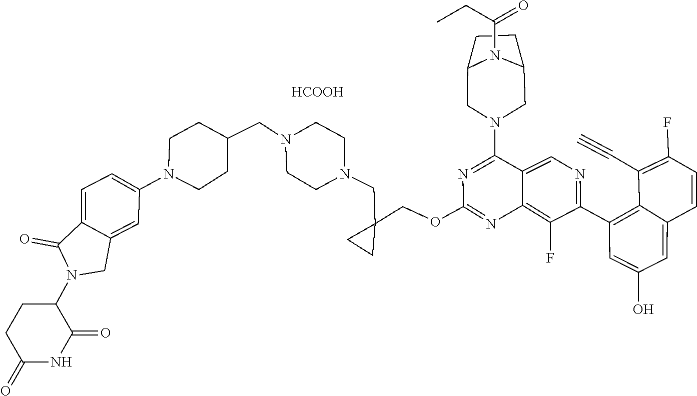 [SMILES: C#Cc1c(F)ccc2cc(O)cc(-c3ncc4c(N5CC6CCC(C5)N6C(=O)CC)nc(OCC5(CN6CCN(CC7CCN(c8ccc9c(c8)CN(C8CCC(=O)NC8=O)C9=O)CC7)CC6)CC5)nc4c3F)c12 ; "HCOOH"] | 1007.8 | 10.94 (s, 1H), 10.38-10.03 (m, 1H), 9.04 (d, J = 2.0 Hz, 1H), 8.20 (s, 1H), 7.98 (dd, J = 6.0 Hz, 1H), 7.50-7.44 (m, 2H), 7.39 (d, J = 2.4 Hz, 1H), 7.17 (s, 1H), 7.05- 7.00 (m, 2H), 5.03 (dd, J = 13.2 Hz, 1H), 4.69 (s, 1H), 4.65-4.57 (m, 1H), 4.48 (s, 1H), 4.45-4.35 (m, 1H), 4.34-4.27 (m, 3H), 4.22-4.16 (m, 1H), 3.95 (d, J = 6.0 Hz, 1H), 3.89-3.76 (m, 3H), 3.70-3.58 (m, 2H), 2.92-2.71 (m, 4H), 2.62-2.54 (m, 2H), 2.44-2.37 (m, 5H), 2.34-2.28 (m, 5H), 2.09 (d, J = 6.8 Hz, 2H), 1.98-1.89 (m, 2H), 1.85-1.70 (m, 6H), 1.20-1.10 (m, 2H), 1.06 (m, 3H), 0.64 (s, 2H), 0.41(s, 2H). |
| 62 | 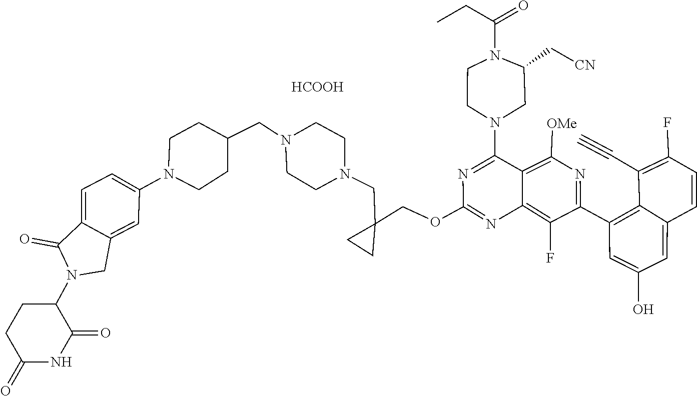 [SMILES: C#Cc1c(F)ccc2cc(O)cc(-c3nc(OC)c4c(N5CCN(C(=O)CC)[C@H](CC#N)C5)nc(OCC5(CN6CCN(CC7CCN(c8ccc9c(c8)CN(C8CCC(=O)NC8=O)C9=O)CC7)CC6)CC5)nc4c3F)c12 ; "HCOOH"] | 1050.48 | 10.96-10.92 (m, 1H), 10.24-10.15 (m, 1H), 8.28-8.22 (m, 1H), 8.02-7.94 (m, 1H), 7.53-7.45 (m, 2H), 7.42-7.38 (m, 1H), 7.26-7.21 (m, 1H), 7.07-7.00 (m, 2H), 5.06-5.02 (m, 1H), 4.36-4.27 (m, 3H), 4.25-4.15 (m, 2H), 4.07-3.99 (m, 1H), 3.96-3.92 (m, 2H), 3.92-3.88 (m, 2H), 3.85 (d, J = 12.8 Hz, 3H), 2.96-2.86 (m, 3H), 2.85-2.73 (m, 3H), 2.70-2.65 (m, 1H), 2.62-2.59 (m, 1H), 2.55 (d, 2H), 2.45-2.37 (m, 4H), 2.37-2.20 (m, 8H), 2.13-2.06 (m, 2H), 2.00-1.92 (m, 1H), 1.79-1.68 (m, 3H), 1.20-1.11 (m, 2H), 1.08-1.01 (m, 3H), 0.70 (s, 2H), 0.42 (s, 2H). |
| 63 | 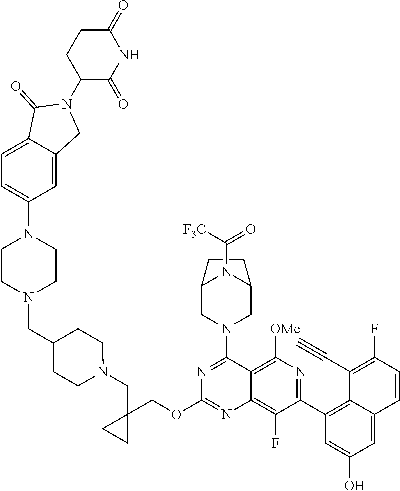 [SMILES: C#Cc1c(F)ccc2cc(O)cc(-c3nc(OC)c4c(N5CC6CCC(C5)N6C(=O)C(F)(F)F)nc(OCC5(CN6CCC(CN7CCN(c8ccc9c(c8)CN(C8CCC(=O)NC8=O)C9=O)CC7)CC6)CC5)nc4c3F)c12] | 1077.9 | 10.93 (s, 1H), 8.19-8.17 (m, 1H), 7.98 (dd, J = 5.8, 9.2 Hz, 1H), 7.52-7.45 (m, 2H), 7.39 (d, J = 2.4 Hz, 1H), 7.23 (s, 1H), 7.04-7.00 (m, 2H), 5.07-5.00 (m, 1H), 4.78-4.60 (m, 2H), 4.34-4.14 (m, 6H), 3.96 (s, 1H), 3.91 (s, 3H), 3.84 (d, J = 11.8 Hz, 3H), 2.94-2.83 (m, 2H), 2.79 (t, J = 11.8 Hz, 2H), 2.60 (s, 1H), 2.54 (s, 1H), 2.39-2.33 (m, 4H), 2.29 (d, J = 4.4 Hz, 4H), 2.08 (d, J = 6.6 Hz, 2H), 2.05- 1.84 (m, 6H), 1.79-1.67 (m, 4H), 1.19- 1.11 (m, 2H), 0.63 (s, 2H), 0.41 (s, 2H). |
| 64 | 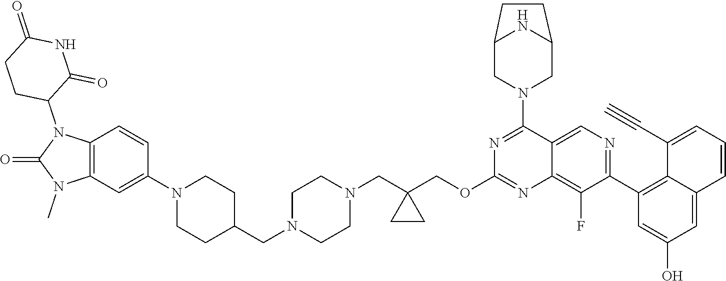 [SMILES: C#Cc1cccc2cc(O)cc(-c3ncc4c(N5CC6CCC(C5)N6)nc(OCC5(CN6CCN(CC7CCN(c8ccc9c(c8)n(C)c(=O)n9C8CCC(=O)NC8=O)CC7)CC6)CC5)nc4c3F)c12] | 948.5 | 11.15 (s, 1H), 10.40-9.94 (m, 1H), 9.17- 8.97 (m, 1H), 7.92-7.86 (m, 1H), 7.48- 7.38 (m, 2H), 7.34 (d, J = 2.4 Hz, 1H), 7.12 (d, J = 2.4 Hz, 1H), 6.92 (d, J = 8.6 Hz, 1H), 6.81 (d, J = 1.8 Hz, 1H), 6.64- 6.60 (m, 1H), 5.33-5.22 (m, 1H), 4.27 (s, 2H), 3.62-3.51 (m, 5H), 3.30 (s, 7H), 2.94-2.85 (m, 1H), 2.66-2.54 (m, 5H), 2.32-2.24 (m, 6H), 2.12 (d, J = 6.6 Hz, 3H), 2.02-1.95 (m, 2H), 1.81-1.73 (m, 3H), 1.64-1.54 (m, 2H), 1.26-1.16 (m, 3H), 0.64 (s, 2H), 0.41 (s, 2H). |
| 65 | 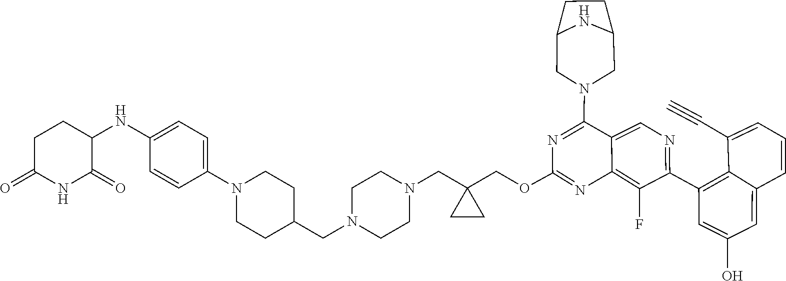 [SMILES: C#Cc1cccc2cc(O)cc(-c3ncc4c(N5CC6CCC(C5)N6)nc(OCC5(CN6CCN(CC7CCN(c8ccc(NC9CCC(=O)NC9=O)cc8)CC7)CC6)CC5)nc4c3F)c12] | 893.9 | 10.74 (s, 1H), 9.03 (s, 1H), 8.19 (s, 2H), 7.88 (dd, J = 1.4, 7.8 Hz, 1H), 7.49-7.37 (m, 2H), 7.36-7.27 (m, 1H), 7.14-7.06 (m, 1H), 6.73 (d, J = 8.8 Hz, 2H), 6.59 (d, J = 9.0 Hz, 2H), 5.43-5.21 (m, 1H), 4.59-4.39 (m, 1H), 4.29-4.24 (m, 2H), 4.17 (dd, J = 2.6 Hz, 1H), 3.73-3.58 (m, 11H), 2.71- 2.67 (m, 1H), 2.62-2.55 (m, 2H), 2.35- 2.25 (m, 7H), 2.15-2.07 (m, 3H), 1.90- 1.82 (m, 1H), 1.76-1.70 (m, 5H), 1.58- 1.49 (m, 1H), 1.28-1.12 (m, 3H), 0.65 (s, 2H), 0.40 (s, 2H). |
| 66 | 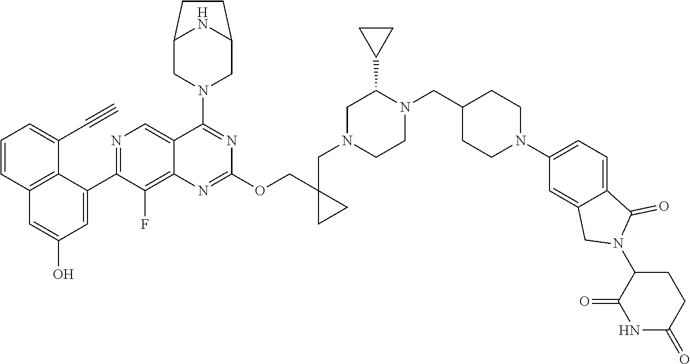 [SMILES: C#Cc1cccc2cc(O)cc(-c3ncc4c(N5CC6CCC(C5)N6)nc(OCC5(CN6CCN(CC7CCN(c8ccc9c(c8)CN(C8CCC(=O)NC8=O)C9=O)CC7)[C@@H](C7CC7)C6)CC5)nc4c3F)c12] | 974.0 | 10.95 (s, 1H), 9.03-8.97 (m, 1H), 8.21- 8.17 (m, 1H), 7.90-7.85 (m, 1H), 7.51- 7.38 (m, 3H), 7.33 (d, J = 2.4 Hz, 1H), 7.12-7.08 (m, 1H), 7.03-6.98 (m, 2H), 5.06-4.99 (m, 1H), 4.55-4.45 (m, 1H), 4.42-4.34 (m, 1H), 4.33-4.29 (m, 1H), 4.29-4.25 (m, 1H), 4.20 (s, 1H), 4.16 (t, J = 5.4 Hz, 1H), 3.87-3.80 (m, 2H), 3.69- 3.59 (m, 2H), 3.58-3.52 (m, 4H), 2.97- 2.68 (m, 9H), 2.15-1.80 (m, 9H), 1.66 (s, 4H), 1.29-1.05 (m, 4H), 0.67-0.60 (m, 2H), 0.58-0.49 (m, 2H), 0.44-0.35 (m, 2H), 0.33-0.26 (m, 1H), 0.17-0.08 (m, 1H), 0.01-0.10 (m, 1H). |
| 67 | 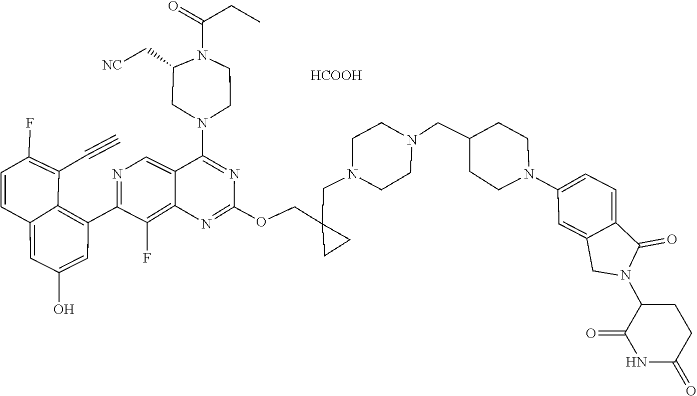 [SMILES: C#Cc1c(F)ccc2cc(O)cc(-c3ncc4c(N5CCN(C(=O)CC)[C@@H](CC#N)C5)nc(OCC5(CN6CCN(CC7CCN(c8ccc9c(c8)CN(C8CCC(=O)NC8=O)C9=O)CC7)CC6)CC5)nc4c3F)c12 ; "HCOOH"] | 1020.7 | 10.93 (s, 1H), 10.39-9.96 (m, 1H), 9.16- 9.04 (m, 1H), 8.21 (s, 1H), 7.98 (dd, J = 5.8, 9.3 Hz, 1H), 7.51-7.43 (m, 2H), 7.40 (d, J = 2.0 Hz, 1H), 7.21-7.15 (m, 1H), 7.05-6.99 (m, 2H), 5.03 (dd, J = 5.0 Hz, 1H), 4.87 (s, 1H), 4.44-4.14 (m, 7H), 4.02-3.75 (m, 6H), 3.05-2.73 (m, 7H), 2.63-2.54 (m, 3H), 2.37 (dd, J = 13.4 Hz, 5H), 2.30 (s, 3H), 2.11-2.07 (m, 2H), 1.98-1.93 (m, 1H), 1.77-1.68 (m, 3H), 1.30-1.09 (m, 3H), 1.06-0.99 (m, 3H), 0.64 (s, 2H), 0.42 (s, 2H). |
| 68 | 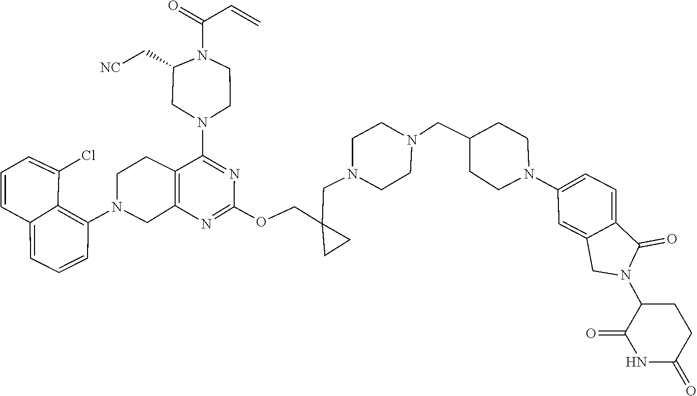 [SMILES: C=CC(=O)N1CCN(c2nc(OCC3(CN4CCN(CC5CCN(c6ccc7c(c6)CN(C6CCC(=O)NC6=O)C7=O)CC5)CC4)CC3)nc3c2CCN(c2cccc4cccc(Cl)c24)C3)C[C@@H]1CC#N] | 982.6 | 10.94 (s, 1H), 7.92 (d, J = 8.0 Hz, 1H), 7.74 (dd, J = 3.6 Hz, 1H), 7.60-7.42 (m, 4H), 7.38-7.29 (m, 1H), 7.06-7.00 (m, 2H), 5.04 (dd, J = 5.0 Hz, 1H), 4.35-4.26 (m, 1H), 4.23-4.06 (m, 4H), 4.03-3.66 (m, 6H), 3.58-3.43 (m, 1H), 3.21-2.98 (m, 7H), 2.94- 2.66(m, 7H), 2.37-1.90 (m, 12H), 1.80-1.71 (m, 3H), 1.27-1.09 (m, 3H), 1.05-0.99 (m, 3H), 0.63-0.51 (m, 2H), 0.43-0.31 (m, 2H). |
| 69 | 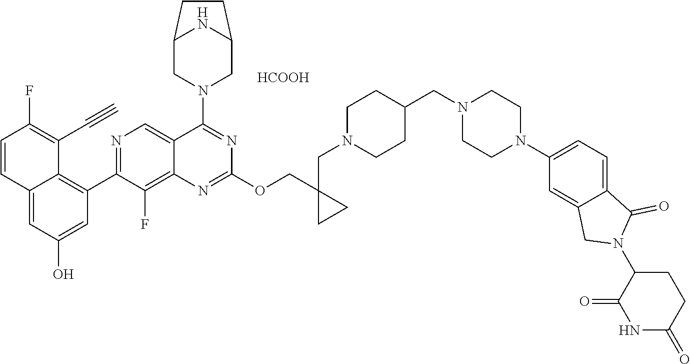 [SMILES: C#Cc1c(F)ccc2cc(O)cc(-c3ncc4c(N5CC6CCC(C5)N6)nc(OCC5(CN6CCC(CN7CCN(c8ccc9c(c8)CN(C8CCC(=O)NC8=O)C9=O)CC7)CC6)CC5)nc4c3F)c12 ; "HCOOH"] | 951.6 | 11.00-10.85 (m, 1H), 9.09-8.97 (m, 1H), 8.22 (s, 1H), 7.98 (dd, J = 6.0, 1H), 7.58- 7.43 (m, 2H), 7.42-7.36 (m, 1H), 7.18 (d, J = 2.4 Hz, 1H), 7.09-6.97 (m, 2H), 5.10- 4.95 (m, 1H), 4.58-4.43 (m, 1H), 4.38- 4.14 (m, 5H), 2.31-2.27 (m, 3H), 2.16- 2.08 (m, 3H), 2.00-1.84 (m, 4H), 1.71- 1.63 (m, 6H), 1.28-1.22 (m, 2H), 1.16- 0.95 (m, 3H), 0.64 (s, 2H), 0.45-0.33 (m, 2H). |
| 70 | 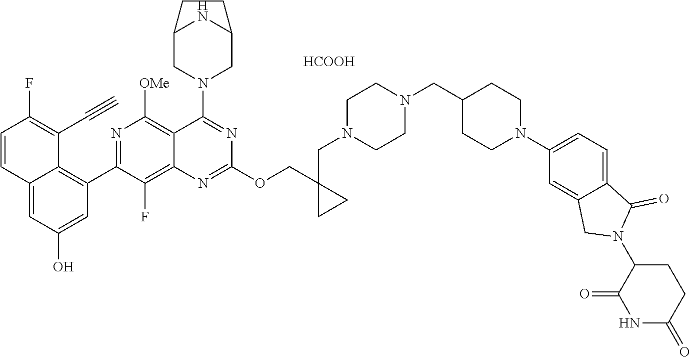 [SMILES: C#Cc1c(F)ccc2cc(O)cc(-c3nc(OC)c4c(N5CC6CCC(C5)N6)nc(OCC5(CN6CCN(CC7CCN(c8ccc9c(c8)CN(C8CCC(=O)NC8=O)C9=O)CC7)CC6)CC5)nc4c3F)c12 ; "HCOOH"] | 981.6 | 10.98-10.90 (m, 1H), 10.94 (s, 1H), 8.22 (s, 1H), 7.98 (dd, J = 6.0, 9.4 Hz, 1H), 7.52-7.43 (m, 2H), 7.39 (d, J = 2.4 Hz, 1H), 7.22 (d, J = 2.4 Hz, 1H), 7.06-6.99 (m, 2H), 5.04 (dd, J = 5.0, 13.3 Hz, 1H), 4.35-4.15 (m, 5H), 3.94-3.81 (m, 8H), 3.59 (s, 4H), 2.94-2.85 (m, 1H), 2.80 (t, J = 11.8 Hz, 2H), 2.63-2.53 (m, 2H), 2.38- 2.24 (m, 8H), 2.13-2.06 (m, 2H), 2.00- 1.92 (m, 1H), 1.77-1.59 (m, 7H), 1.21- 1.09 (m, 2H), 0.63 (s, 2H), 0.41 (s, 2H). |
| 71 | 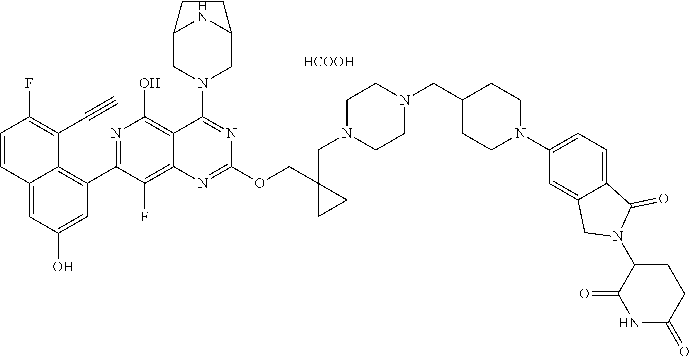 [SMILES: C#Cc1c(F)ccc2cc(O)cc(-c3nc(O)c4c(N5CC6CCC(C5)N6)nc(OCC5(CN6CCN(CC7CCN(c8ccc9c(c8)CN(C8CCC(=O)NC8=O)C9=O)CC7)CC6)CC5)nc4c3F)c12 ; "HCOOH"] | 967.6 | 11.42-11.25 (m, 1H), 10.94 (s, 1H), 8.27 (s, 2H), 7.99 (dd, J = 5.8, 9.2 Hz, 1H), 7.56-7.46 (m, 2H), 7.43 (d, J = 2.4 Hz, 1H), 7.25 (d, J = 2.4 Hz, 1H), 7.08-6.99 (m, 2H), 5.04 (dd, J = 5.2, 13.2 Hz, 1H), 4.35-4.15 (m, 5H), 3.92-3.81 (m, 3H), 3.57 (s, 4H), 2.94-2.77 (m, 4H), 2.55 (s, 1H), 2.32-2.24 (m, 4H), 2.14-2.07 (m, 3H), 2.02-1.91 (m, 2H), 1.79-1.72 (m, 3H), 1.68-1.62 (m, 2H), 1.31-1.21 (m, 3H), 1.21-1.12 (m, 2H), 1.06 (t, J = 7.0 Hz, 1H), 0.62 (s, 2H), 0.40 (s, 2H). |
| 72 | 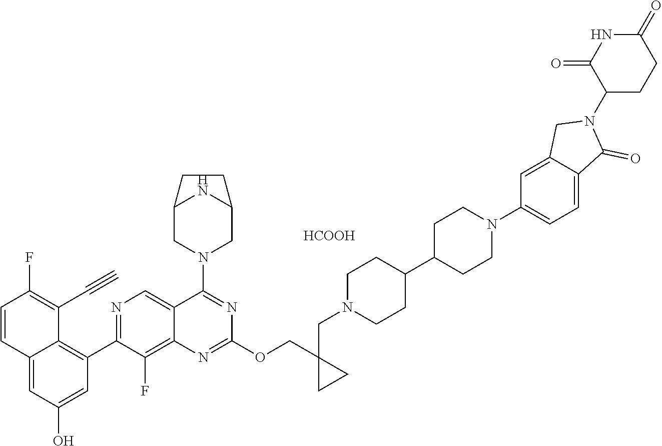 [SMILES: C#Cc1c(F)ccc2cc(O)cc(-c3ncc4c(N5CC6CCC(C5)N6)nc(OCC5(CN6CCC(C7CCN(c8ccc9c(c8)CN(C8CCC(=O)NC8=O)C9=O)CC7)CC6)CC5)nc4c3F)c12 ; "HCOOH"] | 936.4 | 10.96 (s, 1H), 9.04 (s, 1H), 8.19 (s, 1H), 8.01-7.96 (m, 1H), 7.52-7.44 (m, 2H), 7.40 (d, J = 2.4 Hz, 1H), 7.18 (d, J = 2.4 Hz, 1H), 7.03-6.98 (m, 2H), 5.10-4.96 (m, 1H), 4.53-4.46 (m, 1H), 4.34-4.26 (m, 4H), 4.22-4.14 (m, 1H), 3.95 (s, 1H), 3.87 (d, J = 11.8 Hz, 2H), 3.68-3.57 (m, 5H), 3.04-2.96 (m, 3H), 2.93-2.85 (m, 1H), 2.77-2.70 (m, 1H), 2.60 (d, J = 18.3 Hz, 1H), 2.38 (dd, J = 3.6, 4.6 Hz, 1H), 2.31- 2.27 (m, 2H), 1.98-1.92 (m, 1H), 1.84- 1.76 (m, 2H), 1.69 (s, 5H), 1.63-1.58 (m, 2H), 1.24-1.05 (m, 6H), 0.67-0.61 (m, 2H), 0.40 (s, 2H). |
| 73 | 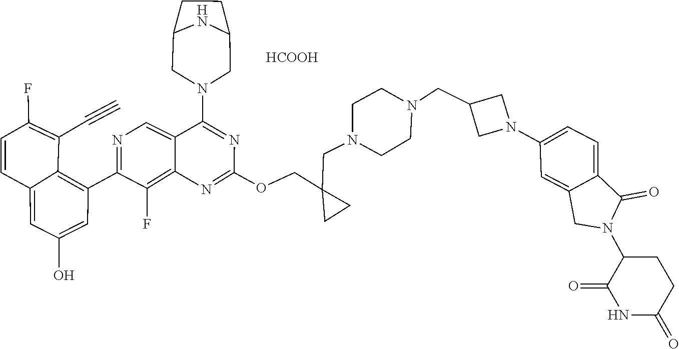 [SMILES: C#Cc1c(F)ccc2cc(O)cc(-c3ncc4c(N5CC6CCC(C5)N6)nc(OCC5(CN6CCN(CC7CN(c8ccc9c(c8)CN(C8CCC(=O)NC8=O)C9=O)C7)CC6)CC5)nc4c3F)c12 ; "HCOOH"] | 923.4 | 10.94 (s, 1H), 9.04 (s, 1H), 8.20 (s, 1H), 7.97 (dd, J = 6.0, 8.9 Hz, 1H), 7.50-7.43 (m, 2H), 7.39 (d, J = 2.4 Hz, 1H), 7.17 (d, J = 2.4 Hz, 1H), 6.52-6.40 (m, 2H), 5.03 (dd, J = 5.0, 13.3 Hz, 1H), 4.51 (d, J = 12.0 Hz, 1H), 4.33-4.24 (m, 4H), 4.20- 4.13 (m, 1H), 3.99 (t, J = 7.4 Hz, 2H), 3.94 (s, 1H), 3.66 (d, J = 13.0 Hz, 1H), 3.62-3.57 (m, 3H), 3.53 (dd, J = 5.8, 11.2 Hz, 5H), 2.97-2.84 (m, 3H), 2.62-2.58 (m, 1H), 2.55 (s, 2H), 2.41-2.35 (m, 4H), 2.30 (d, J = 5.6 Hz, 2H), 2.26 (s, 1H), 1.99- 1.91 (m, 1H), 1.69 (s, 4H), 0.69-0.59 (m, 2H), 0.45-0.37 (m, 2H). |
| 74 | 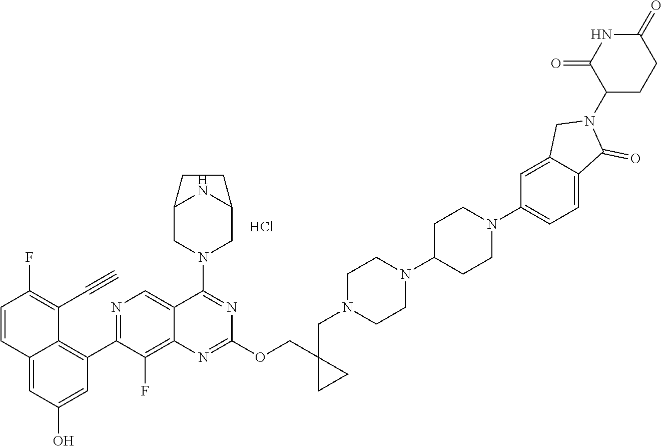 [SMILES: C#Cc1c(F)ccc2cc(O)cc(-c3ncc4c(N5CC6CCC(C5)N6)nc(OCC5(CN6CCN(C7CCN(c8ccc9c(c8)CN(C8CCC(=O)NC8=O)C9=O)CC7)CC6)CC5)nc4c3F)c12 ; Cl] | 937.6 | 10.97-10.92 (m, 1H), 10.30-10.20 (m, 1H), 9.75-9.53 (m, 1H), 9.39-9.23 (m, 1H), 9.17-9.07 (m, 1H), 8.04-7.93 (m, 1H), 7.55-7.45 (m, 2H), 7.43-7.39 (m, 1H), 7.22-7.17 (m, 1H), 7.07-7.04 (m, 1H), 5.08-5.02 (m, 1H), 4.68-4.60 (m, 1H), 4.58-4.47 (m, 1H), 4.40-4.30 (m, 2H), 4.28-4.16 (m, 4H), 4.06-3.81 (m, 6H), 3.10-3.05 (m, 1H), 2.94 (d, J = 5.0 Hz, 1H), 2.92-2.86 (m, 2H), 2.84-2.77 (m, 2H), 2.73 (s, 1H), 2.69-2.65 (m, 1H), 2.61 (s, 1H), 2.58-2.55 (m, 2H), 2.40-2.35 (m, 2H), 2.34-2.27 (m, 2H), 2.17-2.06 (m, 2H), 2.04-1.91 (m, 6H), 1.75-1.61 (m, 2H), 0.89-0.72 (m, 2H), 0.67-0.41 (m, 2H). |
| 75 | 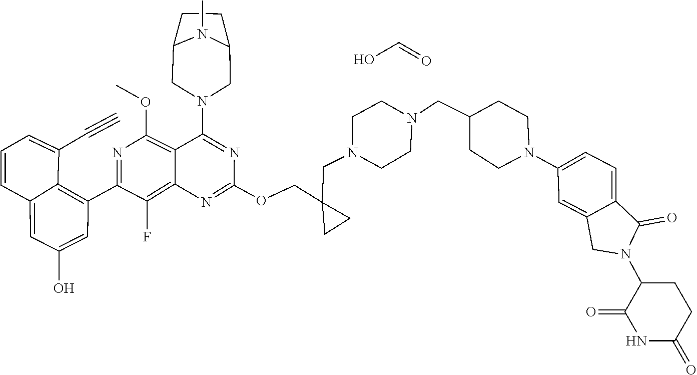 | 978.7 | 10.93 (s, 1H), 8.22 (s, 1H), 7.87 (d, J = 8.2 Hz, 1H), 7.51-7.46 (m, 2H), 7.44-7.39 (m, 1H), 7.33 (d, J = 2.4 Hz, 1H), 7.16 (d, J = 2.4 Hz, 1H), 7.05-7.00 (m, 2H), 5.03 (dd, J = 5.0, 13.2 Hz, 1H), 4.34-4.28 (m, 1H), 4.26 (s, 2H), 4.21-4.16 (m, 1H), 3.87 (s, 3H), 3.83 (s, 2H), 3.53 (s, 3H), 3.51 (s, 1H), 3.20 (s, 4H), 2.91-2.86 (m, 1H), 2.85-2.74 (m, 3H), 2.39 (dd, J = 4.6, 13.6 Hz, 4H), 2.34-2.28 (m, 4H), 2.27 (s, 1H), 2.22 (s, 3H), 2.09 (d, J = 6.4 Hz, 2H), 1.97-1.92 (m, 1H), 1.87 (s, 2H), 1.74 (d, J = 11.8 Hz, 3H), 1.63-1.57 (m, 1H), 1.50- 1.43 (m, 1H), 1.22-1.05 (m, 3H), 0.62 (s, 2H), 0.40 (s, 2H) |
| 76 | 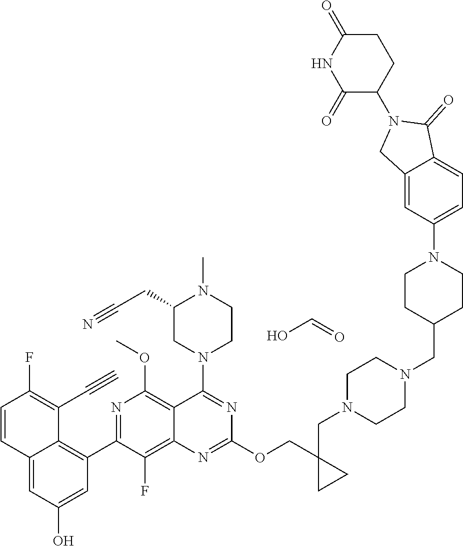 | 1008.7 | 10.93 (s, 1H), 8.16 (s, 1H), 7.97 (dd, J = 5.8, 9.2 Hz, 1H), 7.52-7.45 (m, 2H), 7.39 (d, J = 2.6 Hz, 1H), 7.22 (dd, J = 2.4, 5.4 Hz, 1H), 7.06-6.98 (m, 2H), 5.03 (dd, J = 5.2, 13.3 Hz, 1H), 4.35-4.30 (m, 1H), 4.28 (d, J = 4.0 Hz, 2H), 4.18 (d, J = 17.0 Hz, 1H), 4.14-4.06 (m, 1H), 4.02 (d, J = 1.8 Hz, 1H), 3.95 (s, 1H), 3.90 (d, J = 4.6 Hz, 3H), 3.84 (d, J = 11.6 Hz, 2H), 3.21-3.07 (m, 4H), 2.95-2.87 (m, 4H), 2.86-2.74 (m, 3H), 2.60 (d, J = 2.8 Hz, 1H), 2.54 (s, 2H), 2.44-2.36 (m, 5H), 2.35-2.32 (m, 2H), 2.31 (d, J = 1.4 Hz, 3H), 2.30-2.29 (m, 1H), 2.09 (d, J = 3.8 Hz, 2H), 1.98- 1.92 (m, 1H), 1.77-1.68 (m, 3H), 1.18- 1.09 (m, 2H), 0.63 (s, 2H), 0.41 (s, 2H) |
| 77 | 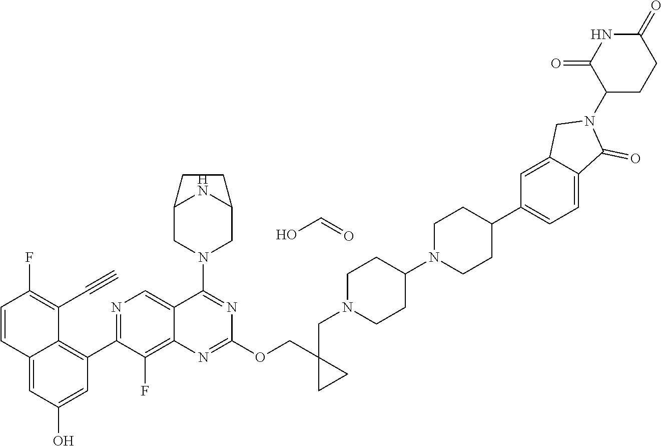 | 936.6 | 11.04-10.86 (m, 1H), 9.04 (s, 1H), 8.20 (s, 2H), 7.97 (dd, J = 5.8, 9.0 Hz, 1H), 7.63 (d, J = 7.8 Hz, 1H), 7.48-7.43 (m, 2H), 7.39-7.35 (m, 2H), 7.17 (d, J = 2.4 Hz, 1H), 5.10 (dd, J = 5.0, 13.2 Hz, 1H), 4.52 (d, J = 11.6 Hz, 1H), 4.44-4.38 (m, 1H), 4.36-4.24 (m, 5H), 3.94 (s, 1H), 3.60 (d, J = 12.0 Hz, 2H), 3.06-2.85 (m, 6H), 2.55 (d, J = 12.0 Hz, 2H), 2.39 (dd, J = 4.0, 12.8 Hz, 1H), 2.30 (s, 4H), 2.03-1.94 (m, 1H), 1.86 (t, J = 11.3 Hz, 2H), 1.80-1.60 (m, 11H), 1.50-1.36 (m, 2H), 0.65 (s, 2H), 0.41 (s, 2H) |
| 78 | 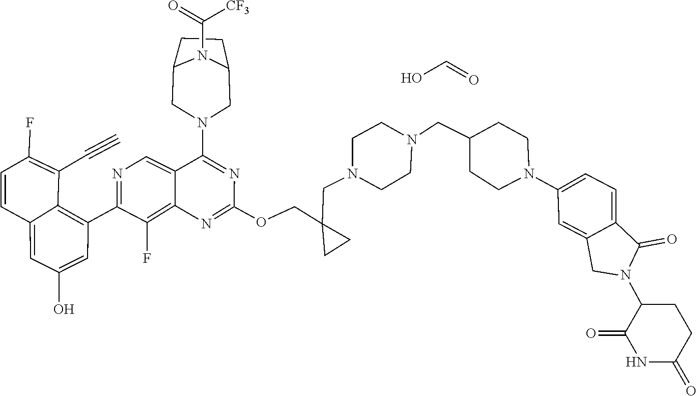 [SMILES: C#Cc1c(F)ccc2cc(O)cc(-c3ncc4c(N5CC6CCC(C5)N6C(=O)C(F)(F)F)nc(OCC5(CN6CCN(CC7CCN(c8ccc9c(c8)CN(C8CCC(=O)NC8=O)C9=O)CC7)CC6)CC5)nc4c3F)c12 ; O=CO] | 1047.3 | 10.93 (s, 1H), 9.05 (s, 1H), 8.15 (s, 1H), 7.98 (dd, J = 5.8, 9.2 Hz, 1H), 7.51-7.43 (m, 2H), 7.40 (d, J = 2.4 Hz, 1H), 7.18 (s, 1H), 7.05-6.98 (m, 2H), 5.03 (dd, J = 5.2, 13.4 Hz, 1H), 4.82-4.70 (m, 2H), 4.65 (d, J = 6.0 Hz, 1H), 4.51 (d, J = 12.2 Hz, 1H), 4.34-4.27 (m, 3H), 4.21-4.15 (m, 1H), 3.93 (d, J = 6.0 Hz, 1H), 3.84 (d, J = 11.8 Hz, 2H), 3.76 (d, J = 12.8 Hz, 1H), 3.67 (d, J = 13.6 Hz, 1H), 2.95-2.84 (m, 2H), 2.82-2.75 (m, 2H), 2.60 (s, 2H), 2.40-2.26 (m, 8H), 2.09 (d, J = 6.4 Hz, 2H), 2.01-1.86 (m, 5H), 1.73 (d, J = 11.0 Hz, 3H), 1.20-1.08 (m, 2H), 0.64 (s, 2H), 0.42 (s, 2H) |
| 79 | 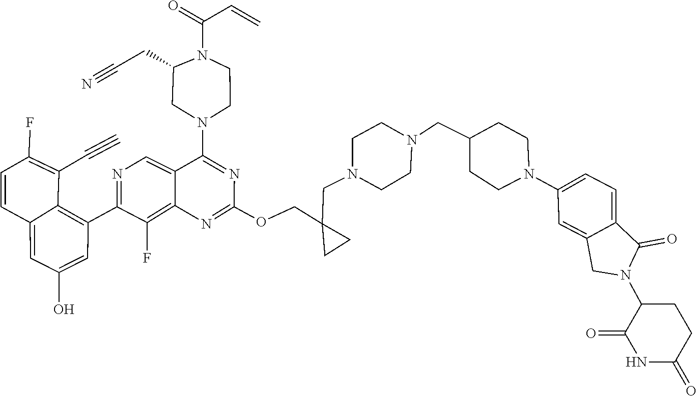 [SMILES: C#Cc1c(F)ccc2cc(O)cc(-c3ncc4c(N5CCN(C(=O)C=C)[C@@H](CC#N)C5)nc(OCC5(CN6CCN(CC7CCN(c8ccc9c(c8)CN(C8CCC(=O)NC8=O)C9=O)CC7)CC6)CC5)nc4c3F)c12] | 1018.6 | δ 10.95 (s, 1H), 10.36-10.04 (m, 1H), 9.17-9.09 (m, 1H), 8.21 (s, 1H), 8.03-7.95 (m, 1H), 7.51-7.44 (m, 2H), 7.41 (d, J = 2.4 Hz, 1H), 7.20 (dd, J = 2.4, 5.6 Hz, 1H), 7.07-6.99 (m, 2H), 6.27-6.18 (m, 1H), 5.87-5.76 (m, 1H), 5.10-4.98 (m, 1H), 4.48-4.26 (m, 6H), 4.22-4.15 (m, 1H), 3.98-3.92 (m, 1H), 3.88-3.81 (m, 3H), 3.20-3.16 (m, 2H), 3.12-3.04 (m, 3H), 2.96-2.75 (m, 6H), 2.58 (dd, J = 2.8, 19.8 Hz, 2H), 2.42-2.36 (m, 3H), 2.31 (s, 2H), 2.14-2.06 (m, 2H), 2.00-1.89 (m, 2H), 1.78-1.70 (m, 3H), 1.26-1.11 (m, 3H), 0.64 (s, 2H), 0.43 (s, 2H) |
| 80 | 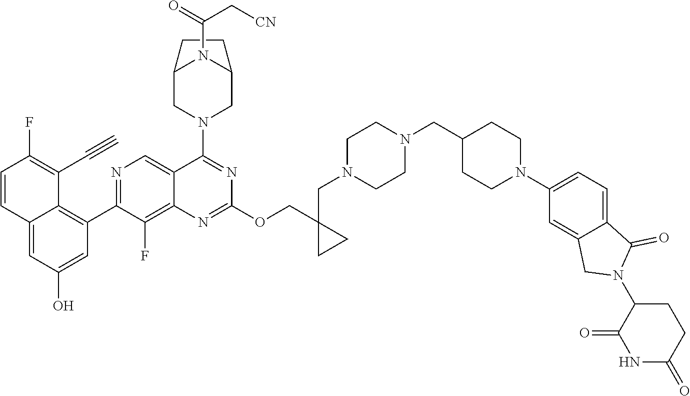 [SMILES: C#Cc1c(F)ccc2cc(O)cc(-c3ncc4c(N5CC6CCC(C5)N6C(=O)CC#N)nc(OCC5(CN6CCN(CC7CCN(c8ccc9c(c8)CN(C8CCC(=O)NC8=O)C9=O)CC7)CC6)CC5)nc4c3F)c12] | 1018.7 | δ 10.94 (s, 1H), 9.02 (d, J = 3.8 Hz, 1H), 8.21 (s, 1H), 7.98 (dd, J = 5.8, 9.4 Hz, 1H), 7.52-7.43 (m, 2H), 7.40 (d, J = 2.4 Hz, 1H), 7.17 (s, 1H), 7.06-6.98 (m, 2H), 5.03 (dd, J = 4.8, 13.4 Hz, 1H), 4.72-4.65 (m, 1H), 4.65-4.54 (m, 1H), 4.44-4.35 (m, 2H), 4.35-4.26 (m, 4H), 4.22-4.19 (m, 1H), 4.17-4.12 (m, 2H), 3.95 (d, J = 7.6 Hz, 1H), 3.88-3.78 (m, 4H), 2.94-2.85 (m, 3H), 2.83-2.75 (m, 4H), 2.34-2.28 (m, 6H), 2.09 (d, J = 6.6 Hz, 2H), 1.98-1.92 (m, 2H), 1.86-1.70 (m, 6H), 1.24-1.06 (m, 3H), 0.64 (s, 2H), 0.41 (s, 2H). |
| 81 | 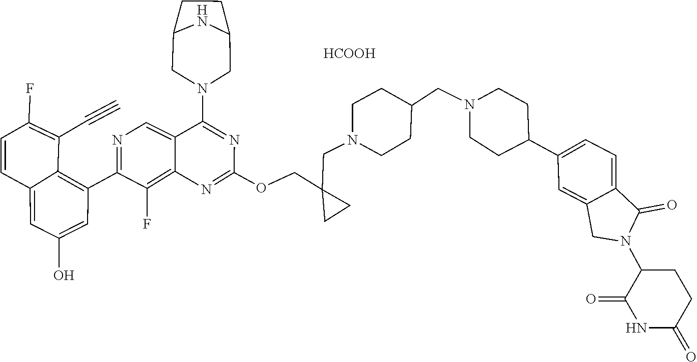 [SMILES: C#Cc1c(F)ccc2cc(O)cc(-c3ncc4c(N5CC6CCC(C5)N6)nc(OCC5(CN6CCC(CN7CCC(c8ccc9c(c8)CN(C8CCC(=O)NC8=O)C9=O)CC7)CC6)CC5)nc4c3F)c12 ; "HCOOH"] | 950.4 | 0.99 (s, 1H), 9.04 (s, 1H), 8.19 (s, 1H), 7.98 (dd, J = 5.8, 9.0 Hz, 1H), 7.64 (d, J = 7.8 Hz, 1H), 7.50-7.44 (m, 2H), 7.41-7.37 (m, 2H), 7.18 (d, J = 2.6 Hz, 1H), 5.10 (dd, J = 5.2, 13.2 Hz, 1H), 4.51 (br d, J = 12.0 Hz, 1H), 4.42 (d, J = 17.4 Hz, 1H), 4.34-4.25 (m, 4H), 3.95 (s, 1H), 3.68 (s, 1H), 3.64 (d, J = 6.0 Hz, 3H), 3.60 (s, 1H), 3.57 (s, 1H), 2.98-2.87 (m, 6H), 2.63-2.57 (m, 2H), 2.34-2.31 (m, 2H), 2.12 (d, J = 6.8 Hz, 2H), 2.01-1.94 (m, 3H), 1.91-1.84 (m, 2H), 1.75-1.65 (m, 9H), 1.52-1.43 (m, 1H), 1.12-1.01 (m, 2H), 0.69-0.59 (m, 2H), 0.46-0.37 (m, 2H) |
| 82 | 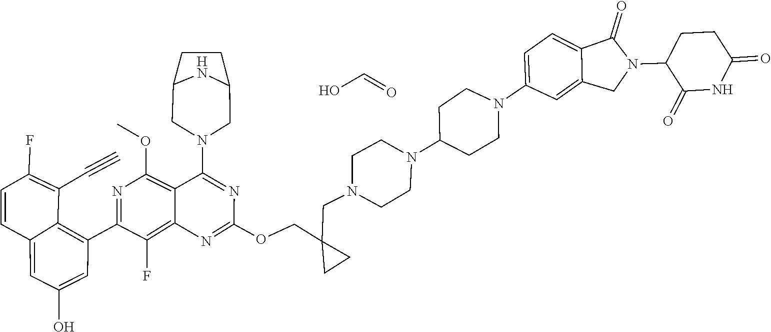 [SMILES: C#Cc1c(F)ccc2cc(O)cc(-c3nc(OC)c4c(N5CC6CCC(C5)N6)nc(OCC5(CN6CCN(C7CCN(c8ccc9c(c8)CN(C8CCC(=O)NC8=O)C9=O)CC7)CC6)CC5)nc4c3F)c12 ; O=CO] | 967.6 | 8.25 (s, 1H), 7.97 (dd, J = 6.0, 9.2 Hz, 1H), 7.53-7.42 (m, 2H), 7.40 (d, J = 2.6 Hz, 1H), 7.23 (d, J = 2.6 Hz, 1H), 7.06-6.95 (m, 2H), 5.01 (dd, J = 5.2, 13.4 Hz, 1H), 4.36-4.23 (m, 3H), 4.22-4.10 (m, 2H), 4.00-3.89 (m, 2H), 3.87 (d, J = 3.6 Hz, 3H), 3.85-3.79 (m, 2H), 3.74 (s, 2H), 3.49-3.38 (m, 4H), 2.94-2.83 (m, 1H), 2.82-2.70 (m, 2H), 2.64-2.54 (m, 2H), 2.46-2.32 (m, 8H), 2.27 (s, 2H), 2.02-1.90 (m, 1H), 1.85-1.61 (m, 6H), 1.49-1.33 (m, 2H), 0.61 (s, 2H), 0.40 (s, 2H) |
| 83 | 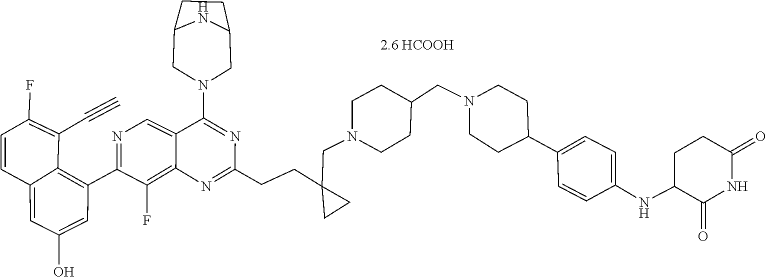 | 910.4 | 10.77 (s, 1H), 9.04 (s, 1H), 8.20 (s, 3H), 7.97 (d, J = 6.2 Hz, 1H), 7.43 (s, 1H), 7.37 (s, 1H), 7.18 (d, J = 2.6 Hz, 1H), 6.94 (d, J = 8.4 Hz, 2H), 6.60 (d, J = 8.4 Hz, 2H), 5.64 (d, J = 7.4 Hz, 1H), 4.51 (d, J = 12.8 Hz, 1H), 4.34-4.24 (m, 4H), 3.94 (s, 1H), 3.62 (s, 3H), 2.98-2.92 (m, 3H), 2.88 (d, J = 10.4 Hz, 3H), 2.79-2.70 (m, 2H), 2.61-2.58 (m, 1H), 2.35-2.30 (m, 3H), 2.13-2.07 (m, 3H), 1.94-1.83 (m, 5H), 1.73-1.60 (m, 9H), 1.13-0.98 (m, 2H), 0.64 (s, 2H), 0.41 (s, 2H) |
| 84 | 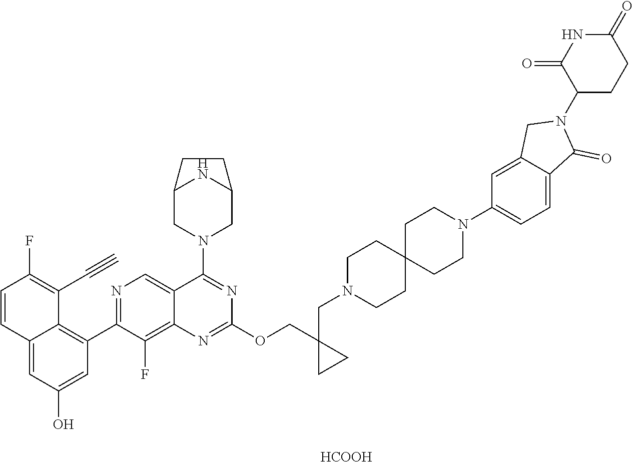 [SMILES: C#Cc1c(F)ccc2cc(O)cc(-c3ncc4c(N5CC6CCC(C5)N6)nc(OCC5(CN6CCC7(CC6)CCN(c6ccc8c(c6)CN(C6CCC(=O)NC6=O)C8=O)CC7)CC5)nc4c3F)c12 ; "HCOOH"] | 922.6 | 11.01-10.87 (m, 1H), 9.04 (s, 1H), 8.19 (s, 2H), 7.97 (dd, J = 6.0, 9.4 Hz, 1H), 7.52- 7.44 (m, 2H), 7.39 (d, J = 2.4 Hz, 1H), 7.17 (d, J = 2.4 Hz, 1H), 7.07-6.94 (m, 2H), 5.03 (dd, J = 5.0, 12.9 Hz, 1H), 4.51 (d, J = 12.0 Hz, 1H), 4.33-4.16 (m, 5H), 3.94 (s, 1H), 3.63 (s, 2H), 3.28 (s, 3H), 2.61-2.54 (m, 2H), 2.44-2.33 (m, 9H), 1.99-1.91 (m, 1H), 1.70 (s, 4H), 1.46 (d, J = 14.0 Hz, 8H), 0.64 (s, 2H), 0.42 (s, 2H) |
| 85 | 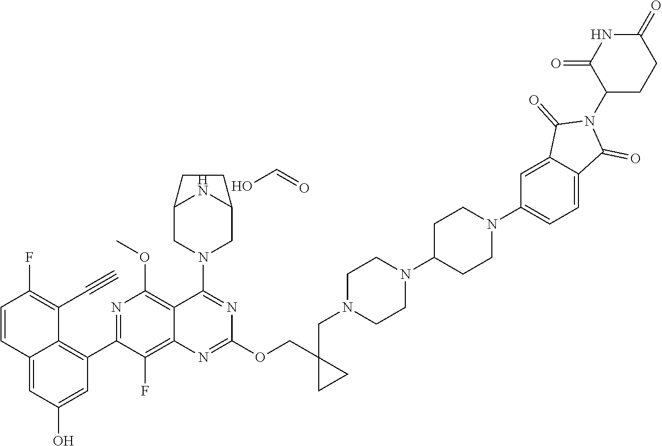 | 981.5 | 11.07 (s, 1H), 8.20 (s, 1H), 7.97 (dd, J = 5.8, 9.4 Hz, 1H), 7.64 (d, J = 8.6 Hz, 1H), 7.46 (t, J = 9.0 Hz, 1H), 7.38 (d, J = 2.6 Hz, 1H), 7.29 (s, 1H), 7.23-7.18 (m, 2H), 5.06 (dd, J = 5.4, 12.4 Hz, 1H), 4.27 (s, 2H), 4.16-4.09 (m, 1H), 4.03-3.98 (m, 2H), 3.89 (s, 1H), 3.88 (s, 3H), 3.56 (s, 6H), 2.98-2.87 (m, 4H), 2.69-2.57 (m, 2H), 2.46-2.35 (m, 8H), 2.27 (s, 2H), 2.03-1.97 (m, 1H), 1.79 (dd, J = 2.2, 13.0 Hz, 2H), 1.71-1.58 (m, 4H), 1.46-1.36 (m, 2H), 0.61 (s, 2H), 0.39 (s, 2H) |
| 86 | 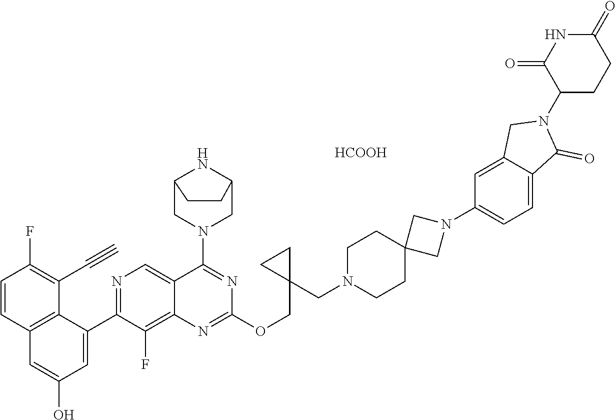 [SMILES: C#Cc1c(F)ccc2cc(O)cc(-c3ncc4c(N5CC6CCC(C5)N6)nc(OCC5(CN6CCC7(CC6)CN(c6ccc8c(c6)CN(C6CCC(=O)NC6=O)C8=O)C7)CC5)nc4c3F)c12 ; "HCOOH"] | 894.5 | 10.94 (s, 1H), 10.16 (s, 1H), 9.05 (s, 1H), 8.17 (s, 1H), 7.98 (dd, J = 6.2, 9.4 Hz, 1H), 7.50-7.44 (m, 2H), 7.40 (d, J = 2.6 Hz, 1H), 7.18 (d, J = 2.4 Hz, 1H), 6.53- 6.41 (m, 2H), 5.03 (dd, J = 4.6, 13.0 Hz, 1H), 4.56-4.48 (m, 1H), 4.35-4.25 (m, 4H), 4.20-4.13 (m, 1H), 3.95 (s, 1H), 3.70-3.56 (m, 9H), 2.95-2.86 (m, 2H), 2.63-2.57 (m, 2H), 2.30 (d, J = 8.6 Hz, 4H), 2.00-1.89 (m, 2H), 1.71 (s, 8H), 0.69-0.63 (m, 2H), 0.46-0.40 (m, 2H) |
| 87 | 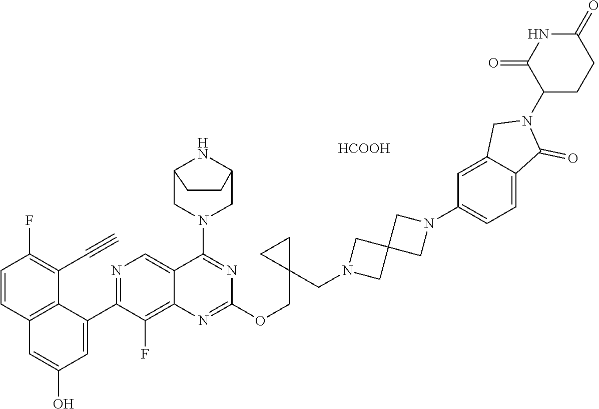 [SMILES: C#Cc1c(F)ccc2cc(O)cc(-c3ncc4c(N5CC6CCC(C5)N6)nc(OCC5(CN6CC7(C6)CN(c6ccc8c(c6)CN(C6CCC(=O)NC6=O)C8=O)C7)CC5)nc4c3F)c12 ; "HCOOH"] | 866.4 | 10.94 (s, 1H), 9.05 (s, 1H), 8.21 (s, 1H), 7.98 (dd, J = 6.0, 9.2 Hz, 1H), 7.50-7.44 (m, 2H), 7.40 (d, J = 2.4 Hz, 1H), 7.19 (d, J = 2.4 Hz, 1H), 6.51-6.42 (m, 2H), 5.06- 4.99 (m, 1H), 4.53-4.45 (m, 1H), 4.34- 4.13 (m, 6H), 3.97-3.95 (m, 4H), 3.68- 3.63 (m, 2H), 3.61-3.55 (m, 5H), 2.96- 2.84 (m, 2H), 2.60 (dd, J = 1.8, 2.8 Hz, 1H), 2.56 (s, 1H), 2.41 (s, 2H), 2.36 (d, J = 4.2 Hz, 1H), 2.01-1.90 (m, 1H), 1.69 (s, 3H), 1.27-1.21 (m, 1H), 0.54 (s, 2H), 0.45 (s, 2H) |
| 88 | 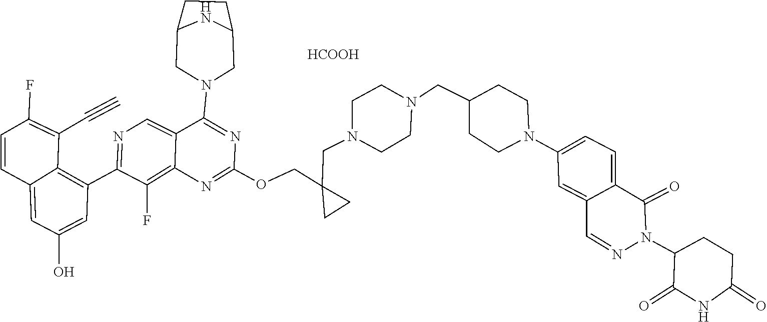 | 965.0 | 10.99 (s, 1H), 10.26-10.07 (m, 1H), 9.03 (s, 1H), 8.26 (s, 1H), 8.23 (s, 1H), 8.04- 7.94 (m, 2H), 7.51-7.42 (m, 2H), 7.39 (d, J = 2.6 Hz, 1H), 7.22 (d, J = 1.8 Hz, 1H), 7.18 (d, J = 2.4 Hz, 1H), 5.74 (dd, J = 5.4, 11.0 Hz, 1H), 4.50 (dd, J = 1.8, 12.4 Hz, 1H), 4.32-4.24 (m, 3H), 4.03-3.96 (m, 2H), 3.94 (s, 1H), 3.68-3.62 (m, 1H), 3.60-3.51 (m, 5H), 2.97-2.86 (m, 4H), 2.65-2.57 (m, 3H), 2.30 (d, J = 6.0 Hz, 5H), 2.14-2.03 (m, 4H), 1.82-1.73 (m, 3H), 1.67 (s, 4H), 1.27-1.07 (m, 3H), 0.64 (s, 2H), 0.41 (s, 2H). |
| 89 | 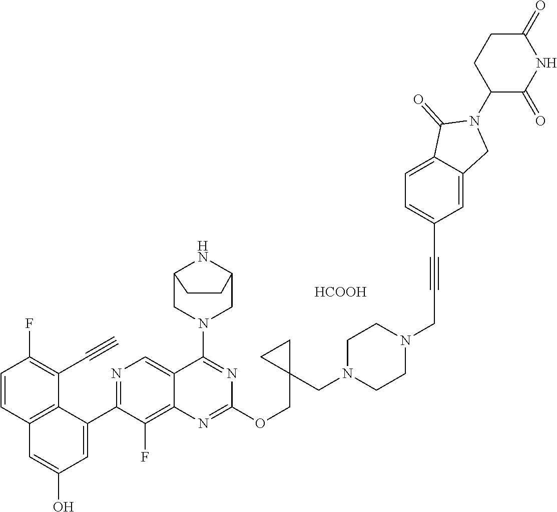 [SMILES: C#Cc1c(F)ccc2cc(O)cc(-c3ncc4c(N5CC6CCC(C5)N6)nc(OCC5(CN6CCN(CC#Cc7ccc8c(c7)CN(C7CCC(=O)NC7=O)C8=O)CC6)CC5)nc4c3F)c12 ; "HCOOH"] | 892.9 | 11.04-10.95 (m, 1H), 10.19-10.11 (m, 1H), 9.04-8.95 (m, 1H), 8.03-7.92 (m, 1H), 7.72-7.63 (m, 2H), 7.56-7.43 (m, 2H), 7.42-7.36 (m, 1H), 7.17 (d, J = 2.4 Hz, 1H), 5.17-4.97 (m, 1H), 4.52-4.38 (m, 2H), 4.31-4.21 (m, 3H), 3.93 (s, 1H), 3.67-3.58 (m, 1H), 3.56-3.50 (m, 5H), 2.96-2.89 (m, 1H), 2.73-2.64 (m, 2H), 2.38-2.29 (m, 5H), 2.06-1.94 (m, 2H), 1.67-1.58 (m, 4H), 1.30-1.21 (m, 4H), 0.69-0.60 (m, 2H), 0.42 (s, 2H). |
| 90 | 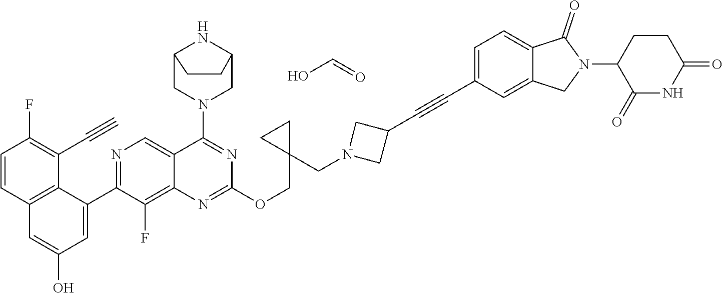 [SMILES: C#Cc1c(F)ccc2cc(O)cc(-c3ncc4c(N5CC6CCC(C5)N6)nc(OCC5(CN6CC(C#Cc7ccc8c(c7)CN(C7CCC(=O)NC7=O)C8=O)C6)CC5)nc4c3F)c12 ; O=CO] | 849.8 | 11.00 (s, 1H), 9.06-8.99 (m, 1H), 8.20 (s, 1H), 8.02-7.96 (m, 1H), 7.62 (s, 2H), 7.54-7.44 (m, 2H), 7.41-7.38 (m, 1H), 7.18 (d, J = 2.4 Hz, 1H), 5.07 (s, 1H), 4.42 (s, 2H), 4.35-4.28 (m, 2H), 4.23-4.19 (m, 2H), 3.95 (s, 1H), 3.66-3.62 (m, 3H), 3.58 (s, 4H), 3.13-3.05 (m, 4H), 2.62-2.56 (m, 1H), 2.46-2.44 (m, 2H), 2.41-2.37 (m, 1H), 1.68 (s, 4H), 0.59-0.52 (m, 2H), 0.49-0.45 (m, 2H). |
| 91 | 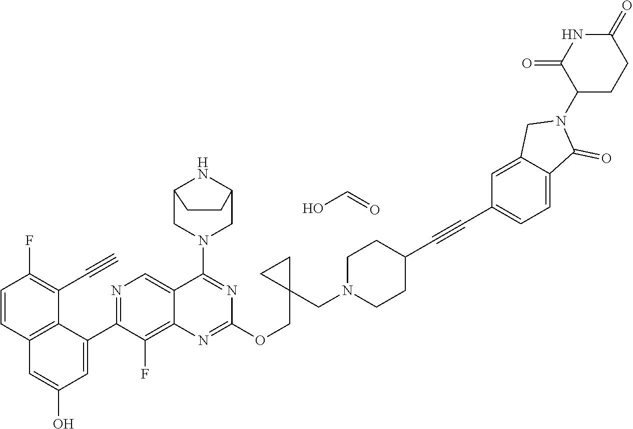 | 877.9 | 11.04-10.92 (m, 1H), 9.06-9.00 (m, 1H), 8.20 (s, 1H), 7.97 (dd, J = 6.0, 9.1 Hz, 1H), 7.67 (d, J = 8.0 Hz, 1H), 7.60 (s, 1H), 7.50-7.43 (m, 2H), 7.38 (d, J = 2.4 Hz, 1H), 7.17 (d, J = 2.4 Hz, 1H), 5.14- 5.06 (m, 1H), 4.49 (d, J = 11.2 Hz, 1H), 4.45-4.39 (m, 1H), 4.32 (s, 1H), 4.28 (d, J = 1.2 Hz, 3H), 3.92 (s, 1H), 3.66 (s, 1H), 3.64-3.61 (m, 1H), 3.58 (s, 4H), 3.54 (s, 1H), 2.95-2.86 (m, 2H), 2.80-2.73 (m, 2H), 2.62 (s, 1H), 2.21-2.14 (m, 2H), 2.06-1.95 (m, 2H), 1.88-1.83 (m, 2H), 1.71-1.65 (m, 4H), 1.63-1.55 (m, 2H), 0.65 (s, 2H), 0.42 (s, 2H). |
| 92 | 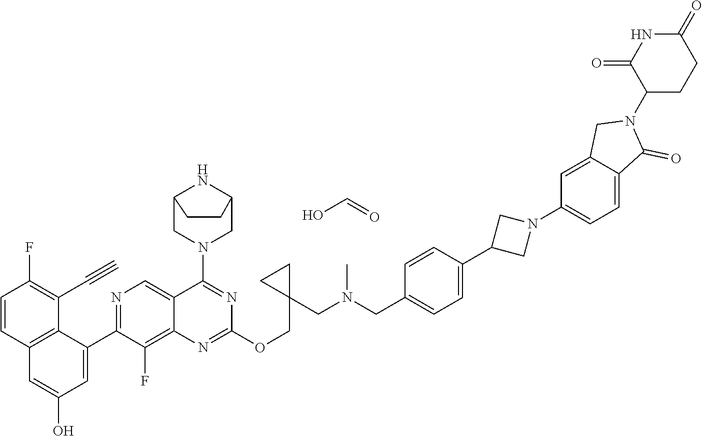 [SMILES: C#Cc1c(F)ccc2cc(O)cc(-c3ncc4c(N5CC6CCC(C5)N6)nc(OCC5(CN(C)Cc6ccc(C7CN(c8ccc9c(c8)CN(C8CCC(=O)NC8=O)C9=O)C7)cc6)CC5)nc4c3F)c12 ; O=CO] | 944.7 | 10.93 (s, 1H), 10.24-10.13 (m, 1H), 9.03 (s, 1H), 8.18 (s, 1H), 7.99 (dd, J = 6.0, 9.2 Hz, 1H), 7.51-7.39 (m, 3H), 7.26-7.13 (m, 5H), 6.53-6.44 (m, 2H), 5.05 (dd, J = 4.8, 13.4 Hz, 1H), 4.48-4.38 (m, 2H), 4.36- 4.25 (m, 5H), 4.20-4.12 (m, 1H), 3.96- 3.88 (m, 2H), 3.84-3.75 (m, 2H), 3.66- 3.42 (m, 7H), 2.98-2.84 (m, 1H), 2.63- 2.59 (m, 1H), 2.44-2.35 (m, 2H), 2.30- 2.25 (m, 1H), 2.19 (s, 3H), 2.01-1.90 (m, 1H), 1.72-1.61 (m, 4H), 0.65 (s, 2H), 0.43 (s, 2H). |
| 93 | 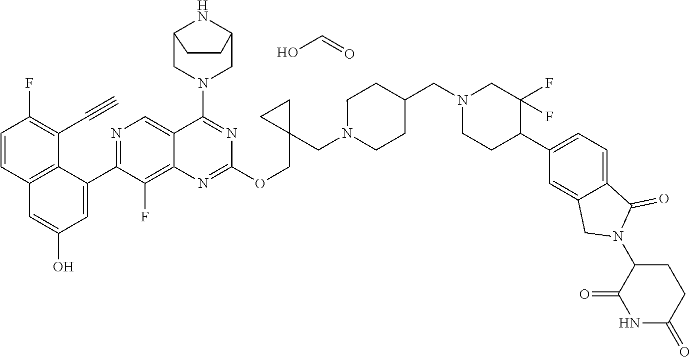 | 986.8 | 10.98 (s, 1H), 9.03 (s, 1H), 8.23 (s, 1H), 7.97 (dd, J = 6.0, 9.2 Hz, 1H), 7.68 (d, J = 8.0 Hz, 1H), 7.56 (s, 1H), 7.49-7.42 (m, 2H), 7.39 (d, J = 2.4 Hz, 1H), 7.17 (d, J = 2.4 Hz, 1H), 5.11 (dd, J = 4.4, 12.6 Hz, 1H), 4.52-4.43 (m, 2H), 4.34-4.31 (m, 1H), 4.30-4.24 (m, 3H), 3.93 (s, 1H), 3.65 (br d, J = 12.0 Hz, 2H), 3.59 (d, J = 6.8 Hz, 4H), 3.15-3.07 (m, 2H), 2.98-2.90 (m, 4H), 2.61 (d, J = 2.8 Hz, 1H), 2.58 (s, 1H), 2.42-2.34 (m, 2H), 2.31 (s, 2H), 2.24 (d, J = 6.0 Hz, 2H), 2.15 (d, J = 9.6 Hz, 2H), 2.03-1.96 (m, 1H), 1.87 (t, J = 11.2 Hz, 2H), 1.69 (s, 4H), 1.66-1.60 (m, 2H), 1.52-1.46 (m, 1H), 1.12-1.01 (m, 2H), 0.64 (s, 2H), 0.41 (s, 2H). |
| 94 | 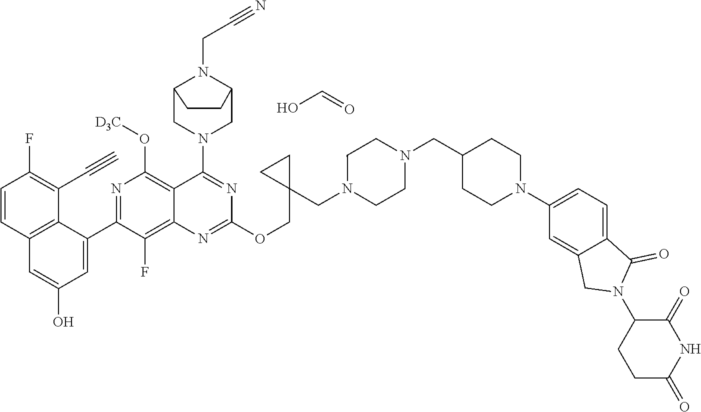 | 1024.1 | 11.07-10.85 (m, 1H), 8.30-8.14 (m, 1H), 8.04-7.87 (m, 1H), 7.55 (J = 4.9 Hz, 2H), 7.43-7.34 (m, 1H), 7.26-7.16 (m, 1H), 7.06-6.94 (m, 2H), 5.11-4.97 (m, 1H), 4.17 (s, 6H), 4.01-3.92 (m, 2H), 3.85 (J = 12.3 Hz, 2H), 3.60-3.56 (m, 3H), 2.96- 2.85 (m, 2H), 2.84-2.77 (m, 2H), 2.64- 2.58 (m, 2H), 2.31-2.29 (m, 2H), 1.90- 1.83 (m, 2H), 1.79-1.74 (m, 2H), 1.64- 1.50 (m, 2H), 1.21-1.09 (m, 2H), 0.63 (s, 2H), 0.44-0.35 (m, 2H). |
| 95 | 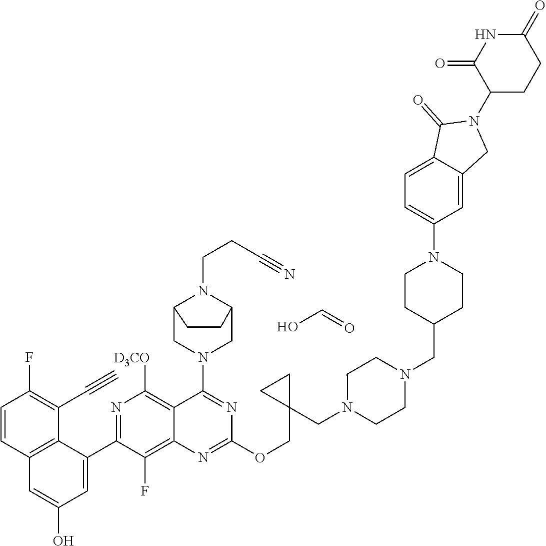 | 1038.2 | 10.97-10.88 (m, 1H), 10.20-10.08 (m, 1H), 8.21-8.14 (m, 1H), 8.02-7.90 (m, 1H), 7.51-7.42 (m, 2H), 7.41-7.37 (m, 1H), 7.23-7.19 (m, 1H), 7.05-6.98 (m, 2H), 5.09-4.98 (m, 1H), 4.34-4.24 (m, 3H), 4.22-4.15 (m, 1H), 4.14-4.05 (m, 1H), 3.96-3.87 (m, 2H), 3.87-3.80 (m, 2H), 3.39 (s, 3H), 2.98-2.84 (m, 2H), 2.83-2.77 (m, 2H), 2.71-2.64 (m, 4H), 2.61-2.54 (m, 6H), 2.31-2.25 (m, 4H), 2.09 ( J = 6.4 Hz, 2H), 2.02-1.90 (m, 2H), 1.83-1.73 (m, 4H), 1.70-1.39 (m, 4H), 1.19 (s, 2H), 1.18-1.06 (m, 2H), 0.66-0.56 (m, 2H), 0.40 (s, 2H). |
| 96 | 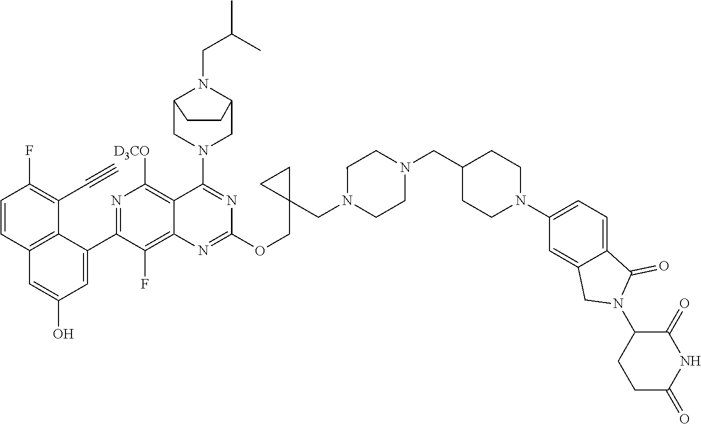 | 1041.1 | 10.94 (s, 1H), 10.14 (s, 1H), 7.97 (dd, J = 5.9, 9.2 Hz, 1H), 7.51-7.43 (m, 2H), 7.38 (d, J = 2.5 Hz, 1H), 7.22 (d, J = 2.5 Hz, 1H), 7.05-6.99 (m, 2H), 5.04 (dd, J = 5.0, 13.3 Hz, 1H), 4.35-4.26 (m, 3H), 4.22- 4.16 (m, 1H), 4.15-4.04 (m, 1H), 3.90 (s, 1H), 3.85 (d, J = 12.4 Hz, 2H), 3.45-3.37 (m, 2H), 3.26 (s, 3H), 2.88 (d, J = 12.4 Hz, 1H), 2.79 (t, J = 11.2 Hz, 2H), 2.60 (s, 1H), 2.56 (s, 1H), 2.41 (d, J = 4.6 Hz, 3H), 2.39-2.28 (m, 7H), 2.11 (d, J = 7.2 Hz, 4H), 1.99-1.93 (m, 1H), 1.82-1.67 (m, 6H), 1.62-1.54 (m, 1H), 1.49 (d, J = 7.5 Hz, 1H), 1.22-1.10 (m, 2H), 0.93 (d, J = 6.5 Hz, 6H), 0.63 (s, 2H), 0.41 (s, 2H). |
| 97 | 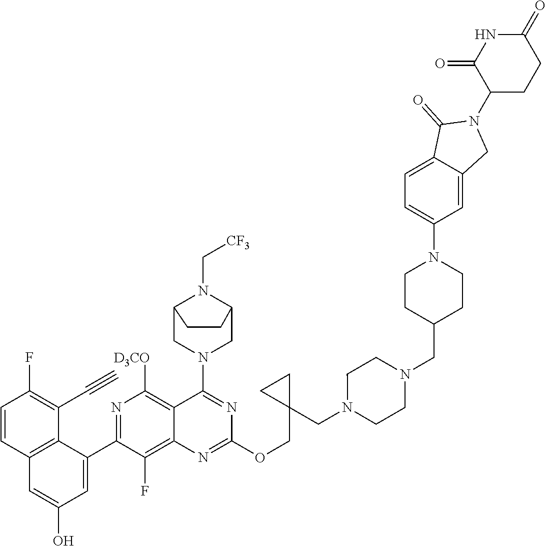 | 1067.1 | 10.95 (s, 1H), 10.16 (s, 1H), 7.98 (dd, J = 6.2, 9.2 Hz, 1H), 7.53-7.43 (m, 2H), 7.39 (d, J = 2.6 Hz, 1H), 7.22 (d, J = 2.6 Hz, 1H), 7.07-6.99 (m, 2H), 5.04 (dd, J = 5.2, 13.2 Hz, 1H), 4.36-4.15 (m, 4H), 4.08 (s, 1H), 3.89-3.80 (m, 2H), 3.99-3.80 (m, 3H), 3.47-3.36 (m, 4H), 3.17 (q, J = 9.8 Hz, 2H), 2.97-2.74 (m, 3H), 2.62-2.54 (m, 1H), 2.32-2.24 (m, 5H), 2.09 (d, J = 6.8 Hz, 2H), 2.02-1.90 (m, 2H), 1.89-1.50 (m, 9H), 1.34-1.04 (m, 4H), 0.63 (s, 2H), 0.40 (s, 2H). |
| 98 | 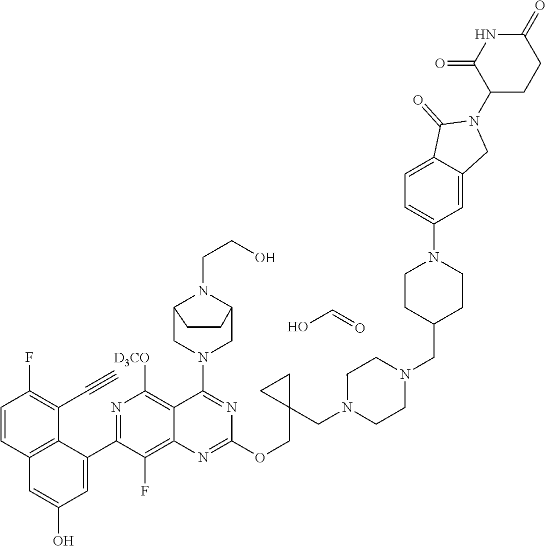 | 1028.5 | 10.05 (J = 8.0 Hz, 1H), 8.23-8.17 (m, 1H), 8.00 (s, 1H), 7.52-7.43 (m, 2H), 7.38 (J = 2.4 Hz, 1H), 7.21 (J = 2.4 Hz, 1H), 7.05-6.99 (m, 2H), 5.11-4.94 (m, 1H), 4.50-4.37 (m, 1H), 4.34-4.15 (m, 4H), 4.13-4.01 (m, 1H), 3.91-3.79 (m, 4H), 3.54-3.49 (m, 2H), 3.49-3.41 (m, 3H), 2.95-2.73 (m, 4H), 2.60 (J = 4.0 Hz, 1H), 2.44-2.39 (m, 5H), 2.31-2.24 (m, 4H), 2.12-2.05 (m, 2H), 2.02-1.89 (m, 2H), 1.81-1.70 (m, 4H), 1.60-1.43 (m, 2H), 1.27-1.05 (m, 3H), 0.66-0.58 (m, 2H), 0.45-0.35 (m, 2H). |
| 99 | 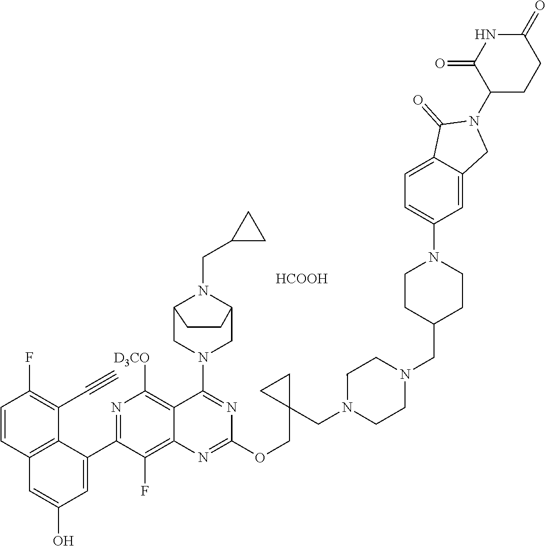 | 1038.5 | 10.99-10.89 (m, 1H), 10.30 (s, 1H), 8.24 (J = 2.4, 4.6 Hz, 1H), 8.02-7.90 (m, 1H), 7.54-7.42 (m, 2H), 7.41-7.33 (m, 1H), 7.22 (d, J = 2.4 Hz, 1H), 7.06-6.97 (m, 2H), 5.17-4.90 (m, 1H), 4.37-4.06 (m, 6H), 3.93-3.77 (m, 4H), 3.50-3.41 (m, 5H), 2.96-2.74 (m, 4H), 2.60-2.56 (m, 1H), 2.39 (J = 3.9, 13.6 Hz, 3H), 2.29 (J = 7.0 Hz, 3H), 2.24 (J = 6.0 Hz, 3H), 2.09 (J = 6.0 Hz, 2H), 1.99-1.90 (m, 2H), 1.81- 1.72 (m, 4H), 1.58 (t, J = 8.0 Hz, 1H), 1.51-1.43 (m, 1H), 1.18-1.10 (m, 2H), 0.92-0.83 (m, 1H), 0.66-0.57 (m, 2H), 0.52-0.36 (m, 5H), 0.15-0.06 (m, 2H). |
| 100 | 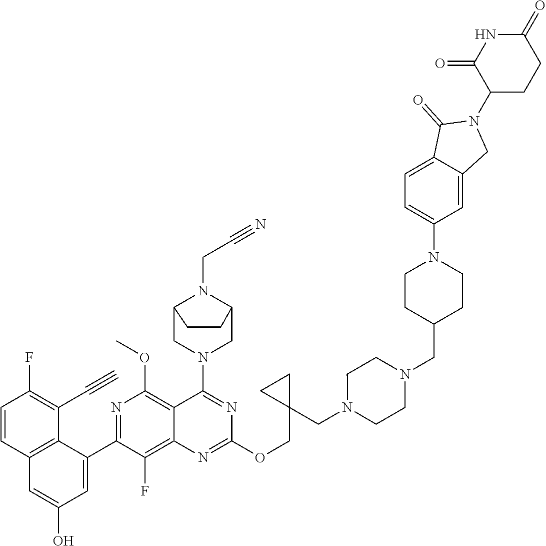 [SMILES: C#Cc1c(F)ccc2cc(O)cc(-c3nc(OC)c4c(N5CC6CCC(C5)N6CC#N)nc(OCC5(CN6CCN(CC7CCN(c8ccc9c(c8)CN(C8CCC(=O)NC8=O)C9=O)CC7)CC6)CC5)nc4c3F)c12] | 1020.4 | 10.98-10.83 (m, 1H), 10.24-10.07 (m, 1H), 8.02 (d, J = 3.0 Hz, 1H), 7.53-7.41 (m, 2H), 7.40-7.34 (m, 1H), 7.22 (d, J = 2.2 Hz, 1H), 7.09-6.95 (m, 2H), 5.10-4.97 (m, 1H), 4.37-4.08 (m, 5H), 3.94-3.81 (m, 6H), 3.57 (s, 2H), 3.40 (s, 3H), 2.98-2.75 (m, 4H), 2.59-2.55 (m, 1H), 2.38 (d, J = 4.0 Hz, 2H), 2.31-2.29 (m, 1H), 2.12-2.05 (m, 2H), 2.00-1.90 (m, 2H), 1.89-1.82 (m, 2H), 1.79-1.52 (m, 6H), 1.24-1.09 (m, 3H), 0.62 (s, 2H), 0.43-0.34 (m, 2H). |
| 101 | 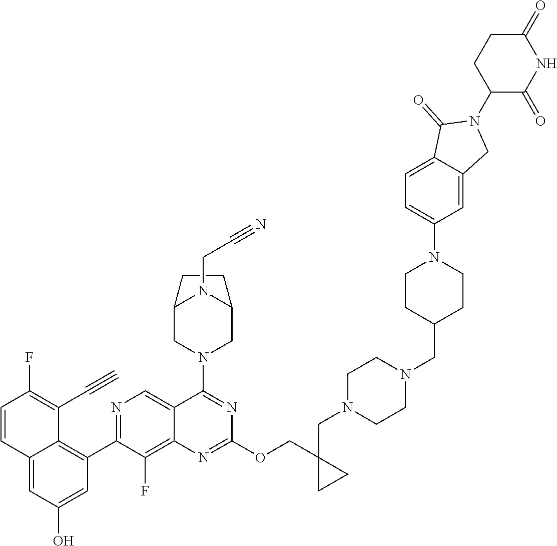 [SMILES: C#Cc1c(F)ccc2cc(O)cc(-c3ncc4c(N5CC6CCC(C5)N6CC#N)nc(OCC5(CN6CCN(CC7CCN(c8ccc9c(c8)CN(C8CCC(=O)NC8=O)C9=O)CC7)CC6)CC5)nc4c3F)c12] | 990.4 | 10.25-10.08 (m, 1H), 9.04 (s, 1H), 7.97 (dd, J = 5.8, 9.2 Hz, 1H), 7.51-7.43 (m, 2H), 7.39 (d, J = 2.4 Hz, 1H), 7.17 (d, J = 2.6 Hz, 1H), 7.06-7.00 (m, 2H), 5.03 (dd, J = 5.0, 13.4 Hz, 1H), 4.51 (s, 1H), 4.36- 4.25 (m, 4H), 4.21-4.16 (m, 1H), 3.94 (s, 1H), 3.88-3.80 (m, 2H), 3.73-3.67 (m, 1H), 3.64-3.58 (m, 3H), 3.47 (s, 2H), 2.96-2.74 (m, 5H), 2.62-2.58 (m, 1H), 2.34-2.28 (m, 6H), 2.09 (d, J = 6.6 Hz, 2H), 2.00-1.88 (m, 4H), 1.77-1.62 (m, 6H), 1.26-1.09 (m, 3H), 0.63 (s, 2H), 0.41 (s, 2H). |
| 102 | 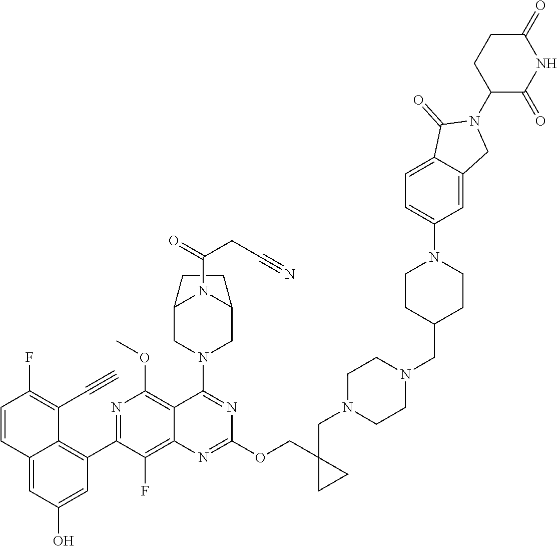 [SMILES: C#Cc1c(F)ccc2cc(O)cc(-c3nc(OC)c4c(N5CC6CCC(C5)N6C(=O)CC#N)nc(OCC5(CN6CCN(CC7CCN(c8ccc9c(c8)CN(C8CCC(=O)NC8=O)C9=O)CC7)CC6)CC5)nc4c3F)c12] | 1048.0 | 11.02-10.88 (m, 1H), 10.33-9.97 (m, 1H), 8.23-8.18 (m, 1H), 7.98 (dd, J = 6.0, 8.8 Hz, 1H), 7.52-7.44 (m, 2H), 7.41-7.37 (m, 1H), 7.24-7.20 (m, 1H), 7.06-7.00 (m, 2H), 5.10-5.00 (m, 1H), 4.68-4.60 (m, 1H), 4.36-4.26 (m, 4H), 4.22-4.16 (m, 2H), 4.12 (d, J = 3.6 Hz, 2H), 3.96 (d, J = 8.0 Hz, 1H), 3.92-3.80 (m, 6H), 3.60-3.52 (m, 2H), 2.95-2.84 (m, 2H), 2.83-2.75 (m, 2H), 2.58 (dd, J = 3.6, 19.4 Hz, 2H), 2.43- 2.36 (m, 4H), 2.31-2.25 (m, 4H), 2.12- 2.05 (m, 2H), 2.00-1.89 (m, 2H), 1.83- 1.65 (m, 7H), 1.20-1.08 (m, 2H), 0.67- 0.59 (m, 2H), 0.41 (s, 2H). |
| 103 | 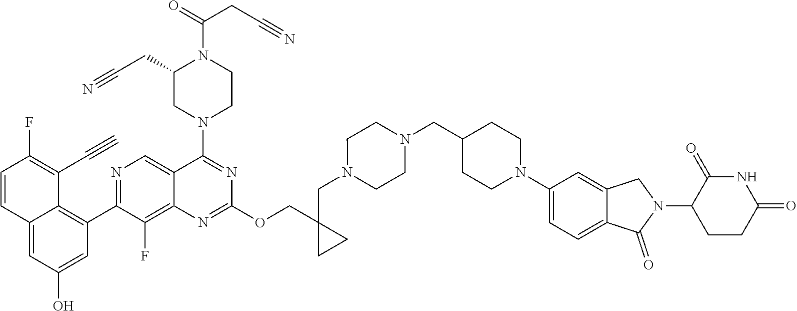 [SMILES: C#Cc1c(F)ccc2cc(O)cc(-c3ncc4c(N5CCN(C(=O)CC#N)[C@@H](CC#N)C5)nc(OCC5(CN6CCN(CC7CCN(c8ccc9c(c8)CN(C8CCC(=O)NC8=O)C9=O)CC7)CC6)CC5)nc4c3F)c12] | 1031.8 | 10.94 (s, 1H), 10.38-10.07 (m, 1H), 9.07 (s, 1H), 8.25-8.21 (m, 1H), 8.04-7.95 (m, 1H), 7.52-7.45 (m, 2H), 7.43-7.39 (m, 1H), 7.22-7.15 (m, 1H), 7.06-7.00 (m, 2H), 5.04 (dd, J = 5.0, 13.4 Hz, 1H), 4.91- 4.80 (m, 1H), 4.53-4.37 (m, 2H), 4.36- 4.26 (m, 5H), 4.23-4.13 (m, 3H), 3.94- 3.76 (m, 6H), 3.11-3.03 (m, 2H), 2.96- 2.74 (m, 5H), 2.60 (dd, J = 1.6, 5.8 Hz, 4H), 2.33-2.25 (m, 4H), 2.14-2.07 (m, 2H), 2.00-1.91 (m, 1H), 1.79-1.68 (m, 3H), 1.26-1.07 (m, 3H), 0.65 (s, 2H), 0.43 (s, 2H) |
| 104 | 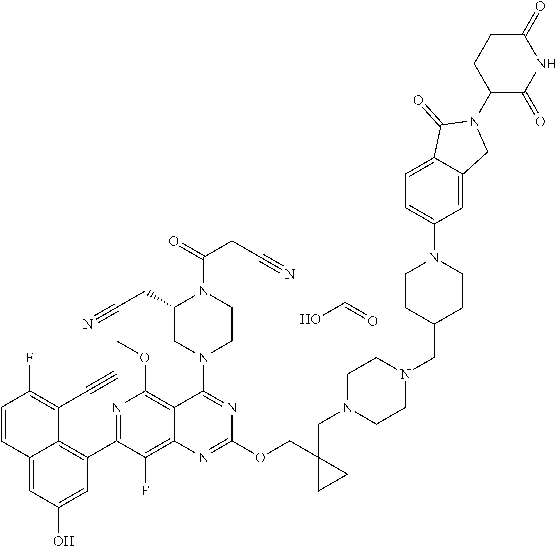 | 1061.2 | 10.99-10.88 (m, 1H), 8.28-8.26 (m, 1H), 7.98 (dd, J = 6.0, 9.3 Hz, 1H), 7.51-7.45 (m, 2H), 7.39 (s, 1H), 7.25-7.21 (m, 1H), 7.05-7.00 (m, 2H), 5.04 (dd, J = 4.8, 13.3 Hz, 1H), 5.01-4.90 (m, 1H), 4.54-4.44 (m, 1H), 4.38-4.26 (m, 4H), 4.22-4.11 (m, 3H), 4.11-4.06 (m, 1H), 4.04-3.99 (m, 1H), 3.92 (s, 1H), 3.88 (s, 1H), 3.87-3.80 (m, 3H), 3.04-2.98 (m, 2H), 2.96-2.89 (m, 3H), 2.85-2.74 (m, 4H), 2.38-2.32 (m, 5H), 2.29 (s, 2H), 2.14-2.05 (m, 3H), 2.01-1.90 (m, 2H), 1.79-1.66 (m, 4H), 1.26-1.20 (m, 1H), 1.17-1.12 (m, 2H), 0.63 (s, 2H), 0.41 (s, 2H). |
| 105 | 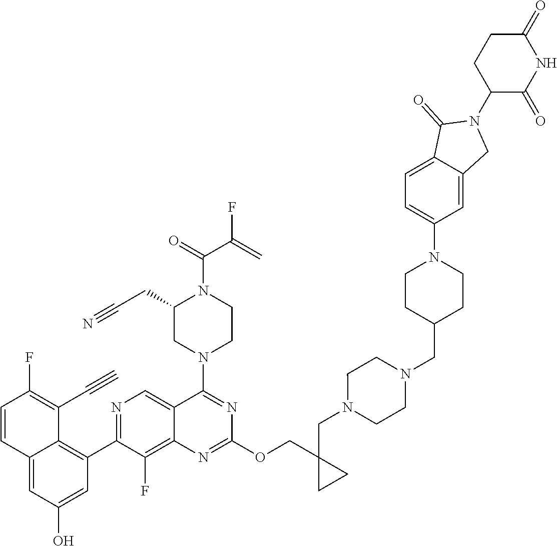 [SMILES: C#Cc1c(F)ccc2cc(O)cc(-c3ncc4c(N5CCN(C(=O)C(=C)F)[C@@H](CC#N)C5)nc(OCC5(CN6CCN(CC7CCN(c8ccc9c(c8)CN(C8CCC(=O)NC8=O)C9=O)CC7)CC6)CC5)nc4c3F)c12] | 1036.7 | 10.96-10.91 (m, 1H), 10.26-10.14 (m, 1H), 9.17-9.07 (m, 1H), 8.24-8.17 (m, 1H), 8.02-7.96 (m, 1H), 7.52-7.44 (m, 2H), 7.41 (d, J = 2.2 Hz, 1H), 7.20 (s, 1H), 7.05-7.01 (m, 2H), 5.47-5.36 (m, 1H), 5.08-5.00 (m, 1H), 4.49-4.40 (m, 1H), 4.35-4.26 (m, 3H), 4.23-4.16 (m, 1H), 3.96-3.81 (m, 3H), 2.95-2.76 (m, 5H), 2.62-2.58 (m, 1H), 2.55-2.54 (m, 2H), 2.39-2.36 (m, 3H), 2.31 (s, 2H), 2.13-2.06 (m, 3H), 2.04-1.91 (m, 3H), 1.79-1.65 (m, 4H), 1.30-1.12 (m, 8H), 0.88-0.81 (m, 1H), 0.67-0.62 (m, 2H), 0.43 (s, 2H). |
| 106 | 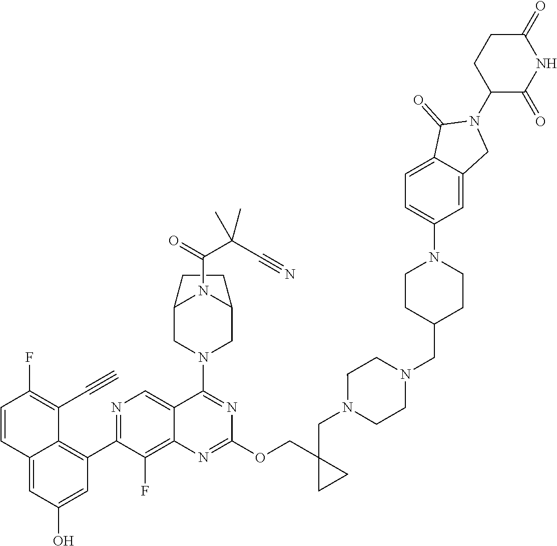 [SMILES: C#Cc1c(F)ccc2cc(O)cc(-c3ncc4c(N5CC6CCC(C5)N6C(=O)C(C)(C)C#N)nc(OCC5(CN6CCN(CC7CCN(c8ccc9c(c8)CN(C8CCC(=O)NC8=O)C9=O)CC7)CC6)CC5)nc4c3F)c12] | 1046.5 | 10.93 (s, 1H), 9.07-9.03 (m, 1H), 8.23- 8.21 (m, 1H), 8.02-7.94 (m, 1H), 7.51- 7.43 (m, 2H), 7.42-7.38 (m, 1H), 7.18 (d, J = 2.4 Hz, 1H), 7.06-6.99 (m, 2H), 5.03 (dd, J = 4.8, 13.6 Hz, 1H), 4.96-4.79 (m, 2H), 4.76-4.67 (m, 1H), 4.50-4.43 (m, 1H), 4.34-4.28 (m, 3H), 4.21-4.15 (m, 1H), 3.94 (s, 1H), 3.89-3.79 (m, 3H), 2.83-2.75 (m, 2H), 2.59 (d, J = 3.2 Hz, 2H), 2.56 (d, J = 1.6 Hz, 4H), 2.35-2.28 (m, 6H), 2.12-2.06 (m, 2H), 2.01-1.81 (m, 6H), 1.79-1.67 (m, 4H), 1.64-1.55 (m, 6H), 1.19-1.10 (m, 2H), 0.68-0.61 (m, 2H), 0.45-0.38 (m, 2H). |
| 107 | 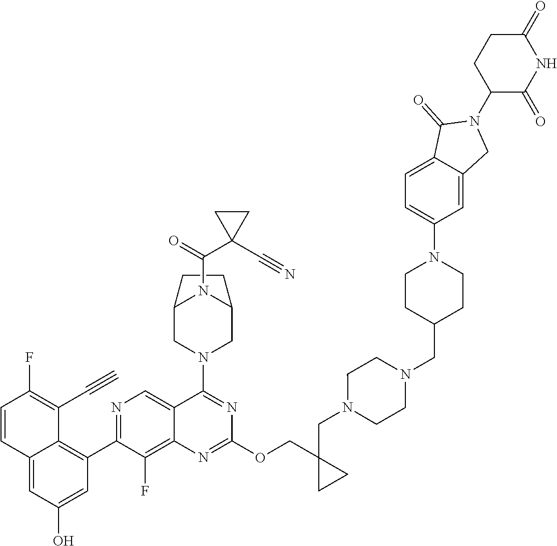 [SMILES: C#Cc1c(F)ccc2cc(O)cc(-c3ncc4c(N5CC6CCC(C5)N6C(=O)C5(C#N)CC5)nc(OCC5(CN6CCN(CC7CCN(c8ccc9c(c8)CN(C8CCC(=O)NC8=O)C9=O)CC7)CC6)CC5)nc4c3F)c12] | 1045.1 | 10.94 (s, 1H), 9.07 (s, 1H), 8.18 (s, 1H), 7.99 (dd, J = 5.6, 9.2 Hz, 1H), 7.52-7.44 (m, 2H), 7.40 (d, J = 2.4 Hz, 1H), 7.19 (d, J = 2.4 Hz, 1H), 7.07-7.00 (m, 2H), 5.04 (dd, J = 5.2, 13.3 Hz, 1H), 4.88-4.74 (m, 1H), 4.74-4.66 (m, 1H), 4.51-4.44 (m, 1H), 4.35-4.28 (m, 3H), 4.22-4.16 (m, 1H), 3.95 (s, 1H), 3.85 (d, J = 11.2 Hz, 2H), 3.75-3.67 (m, 1H), 2.88 (d, J = 13.2 Hz, 2H), 2.80 (t, J = 11.2 Hz, 2H), 2.63- 2.59 (m, 1H), 2.58-2.54 (m, 1H), 2.47- 2.37 (m, 5H), 2.36-2.28 (m, 6H), 2.10 (d, J = 5.6 Hz, 2H), 2.03-1.80 (m, 6H), 1.74 (d, J = 11.2 Hz, 3H), 1.67-1.60 (m, 4H), 1.20-1.10 (m, 2H), 0.69-0.62 (m, 2H), 0.43 (s, 2H). |
| 108 | 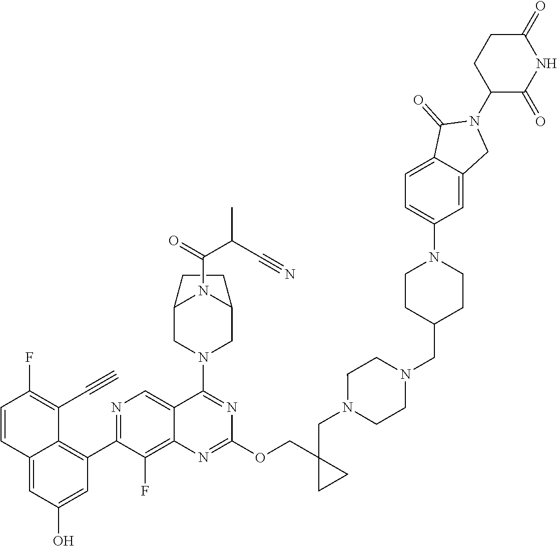 [SMILES: C#Cc1c(F)ccc2cc(O)cc(-c3ncc4c(N5CC6CCC(C5)N6C(=O)C(C)C#N)nc(OCC5(CN6CCN(CC7CCN(c8ccc9c(c8)CN(C8CCC(=O)NC8=O)C9=O)CC7)CC6)CC5)nc4c3F)c12] | 1033.2 | 10.98-10.87 (m, 1H), 9.07-9.00 (m, 1H), 8.22 (s, 1H), 7.98 (dd, J = 6.2, 9.2 Hz, 1H), 7.51-7.43 (m, 2H), 7.40 (d, J = 2.4 Hz, 1H), 7.18 (d, J = 2.2 Hz, 1H), 7.05- 6.99 (m, 2H), 5.07-4.98 (m, 1H), 4.75- 4.69 (m, 1H), 4.64 (s, 2H), 4.45-4.35 (m, 2H), 4.29 (s, 3H), 4.21 (s, 1H), 3.93 (d, J = 5.2 Hz, 1H), 3.84 (d, J = 11.4 Hz, 3H), 3.75-3.68 (m, 1H), 3.62 (d, J = 12.4 Hz, 1H), 3.56-3.52 (m, 1H), 2.91-2.74 (m, 4H), 2.60 (d, J = 1.4 Hz, 1H), 2.55 (d, J = 2.2 Hz, 1H), 2.35-2.27 (m, 6H), 2.09 (d, J = 6.2 Hz, 2H), 2.04-1.91 (m, 3H), 1.90- 1.79 (m, 3H), 1.77-1.69 (m, 3H), 1.52- 1.41 (m, 3H), 1.21-1.10 (m, 2H), 0.68- 0.60 (m, 2H), 0.45-0.38 (m, 2H). |
| 109 | 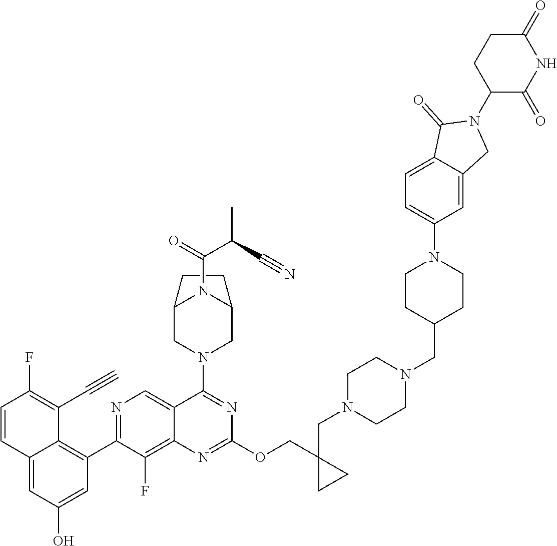 [SMILES: C#Cc1c(F)ccc2cc(O)cc(-c3ncc4c(N5CC6CCC(C5)N6C(=O)[C@@H](C)C#N)nc(OCC5(CN6CCN(CC7CCN(c8ccc9c(c8)CN(C8CCC(=O)NC8=O)C9=O)CC7)CC6)CC5)nc4c3F)c12] | | |
| 110 | 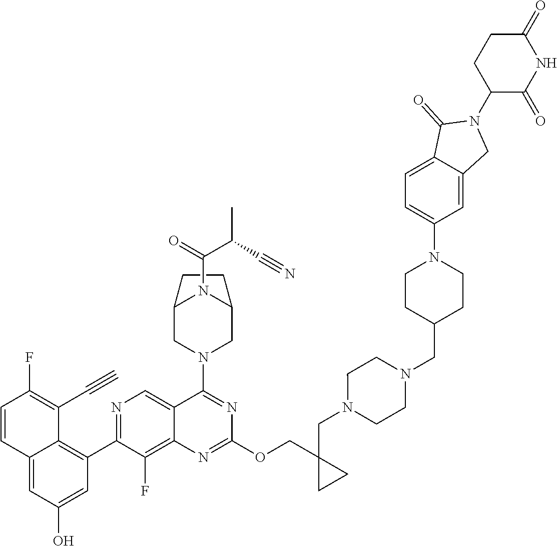 [SMILES: C#Cc1c(F)ccc2cc(O)cc(-c3ncc4c(N5CC6CCC(C5)N6C(=O)[C@H](C)C#N)nc(OCC5(CN6CCN(CC7CCN(c8ccc9c(c8)CN(C8CCC(=O)NC8=O)C9=O)CC7)CC6)CC5)nc4c3F)c12] | | |
| 111 | 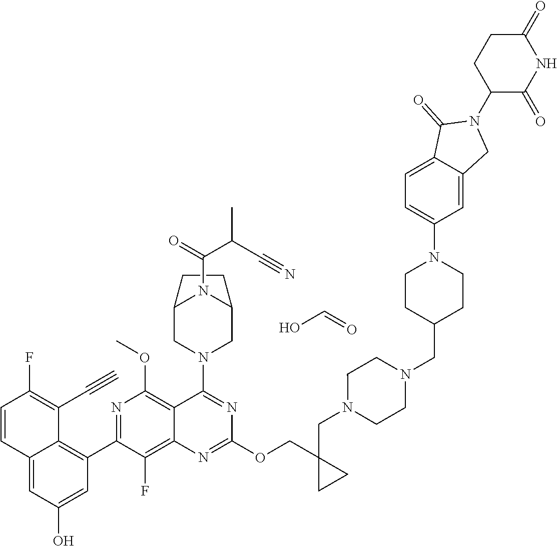 | 1066.3 | 10.93 (s, 1H), 10.15 (s, 1H), 8.16 (s, 1H), 7.97 (dd, J = 6.0, 9.3 Hz, 1H), 7.50-7.43 (m, 2H), 7.39 (d, J = 2.4 Hz, 1H), 7.23 (s, 1H), 7.04-7.00 (m, 2H), 5.03 (dd, J = 4.8, 13.1 Hz, 1H), 4.70 (d, J = 2.4 Hz, 1H), 4.62-4.50 (m, 1H), 4.29 (s, 3H), 4.21-4.16 (m, 1H), 3.99-3.93 (m, 1H), 3.86 (s, 2H), 2.95-2.85 (m, 1H), 2.83-2.75 (m, 2H), 2.57-2.53 (m, 2H), 2.41 (dt, J = 2.0, 5.5 Hz, 4H), 2.38-2.36 (m, 2H), 2.30 (s, 4H), 2.12-2.04 (m, 3H), 1.99-1.90 (m, 3H), 1.83-1.69 (m, 6H), 1.52-1.44 (m, 2H), 1.44-1.38 (m, 2H), 1.26-1.21 (m, 1H), 1.19-1.10 (m, 3H), 0.66-0.61 (m, 2H), 0.44-0.39 (m, 2H). |
| 112 | 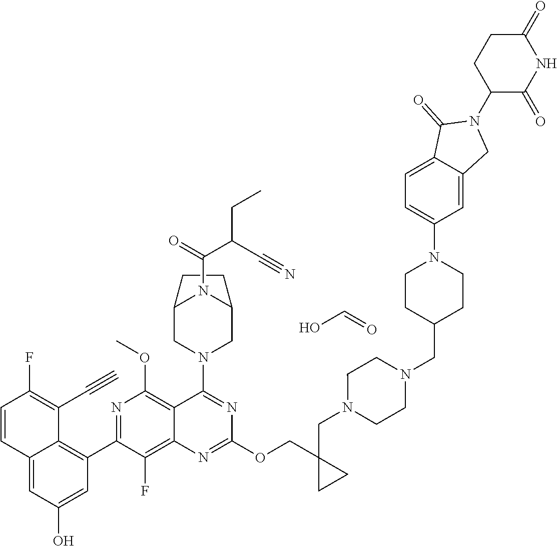 | 1079.9 | 10.93 (s, 1H), 10.22-10.12 (m, 1H), 8.19 (s, 1H), 7.97 (dd, J = 6.0, 9.3 Hz, 1H), 7.51-7.43 (m, 2H), 7.39 (d, J = 2.4 Hz, 1H), 7.23 (s, 1H), 7.05-6.99 (m, 2H), 5.07-5.00 (m, 1H), 4.71-4.53 (m, 2H), 4.36-4.23 (m, 5H), 4.20 (s, 1H), 3.96 (s, 1H), 3.88-3.81 (m, 2H), 3.63-3.41 (m, 3H), 2.94-2.86 (m, 1H), 2.82-2.75 (m, 2H), 2.60 (s, 2H), 2.43-2.35 (m, 4H), 2.29 (d, J = 4.0 Hz, 4H), 2.11-2.06 (m, 2H), 1.99-1.86 (m, 4H), 1.85-1.69 (m, 7H), 1.26-1.10 (m, 3H), 1.07-1.00 (m, 3H), 0.67-0.60 (m, 2H), 0.44-0.37 (m, 2H). |
| 113 | 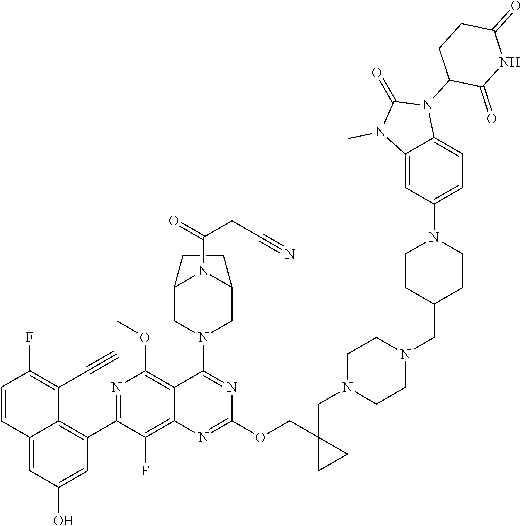 [SMILES: C#Cc1c(F)ccc2cc(O)cc(-c3nc(OC)c4c(N5CC6CCC(C5)N6C(=O)CC#N)nc(OCC5(CN6CCN(CC7CCN(c8ccc9c(c8)n(C)c(=O)n9C8CCC(=O)NC8=O)CC7)CC6)CC5)nc4c3F)c12] | 1066.5 | 11.06 (s, 1H), 10.21-10.12 (m, 1H), 8.22 (s, 1H), 7.98 (dd, J = 5.8, 9.3 Hz, 1H), 7.47 (t, J = 8.8 Hz, 1H), 7.39 (d, J = 2.1 Hz, 1H), 7.23 (s, 1H), 6.92 (d, J = 8.9 Hz, 1H), 6.81 (s, 1H), 6.64-6.59 (m, 1H), 5.33-5.23 (m, 1H), 4.68-4.60 (m, 1H), 4.37-4.27 (m, 4H), 4.15-4.08 (m, 3H), 3.95 (d, J = 8.0 Hz, 1H), 3.60-3.50 (m, 4H), 2.63 (d, J = 1.6 Hz, 2H), 2.60 (s, 2H), 2.31-2.27 (m, 5H), 2.14-2.07 (m, 4H), 2.02-1.89 (m, 4H), 1.80-1.72 (m, 5H), 1.64-1.51 (m, 2H), 1.27-1.18 (m, 4H), 0.67-0.59 (m, 3H), 0.47-0.38 (m, 3H) |
| 114 | 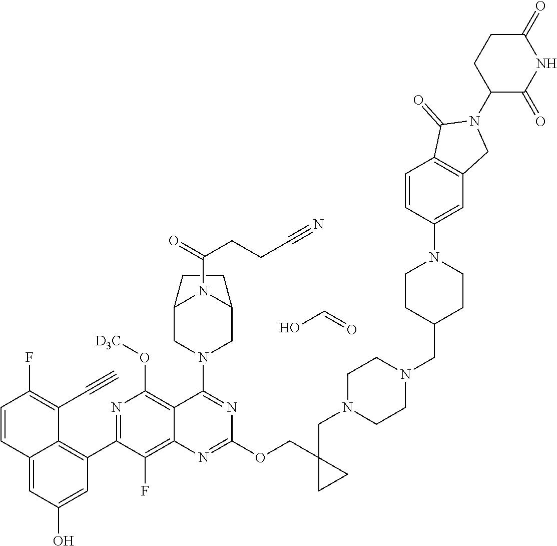 | 1065.3 | 10.94 (s, 1H), 10.35-9.95 (m, 1H), 8.15 (s, 1H), 7.98 (dd, J = 6.0, 9.2 Hz, 1H), 7.51- 7.44 (m, 2H), 7.39 (d, J = 2.6 Hz, 1H), 7.23 (d, J = 2.6 Hz, 1H), 7.06-6.99 (m, 2H), 5.04 (dd, J = 5.2, 13.2 Hz, 1H), 4.69- 4.62 (m, 1H), 4.45 (t, J = 6.4 Hz, 1H), 4.35-4.25 (m, 3H), 4.25-4.16 (m, 2H), 4.08-3.92 (m, 2H), 3.85 (d, J = 12.4 Hz, 2H), 3.57-3.44 (m, 4H), 2.96-2.85 (m, 2H), 2.84-2.76 (m, 4H), 2.72-2.67 (m, 2H), 2.59-2.53 (m, 1H), 2.39-2.25 (m, 8H), 2.14-2.07 (m, 2H), 1.99-1.89 (m, 2H), 1.81-1.64 (m, 6H), 1.26-1.10 (m, 2H), 0.69-0.59 (m, 2H), 0.46-0.37 (m, 2H). |
| 115 | 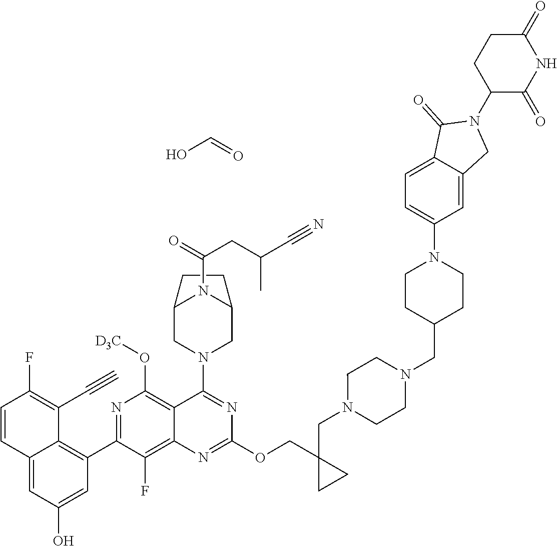 [SMILES: [2H]C([2H])([2H])Oc1nc(-c2cc(O)cc3ccc(F)c(C#C)c23)c(F)c2nc(OCC3(CN4CCN(CC5CCN(c6ccc7c(c6)CN(C6CCC(=O)NC6=O)C7=O)CC5)CC4)CC3)nc(N3CC4CCC(C3)N4C(=O)CC(C)C#N)c12 ; O=CO] | 1079.6 | 10.93 (s, 1H), 10.29-10.06 (m, 1H), 8.18 (s, 1H), 7.97 (dd, J = 6.0, 9.2 Hz, 1H), 7.53-7.42 (m, 2H), 7.39 (d, J = 2.4 Hz, 1H), 7.25-7.19 (m, 1H), 7.07-6.99 (m, 2H), 5.03 (dd, J = 5.0, 13.4 Hz, 1H), 4.65 (d, J = 4.4 Hz, 1H), 4.50-4.44 (m, 1H), 4.33-4.16 (m, 5H), 4.07-3.92 (m, 2H), 3.84 (d, J = 11.6 Hz, 2H), 3.54-3.41 (m, 3H), 3.15 (dd, J = 6.8, 13.4 Hz, 1H), 2.93- 2.84 (m, 2H), 2.83-2.74 (m, 4H), 2.60- 2.53 (m, 5H), 2.35-2.28 (m, 5H), 2.08 (d, J = 5.8 Hz, 2H), 1.98-1.89 (m, 2H), 1.74 (d, J = 12.4 Hz, 6H), 1.30 (t, J = 7.2 Hz, 3H), 1.19-1.09 (m, 2H), 0.63 (s, 2H), 0.41 (s, 2H). |
| 116 | 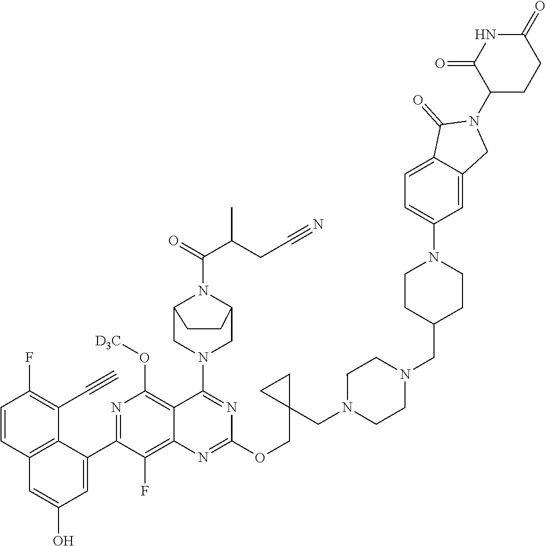 [SMILES: [2H]C([2H])([2H])Oc1nc(-c2cc(O)cc3ccc(F)c(C#C)c23)c(F)c2nc(OCC3(CN4CCN(CC5CCN(c6ccc7c(c6)CN(C6CCC(=O)NC6=O)C7=O)CC5)CC4)CC3)nc(N3CC4CCC(C3)N4C(=O)C(C)CC#N)c12] | 1079.5 | 10.97-10.91 (m, 1H), 10.25-10.12 (m, 1H), 7.97 (dd, J = 6.0, 9.3 Hz, 1H), 7.51- 7.42 (m, 2H), 7.39 (d, J = 2.4 Hz, 1H), 7.23 (d, J = 2.1 Hz, 1H), 7.06-6.98 (m, 2H), 5.03 (dd, J = 4.8, 13.4 Hz, 1H), 4.72- 4.52 (m, 2H), 4.35-4.26 (m, 3H), 4.23- 4.18 (m, 1H), 3.95 (s, 1H), 3.89-3.79 (m, 2H), 2.99-2.86 (m, 2H), 2.85-2.73 (m, 3H), 2.68-2.58 (m, 3H), 2.55 (s, 2H), 2.37 (d, J = 5.4 Hz, 3H), 2.33 (d, J = 1.6 Hz, 2H), 2.31-2.23 (m, 5H), 2.08 (d, J = 6.3 Hz, 2H), 1.99-1.88 (m, 3H), 1.80-1.66 (m, 6H), 1.23 (d, J = 3.4 Hz, 2H), 1.13 (d, J = 7.0 Hz, 3H), 0.67-0.59 (m, 2H), 0.43-0.36 (m, 2H). |
| 117 | 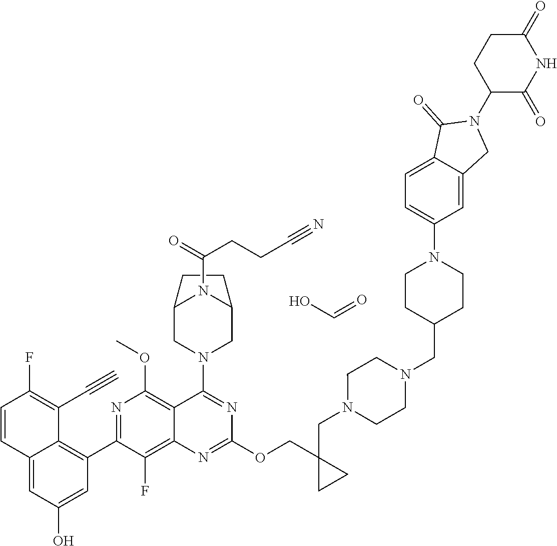 | 1062.5 | 10.98 (s, 1H), 8.26-8.14 (m, 1H), 8.03- 7.93 (m, 1H), 7.52-7.44 (m, 2H), 7.41- 7.33 (m, 1H), 7.22 (d, J = 1.6 Hz, 1H), 7.06-6.97 (m, 2H), 5.09-5.00 (m, 1H), 4.68-4.61 (m, 1H), 4.48-4.40 (m, 1H), 4.35-4.14 (m, 6H), 3.95 (d, J = 2.4 Hz, 1H), 3.89 (s, 3H), 3.87-3.80 (m, 2H), 2.84-2.75 (m, 4H), 2.72-2.65 (m, 3H), 2.35-2.26 (m, 8H), 2.13-2.05 (m, 4H), 1.99-1.87 (m, 4H), 1.80-1.67 (m, 7H), 1.19-1.10 (m, 2H), 0.67-0.59 (m, 2H), 0.44-0.36 (m, 2H). |
| 118 | 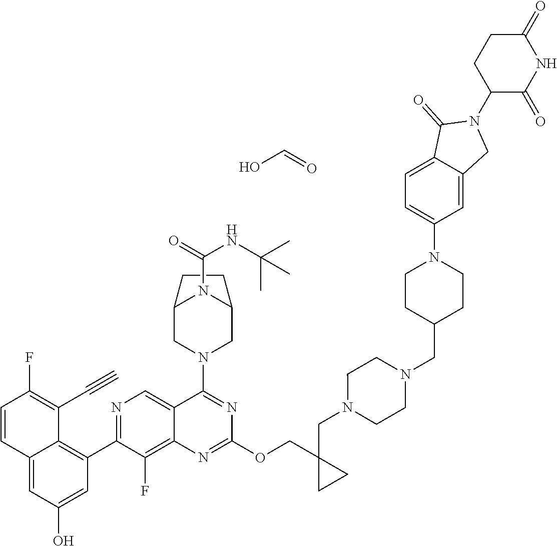 [SMILES: C#Cc1c(F)ccc2cc(O)cc(-c3ncc4c(N5CC6CCC(C5)N6C(=O)NC(C)(C)C)nc(OCC5(CN6CCN(CC7CCN(c8ccc9c(c8)CN(C8CCC(=O)NC8=O)C9=O)CC7)CC6)CC5)nc4c3F)c12 ; O=CO] | 1051.1 | 10.93 (s, 1H), 10.33-10.04 (m, 1H), 9.05 (s, 1H), 8.21 (s, 1H), 7.97 (dd, J = 6.0, 9.1 Hz, 1H), 7.51-7.43 (m, 2H), 7.39 (d, J = 2.4 Hz, 1H), 7.17 (d, J = 2.4 Hz, 1H), 7.06-6.98 (m, 2H), 6.15 (s, 1H), 5.03 (dd, J = 4.8, 13.0 Hz, 1H), 4.52-4.43 (m, 1H), 4.41-4.36 (m, 2H), 4.34-4.26 (m, 4H), 4.22-4.15 (m, 1H), 3.94 (s, 1H), 3.84 (d, J = 13.2 Hz, 2H), 3.71-3.60 (m, 2H), 2.96- 2.74 (m, 4H), 2.63-2.58 (m, 1H), 2.57- 2.52 (m, 2H), 2.35-2.26 (m, 6H), 2.09 (d, J = 6.6 Hz, 2H), 1.98-1.92 (m, 1H), 1.83- 1.63 (m, 8H), 1.30 (s, 9H), 1.23 (s, 1H), 1.21-1.10 (m, 2H), 0.68-0.59 (m, 2H), 0.41 (s, 2H). |
| 119 | 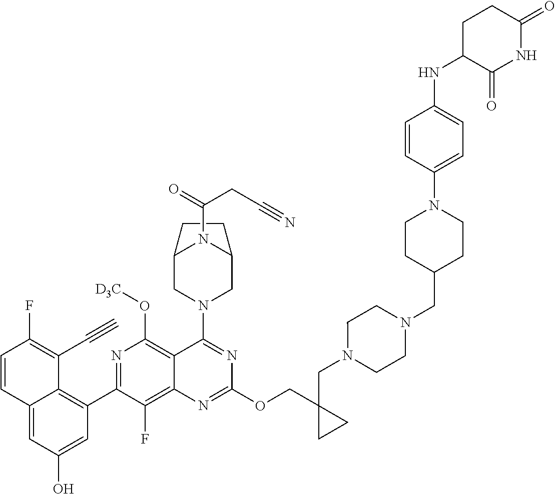 [SMILES: [2H]C([2H])([2H])Oc1nc(-c2cc(O)cc3ccc(F)c(C#C)c23)c(F)c2nc(OCC3(CN4CCN(CC5CCN(c6ccc(NC7CCC(=O)NC7=O)cc6)CC5)CC4)CC3)nc(N3CC4CCC(C3)N4C(=O)CC#N)c12] | 1011.8 | 10.74 (s, 1H), 8.24 (s, 1H), 7.97 (dd, J = 6.0, 9.3 Hz, 1H), 7.51-7.43 (m, 1H), 7.41- 7.35 (m, 1H), 7.25-7.19 (m, 1H), 6.73 (d, J = 8.8 Hz, 2H), 6.63-6.54 (m, 2H), 5.39- 5.29 (m, 1H), 4.70-4.58 (m, 1H), 4.37- 4.31 (m, 1H), 4.30-4.27 (m, 2H), 4.23- 4.15 (m, 2H), 4.11 (d, J = 3.2 Hz, 2H), 3.95 (d, J = 9.2 Hz, 1H), 3.61-3.49 (m, 2H), 2.79-2.70 (m, 2H), 2.58 (d, J = 4.4 Hz, 4H), 2.31-2.24 (m, 6H), 2.15-2.04 (m, 5H), 1.90-1.67 (m, 8H), 1.30-1.10 (m, 4H), 0.64-0.59 (m, 2H), 0.43-0.37 (m, 2H). |
| 120 | 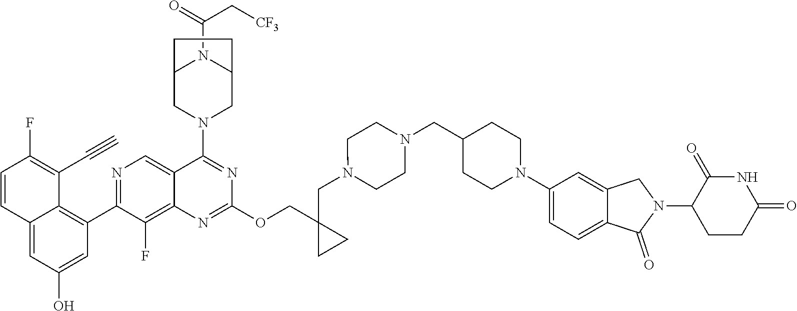 [SMILES: C#Cc1c(F)ccc2cc(O)cc(-c3ncc4c(N5CC6CCCC(C5)N6C(=O)CC(F)(F)F)nc(OCC5(CN6CCN(CC7CCN(c8ccc9c(c8)CN(C8CCC(=O)NC8=O)C9=O)CC7)CC6)CC5)nc4c3F)c12] | 1062.1 | 10.93 (s, 1H), 10.35-10.01 (m, 1H), 9.03 (d, J = 2.8 Hz, 1H), 7.98 (dd, J = 6.0, 9.3 Hz, 1H), 7.54-7.32 (m, 3H), 7.18 (t, J = 2.0 Hz, 1H), 7.07-6.97 (m, 2H), 5.03 (dd, J = 5.2, 13.2 Hz, 1H), 4.73 (s, 1H), 4.66- 4.52 (m, 2H), 4.48-4.35 (m, 1H), 4.34- 4.26 (m, 3H), 4.22-4.15 (m, 1H), 3.94 (d, J = 7.6 Hz, 1H), 3.88-3.79 (m, 3H), 3.79- 3.75 (m, 1H), 3.74-3.70 (m, 1H), 3.69- 3.62 (m, 2H), 3.56 (d, J = 12.0 Hz, 2H), 2.95-2.84 (m, 2H), 2.79 (t, J = 12.0 Hz, 2H), 2.60 (d, J = 2.4 Hz, 1H), 2.54 (s, 1H), 2.34-2.28 (m, 5H), 2.09 (d, J = 6.0 Hz, 2H), 1.99-1.91 (m, 2H), 1.86-1.68 (m, 6H), 1.28-1.05 (m, 3H), 0.64 (s, 2H), 0.41 (s, 2H). |
| 121 | 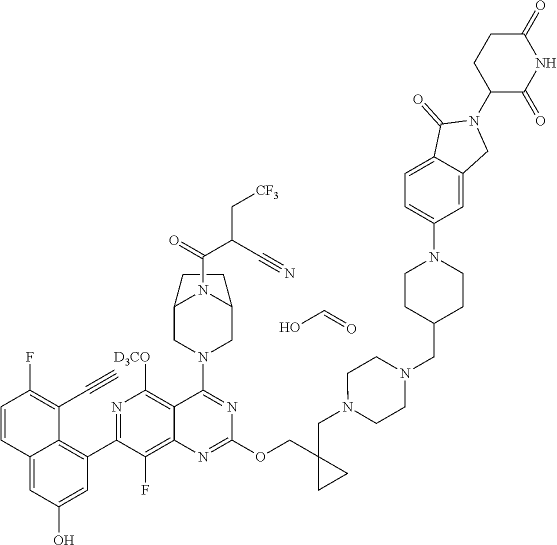 | 1133.7 | 10.94 (s, 1H), 10.29-10.10 (m, 1H), 8.31 (s, 1H), 7.98 (dd, J = 6.2, 9.0 Hz, 1H), 7.53-7.43 (m, 2H), 7.40 (d, J = 2.5 Hz, 1H), 7.23 (d, J = 2.8 Hz, 1H), 7.06-6.99 (m, 2H), 5.04 (dd, J = 5.2, 13.2 Hz, 1H), 4.77-4.63 (m, 3H), 4.34-4.16 (m, 5H), 4.00-3.94 (m, 1H), 3.85 (d, J = 11.4 Hz, 2H), 3.67-3.50 (m, 1H), 3.13-2.98 (m, 4H), 2.97-2.88 (m, 2H), 2.87-2.75 (m, 3H), 2.61 (s, 1H), 2.56 (d, J = 3.4 Hz, 2H), 2.39-2.37 (m, 1H), 2.31 (s, 3H), 2.11-2.06 (m, 2H), 2.01-1.91 (m, 3H), 1.89-1.81 (m, 2H), 1.78-1.69 (m, 4H), 1.29-1.05 (m, 4H), 0.64 (s, 2H), 0.42 (s, 2H). |
| 122 | 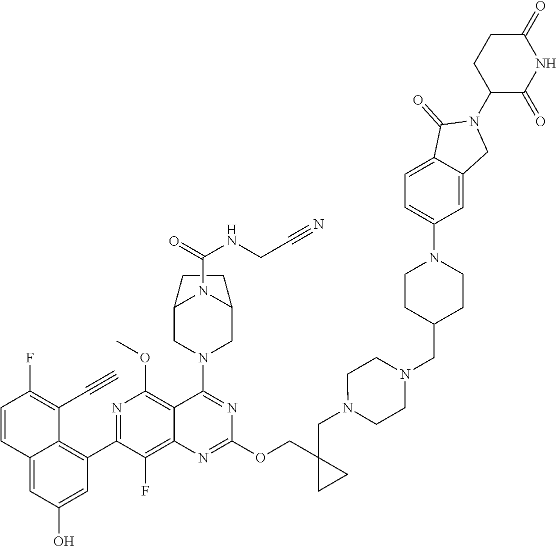 [SMILES: C#Cc1c(F)ccc2cc(O)cc(-c3nc(OC)c4c(N5CC6CCC(C5)N6C(=O)NCC#N)nc(OCC5(CN6CCN(CC7CCN(c8ccc9c(c8)CN(C8CCC(=O)NC8=O)C9=O)CC7)CC6)CC5)nc4c3F)c12] | | |
| 123 | 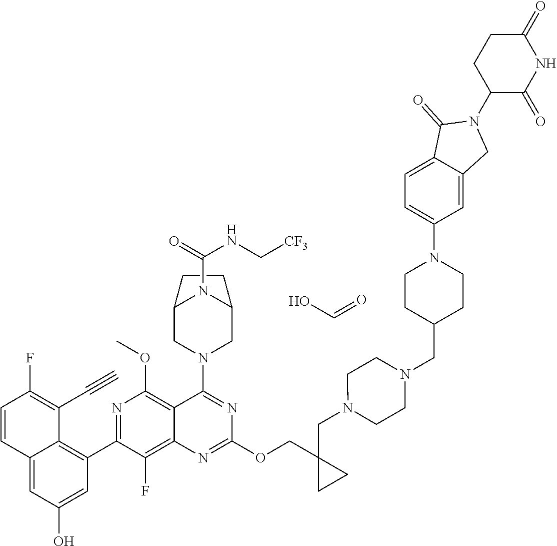 | | |
| 124 | 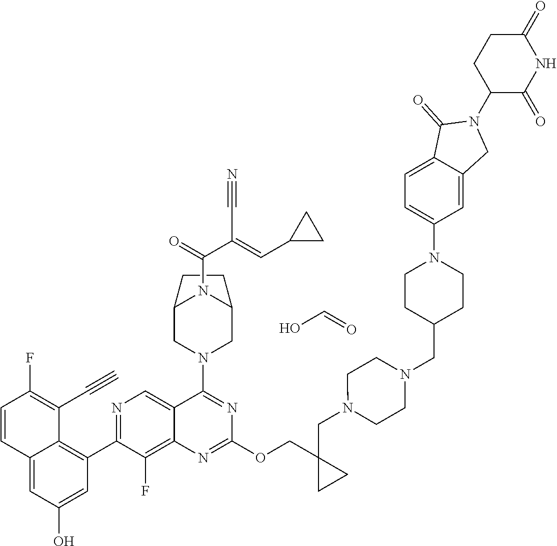 | 1071.1 | 10.94 (s, 1H), 10.31-10.08 (m, 1H), 9.06 (s, 1H), 8.32 (s, 1H), 7.98 (dd, J = 6.0, 9.3 Hz, 1H), 7.53-7.42 (m, 2H), 7.40 (d, J = 2.5 Hz, 1H), 7.18 (d, J = 2.5 Hz, 1H), 7.07-6.97 (m, 2H), 6.86 (d, J = 11.2 Hz, 1H), 5.04 (dd, J = 5.2, 13.4 Hz, 1H), 4.76- 4.62 (m, 3H), 4.46 (d, J = 13.4 Hz, 1H), 4.37-4.26 (m, 3H), 4.23-4.15 (m, 1H), 3.94 (d, J = 0.8 Hz, 1H), 3.88-3.80 (m, 2H), 3.79-3.61 (m, 3H), 2.95-2.84 (m, 3H), 2.84-2.74 (m, 4H), 2.60 (s, 2H), 2.09 (d, J = 5.6 Hz, 2H), 2.01-1.82 (m, 8H), 1.79-1.68 (m, 4H), 1.28-1.23 (m, 3H), 1.20-1.08 (m, 3H), 1.06-1.01 (m, 2H), 0.65 (s, 2H), 0.42 (s, 2H). |
| 125 | 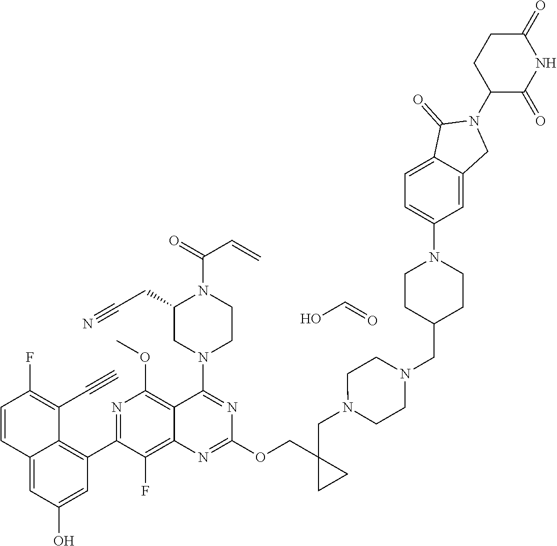 | 1049.0 | 10.93 (s, 1H), 10.16 (s, 1H), 8.14 (s, 1H), 7.98 (dd, J = 6.2, 9.0 Hz, 1H), 7.51-7.43 (m, 2H), 7.39 (t, J = 2.4 Hz, 1H), 7.23 (dd, J = 2.4, 5.8 Hz, 1H), 7.05-6.99 (m, 2H), 6.92-6.81 (m, 1H), 6.20 (dd, J = 2.0, 16.6 Hz, 1H), 5.79 (d, J = 11.0 Hz, 1H), 5.14-4.84 (m, 2H), 4.58-4.38 (m, 1H), 4.34-4.26 (m, 3H), 4.24-4.15 (m, 2H), 4.02 (d, J = 2.8 Hz, 1H), 3.94-3.81 (m, 6H), 3.77-3.58 (m, 1H), 3.53-3.39 (m, 2H), 3.00-2.73 (m, 6H), 2.60 (d, J = 2.4 Hz, 1H), 2.54 (s, 2H), 2.41 (s, 2H), 2.37 (d, J = 4.8 Hz, 2H), 2.30 (s, 2H), 2.26- 2.21 (m, 1H), 2.10 (t, J = 6.8 Hz, 2H), 1.98-1.91 (m, 1H), 1.74 (d, J = 10.9 Hz, 3H), 1.22-1.03 (m, 3H), 0.64 (s, 2H), 0.42 (s, 2H). |
| 126 | 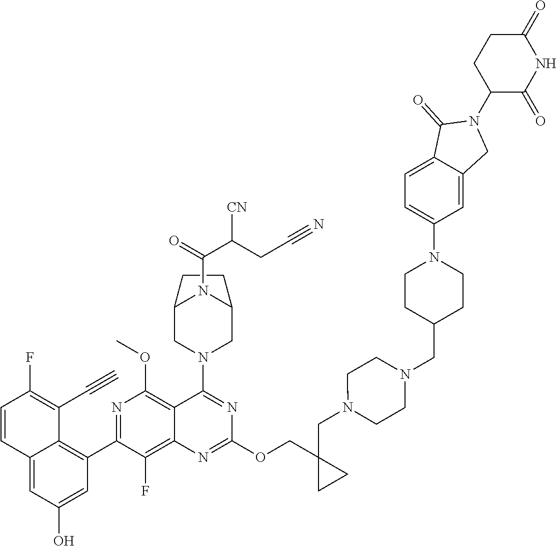 [SMILES: C#Cc1c(F)ccc2cc(O)cc(-c3nc(OC)c4c(N5CC6CCC(C5)N6C(=O)C(C#N)CC#N)nc(OCC5(CN6CCN(CC7CCN(c8ccc9c(c8)CN(C8CCC(=O)NC8=O)C9=O)CC7)CC6)CC5)nc4c3F)c12] | 1087.5 | δ 10.97-10.91 (m, 1H), 10.19-10.12 (m, 1H), 8.01-7.94 (m, 1H), 7.51-7.44 (m, 2H), 7.39 (d, J = 2.4 Hz, 1H), 7.22 (d, J = 2.6 Hz, 1H), 7.05-6.99 (m, 2H), 5.08-4.99 (m, 1H), 4.89-4.79 (m, 1H), 4.75-4.60 (m, 2H), 4.29 (s, 3H), 4.21-4.15 (m, 1H), 4.00- 3.94 (m, 1H), 3.90 (s, 3H), 3.87-3.81 (m, 2H), 3.65-3.51 (m, 1H), 3.20 (dd, J = 6.4, 9.8 Hz, 3H), 2.94-2.86 (m, 1H), 2.79 (t, J = 11.6 Hz, 2H), 2.61-2.54 (m, 2H), 2.39- 2.36 (m, 2H), 2.36-2.25 (m, 8H), 2.08 (d, J = 6.3 Hz, 2H), 2.00-1.90 (m, 3H), 1.89- 1.81 (m, 2H), 1.78-1.68 (m, 5H), 1.17- 1.09 (m, 2H), 0.63 (s, 2H), 0.41 (s, 2H). |
| 127 | 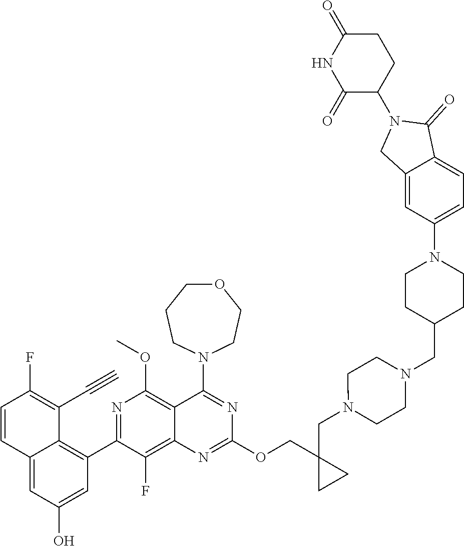 [SMILES: C#Cc1c(F)ccc2cc(O)cc(-c3nc(OC)c4c(N5CCCOCC5)nc(OCC5(CN6CCN(CC7CCN(c8ccc9c(c8)CN(C8CCC(=O)NC8=O)C9=O)CC7)CC6)CC5)nc4c3F)c12] | 970.5 | δ 10.93 (s, 1H), 10.30-10.01 (m, 1H), 8.22 (s, 1H), 7.97 (dd, J = 5.8, 9.2 Hz, 1H), 7.53-7.41 (m, 2H), 7.38 (d, J = 2.6 Hz, 1H), 7.22 (d, J = 2.5 Hz, 1H), 7.06-6.97 (m, 2H), 5.03 (dd, J = 5.0, 13.4 Hz, 1H), 4.34-4.16 (m, 4H), 4.00 (d, J = 0.9 Hz, 1H), 3.92-3.77 (m, 11H), 3.72 (t, J = 5.2 Hz, 2H), 3.49-3.39 (m, 2H), 3.00-2.70 (m, 4H), 2.61-2.56 (m, 1H), 2.42-2.36 (m, 3H), 2.29 (d, J = 12.4 Hz, 4H), 2.13-1.91 (m, 6H), 1.79-1.65 (m, 3H), 1.14 (d, J = 10.4 Hz, 2H), 0.62 (s, 2H), 0.40 (s, 2H). |
| 128 | 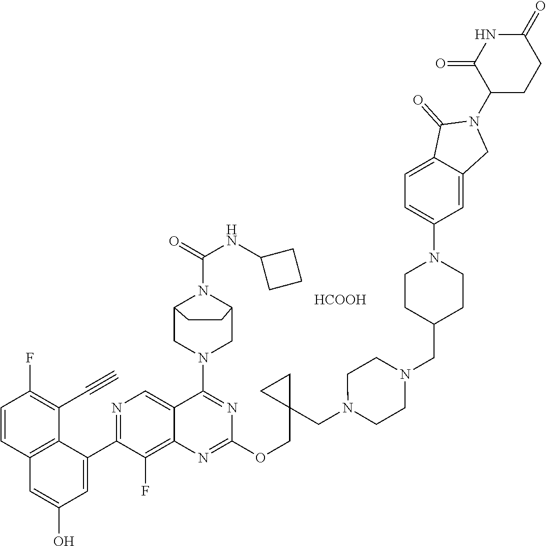 [SMILES: C#Cc1c(F)ccc2cc(O)cc(-c3ncc4c(N5CC6CCC(C5)N6C(=O)NC5CCC5)nc(OCC5(CN6CCN(CC7CCN(c8ccc9c(c8)CN(C8CCC(=O)NC8=O)C9=O)CC7)CC6)CC5)nc4c3F)c12 ; "HCOOH"] | 1048.5 [M − HCOOH + H]+. | δ 10.94 (s, 1H), 10.27-10.08 (m, 1H), 9.05 (s, 1H), 8.17 (s, 1H), 7.98 (dd, J = 6.0, 9.2 Hz, 1H), 7.51-7.43 (m, 2H), 7.39 (d, J = 2.4 Hz, 1H), 7.17 (d, J = 2.4 Hz, 1H), 7.06-6.98 (m, 2H), 6.90 (d, J = 7.8 Hz, 1H), 5.03 (dd, J = 5.0, 13.2 Hz, 1H), 4.50 (d, J = 11.6 Hz, 1H), 4.41 (s, 2H), 4.34- 4.25 (m, 4H), 4.22-4.13 (m, 2H), 3.95 (d, J = 0.8 Hz, 1H), 3.84 (d, J = 12.0 Hz, 2H), 3.67 (d, J = 11.4 Hz, 1H), 3.60 (d, J = 12.4 Hz, 1H), 2.97-2.73 (m, 4H), 2.58 (d, J = 16.2 Hz, 2H), 2.40-2.36 (m, 2H), 2.35- 2.25 (m, 6H), 2.21-2.06 (m, 5H), 2.03- 1.87 (m, 4H), 1.79-1.70 (m, 6H), 1.63- 1.53 (m, 2H), 1.21-1.09 (m, 2H), 0.64 (s, 2H), 0.41 (s, 2H). |
| 129 | 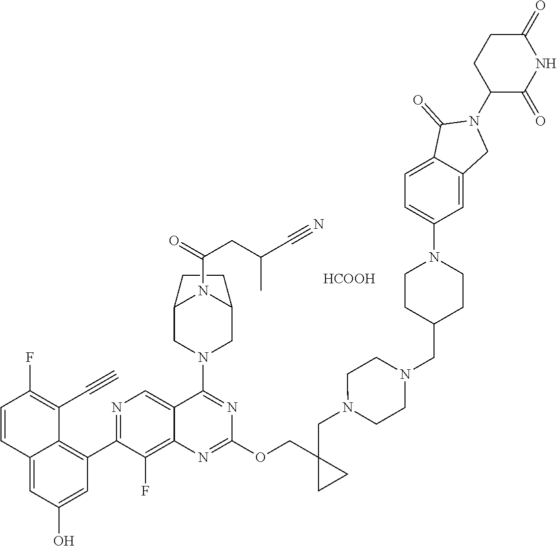 [SMILES: C#Cc1c(F)ccc2cc(O)cc(-c3ncc4c(N5CC6CCC(C5)N6C(=O)CC(C)C#N)nc(OCC5(CN6CCN(CC7CCN(c8ccc9c(c8)CN(C8CCC(=O)NC8=O)C9=O)CC7)CC6)CC5)nc4c3F)c12 ; "HCOOH"] | 1046.5 [M − HCOOH + H]+ | δ 10.93 (s, 1H), 8.99-9.10 (m, 1H), 8.21 (s, 1H), 7.98 (dd, J = 9.10, 5.90 Hz, 1H), 7.44-7.51 (m, 2H), 7.39 (d, J = 2.40 Hz, 1H), 7.18 (s, 1H), 6.99-7.05 (m, 2H), 4.96- 5.09 (m, 1H), 4.68-4.75 (m, 1H), 4.43- 4.66 (m, 3H), 4.33 (s, 1H), 4.29 (s, 3H), 4.15-4.22 (m, 1H), 3.91-3.98 (m, 1H), 3.77-3.89 (m, 3H), 3.52-3.66 (m, 2H), 2.84-2.96 (m, 3H), 2.75-2.83 (m, 3H), 2.28-2.35 (m, 6H), 2.09 (d, J = 6.00 Hz, 3H), 1.90-1.99 (m, 3H), 1.65-1.87 (m, 8H), 1.31 (t, J = 7.60 Hz, 3H), 1.10-1.18 (m, 2H), 0.62-0.67 (m, 2H), 0.42 (s, 2H) |
| 130 | 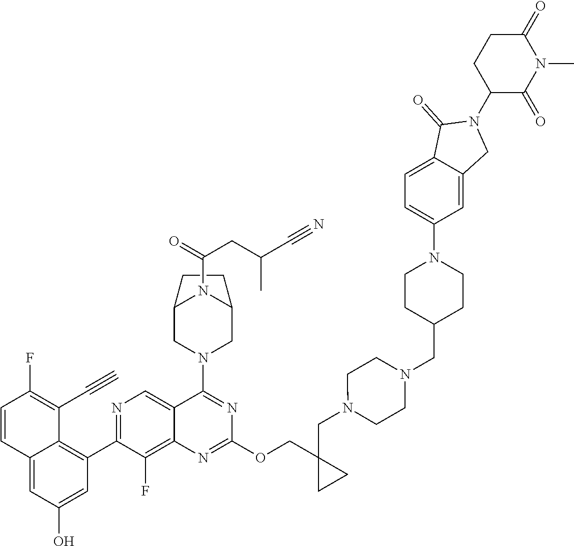 [SMILES: C#Cc1c(F)ccc2cc(O)cc(-c3ncc4c(N5CC6CCC(C5)N6C(=O)CC(C)C#N)nc(OCC5(CN6CCN(CC7CCN(c8ccc9c(c8)CN(C8CCC(=O)N(C)C8=O)C9=O)CC7)CC6)CC5)nc4c3F)c12] | 1060.5 | δ 10.21-10.08 (m, 1H), 9.07-9.00 (m, 1H), 8.01-7.94 (m, 1H), 7.52-7.44 (m, 2H), 7.39 (d, J = 2.4 Hz, 1H), 7.21-7.15 (m, 1H), 7.04-6.99 (m, 2H), 5.10 (dd, J = 5.2, 13.5 Hz, 1H), 4.74-4.68 (m, 1H), 4.65- 4.56 (m, 1H), 4.52 (d, J = 3.8 Hz, 1H), 4.36-4.26 (m, 4H), 4.20-4.14 (m, 1H), 3.95 (d, J = 9.6 Hz, 1H), 3.84 (d, J = 12.4 Hz, 2H), 2.99 (s, 3H), 2.80-2.70 (m, 4H), 2.40-2.24 (m, 11H), 2.09 (d, J = 5.8 Hz, 3H), 2.02-1.93 (m, 3H), 1.88-1.66 (m, 8H), 1.31 (t, J = 7.6 Hz, 3H), 1.20-1.07 (m, 3H), 0.64 (s, 2H), 0.41 (s, 2H) |
| 131 | 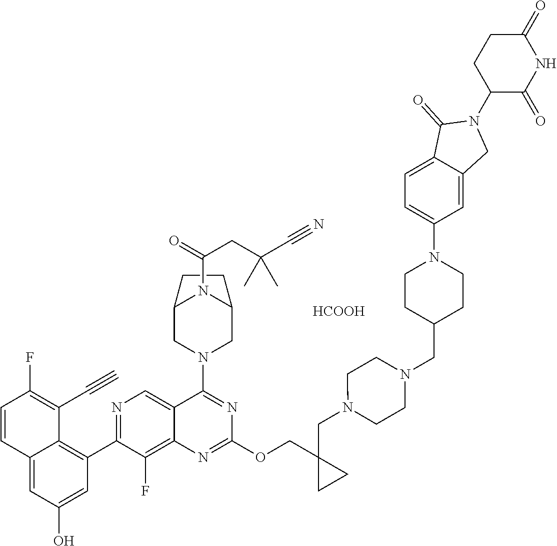 [SMILES: C#Cc1c(F)ccc2cc(O)cc(-c3ncc4c(N5CC6CCC(C5)N6C(=O)CC(C)(C)C#N)nc(OCC5(CN6CCN(CC7CCN(c8ccc9c(c8)CN(C8CCC(=O)NC8=O)C9=O)CC7)CC6)CC5)nc4c3F)c12 ; "HCOOH"] | 1061.6 | δ 10.99-10.91 (m, 1H), 9.07-9.03 (m, 1H), 8.22-8.20 (m, 1H), 8.02-7.95 (m, 1H), 7.52-7.44 (m, 2H), 7.40 (d, J = 2.5 Hz, 1H), 7.21-7.17 (m, 1H), 7.06-7.01 (m, 2H), 5.08-5.00 (m, 1H), 4.73 ( s, 1H), 4.67-4.58 (m, 1H), 4.56-4.50 (m, 1H), 4.49-4.38 (m, 1H), 4.30 (s, 3H), 4.23-4.19 (m, 1H), 3.96 (d, J = 8.8 Hz, 1H), 3.85 (d, J = 12.8 Hz, 2H), 3.76-3.61 (m, 2H), 3.56 (d, J = 12.0 Hz, 1H), 2.95-2.74 (m, 6H), 2.73-2.66 (m, 1H), 2.62-2.54 (m, 2H), 2.38 (d, J = 4.0 Hz, 2H), 2.36-2.28 (m, 6H), 2.10 (d, J = 6.0 Hz, 2H), 1.95 (dd, J = 5.0, 10.4 Hz, 2H), 1.86-1.70 (m, 6H), 1.45 (s, 3H), 1.42 (s, 3H), 1.20-1.11 (m, 2H), 0.67-0.61 (m, 2H), 0.46-0.39 (m, 2H). |
| 132 | 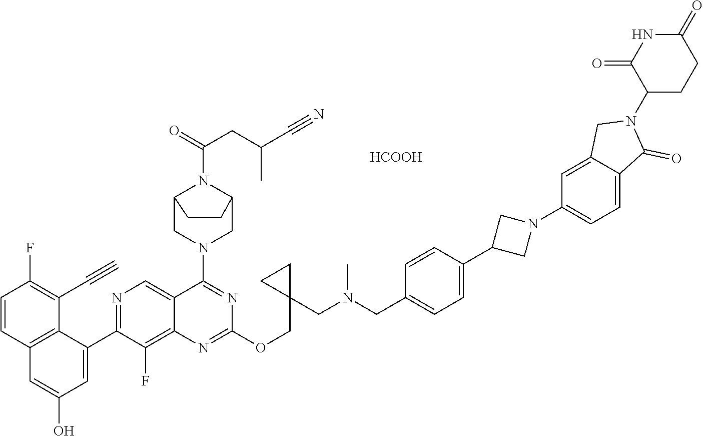 [SMILES: C#Cc1c(F)ccc2cc(O)cc(-c3ncc4c(N5CC6CCC(C5)N6C(=O)CC(C)C#N)nc(OCC5(CN(C)Cc6ccc(C7CN(c8ccc9c(c8)CN(C8CCC(=O)NC8=O)C9=O)C7)cc6)CC5)nc4c3F)c12 ; "HCOOH"] | 1039.4 [M − HCOOH + H]+ | δ 10.94 (s, 1H), 10.30-10.11 (m, 1H), 9.04 (s, 1H), 8.35 (s, 1H), 8.03-7.94 (m, 1H), 7.5-7.37 (m, 3H), 7.28-7.09 (m, 5H), 6.55- 6.38 (m, 2H), 5.04 (dd, J = 5.0, 13.4 Hz, 1H), 4.73-4.66 (m, 1H), 4.54-4.42 (m, 3H), 4.32-4.23 (m, 4H), 4.19-4.11 (m, 1H), 3.91 (d, J = 10.6 Hz, 2H), 3.85-3.74 (m, 3H), 3.52-3.42 (m, 3H), 3.14 (s, 2H), 2.94-2.84 (m, 2H), 2.95-2.71 (m, 2H), 2.61-2.56 (m, 2H), 2.35-2.28 (m, 2H), 2.17 (s, 3H), 1.99-1.70 (m, 5H), 1.31 (t, J = 7.2 Hz, 2H), 0.66 (s, 2H), 0.44 (s, 2H). |
| 133 | 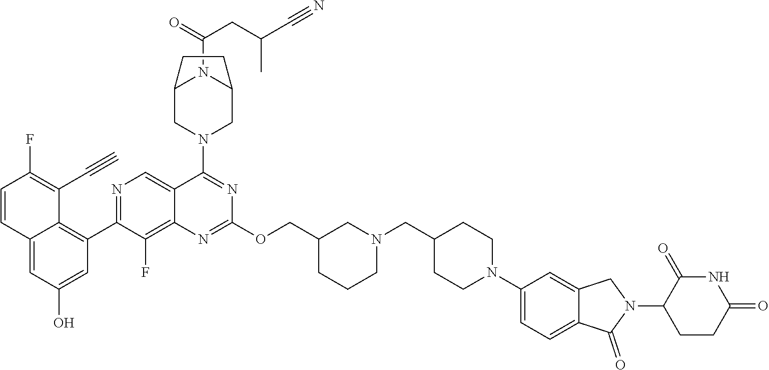 [SMILES: C#Cc1c(F)ccc2cc(O)cc(-c3ncc4c(N5CC6CCC(C5)N6C(=O)CC(C)C#N)nc(OCC5CCCN(CC6CCN(c7ccc8c(c7)CN(C7CCC(=O)NC7=O)C8=O)CC6)C5)nc4c3F)c12] | 991.5 | δ 10.97-10.90 (m, 1H), 9.08-9.00 (m, 1H), 8.42-8.37 (m, 1H), 8.02-7.93 (m, 1H), 7.52-7.43 (m, 2H), 7.41-7.36 (m, 1H), 7.22-7.15 (m, 1H), 7.05-6.96 (m, 2H), 5.07-4.99 (m, 1H), 4.74-4.67 (m, 1H), 4.56-4.45 (m, 2H), 4.34-4.24 (m, 3H), 4.20-4.15 (m, 1H), 3.95 (d, J = 9.5 Hz, 1H), 3.89-3.70 (m, 4H), 3.67-3.52 (m, 2H), 3.20-3.12 (m, 2H), 2.96-2.81 (m, 4H), 2.76 (s, 2H), 2.62-2.57 (m, 1H), 2.14 (d, J = 5.3 Hz, 2H), 1.94 (d, J = 7.8 Hz, 4H), 1.76 (d, J = 1.8 Hz, 6H), 1.71-1.59 (m, 2H), 1.54-1.45 (m, 1H), 1.30 (t, J = 7.6 Hz, 3H), 1.25-1.05 (m, 4H). |
| 134 | 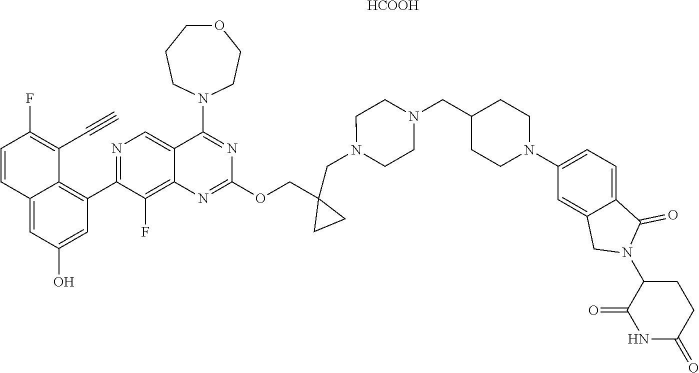 [SMILES: "HCOOH" ; C#Cc1c(F)ccc2cc(O)cc(-c3ncc4c(N5CCCOCC5)nc(OCC5(CN6CCN(CC7CCN(c8ccc9c(c8)CN(C8CCC(=O)NC8=O)C9=O)CC7)CC6)CC5)nc4c3F)c12] | 940.4 [M − COOH + H]+ | δ 10.93 (s, 1H), 10.36-9.99 (m, 1H), 8.88 (s, 1H), 8.22 (s, 1H), 7.96 (dd, J = 6.0, 9.4 Hz, 1H), 7.53-7.40 (m, 2H), 7.37 (d, J = 2.6 Hz, 1H), 7.13 (d, J = 2.0 Hz, 1H), 7.06-6.97 (m, 2H), 5.03 (dd, J = 5.2, 13.4 Hz, 1H), 4.74 (dd, J = 11.4, 16.9 Hz, 1H), 4.37-4.26 (m, 2H), 4.22-4.05 (m, 3H), 3.97 (s, 1H), 3.67-3.59 (m, 1H), 3.93-3.58 (m, 7H), 2.98-2.70 (m, 4H), 2.58 (dd, J = 2.2, 15.6 Hz, 2H), 2.45-2.25 (m, 9H), 2.08 (d, J = 6.0 Hz, 2H), 1.98-1.87 (m, 3H), 1.81-1.64 (m, 3H), 1.22-1.07 (m, 2H), 0.82-0.67 (m, 2H), 0.50 (d, J = 2.6 Hz, 2H). |
| 135 | 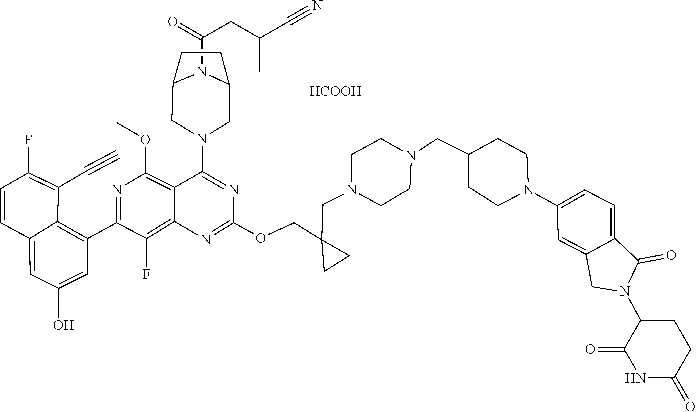 [SMILES: C#Cc1c(F)ccc2cc(O)cc(-c3nc(OC)c4c(N5CC6CCC(C5)N6C(=O)CC(C)C#N)nc(OCC5(CN6CCN(CC7CCN(c8ccc9c(c8)CN(C8CCC(=O)NC8=O)C9=O)CC7)CC6)CC5)nc4c3F)c12 ; "HCOOH"] | 1076.7 [M − COOH + H]+ | δ 10.99-10.87 (m, 1H), 8.27-8.21 (m, 1H), 8.01-7.93 (m, 1H), 7.51-7.43 (m, 2H), 7.39 (d, J = 2.4 Hz, 1H), 7.25-7.20 (m, 1H), 7.06-6.97 (m, 2H), 5.08-4.99 (m, 1H), 4.66 (s, 1H), 4.51-4.41 (m, 1H), 4.28 (s, 2H), 4.23-4.14 (m, 2H), 3.95 (s, 1H), 3.89 (s, 3H), 3.84 (d, J = 12.4 Hz, 2H), 3.49 (s, 2H), 3.18-3.11 (m, 2H), 2.97-2.72 (m, 6H), 2.65-2.53 (m, 2H), 2.45-2.39 (m, 3H), 2.37 (d, J = 4.2 Hz, 1H), 2.34 (d, J = 4.8 Hz, 2H), 2.29 (s, 4H), 2.11-2.05 (m, 2H), 1.98-1.89 (m, 2H), 1.80-1.66 (m, 6H), 1.30 (t, J = 7.2 Hz, 3H), 1.18-1.09 (m, 2H), 0.67-0.58 (m, 2H), 0.45-0.38 (m, 2H). |
| 136 | 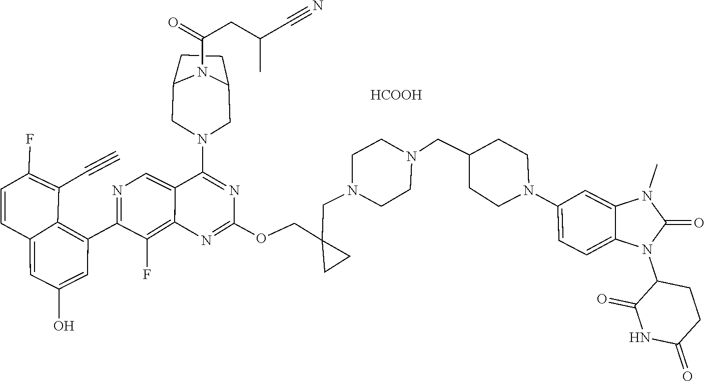 [SMILES: C#Cc1c(F)ccc2cc(O)cc(-c3ncc4c(N5CC6CCC(C5)N6C(=O)CC(C)C#N)nc(OCC5(CN6CCN(CC7CCN(c8ccc9c(c8)n(C)c(=O)n9C8CCC(=O)NC8=O)CC7)CC6)CC5)nc4c3F)c12 ; "HCOOH"] | 1061.5 [M − HCOOH + H]+. | δ 11.00-11.09 (m, 1H), 9.04 (t, J = 3.20 Hz, 1H), 8.30-8.34 (m, 1H), 7.98 (dd, J = 9.40, 5.88 Hz, 1H), 7.46 (t, J = 8.90 Hz, 1H), 7.39 (d, J = 2.40 Hz, 1H), 7.18 (br s, 1H), 6.88-6.94 (m, 1H), 6.80 (d, J = 1.88 Hz, 1H), 6.58-6.64 (m, 1H), 5.21-5.34 (m, 1H), 4.68-4.75 (m, 1H), 4.32-4.68 (m, 4 H) 4.26-4.32 (m, 2H), 3.92-3.98 (m, 1H), 3.61-3.80 (m, 2H), 3.51-3.58 (m, 3H), 2.82-3.01 (m, 3H), 2.69-2.82 (m, 2H), 2.54-2.65 (m, 6H), 2.21-2.31 (m, 5H), 2.11 (br d, J = 7.00 Hz, 2H), 1.89-2.04 (m, 3H), 1.65-1.88 (m, 6H), 1.52-1.63 (m, 1H), 1.31 (t, J = 7.60 Hz, 3H), 1.17-1.27 (m, 3H), 0.60-0.68 (m, 2H), 0.37-0.44 (m, 2H). |
| 137 | 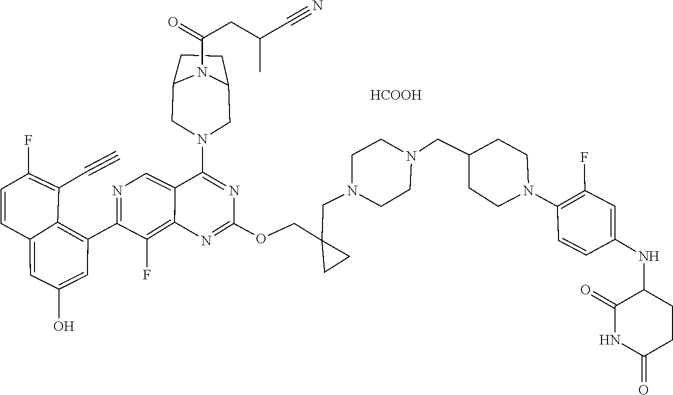 [SMILES: C#Cc1c(F)ccc2cc(O)cc(-c3ncc4c(N5CC6CCC(C5)N6C(=O)CC(C)C#N)nc(OCC5(CN6CCN(CC7CCN(c8ccc(NC9CCC(=O)NC9=O)cc8F)CC7)CC6)CC5)nc4c3F)c12 ; "HCOOH"] | 1024.6 [M − HCOOH + H]+ | δ 10.7 (s, 1H), 8.99-9.08 (m, 1H), 8.26- 8.33 (m, 1H), 7.92-8.03 (m, 1H), 7.43- 7.51 (m, 1H), 7.39 (d, J = 2.50 Hz, 1H), 7.15-7.19 (m, 1H), 6.76-6.84 (m, 1H), 6.46-6.51 (m, 1H), 6.37-6.42 (m, 1H), 5.77 (d, J = 7.00 Hz, 1H), 4.36-4.72 (m, 5H), 4.29 (s, 2H), 4.20-4.25 (m, 1H), 3.90- 3.98 (m, 1H), 3.66-3.81 (m, 2H), 2.70- 2.89 (m, 5H), 2.24-2.31 (m, 5H), 2.05- 2.13 (m, 4H), 1.90-2.00 (m, 2H), 1.67- 1.87 (m, 8H), 1.44-1.57 (m, 2H), 1.31 (t, J = 7.70 Hz, 3H), 1.14-1.25 (m, 4H), 1.01- 1.10 (m, 1H), 0.61-0.66 (m, 2H), 0.39- 0.43 (m, 2H). |
| 138 | 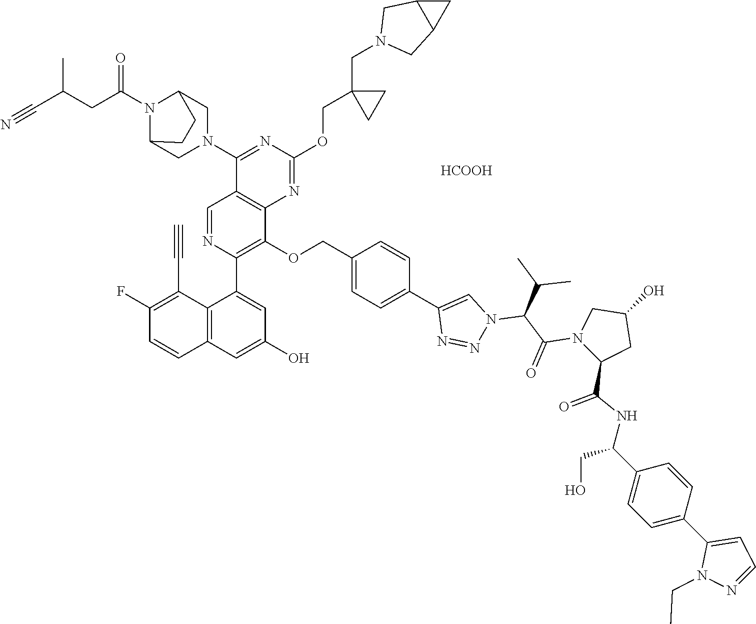 [SMILES: C#Cc1c(F)ccc2cc(O)cc(-c3ncc4c(N5CC6CCC(C5)N6C(=O)CC(C)C#N)nc(OCC5(CN6CC7CC7C6)CC5)nc4c3OCc3ccc(-c4cn([C@H](C(=O)N5C[C@H](O)C[C@H]5C(=O)N[C@@H](CO)c5ccc(-c6ccnn6CC)cc5)C(C)C)nn4)cc3)c12 ; "HCOOH"] | 1285.7 [M − HCOOH + H]+ | δ 8.96 (t, J = 2.8 Hz, 1H), 8.62 (s, 1H), 8.48 (d, J = 9.0 Hz, 1H), 8.38-8.30 (m, 1H), 7.95 (dd, J = 5.8, 9.4 Hz, 1H), 7.70- 7.63 (m, 2H), 7.49 (d, J = 1.8 Hz, 1H), 7.43-7.34 (m, 6H), 7.12 (s, 1H), 6.96 (s, 2H), 6.31 (d, J = 1.8 Hz, 1H), 5.36-5.10 (m, 4H), 4.96-4.82 (m, 2H), 4.74-4.69 (m, 1H), 4.67-4.56 (m, 1H), 4.54-4.37 (m, 3H), 4.32-4.08 (m, 4H), 3.89 (d, J = 12.0 Hz, 1H), 3.83-3.76 (m, 2H), 3.69-3.60 (m, 4H), 2.96-2.85 (m, 4H), 2.80-2.73 (m, 1H), 2.40-2.36 (m, 2H), 2.21 (d, J = 7.8 Hz, 2H), 2.11-2.04 (m, 1H), 1.96-1.77 (m, 5H), 1.35-1.28 (m, 8H), 1.11-1.05 (m, 3H), 0.72 (d, J = 6.6 Hz, 3H), 0.55 (s, 2H), 0.52-0.47 (m, 1H), 0.38 (s, 2H), 0.20 (dt, J = 3.6, 7.6 Hz, 1H). |
| 139 | 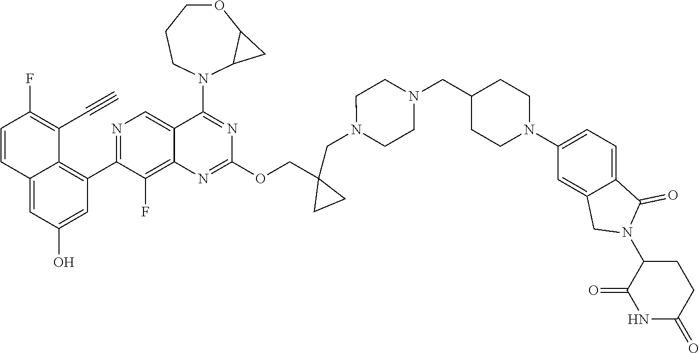 [SMILES: C#Cc1c(F)ccc2cc(O)cc(-c3ncc4c(N5CCCOC6CC65)nc(OCC5(CN6CCN(CC7CCN(c8ccc9c(c8)CN(C8CCC(=O)NC8=O)C9=O)CC7)CC6)CC5)nc4c3F)c12] | 952.5 [M − HCOOH + H]+ | δ 10.92 (s, 1H), 10.25-10.04 (m, 1H), 8.90 (s, 1H), 8.28 (s, 1H), 8.05-7.88 (m, 1H), 7.54-7.41 (m, 2H), 7.40-7.34 (m, 1H), 7.13 (d, J = 2.2 Hz, 1H), 7.06-6.96 (m, 2H), 5.03 (dd, J = 5.4, 13.4 Hz, 1H), 4.88- 4.65 (m, 1H), 4.57-4.44 (m, 1H), 4.38- 4.23 (m, 2H), 4.22-4.14 (m, 1H), 4.03- 3.67 (m, 6H), 3.58-3.40 (m, 4H), 3.12- 3.05 (m, 1H), 2.79 (t, J = 12.6 Hz, 4H), 2.66-2.52 (m, 4H), 2.42-2.36 (m, 4H), 2.11-2.04 (m, 2H), 2.00-1.90 (m, 2H), 1.86-1.67 (m, 4H), 1.39-1.22 (m, 1H), 1.21-1.08 (m, 2H), 1.02-0.89 (m, 1H), 0.83-0.66 (m, 2H), 0.57-0.42 (m, 2H). |
| 140 | 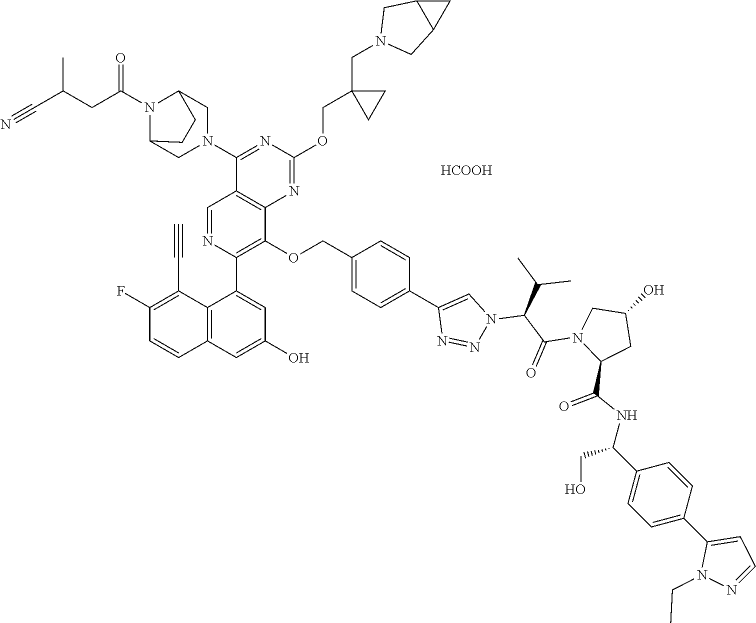 [SMILES: C#Cc1c(F)ccc2cc(O)cc(-c3ncc4c(N5CC6CCC(C5)N6C(=O)CC(C)C#N)nc(OCC5(CN6CC7CC7C6)CC5)nc4c3OCc3ccc(-c4cn([C@H](C(=O)N5C[C@H](O)C[C@H]5C(=O)N[C@@H](CO)c5ccc(-c6ccnn6CC)cc5)C(C)C)nn4)cc3)c12 ; "HCOOH"] | 1285.7 [M − HCOOH + H]+ | δ 8.96 (t, J = 2.8 Hz, 1H), 8.62 (s, 1H), 8.48 (d, J = 9.0 Hz, 1H), 8.38-8.30 (m, 1H), 7.95 (dd, J = 5.8, 9.4 Hz, 1H), 7.70- 7.63 (m, 2H), 7.49 (d, J = 1.8 Hz, 1H), 7.43-7.34 (m, 6H), 7.12 (s, 1H), 6.96 (s, 2H), 6.31 (d, J = 1.8 Hz, 1H), 5.36-5.10 (m, 4H), 4.96-4.82 (m, 2H), 4.74-4.69 (m, 1H), 4.67-4.56 (m, 1H), 4.54-4.37 (m, 3H), 4.32-4.08 (m, 4H), 3.89 (d, J = 12.0 Hz, 1H), 3.83-3.76 (m, 2H), 3.69-3.60 (m, 4H), 2.96-2.85 (m, 4H), 2.80-2.73 (m, 1H), 2.40-2.36 (m, 2H), 2.21 (d, J = 7.8 Hz, 2H), 2.11-2.04 (m, 1H), 1.96-1.77 (m, 5H), 1.35-1.28 (m, 8H), 1.11-1.05 (m, 3H), 0.72 (d, J = 6.6 Hz, 3H), 0.55 (s, 2H), 0.52-0.47 (m, 1H), 0.38 (s, 2H), 0.20 (dt, J = 3.6, 7.6 Hz, 1H). |
| 141 | 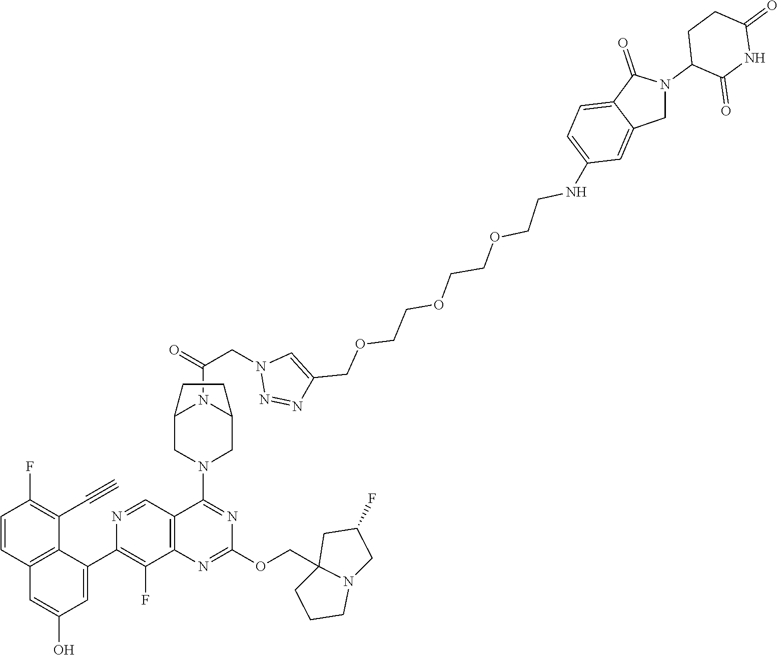 [SMILES: C#Cc1c(F)ccc2cc(O)cc(-c3ncc4c(N5CC6CCC(C5)N6C(=O)Cn5cc(COCCOCCOCCNc6ccc7c(c6)CN(C6CCC(=O)NC6=O)C7=O)nn5)nc(OCC56CCCN5C[C@@H](F)C6)nc4c3F)c12] | 1135.5 [M − HCOOH + Na]+ | δ 9.06 (s, 1H), 8.21-8.17 (m, 1H), 8.08- 8.02 (m, 1H), 8.00-7.95 (m, 1H), 7.46 (t, J = 9.0 Hz, 1H), 7.41-7.38 (m, 1H), 7.36- 7.33 (m, 1H), 7.19 (s, 1H), 6.64-6.60 (m, 2H), 5.80 (s, 1H), 5.54 (s, 2H), 5.22 (s, 1H), 5.07 (dd, J = 5.2 Hz, 1H), 4.68 (d, J = 4.8 Hz, 2H), 4.62 (d, J = 13.6 Hz, 1H), 4.56 (s, 2H), 4.49-4.41 (m, 1H), 4.27-4.21 (m, 1H), 4.16-4.12 (m, 1H), 4.09 (s, 1H), 4.06-4.03 (m, 1H), 3.95 (d, J = 9.8 Hz, 1H), 3.89-3.75 (m, 4H), 3.60-3.56 (m, 4H), 3.55-3.52 (m, 4H), 3.49 (s, 2H), 3.10 (d, J = 9.6 Hz, 2H), 3.06-3.00 (m, 2H), 2.98-2.93 (m, 1H), 2.83 (d, J = 6.4 Hz, 1H), 2.75-2.70 (m, 1H), 2.31-2.23 (m, 1H), 2.16-2.12 (m, 1H), 2.07 (s, 1H), 2.05- 1.98 (m, 2H), 1.88-1.75 (m, 6H). |
| 142 | 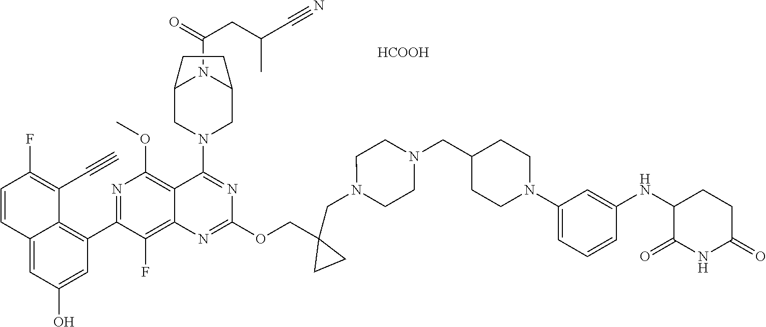 [SMILES: C#Cc1c(F)ccc2cc(O)cc(-c3nc(OC)c4c(N5CC6CCC(C5)N6C(=O)CC(C)C#N)nc(OCC5(CN6CCN(CC7CCN(c8cccc(NC9CCC(=O)NC9=O)c8)CC7)CC6)CC5)nc4c3F)c12 ; "HCOOH"] | 1036.6 [M − HCOOH + H]+ | δ 10.74 (s, 1H), 10.26-10.04 (m, 1H), 8.19 (s, 1H), 7.97 (dd, J = 6.0, 9.2 Hz, 1H), 7.46 (t, J = 9.0 Hz, 1H), 7.39 (d, J = 2.4 Hz, 1H), 7.23 (d, J = 2.4 Hz, 1H), 6.88 (t, J = 8.0 Hz, 1H), 6.24 (s, 1H), 6.19-6.14 (m, 1H), 6.10 (d, J = 6.6 Hz, 1H), 5.57 (d, J = 7.2 Hz, 1H), 4.69-4.62 (m, 1H), 4.53- 4.42 (m, 1H), 4.33-4.19 (m, 4H), 4.08- 3.98 (m, 1H), 3.94 (s, 1H), 3.90 (s, 3H), 3.56 (d, J = 12.4 Hz, 2H), 3.18-3.11 (m, 4H), 2.94-2.81 (m, 2H), 2.77-2.69 (m, 2H), 2.60-2.54 (m, 4H), 2.42-2.38 (m, 2H), 2.30-2.24 (m, 4H), 2.08 (d, J = 6.4 Hz, 4H), 1.88-1.79 (m, 2H), 1.77-1.68 (m, 4H), 1.67-1.49 (m, 2H), 1.30 (t, J = 7.0 Hz, 3H), 1.19-1.09 (m, 2H), 0.63 (s, 2H), 0.41 (s, 2H). |
| 143 | 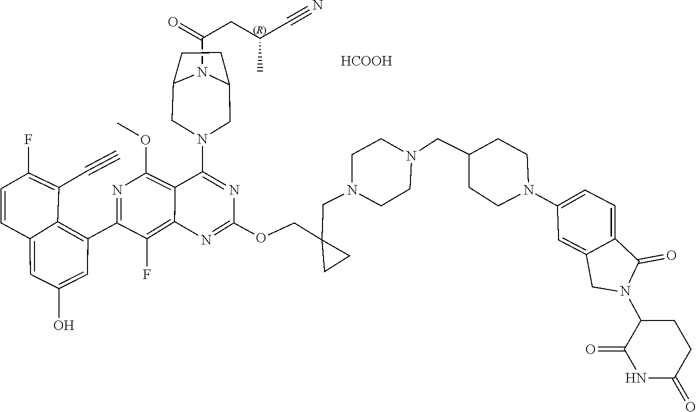 | 1076.5 [M − HCOOH + H]+ | δ 10.94 (s, 1H), 10.17 (s, 1H), 8.15 (s, 1H), 7.98 (dd, J = 6.0, 9.4 Hz, 1H), 7.53- 7.43 (m, 2H), 7.40 (d, J = 2.4 Hz, 1H), 7.23 (d, J = 2.4 Hz, 1H), 7.06-6.99 (m, 2H), 5.04 (dd, J = 5.2, 13.2 Hz, 1H), 4.70- 4.62 (m, 1H), 4.50-4.44 (m, 1H), 4.34- 4.26 (m, 3H), 4.25-4.15 (m, 2H), 4.12- 3.99 (m, 1H), 3.95 (s, 1H), 3.92-3.82 (m, 5H), 3.57-3.45 (m, 2H), 3.20-3.13 (m, 2H), 2.95-2.70 (m, 6H), 2.62-2.54 (m, 2H), 2.45-2.41 (m, 2H), 2.38-2.29 (m, 6H), 2.11 (d, J = 5.6 Hz, 2H), 2.00-1.90 (m, 2H), 1.82-1.66 (m, 6H), 1.34-1.27 (m, 3H), 1.20-1.10 (m, 2H), 0.64 (s, 2H), 0.42 (s, 2H). |
| 144 | 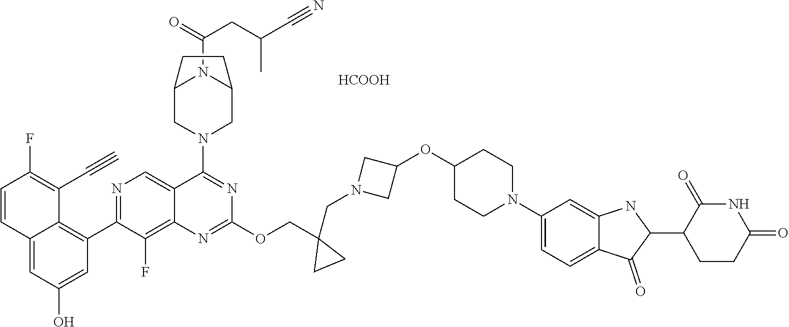 [SMILES: C#Cc1c(F)ccc2cc(O)cc(-c3ncc4c(N5CC6CCC(C5)N6C(=O)CC(C)C#N)nc(OCC5(CN6CC(OC7CCN(c8ccc9c(c8)NC(C8CCC(=O)NC8=O)C9=O)CC7)C6)CC5)nc4c3F)c12 ; "HCOOH"] | 1049.4 [M− HCOOH + H]+ | δ 11.11-10.78 (m, 1H), 8.22-8.18 (m, 1H), 7.99-7.95 (m, 1H), 7.50 (s, 2H), 7.40-7.38 (m, 1H), 7.24-7.22 (m, 1H), 7.05-7.01 (m, 2H), 5.06-5.01 (m, 1H), 4.68-4.63 (m, 1H), 4.48-4.43 (m, 1H), 4.33-4.28 (m, 1H), 4.20 (s, 3H), 4.16 (t, J = 5.8 Hz, 2H), 3.96 (s, 1H), 3.89 (s, 3H), 3.69-3.56 (m, 6H), 3.17 (s, 2H), 3.02-2.98 (m, 2H), 2.90- 2.84 (m, 2H), 2.77 (d, J = 6.8 Hz, 2H), 2.41 (s, 2H), 2.35 (d, J = 8.8 Hz, 1H), 1.98-1.70 (m, 9H), 1.47-1.42 (m, 2H), 1.35-1.25 (m, 4H), 0.52 (s, 2H), 0.44 (s, 2H). |
| 145 | 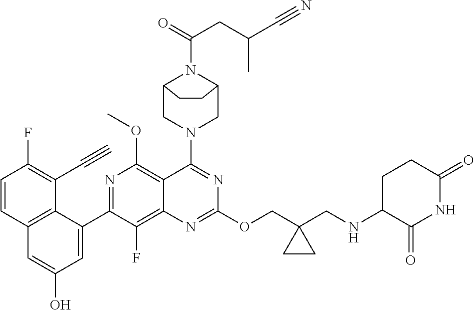 [SMILES: C#Cc1c(F)ccc2cc(O)cc(-c3nc(OC)c4c(N5CC6CCC(C5)N6C(=O)CC(C)C#N)nc(OCC5(CNC6CCC(=O)NC6=O)CC5)nc4c3F)c12] | 779.3[M + H]+ | δ 10.62-10.69 (m, 1H), 10.17 (d, J = 2.8 Hz, 1H), 7.93-8.01 (m, 1H), 7.42-7.50 (m, 1H), 7.38 (d, J = 2.5 Hz, 1H), 7.23 (d, J = 1.8 Hz, 1H), 4.62-4.69 (m, 1H), 4.36-4.47 (m, 2H), 4.00-4.27 (m, 4H), 3.92-3.96 (m, 1H), 3.89 (s, 3H), 3.42-3.55 (m, 2H), 3.38 (d, J = 4.5 Hz, 2H), 3.11-3.21 (m, 2H), 2.86-2.96 (m, 1H), 2.67-2.75 (m, 3H), 1.87-2.02 (m, 2H), 1.65-1.81 (m, 4H), 1.30 (t, J = 7.2 Hz, 3H), 0.50-0.59 (m, 4H). |
| 146 | 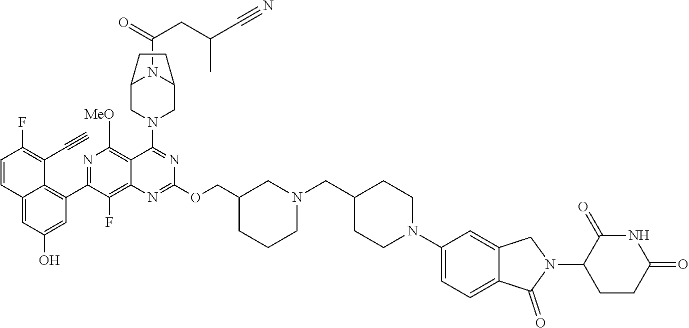 [SMILES: C#Cc1c(F)ccc2cc(O)cc(-c3nc(OC)c4c(N5CC6CCC(C5)N6C(=O)CC(C)C#N)nc(OCC5CCCN(CC6CCN(c7ccc8c(c7)CN(C7CCC(=O)NC7=O)C8=O)CC6)C5)nc4c3F)c12] | 1021.5 [M + H]+ | δ 10.93 (s, 1H), 10.23-10.08 (m, 1H), 7.97 (dd, J = 6.0, 9.0 Hz, 1H), 7.51-7.43 (m, 2H), 7.38 (d, J = 2.4 Hz, 1H), 7.26-7.20 (m, 1H), 7.04-6.97 (m, 2H), 5.03 (dd, J = 5.0, 13.2 Hz, 1H), 4.69-4.61 (m, 1H), 4.51-4.40 (m, 1H), 4.34-4.25 (m, 3H), 4.22-4.06 (m, 3H), 3.95 (s, 1H), 3.89 (s, 3H), 3.86-3.79 (m, 2H), 3.54-3.46 (m, 1H), 3.17-3.12 (m, 1H), 2.96-2.85 (m, 2H), 2.83-2.73 (m, 4H), 2.62-2.54 (m, 2H), 2.41-2.34 (m, 1H), 2.14 (d, J = 4.6 Hz, 2H), 2.07-1.85 (m, 6H), 1.79-1.64 (m, 8H), 1.54-1.44 (m, 1H), 1.29 (t, J = 7.2 Hz, 3H), 1.21-1.11 (m, 3H). |
| 147 | 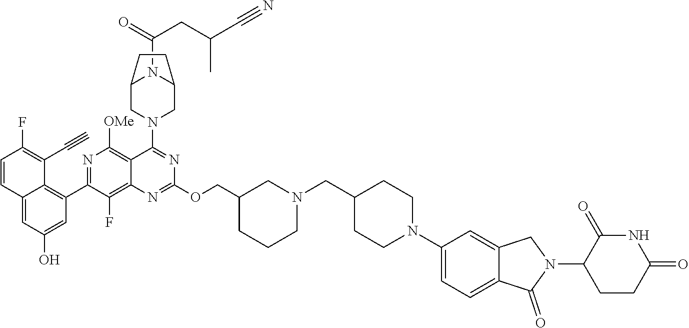 | 1007.5 [M + H]+ | δ 10.93 (s, 1H), 10.16 (d, J = 4.0 Hz, 1H), 7.97 (dd, J = 6.0, 9.0 Hz, 1H), 7.51-7.43 (m, 2H), 7.38 (d, J = 2.4 Hz, 1H), 7.26- 7.19 (m, 1H), 7.04-6.97 (m, 2H), 5.03 (dd, J = 5.0, 13.0 Hz, 1H), 4.68-4.60 (m, 1H), 4.48-4.39 (m, 1H), 4.34-4.25 (m, 3H), 4.21-4.04 (m, 3H), 3.95 (s, 1H), 3.89 (s, 3H), 3.86-3.78 (m, 2H), 3.54-3.47 (m, 1H), 2.95-2.83 (m, 2H), 2.82-2.75 (m, 4H), 2.69 (d, J = 6.4 Hz, 2H), 2.62-2.54 (m, 2H), 2.41-2.34 (m, 1H), 2.14 (d, J = 4.6 Hz, 2H), 2.08-1.85 (m, 6H), 1.78-1.63 (m, 8H), 1.55-1.45 (m, 1H), 1.15 (dd, J = 2.0, 4.6 Hz, 3H). |
| 148 | 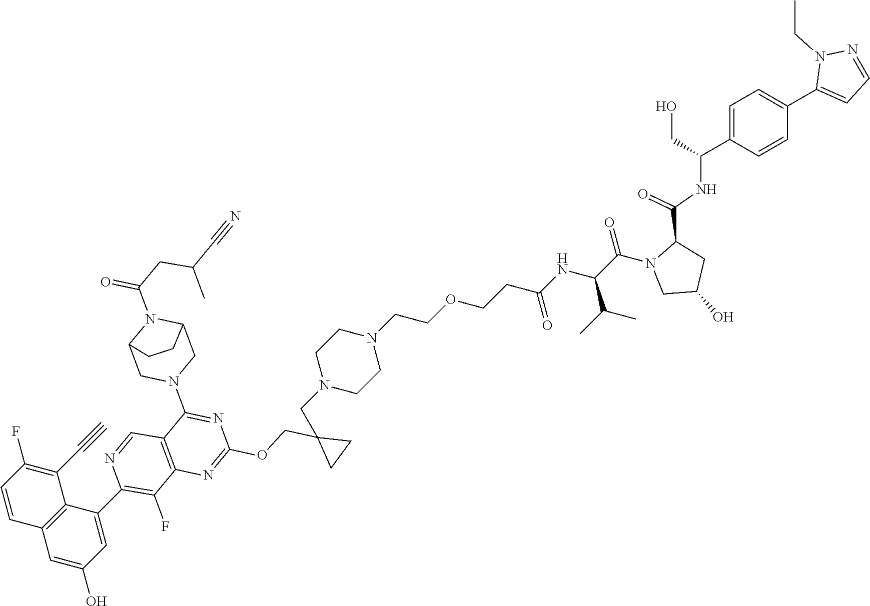 | 1278.7 [M + H]+ | δ 10.27-10.07 (m, 1H), 8.37-8.30 (m, 1H), 8.03-7.94 (m, 2H), 7.49 (d, J = 1.6 Hz, 1H), 7.47-7.44 (m, 1H), 7.43-7.41 (m, 1H), 7.41-7.38 (m, 4H), 7.23 (d, J = 2.5 Hz, 1H), 6.31 (d, J = 1.9 Hz, 1H), 5.11- 5.06 (m, 1H), 4.92-4.84 (m, 1H), 4.83- 4.74 (m, 1H), 4.70-4.61 (m, 1H), 4.51- 4.41 (m, 2H), 4.34 (t, J = 8.7 Hz, 1H), 4.30-4.24 (m, 3H), 4.13-4.08 (m, 2H), 3.97-3.94 (m, 1H), 3.89 (s, 3H), 3.63-3.57 (m, 4H), 3.53 (dd, J = 4.4, 5.6 Hz, 1H), 3.47-3.42 (m, 2H), 3.19-3.11 (m, 4H), 2.95-2.83 (m, 2H), 2.75-2.70 (m, 1H), 2.69-2.65 (m, 1H), 2.41-2.35 (m, 8H), 2.34-2.32 (m, 3H), 2.28 (s, 2H), 2.06-1.98 (m, 2H), 1.94-1.87 (m, 2H), 1.84-1.73 (m, 4H), 1.31 (t, J = 7.1 Hz, 6H), 0.88 (d, J = 6.8 Hz, 3H), 0.83 (d, J = 6.6 Hz, 3H), 0.63 (s, 2H), 0.40 (s, 2H). |
| 149 | 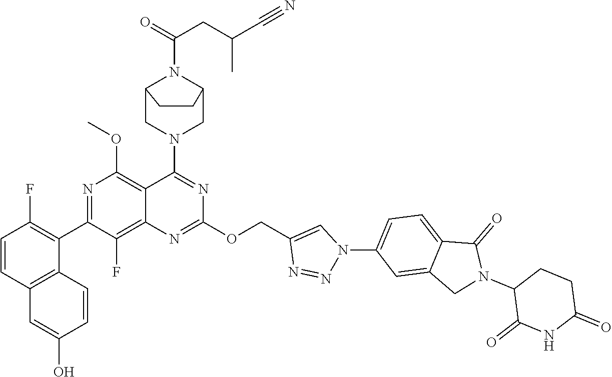 | m/z 908.4 [M + H]+ | δ 11.09-10.95 (m, 1H), 10.33-10.13 (m, 1H), 9.03 (s, 1H), 8.21 (s, 1H), 8.10-8.04 (m, 1H), 8.02-7.90 (m, 2H), 7.47 (t, J = 8.9 Hz, 1H), 7.39 (d, J = 2.5 Hz, 1H), 7.24 (d, J = 1.9 Hz, 1H), 5.67-5.55 (m, 2H), 5.15 (dd, J = 5.1, 13.2 Hz, 1H), 4.69-4.61 (m, 1H), 4.60-4.53 (m, 1H), 4.49-4.40 (m, 2H), 4.29-4.09 (m, 2H), 3.96 (s, 1H), 3.93- 3.88 (m, 3H), 3.56-3.47 (m, 1H), 3.43- 3.38 (m, 1H), 3.15 (dd, J = 6.5, 13.5 Hz, 1H), 2.98-2.85 (m, 2H), 2.73 (td, J = 5.6, 16.4 Hz, 1H), 2.66-2.58 (m, 1H), 2.46- 2.40 (m, 1H), 2.08-2.00 (m, 1H), 1.93- 1.80 (m, 1H), 1.75-1.60 (m, 3H), 1.29 ( t, J = 7.6 Hz, 3H). |
| 150 | 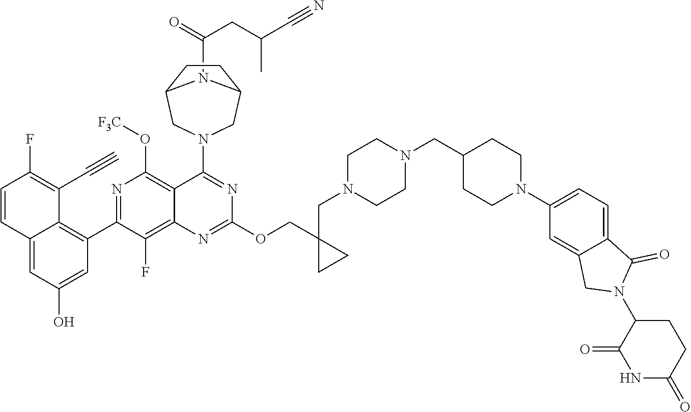 | 1130.5 [M + H]+ | δ 10.93 (s, 1H), 10.33-10.17 (m, 1H), 8.03-7.96 (m, 1H), 7.52-7.46 (m, 2H), 7.43 (d, J = 2.4 Hz, 1H), 7.23 (d, J = 2.1 Hz, 1H), 7.05-6.99 (m, 2H), 5.08-5.00 (m, 1H), 4.72-4.63 (m, 1H), 4.56-4.48 (m, 1H), 4.34 (d, J = 7.8 Hz, 2H), 4.29 (s, 1H), 4.22-4.19 (m, 1H), 4.02 (d, J = 3.6 Hz, 1H), 3.87-3.80 (m, 2H), 3.44 (s, 4H), 3.20- 3.13 (m, 2H), 2.93-2.84 (m, 2H), 2.80- 2.75 (m, 2H), 2.59 (dd, J = 2.3, 6.4 Hz, 4H), 2.31-2.21 (m, 8H), 2.08-2.04 (m, 2H), 2.00-1.84 (m, 3H), 1.77-1.67 (m, 5H), 1.30 (t, J = 7.6 Hz, 3H), 1.18-1.09 (m, 2H), 0.64 (s, 2H), 0.42 (s, 2H) |
| 151 | 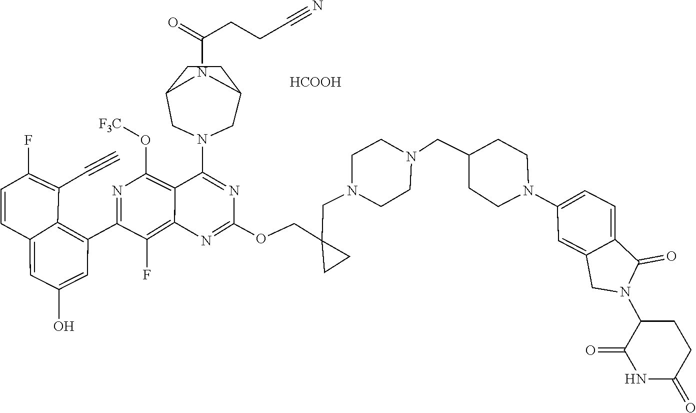 [SMILES: C#Cc1c(F)ccc2cc(O)cc(-c3nc(OC(F)(F)F)c4c(N5CC6CCC(C5)N6C(=O)CCC#N)nc(OCC5(CN6CCN(CC7CCN(c8ccc9c(c8)CN(C8CCC(=O)NC8=O)C9=O)CC7)CC6)CC5)nc4c3F)c12 ; "HCOOH"] | m/z 1116.5 [M − HCOOH + H]+ | δ 11.02-10.86 (m, 1H), 8.34 (s, 1H), 8.03- 7.96 (m, 1H), 7.49 (td, J = 4.4, 9.0 Hz, 2H), 7.43 (d, J = 2.5 Hz, 1H), 7.23 (s, 1H), 7.05-6.99 (m, 2H), 5.08-4.99 (m, 1H), 4.72-4.62 (m, 1H), 4.53-4.44 (m, 1H), 4.36-4.32 (m, 2H), 4.30-4.27 (m, 1H), 4.20 (s, 1H), 4.02 (s, 1H), 3.84 (d, J = 12.3 Hz, 2H), 3.61-3.42 (m, 7H), 2.84-2.76 (m, 4H), 2.72-2.65 (m, 3H), 2.60 (d, J = 3.9 Hz, 2H), 2.35-2.19 (m, 8H), 2.06 (s, 2H), 2.00-1.81 (m, 3H), 1.77-1.67 (m, 5H), 1.18-1.08 (m, 2H), 0.68-0.60 (m, 2H), 0.46-0.38 (m, 2H). |
| 152 | 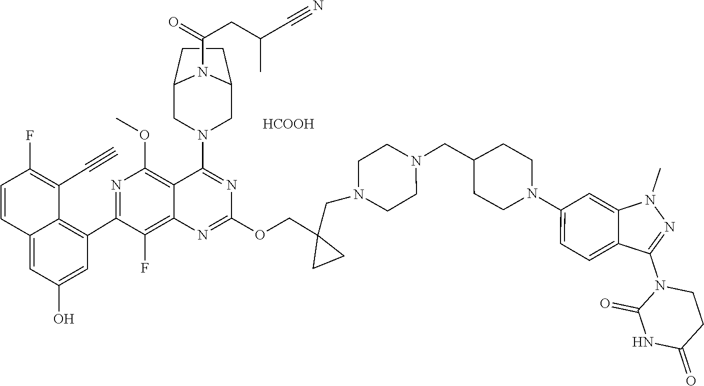 [SMILES: C#Cc1c(F)ccc2cc(O)cc(-c3nc(OC)c4c(N5CC6CCC(C5)N6C(=O)CC(C)C#N)nc(OCC5(CN6CCN(CC7CCN(c8ccc9c(N%10CCC(=O)NC%10=O)nn(C)c9c8)CC7)CC6)CC5)nc4c3F)c12 ; "HCOOH"] | 1076.6 [M − HCOOH + H]+ | δ 10.4-10.5 (m, 1H), 8.32-8.39 (m, 1H), 7.94-8.01 (m, 1H), 7.38-7.49 (m, 3H), 7.21-7.25 (m, 1H), 6.88 (d, J = 8.4 Hz, 1H), 6.77-6.80 (m, 1H), 6.57-6.61 (m, 1H), 5.32 (t, J = 4.8 Hz, 1H), 4.64-4.69 (m, 1H), 4.44-4.49 (m, 1H), 4.26-4.31 (m, 2H), 3.95 (s, 1H), 3.90 (s, 3H), 3.88 (s, 1H), 3.87 (s, 3H), 3.74-3.80 (m, 2H), 2.69- 2.75 (m, 5H), 2.26-2.31, (m, 5H), 2.08- 2.12 (m, 2H), 1.96-2.03 (m, 5H), 1.73- 1.80 (m, 4H), 1.43-1.48 (m, 2H), 1.27- 1.33 (m, 8H), 0.85 (t, J = 6.4 Hz, 3H), 0.61-0.66 (m, 2H), 0.41 (s, 2H). |
| 153 | 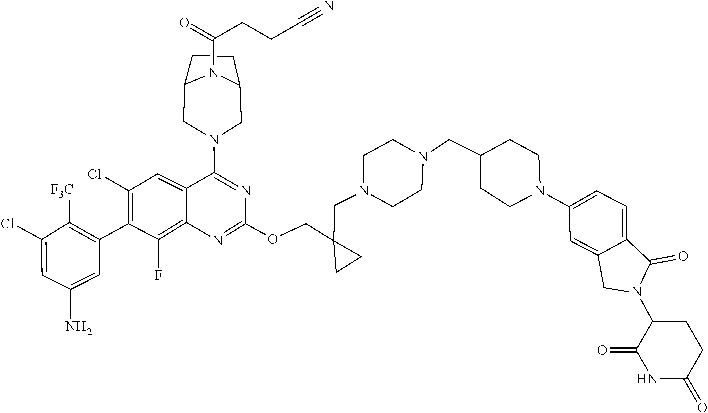 [SMILES: N#CCCC(=O)N1C2CCC1CN(c1nc(OCC3(CN4CCN(CC5CCN(c6ccc7c(c6)CN(C6CCC(=O)NC6=O)C7=O)CC5)CC4)CC3)nc3c(F)c(-c4cc(N)cc(Cl)c4C(F)(F)F)c(Cl)cc13)C2] | 1074.1 [M − HCOOH + H]+ | δ 10.95-10.91 (m, 1H), 8.28-8.27 (m, 1H), 7.87-7.84 (m, 1H), 7.51-7.46 (m, 1H), 7.04-7.00 (m, 2H), 6.91-6.88 (m, 1H), 6.37-6.31 (m, 3H), 5.06-5.00 (m, 1H), 4.69-4.63 (m, 1H), 4.50-4.38 (m, 2H), 4.36-4.32 (m, 1H), 4.30-4.15 (m, 5H), 3.88-3.82 (m, 2H), 3.76-3.61 (m, 2H), 3.55-3.48 (m, 2H), 2.85 (s, 2H), 2.83-2.76 (m, 4H), 2.71-2.67 (m, 2H), 2.39-2.32 (m, 4H), 2.29 (s, 4H), 2.09-2.05 (m, 2H), 1.98- 1.91 (m, 2H), 1.79-1.69 (m, 6H), 1.17- 1.09 (m, 2H), 0.63 (s, 2H), 0.42-0.37 (m, 2H). |
| 154 | 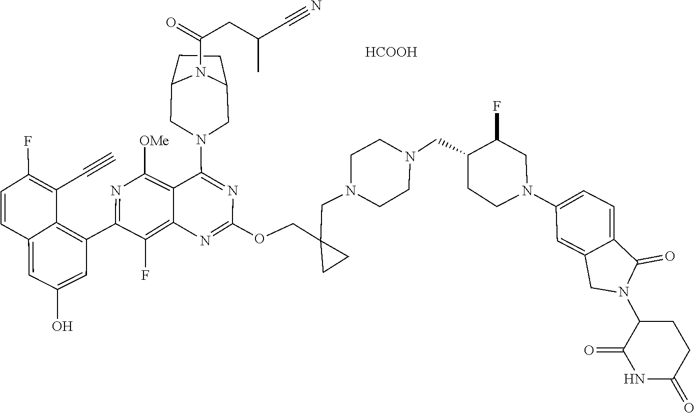 [SMILES: C#Cc1c(F)ccc2cc(O)cc(-c3nc(OC)c4c(N5CC6CCC(C5)N6C(=O)CC(C)C#N)nc(OCC5(CN6CCN(C[C@H]7CCN(c8ccc9c(c8)CN(C8CCC(=O)NC8=O)C9=O)C[C@@H]7F)CC6)CC5)nc4c3F)c12 ; "HCOOH"] | m/z 1094.5 [M − HCOOH + H]+ | δ 10.94 (s, 1H), 8.25 (s, 1H), 7.97 (dd, J = 5.8, 9.4 Hz, 1H), 7.53-7.43 (m, 2H), 7.39 (d, J = 2.6 Hz, 1H), 7.23 (d, J = 2.4 Hz, 1H), 7.11-7.04 (m, 2H), 5.04 (dd, J = 5.0, 13.4 Hz, 1H), 4.71-4.62 (m, 1H), 4.55- 4.41 (m, 2H), 4.37-4.16 (m, 6H), 3.98- 3.88 (m, 5H), 3.72-3.61 (m, 2H), 3.17- 3.13 (m, 2H), 3.08-3.00 (m, 2H), 2.96- 2.83 (m, 4H), 2.77-2.70 (m, 1H), 2.59 (s, 2H), 2.44-2.44 (m, 1H), 2.43-2.37 (m, 4H), 2.27 (d, J = 16.4 Hz, 4H), 2.02-1.88 (m, 5H), 1.79-1.72 (m, 2H), 1.35-1.22 (m, 5H), 0.63 (s, 2H), 0.41 (s, 2H). |
| 155 | 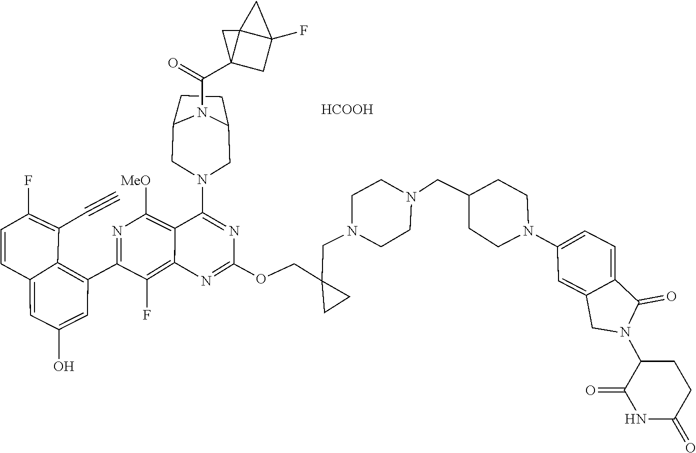 [SMILES: C#Cc1c(F)ccc2cc(O)cc(-c3nc(OC)c4c(N5CC6CCC(C5)N6C(=O)C56CC7(F)CC75C6)nc(OCC5(CN6CCN(CC7CCN(c8ccc9c(c8)CN(C8CCC(=O)NC8=O)C9=O)CC7)CC6)CC5)nc4c3F)c12 ; "HCOOH"] | 1093.2 [M − HCOOH + H]+ | δ 10.95-10.92 (m, 1H), 8.22-8.21 (m, 1H), 8.00-7.94 (m, 1H), 7.50-7.44 (m, 2H), 7.40-7.38 (m, 1H), 7.24-7.21 (m, 1H), 7.04-7.00 (m, 2H), 5.07-5.01 (m, 1H), 4.64-4.56 (m, 2H), 4.35-4.15 (m, 6H), 3.98-3.80 (m, 8H), 2.96-2.82 (m, 2H), 2.81-2.74 (m, 2H), 2.58 (d, J = 17.4 Hz, 2H), 2.47-2.40 (m, 8H), 2.37-2.24 (m, 8H), 2.08 (d, J = 5.8 Hz, 2H), 1.99-1.88 (m, 3H), 1.78-1.69 (m, 5H), 1.18-1.09 (m, 2H), 0.65-0.61 (m, 2H), 0.43-0.39 (m, 2H). |
| 156 | 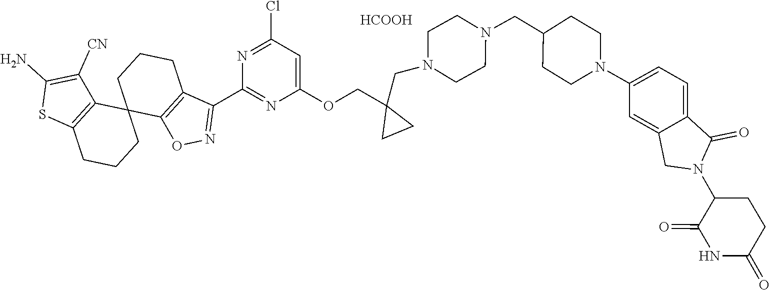 [SMILES: N#Cc1c(N)sc2c1C1(CCC2)CCCc2c(-c3nc(Cl)cc(OCC4(CN5CCN(CC6CCN(c7ccc8c(c7)CN(C7CCC(=O)NC7=O)C8=O)CC6)CC5)CC4)n3)noc21 ; "HCOOH"] | m/z 905.4 [M − HCOOH + H]+ | δ 10.96-10.92 (m, 1H), 8.26-8.24 (m, 1H), 7.48 (d, J = 8.8 Hz, 1H), 7.26-7.24 (m, 1H), 7.04-7.01 (m, 2H), 6.99-6.95 (m, 2H), 5.06-5.01 (m, 1H), 4.42 (d, J = 3.8 Hz, 2H), 4.33-4.28 (m, 1H), 4.21-4.16 (m, 1H), 3.84 (d, J = 12.2 Hz, 2H), 3.06-3.01 (m, 1H), 2.93-2.86 (m, 1H), 2.79 (t, J = 11.8 Hz, 2H), 2.67-2.60 (m, 2H), 2.39- 2.29 (m, 6H), 2.27-2.18 (m, 4H), 2.07 (d, J = 6.4 Hz, 4H), 2.01-1.91 (m, 5H), 1.85 (d, J = 3.2 Hz, 4H), 1.77-1.65 (m, 4H), 1.18- 1.10 (m, 2H), 0.70-0.65 (m, 2H), 0.45- 0.40 (m, 2H). |
| 157 | 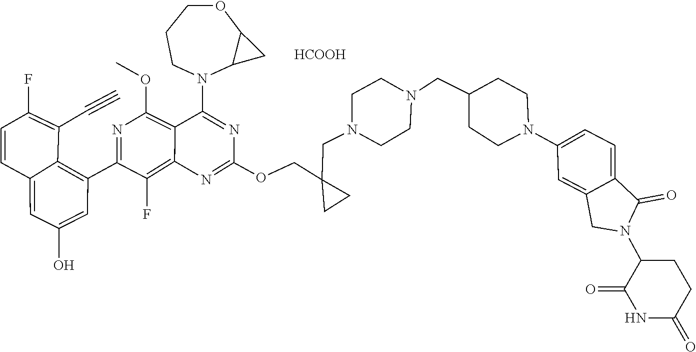 [SMILES: C#Cc1c(F)ccc2cc(O)cc(-c3nc(OC)c4c(N5CCCOC6CC65)nc(OCC5(CN6CCN(CC7CCN(c8ccc9c(c8)CN(C8CCC(=O)NC8=O)C9=O)CC7)CC6)CC5)nc4c3F)c12 ; "HCOOH"] | 982.4 [M − HCOOH + H]+ | δ 10.94 (s, 1H), 10.43-9.93 (m, 1H), 8.16 (s, 1H), 7.97 (d, J = 3.4 Hz, 1H), 7.50-7.44 (m, 2H), 7.38 (d, J = 2.2 Hz, 1H), 7.26- 7.23 (m, 1H), 7.17-7.14 (m, 1H), 7.04- 7.00 (m, 2H), 5.03 (d, J = 5.2 Hz, 1H), 4.52-4.43 (m, 1H), 4.34-4.18 (m, 4H), 4.14-4.02 (m, 1H), 3.90-3.82 (m, 6H), 3.71 (d, J = 11.8 Hz, 1H), 3.56-3.52 (m, 2H), 3.17 (d, J = 5.2 Hz, 2H), 2.95-2.82 (m, 2H), 2.81-2.73 (m, 2H), 2.63-2.55 (m, 2H), 2.42-2.27 (m, 9H), 2.09 (d, J = 10.8 Hz, 2H), 1.99-1.93 (m, 1H), 1.74 (d, J = 12.2 Hz, 4H), 1.19-1.09 (m, 2H), 0.97- 0.88 (m, 1H), 0.62 (d, J = 4.2 Hz, 2H), 0.40 (s, 2H). |
| 158 | 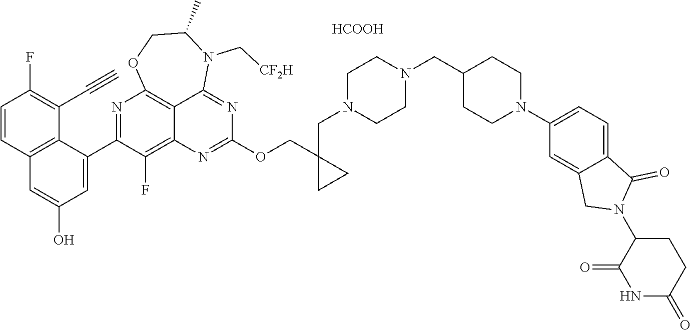 [SMILES: C#Cc1c(F)ccc2cc(O)cc(-c3nc4c5c(nc(OCC6(CN7CCN(CC8CCN(c9ccc%10c(c9)CN(C9CCC(=O)NC9=O)C%10=O)CC8)CC7)CC6)nc5c3F)N(CC(F)F)[C@@H](C)CO4)c12 ; "HCOOH"] | 976.3 [M − HCOOH + H]+ | δ 10.94 (s, 1H), 10.58-9.85 (m, 1H), 8.16 (s, 1H), 7.96 (dd, J = 6.0, 9.0 Hz, 1H), 7.53-7.42 (m, 2H), 7.37 (d, J = 2.4 Hz, 1H), 7.23-7.10 (m, 1H), 7.06-6.98 (m, 2H), 6.61-6.27 (m, 1H), 5.04 (dd, J = 5.0, 13.2 Hz, 1H), 4.67-4.54 (m, 1H), 4.43 (d, J = 13.0 Hz, 2H), 4.31 (d, J = 12.0 Hz, 1H), 4.28 (d, J = 6.4 Hz, 2H), 4.22-4.09 (m, 3H), 4.08-3.99 (m, 1H), 3.85 (d, J = 12.4 Hz, 2H), 3.00-2.74 (m, 4H), 2.61- 2.55 (m, 2H), 2.45-2.28 (m, 9H), 2.10 (d, J = 5.6 Hz, 2H), 1.99-1.91 (m, 1H), 1.74 (d, J = 11.4 Hz, 3H), 1.33-1.25 (m, 3H), 1.21- 1.08 (m, 2H), 0.63 (s, 2H), 0.42 (s, 2H) |
| 159 | 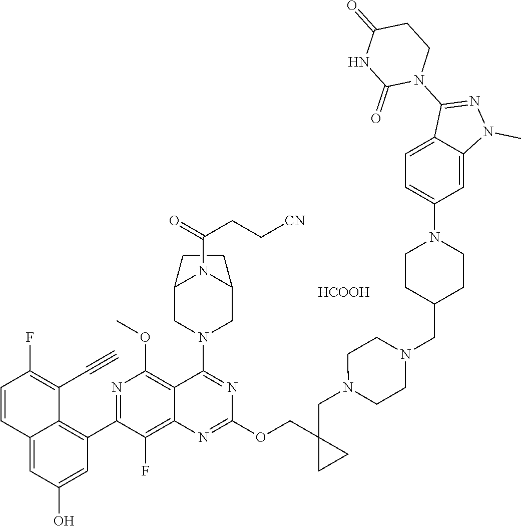 [SMILES: C#Cc1c(F)ccc2cc(O)cc(-c3nc(OC)c4c(N5CC6CCC(C5)N6C(=O)CCC#N)nc(OCC5(CN6CCN(CC7CCN(c8ccc9c(N%10CCC(=O)NC%10=O)nn(C)c9c8)CC7)CC6)CC5)nc4c3F)c12 ; "HCOOH"] | m/z 1062.4 [M − HCOOH + H]+ | δ 10.50 (s, 1H), 8.19 (s, 1H), 7.97 (dd, J = 5.8, 9.2 Hz, 1H), 7.49-7.44 (m, 1H), 7.42 (d, J = 9.0 Hz, 1H), 7.39 (d, J = 2.4 Hz, 1H), 7.23 (d, J = 2.2 Hz, 1H), 6.88 (d, J = 9.2 Hz, 1H), 6.78 (s, 1H), 4.68-4.63 (m, 1H), 4.45 (s, 1H), 4.29 (s, 2H), 4.25-4.17 (m, 1H), 4.06-3.99 (m, 1H), 3.95 (d, J = 2.6 Hz, 1H), 3.90-3.86 (m, 8H), 3.77 (d, J = 11.8 Hz, 2H), 3.54-3.43 (m, 4H), 3.31 (s, 2H), 2.82-2.77 (m, 2H), 2.71 (dd, J = 7.2, 9.4 Hz, 6H), 2.43-2.38 (m, 2H), 2.34- 2.24 (m, 6H), 2.11 (d, J = 6.6 Hz, 2H), 1.95-1.89 (m, 1H), 1.76 (d, J = 8.6 Hz, 4H), 1.25-1.17 (m, 2H), 0.63 (s, 2H), 0.41 (s, 2H). |
| 160 | 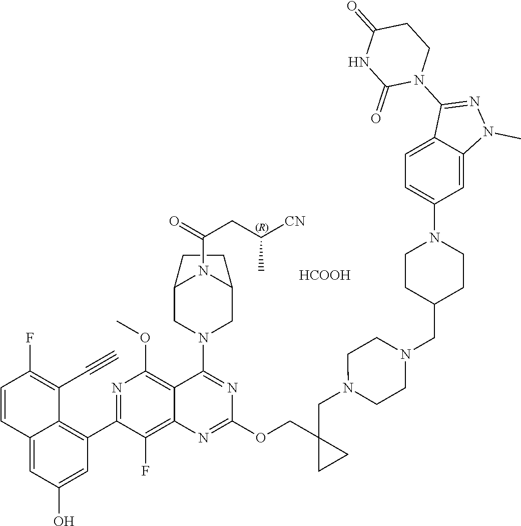 | m/z 1076.4 [M − HCOOH + H]+ | δ 10.49 (s, 1H), 10.30-10.13 (m, 1H), 8.30 (s, 1H), 7.97 (dd, J = 6.0, 9.2 Hz, 1H), 7.51-7.37 (m, 3H), 7.23 (d, J = 2.4 Hz, 1H), 6.93-6.84 (m, 1H), 6.79 (s, 1H), 4.73- 4.59 (m, 1H), 4.47 (t, J = 7.0 Hz, 1H), 4.33-4.25 (m, 2H), 4.25-4.14 (m, 1H), 4.12-3.99 (m, 1H), 3.95 (s, 1H), 3.90 (s, 3H), 3.88 (s, 1H), 3.87 (s, 3H), 3.77 (d, J = 12.0 Hz, 2H), 3.56-3.42 (m, 2H), 3.15 (dd, J = 6.4, 13.6 Hz, 1H), 2.92-2.82 (m, 1H), 2.78-2.68 (m, 5H), 2.45-2.35 (m, 4H), 2.31-2.23 (m, 4H), 2.17-2.02 (m, 3H), 1.98-1.83 (m, 2H), 1.80-1.63 (m, 6H), 1.30 (t, J = 7.2 Hz, 3H), 1.27-1.14 (m, 3H), 0.63 (s, 2H), 0.41 (s, 2H). |
| 161 | 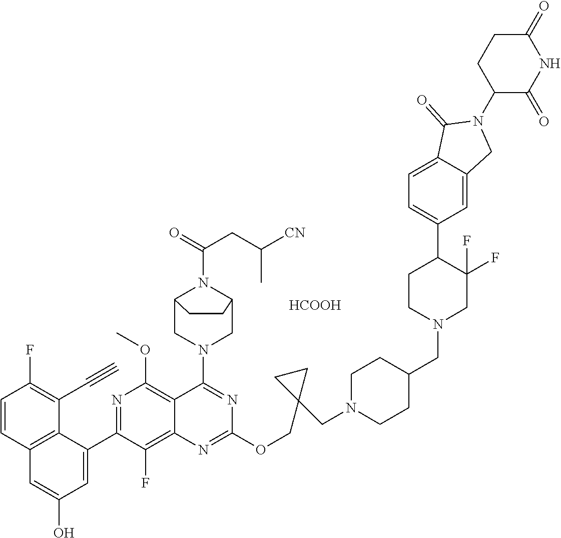 [SMILES: C#Cc1c(F)ccc2cc(O)cc(-c3nc(OC)c4c(N5CC6CCC(C5)N6C(=O)CC(C)C#N)nc(OCC5(CN6CCC(CN7CCC(c8ccc9c(c8)CN(C8CCC(=O)NC8=O)C9=O)C(F)(F)C7)CC6)CC5)nc4c3F)c12 ; "HCOOH"] | 1111.3 [M − HCOOH + H]+ | δ 10.99 (s, 1H), 10.18 (s, 1H), 8.26 (s, 1H), 7.97 (dd, J = 6.0, 9.4 Hz, 1H), 7.68 (d, J = 7.8 Hz, 1H), 7.56 (s, 1H), 7.50-7.42 (m, 2H), 7.39 (d, J = 2.4 Hz, 1H), 7.23 (d, J = 2.6 Hz, 1H), 5.11 (dd, J = 5.2, 13.2 Hz, 1H), 4.67 (d, J = 7.8 Hz, 1H), 4.51-4.41 (m, 2H), 4.37-4.20 (m, 4H), 4.13-4.00 (m, 1H), 3.98-3.87 (m, 4H), 3.17-3.11 (m, 2H), 3.11-3.11 (m, 1H), 2.95-2.84 (m, 5H), 2.78-2.69 (m, 1H), 2.63-2.58 (m, 1H), 2.39 (d, J = 9.4 Hz, 2H), 2.29-2.14 (m, 6H), 2.04-2.03 (m, 1H), 2.00 (d, J = 3.8 Hz, 1H), 1.90-1.75 (m, 6H), 1.68-1.61 (m, 2H), 1.51-1.44 (m, 1H), 1.35-1.19 (m, 4H), 1.12-0.96 (m, 3H), 0.63 (s, 2H), 0.41 (s, 2H) |
| 162 | 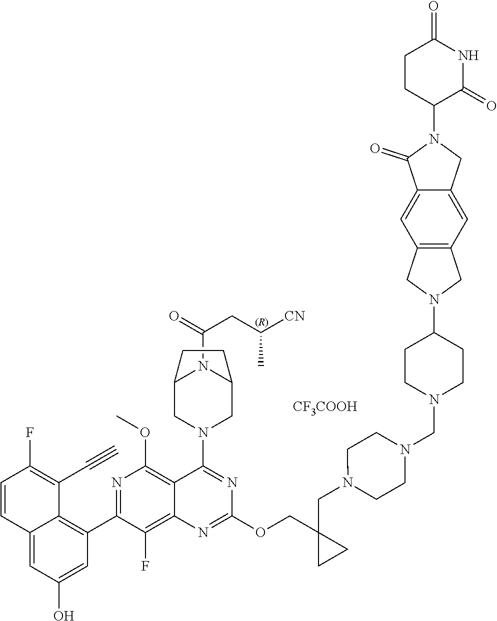 | 1116.5 [M − CF₃COOH + H]⁺ | δ 11.00 (s, 1H), 10.21 (s, 1H), 8.95-8.73 (m, 1H), 7.99 (dd, J = 6.2, 9.2 Hz, 1H), 7.76 (s, 1H), 7.63 (s, 1H), 7.48 (t, J = 9.0 Hz, 1H), 7.40 (d, J = 2.5 Hz, 1H), 7.22 (d, J = 1.8 Hz, 1H), 5.11 (dd, J = 4.8, 13.6 Hz, 1H), 4.73-4.63 (m, 2H), 4.78-4.42 (m, 3H), 4.40-4.26 (m, 4H), 4.19-4.10 (m, 1H), 3.96-3.85 (m, 5H), 3.81-3.67 (m, 4H), 3.24-3.14 (m, 4H), 3.06-2.83 (m, 8H), 2.79-2.68 (m, 2H), 2.65-2.58 (m, 2H), 2.44-2.34 (m, 2H), 2.10-1.84 (m, 8H), 1.82-1.63 (m, 4H), 1.53-1.40 (m, 2H), 1.31 (t, J = 7.4 Hz, 3H), 0.89 (s, 2H), 0.78 (s, 2H) |
| 163 | 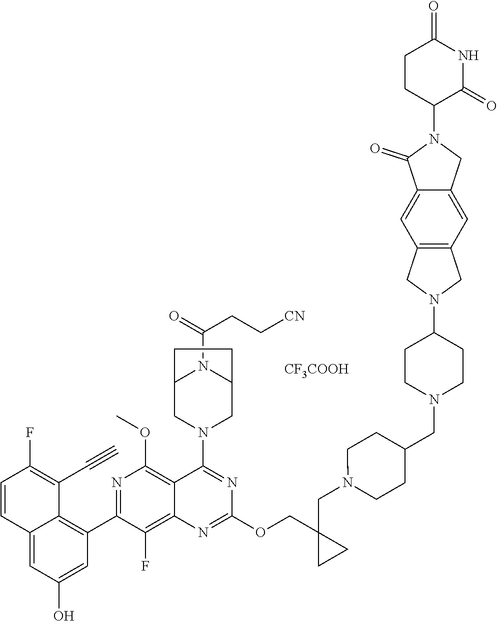 [SMILES: C#Cc1c(F)ccc2cc(O)cc(-c3nc(OC)c4c(N5CC6CCC(C5)N6C(=O)CCC#N)nc(OCC5(CN6CCC(CN7CCC(N8Cc9cc%10c(cc9C8)C(=O)N(C8CCC(=O)NC8=O)C%10)CC7)CC6)CC5)nc4c3F)c12 ; "CF3COOH"] | 1102.5 [M − CF3COOH + H]+ | δ 11.00 (s, 1H), 10.20 (s, 1H), 8.97-8.70 (m, 1H), 7.99 (dd, J = 6.0, 9.4 Hz, 1H), 7.83-7.70 (m, 1H), 7.63 (s, 1H), 7.48 (t, J = 9.2 Hz, 1H), 7.40 (d, J = 2.6 Hz, 1H), 7.22 (d, J = 2.4 Hz, 1H), 5.11 (dd, J = 4.8, 13.2 Hz, 1H), 4.68 (d, J = 8.4 Hz, 2H), 4.57-4.41 (m, 3H), 4.40-4.27 (m, 4H), 4.19-4.10 (m, 1H), 3.96-3.86 (m, 5H), 3.80-3.67 (m, 4H), 3.24 (d, J = 1.6 Hz, 3H), 3.06-2.86 (m, 7H), 2.81 (s, 3H), 2.72- 2.68 (m, 2H), 2.64-2.58 (m, 2H), 2.43- 2.35 (m, 2H), 2.09-1.85 (m, 8H), 1.81- 1.63 (m, 4H), 1.53-1.40 (m, 2H), 0.89 (s, 2H), 0.78 (s, 2H). |
| 164 | 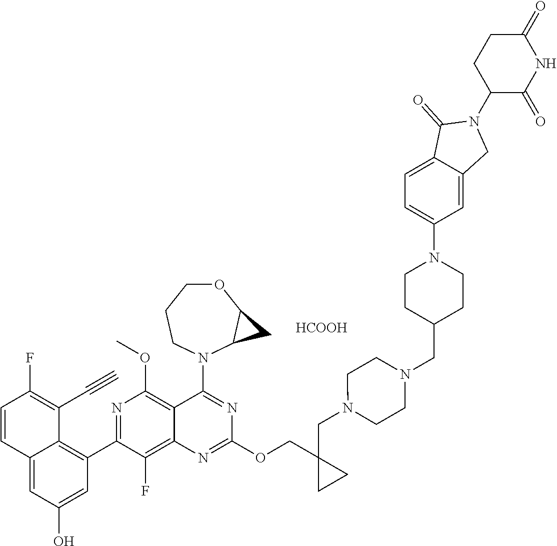 [SMILES: C#Cc1c(F)ccc2cc(O)cc(-c3nc(OC)c4c(N5CCCO[C@H]6C[C@H]65)nc(OCC5(CN6CCN(CC7CCN(c8ccc9c(c8)CN(C8CCC(=O)NC8=O)C9=O)CC7)CC6)CC5)nc4c3F)c12 ; "HCOOH"] | 982.4 [M − HCOOH + H]+ | δ 10.93 (s, 1H), 10.18-10.12 (m, 1H), 8.30-8.26 (m, 1H), 7.96 (d, J = 9.4 Hz, 1H), 7.51-7.44 (m, 2H), 7.38 (d, J = 2.4 Hz, 1H), 7.25-7.23 (m, 1H), 7.15 (d, J = 2.8 Hz, 1H), 7.05-7.00 (m, 2H), 5.03 (dd, J = 4.8, 13.2 Hz, 1H), 4.53-4.42 (m, 1H), 4.34-4.17 (m, 4H), 4.13-4.01 (m, 1H), 3.89-3.81 (m, 6H), 3.72-3.68 (m, 1H), 3.54 (d, J = 5.4 Hz, 2H), 3.16 (s, 2H), 2.90-2.83 (m, 2H), 2.78-2.69 (m, 2H), 2.67-2.59 (m, 2H), 2.38-2.30 (m, 7H), 2.25-2.18 (m, 2H), 2.10-2.05 (m, 2H), 1.99-1.94 (m, 1H), 1.74 (d, J = 13.0 Hz, 4H), 1.18-1.11 (m, 2H), 0.95-0.89 (m, 1H), 0.62 (d, J = 4.0 Hz, 2H), 0.39 (s, 2H). |
| 165 | 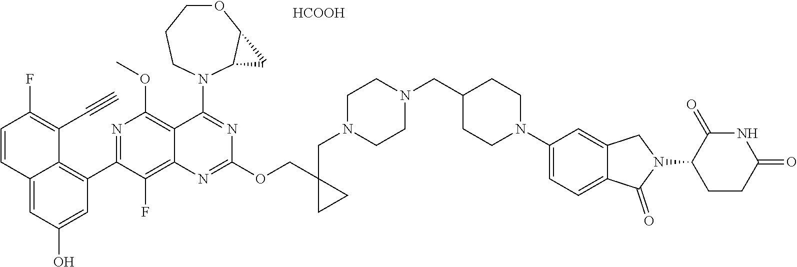 [SMILES: C#Cc1c(F)ccc2cc(O)cc(-c3nc(OC)c4c(N5CCCO[C@@H]6C[C@@H]65)nc(OCC5(CN6CCN(CC7CCN(c8ccc9c(c8)CN([C@H]8CCC(=O)NC8=O)C9=O)CC7)CC6)CC5)nc4c3F)c12 ; "HCOOH"] | 982.4 [M − HCOOH + H]+ | δ 10.69 (s, 1H), 10.15-9.70 (m, 1H), 7.97- 7.94 (m, 1H), 7.72 (d, J = 9.4 Hz, 1H), 7.27-7.19 (m, 2H), 7.15-7.12 (m, 1H), 7.00 (d, J = 2.4 Hz, 1H), 6.93-6.87 (m, 1H), 6.80-6.75 (m, 2H), 4.78 (dd, J = 5.0, 13.4 Hz, 1H), 4.28-4.18 (m, 1H), 4.12- 3.93 (m, 4H), 3.88 (s, 1H), 3.66-3.58 (m, 6H), 3.49-3.44 (m, 1H), 3.31-3.28 (m, 2H), 2.95-2.90 (m, 2H), 2.70-2.59 (m, 2H), 2.55 (t, J = 11.8 Hz, 2H), 2.48-2.34 (m, 2H), 2.14-2.04 (m, 7H), 2.02-1.94 (m, 2H), 1.84 (dd, J = 6.8, 11.4 Hz, 2H), 1.75- 1.69 (m, 1H), 1.49 (d, J = 12.6 Hz, 4H), 0.95-0.86 (m, 2H), 0.71-0.63 (m, 1H), 0.41-0.34 (m, 2H), 0.15 (s, 2H). |
| 166 | 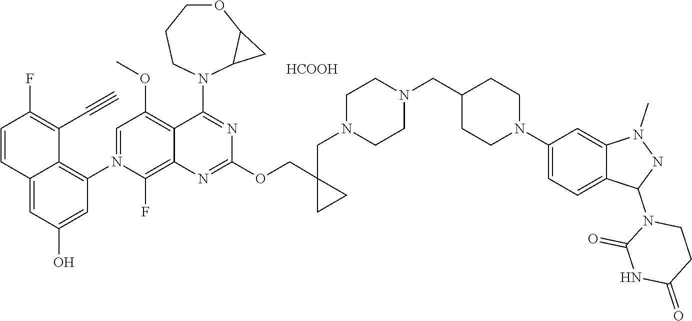 | 982.4 [M − HCOOH + H]+ | δ 10.55-10.45 (m, 1H), 8.20-8.18 (m, 1H), 7.97 (d, J = 9.4 Hz, 1H), 7.50-7.41 (m, 2H), 7.39 (t, J = 2.2 Hz, 1H), 7.27-7.25 (m, 1H), 7.16 (d, J = 2.5 Hz, 1H), 6.89 (dd, J = 1.4, 9.2 Hz, 1H), 6.79 (s, 1H), 4.55-4.42 (m, 1H), 4.35-4.18 (m, 2H), 4.15-4.03 (m, 1H), 3.92-3.85 (m, 9H), 3.81-3.67 (m, 4H), 3.60-3.50 (m, 3H), 3.19-3.13 (m, 1H), 2.74 (t, J = 6.6 Hz, 4H), 2.41-2.30 (m, 6H), 2.29-2.20 (m, 2H), 2.12 (dd, J = 7.4, 11.4 Hz, 2H), 1.80- 1.65 (m, 4H), 1.26-1.16 (m, 2H), 0.98- 0.87 (m, 1H), 0.63 (d, J = 4.4 Hz, 2H), 0.46-0.38 (m, 2H). |
| 167 | 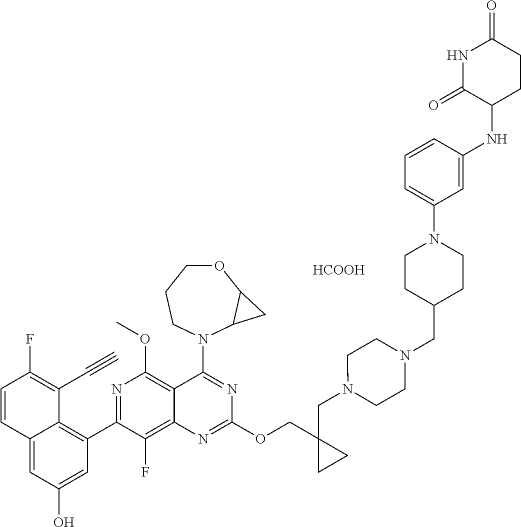 [SMILES: C#Cc1c(F)ccc2cc(O)cc(-c3nc(OC)c4c(N5CCCOC6CC65)nc(OCC5(CN6CCN(CC7CCN(c8cccc(NC9CCC(=O)NC9=O)c8)CC7)CC6)CC5)nc4c3F)c12 ; "HCOOH"] | 942.4 [M − HCOOH + H]+ | δ 10.75 (s, 1H), 8.20 (s, 1H), 7.97 (d, J = 9.2 Hz, 1H), 7.51-7.44 (m, 1H), 7.39 (d, J = 2.4 Hz, 1H), 7.25 (d, J = 2.4 Hz, 1H), 7.16 (d, J = 2.4 Hz, 1H), 6.89 (t, J = 8.0 Hz, 1H), 6.29-6.24 (m, 1H), 6.18 (d, J = 8.2 Hz, 1H), 6.10 (d, J = 7.8 Hz, 1H), 5.59 (d, J = 7.6 Hz, 1H), 4.49 (d, J = 11.8 Hz, 1H), 4.39-4.27 (m, 2H), 4.25-4.18 (m, 1H), 4.14-4.02 (m, 1H), 3.89 (s, 3H), 3.85 (s, 1H), 3.77-3.66 (m, 2H), 3.60-3.52 (m, 6H), 3.18 (d, J = 5.4 Hz, 2H), 2.77-2.70 (m, 1H), 2.59 (s, 2H), 2.39-2.28 (m, 6H), 2.27-2.19 (m, 2H), 2.13-2.07 (m, 3H), 1.94-1.77 (m, 2H), 1.72 (d, J = 11.2 Hz, 2H), 1.63-1.55 (m, 1H), 1.15 (q, J = 10.9 Hz, 2H), 0.97-0.87 (m, 1H), 0.63 (d, J = 4.2 Hz, 2H), 0.40 (s, 2H). |
| 168 | 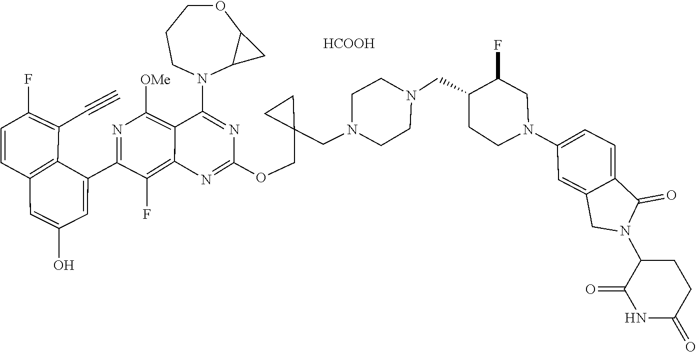 [SMILES: C#Cc1c(F)ccc2cc(O)cc(-c3nc(OC)c4c(N5CCCOC6CC65)nc(OCC5(CN6CCN(C[C@H]7CCN(c8ccc9c(c8)CN(C8CCC(=O)NC8=O)C9=O)C[C@@H]7F)CC6)CC5)nc4c3F)c12 ; "HCOOH"] | 1000.4 [M − HCOOH + H]+ | δ 10.94 (s, 1H), 10.28-9.97 (m, 1H), 8.16 (s, 1H), 7.97 (ddd, J = 3.2, 5.8, 9.2 Hz, 1H), 7.59-7.43 (m, 2H), 7.38 (d, J = 2.4 Hz, 1H), 7.26-7.13 (m, 1H), 7.12-7.02 (m, 2H), 5.04 (dd, J = 5.2, 13.2 Hz, 1H), 4.52- 4.46 (m, 1H), 4.54-4.10 (m, 6H), 4.03- 3.94 (m, 1H), 3.92-3.83 (m, 4H), 3.76- 3.64 (m, 2H), 3.57-3.51 (m, 2H), 3.20- 3.14 (m, 2H), 3.04 (d, J = 5.6 Hz, 1H), 2.99-2.83 (m, 3H), 2.58 (d, J = 17.2 Hz, 2H), 2.43-2.25 (m, 10H), 2.11-1.84 (m, 4H), 1.80-1.71 (m, 1H), 1.33-1.24 (m, 1H), 0.98-0.86 (m, 1H), 0.63 (d, J = 3.6 Hz, 2H), 0.40 (s, 3H) |
| 169 | 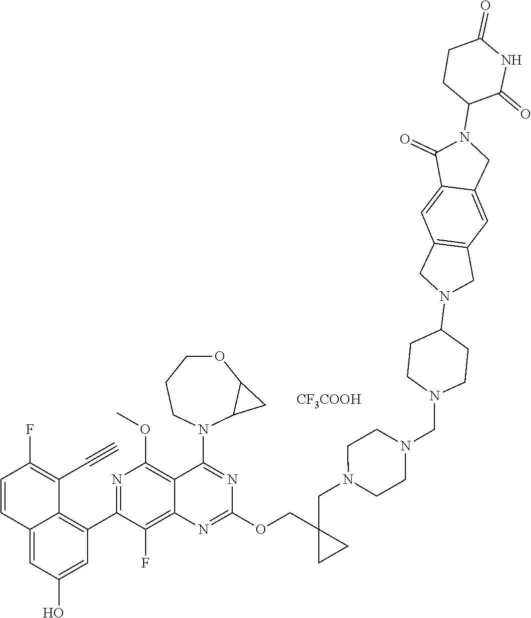 [SMILES: C#Cc1c(F)ccc2cc(O)cc(-c3nc(OC)c4c(N5CCCOC6CC65)nc(OCC5(CN6CCN(CN7CCC(N8Cc9cc%10c(cc9C8)C(=O)N(C8CCC(=O)NC8=O)C%10)CC7)CC6)CC5)nc4c3F)c12 ; "CF3COOH"] | 1022.3 [M − CF3COOH + H]+ | δ 11.01 (s, 1H), 10.34-10.05 (m, 1H), 9.06-8.64 (m, 1H), 8.08-7.91 (m, 1H), 7.83-7.58 (m, 2H), 7.54-7.44 (m, 1H), 7.40 (d, J = 2.4 Hz, 1H), 7.28-7.14 (m, 1H), 5.12 (dd, J = 4.8, 13.4 Hz, 1H), 5.03- 4.65 (m, 3H), 4.54-4.45 (m, 2H), 4.42- 4.20 (m, 4H), 4.06-3.95 (m, 2H), 3.89 (d, J = 17.4 Hz, 5H), 3.82-3.77 (m, 2H), 3.73 (d, J = 1.8 Hz, 4H), 3.31-3.16 (m, 5H), 3.09-2.83 (m, 7H), 2.61 (d, J = 17.0 Hz, 1H), 2.44-2.35 (m, 2H), 1.79-1.71 (m, 1H), 2.07-1.70 (m, 7H), 1.57-1.39 (m, 2H), 1.09-0.65 (m, 6H), 0.43-0.16 (m, 1H). |

### Biological Assays

### Materials and Methods

### Cell Lines

The following cancer cell lines were employed:
AsPC-1 human pancreatic adenocarcinoma, homozygous KRAS(G12D), (ATCC, #CRL-1682); A-427 human lung carcinoma, heterozygous KRAS(G12D), (ATCC, #HTB-53); AGS human gastric adenocarcinoma, heterozygous KRAS(G12D), (ATCC, #CRL-1739); GP2D human colon adenocarcinoma, heterozygous KRAS(G12D), (Sigma Aldrich, 95090714); SW1990 human pancreatic adenocarcinoma, homozygous KRAS(G12D), (ATCC, #CRL2172); LU65A human lung giant cell carcinoma, heterozygous KRAS(G12C), (JCRB Cell Bank, #JCRB0054); NCIH727 human lung carcinoid, heterozygous KRAS(G12V), (ATCC, #CRL-5815); MKN1 human gastric adenosquamous carcinoma KRAS (wt), (JCRB Cell Bank, #JCRB0252). Cell lines were cultured according to ATCC, JCRB and Sigma Aldrich recommendations.

### KRAS(G12D)/SOS1 Homogeneous Time-Resolved Fluorescence (HTRF) Assay

Binding of test compounds to KRAS(G12D) protein, which in turn blocks KRAS(G12D) interaction with the SOS1 protein, was measured in the absences of GTP by homogeneous time-resolved fluorescence (HTRF) using the KRAS-(G12D)/SOS1 Binding Assay Kit (Cisbio, #63ADK000CB16PEG), following the manufacturer's instructions, except as noted. 3-fold serial dilutions of each test compound were prepared ranging from 20 μM to 1.02 nM. The test compound was mixed and incubated with reaction components, incubated in a sealed plate at 4° C. for 3 hr and fluorescence was measured using a PerkinElmer Envision plate reader. The % inhibition and IC50 values (the concentration at which 50% of the maximal inhibition occurs) were calculated and plotted using GraphPad Prism 7 software.

### CellTiter-Glo® Reagent Cancer Cell Line Proliferation Assays

Cells were plated in 96-well tissue culture plates at 4,000 cells/well and incubated at 37° C./5% CO₂ for 24 hr in 100 μl of media. 3-fold serial dilutions of each test compound were prepared ranging from 20 μM to 1.02 nM. Cells were then treated with test compounds at various concentrations with a final concentration of 0.5% DMSO/well, and then incubated at 37° C./5% CO₂ for 24 hr. 100 μl of CellTiter-Glo® Reagent (Promega Corporation, Madison, WI) was added to each well and processed according manufacturer's protocol. Results were analyzed and IC50-values were calculated in GraphPad 7 software.

### KRAS(G12D/C/V) Western Blot Protein Degradation Assay

Cell lines SW1990 and GP2D that express KRAS(G12D), LU65A that expresses KRAS(G12C), and NCIH727 that expresses KRAS(G12V) were seeded in 6- or 12-well tissue culture plates, and after 1 hr, test compounds were added at various concentrations and incubated at 37° C./5% CO₂ for 24 hr. Cells were then washed with cold PBS, aspirated and cold RIPA buffer containing a protease/phosphatase inhibitor cocktail was added to lyse cells. After centrifugation, the total protein concentrations of cell lysates were determined using the BCA protein assay. Samples were normalized for equivalent protein concentrations, 5× SDS-PAGE loading buffer added and denatured at 100° C. for 10 min. 20 μl of each sample/well was loaded on a SDS-PAGE gel and electrophoresed for 20 min at 80 V, then 120 V for 1.5 hr. Gels were then electroblotted to nitrocellulose membranes using a wet-transfer method at 250 mA for 2.5 hr. Membranes were incubated with blocking buffer for 1 hr and washed 3 times with TBST for 5 min. Membranes were then incubated with anti-KRAS (Sigma-Aldrich, #SAB1404011) and anti-β-Actin monoclonal antibodies (Cell Signaling Technology, #3700) diluted in blocking buffer at 4° C. overnight per the manufacturer's recommendations. After washing 3 times, blots were incubated with appropriately labeled secondary antibodies for 1 hr at room temperature and washed again. Fluorescence imaging and quantitation was performed using a LI-COR Odyssey. Results was analyzed using GraphPad Prism 7 software. Compound concentrations that induced KRAS degradation (DC₅₀ and DCₘₐₓ) were calculated using GraphPad Prism 7 software.

### Results

A number of synthetic schemes have been developed to construct various PROTAC molecules designed to degrade KRAS(G12D/C/V), which are termed KRAS(G12D/C/V)-PROTAC molecules. Representative examples are shown, each consisting of a E3 ubiquitin-ligase (CRBN or VHL) binder linked to a KRAS(G12D/C/V) binder. Similar chemistry can be applied to other PROTAC molecules not limited to these specific E3 ubiquitin-ligase- and KRAS(G12D/C/V)-binding moieties.

Binding by a variety of PROTAC molecules to KRAS(G12D) was assessed by measuring inhibition of KRAS(G12D) interaction with SOS1 in a HTRF biochemical assay, as shown in Table 2. PROTAC molecules containing KRAS(G12D)-binding moieties documented in the literature were generally in agreement with the published SAR.

KRAS(G12D/C/V)-PROTAC molecules inhibited the growth and/or survival of a panel of cancer cell lines as measured by CellTiter-Glo® Reagent cancer cell line proliferation assay as shown in Table 2, 3, and 4.

KRAS(G12D/C/V)-PROTAC molecules also induced KRAS protein degradation as determined by Western blot analysis in a panel of cell lines: SW1990 in Table 2, GP2D in Table 3, and LU65A and NCIH727 in Table 4.

TABLE 2
Biochemical and Cell-based Assays of Compounds

| | ¹HTRF of | | | | | | ³WB data |
|---|---|---|---|---|---|---|---|
| | KRAS | | | | | | SW1990 |
| | G12D-SOS1 | | | | | | DC₅₀; |
| Compound | (nM) | AGS | A427 | ASPC-1 | SW1990 | GP2D | D_{Max} |
| 1 | A | C | C | C | | | |
| 2 | B | C | C | C | | | |
| 3 | A | C | C | C | | | |
| 4 | A | C | C | C | | | |
| 5 | A | C | C | C | | | |
| 6 | A | C | C | C | | | |
| 7 | A | C | C | C | | | |
| 8 | A | C | C | C | | | |
| 9 | A | B | C | C | | | |
| 10 | A | B | C | C | | | |
| 11 | A | C | C | C | | | |
| 12 | A | B | C | C | | | |
| 13 | A | B | C | C | | | |
| 14 | A | C | C | C | C | | |
| 15 | A | C | C | C | C | | |
| 16 | A | C | C | C | C | | |
| 17 | A | B | C | C | | | |
| 18 | A | C | C | C | | | |
| 19 | B | — | — | — | | | |
| 20 | A | C | C | C | | | |
| 21 | A | C | C | C | | | |
| 22 | A | A | B | — | | | |
| 23 | A | C | C | — | | | |
| 24 | A | C | C | — | | | |
| 25 | A | C | C | — | | | |
| 26 | A | A | B | — | A | | A; A |
| 27 | A | A | A | A | A | A | A; A |
| 28 | A | C | — | — | | | |
| 29 | A | A | B | B | A | | |
| 30 | A | C | C | — | | | |
| 31 | A | B | B | — | | | |
| 32 | A | C | B | — | | | |
| 33 | A | C | C | — | | | |
| 34 | A | B | C | — | | | |
| 35 | A | B | C | — | | | |
| 36 | A | B | B | B | B | | |
| 37 | A | C | B | — | | | |
| 38 | A | B | C | — | | | |
| 39 | A | A | C | — | | | |
| 40 | C | C | C | — | | | |
| 41 | A | A | B | — | A | | C; B |
| 42 | A | A | B | — | B | | C; B |
| 43 | A | A | A | A | A | A | A; A |
| 44 | A | C | C | — | | | |
| 45 | A | B | C | — | | | |
| 46 | A | C | C | — | | | |
| 47 | A | C | C | C | C | | |
| 48 | A | A | A | A | A | A | A; A |
| 49 | A | C | C | C | C | | |
| 50 | A | C | C | C | C | | |
| 51 | A | A | B | B | A | | C; B |
| 52 | A | B | C | C | C | | A; A |
| 53 | A | B | C | C | C | | C; B |
| 54 | — | A | C | C | B | | B; A |
| 55 | — | A | A | C | B | | B; A |
| 56 | — | A | A | A | A | | A; B |
| 57 | — | A | B | B | B | | C; B |
| 58 | — | A | C | B | B | | |
| 59 | — | A | C | C | B | | |
| 60 | — | A | A | A | A | | |
| 61 | — | C | C | C | C | | C; C |
| 62 | A | C | C | C | C | | |
| 63 | A | A | C | B | B | A | B; B |
| 64 | A | A | B | A | A | A | A; A |
| 65 | A | A | B | B | A | A | C; C |
| 66 | A | A | B | A | A | A | C; B |
| 67 | A | C | C | C | C | C | |
| 68 | C | C | C | C | C | C | C; C |
| 69 | A | A | C | A | A | A | A; A |
| 70 | A | A | B | A | A | A | C; B |
| 71 | — | B | C | B | B | A | C; C |
| 72 | A | A | B | A | A | A | B; B |
| 73 | A | A | C | B | A | A | C; B |
| 74 | A | A | — | B | A | A | A; A |
| 75 | A | C | — | C | C | B | |
| 76 | A | C | — | C | C | C | |
| 77 | A | A | — | A | A | A | A; A |
| 78 | A | A | — | — | A | A | B; B |
| 81 | A | A | — | — | A | A | A; A |
| 82 | A | A | — | — | A | A | A; A |
| 83 | A | A | — | — | A | A | |
| 84 | A | A | — | — | A | A | |
| 85 | A | A | — | — | A | A | |
| 86 | A | A | — | — | A | A | |
| 87 | A | A | — | — | C | A | |
| 88 | — | A | — | — | A | A | C; B |
| 89 | | A | | | B | A | C; B |
| 90 | | A | | | A | A | C; B |
| 91 | | A | | | B | A | |
| 92 | | A | | | A | A | |
| 93 | | A | | | | A | |
| | | | | | | ³WB data | ³WB data |
| | | | | | | LU65A | NCIH727 |
| | | | | | | DC₅₀; | DC₅₀; |
| Compound | AGS | GP2D | LU65A | NCIH727 | MKN1 | D_{Max} | D_{Max} |
| 126 | A | | A | A | | | |
| 127 | A | | A | A | | | |
| 128 | A | | A | A | | | |
| 129 | A | | A | A | | | |
| 130 | B | | B | B | | | |
| 131 | A | | A | A | | | |
| 132 | A | | A | A | | | |
| 133 | A | | A | A | | | |
| 134 | A | | A | A | | | |
| 135 | A | | A | A | | | |
| 136 | A | | A | A | | | |
| 137 | A | | A | A | | | |
| 138 | C | | C | C | | | |
| 139 | C | | C | C | | | |
| 140 | A | | A | A | | | |
| 141 | A | | A | A | | | |
| 142 | A | | A | A | | | |
| 143 | A | | A | A | | | |
| 144 | A | | A | A | | | |
| 145 | A | | A | A | | | |
| 146 | A | | A | A | | | |
| 147 | A | | A | A | | | |
| 148 | A | | A | A | | | |
| 149 | A | | A | A | | | |
| 150 | A | | A | A | | | |
| 151 | A | | A | A | | | |
| 152 | A | | A | A | | | |
| 153 | A | | A | A | | | |
| 154 | A | | A | A | | | |
| 155 | A | | A | A | | | |
| 156 | A | | A | A | | | |
| 157 | A | | A | A | | | |
| 158 | A | | A | A | | | |
| 159 | A | | A | A | | | |
| 160 | A | | A | A | | | |
| 161 | A | | A | A | | | |
| 162 | A | | A | A | | | |
| 163 | A | | A | A | | | |
| 164 | A | | A | A | | | |
| 165 | A | | A | A | | | |
| 166 | A | | A | A | | | |
| 167 | | | | | | | |
| 168 | | | | | | | |
| 169 | | | | | | | |

TABLE 3
Biochemical and Cell-based Assays of Compounds

| | | | ³WB data |
|---|---|---|---|
| | | | GP2D |
| Compound | AGS | GP2D | DC₅₀; D_{Max} |
| 94 | A | A | A; A |
| 95 | B | A | C; C |
| 96 | C | B | C; B |
| 97 | B | B | C; B |
| 98 | A | A | A; A |
| 99 | C | B | — |
| 100 | A | A | A; A |
| 101 | A | A | A; A |

TABLE 4
Biochemical and Cell-based Assays of Compounds

| | | | | | | ³WB data | ³WB data |
|---|---|---|---|---|---|---|---|
| | | | | | | LU65A | NCIH727 |
| | | | | | | DC₅₀; | DC₅₀; |
| Compound | AGS | GP2D | LU65A | NCIH727 | MKN1 | D_{Max} | D_{Max} |
| 79 | B | B | A | C | C | A; A | — |
| 80 | C | B | B | A | — | A; A | B; A |
| 102 | C | B | B | B | B | C; B | A; A |
| 103 | C | C | C | C | C | C; B | C; A |
| 104 | C | C | B | C | B | — | — |
| 105 | — | — | A | — | C | A; A | — |
| 106 | C | B | C | C | A | C; B | C; B |
| 107 | C | B | B | C | B | B; A | A; A |
| 108 | B | B | A | A | B | A; A | A; A |
| 109 | B | B | A | B | C | | A; A |
| 110 | C | C | A | B | B | — | — |
| 111 | — | — | A | B | C | — | — |
| 112 | | | | | | | |
| 113 | B | A | B | B | B | A; B | A; A |
| 114 | A | A | A | A | B | A; A | A; A |
| 115 | A | A | A | A | C | A; A | A; A |
| 116 | B | B | B | C | B | — | — |
| 117 | A | A | A | A | B | — | — |
| 118 | B | A | B | C | B | — | — |
| 119 | B | B | B | B | B | — | — |
| 120 | C | C | C | C | C | — | — |
| 121 | C | B | A | C | C | — | — |
| 122 | A | A | A | B | C | — | — |
| 123 | A | A | A | A | C | — | — |
| 124 | B | B | A | C | C | — | — |
| 125 | — | — | A | — | A | — | — |

¹KRAS(G12D)/SOS1 homogeneous time-resolved fluorescence (HTRF) assay: A. IC50 <100 nM; B. IC50 = 100-1000 nM; C. IC50 >1000 nM;
²CellTiter-Glo ® Reagent cancer cell line proliferation assays: A. IC50 <100 nM; B. IC50 = 100-1000 nM; C. IC50 >1000 nM;
³WB assay: A. DC₅₀ <100 nM; B. DC₅₀ = 100-1000 nM; C. DC₅₀ >1000 nM; A. Dₘₐₓ >50%; B. Dₘₐₓ = 10-50%; C. Dₘₐₓ <10%;

Modifications and variations of the described methods and compositions of the present disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure are intended and understood by those skilled in the relevant field in which this disclosure resides to be within the scope of the disclosure as represented by the following claims.

### INCORPORATION BY REFERENCE

All patents and publications mentioned in this specification are herein incorporated by reference to the same extent as if each independent patent and publication was specifically and individually indicated to be incorporated by reference.

1. A compound of the Formula Ia: 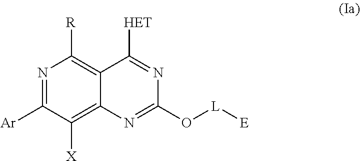
or a pharmaceutically acceptable salt thereof, wherein
HET is an optionally substituted heterocyclyl;
Ar is an optionally substituted aryl or optionally substituted heteroaryl;
R is selected from (C₁-C₄)alkyl, (C₁-C₄)haloalkyl, (C₁-C₄)alkoxy, deuterated(C₁-C₄)alkoxy, (C₁-C₄)haloalkoxy, (C₁-C₄)alkynyl, (C₁-C₄)alkenyl, halo, OH, (C₃-C₆)cycloalkyl, —O(C₃-C₆)cycloalkyl, cyano, NH₂, —NH(C₁-C₄)alkyl, —N[(C₁-C₄)alkyl]₂, —P(O)[(C₁-C₄)alkyl]₂, and —S(C₁-C₄)alkyl, wherein said (C₃-C₆)cycloalkyl and said (C₃-C₆)cycloalkyl of —O(C₃-C₆)cycloalkyl are optionally substituted with 1 to 3 groups selected from halo, (C₁-C₄)alkyl, (C₁-C₄)alkoxy, halo(C₁-C₄)alkoxy, and cyano;
X is hydrogen or halo;
L is a linker; and
E is a chemical moiety that targets E3 ligase.

2. The compound of claim 1, or a pharmaceutically acceptable salt thereof, wherein R is selected from (C₁-C₄)alkyl, (C₁-C₄)alkoxy, deuterated(C₁-C₄)alkoxy, (C₁-C₄)alkynyl, (C₁-C₄)alkenyl, OH, O(C₃-C₆)cycloalkyl, NH₂, —NH(C₁-C₄)alkyl, and —N[(C₁-C₄)alkyl]₂.

3. The compound of claim 1 or 2, or a pharmaceutically acceptable salt thereof, wherein R is selected from ethyl, —CHCH₂, —CCH, NH₂, NHMe, NMe₂, OCH₃, OCD₃, OCF₃, OEt, OCH(CH₃)₂, and O(cyclopropyl).

4. The compound of any one of claims 1 to 3, or a pharmaceutically acceptable salt thereof, wherein R is selected from (C₁-C₄)alkoxy, deuterated(C₁-C₄)alkoxy, and OH.

5. The compound of any one of claims 1 to 4, or a pharmaceutically acceptable salt thereof, wherein R is selected from OCH₃, OH, and OCD₃.

6. The compound of any one of claims 1 to 5, or a pharmaceutically acceptable salt thereof, wherein the compound is of the Formula II: 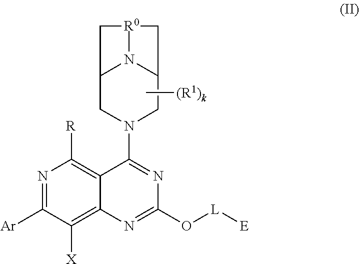
or a pharmaceutically acceptable salt thereof, wherein
R¹ is selected from halo, (C₁-C₄)alkyl, hydroxy(C₁-C₄)alkyl, cyano (C₁-C₄)alkyl, —CH(═O), —C(O)₂H, —C(O)₂ (C₁-C₄)alkyl, C(O)₂NH₂, —C(O)₂NH(C₁-C₄)alkyl, —C(O)₂N [(C₁-C₄)alkyl]₂, and 5- to 6-membered optionally substituted heteroaryl;
k is 0, 1, 2, or 3;
R⁰ is selected from hydrogen, CN, —C(O)CN, —C(O)R^{a′}, —C(O)NHR^{a′}, and (C₁-C₄)alkyl, wherein said (C₁-C₄)alkyl is optionally substituted with 1 to 3 groups selected from CN, OH, (C₃-C₆)cycloalkyl, halo, O(C₁-C₄)alkyl, (C₂-C₄)alkenyl, (C₂-C₄)alkynyl, —COR^{a′}, and —C(O)OR^{a′}; and
R^{a′} is selected from (C₁-C₄)alkyl and (C₁-C₄)alkenyl, wherein said alkyl is opt substituted with 1 to 3 groups selected from CN and halo.

7. The compound of any one of claims 1 to 6, or a pharmaceutically acceptable salt thereof, wherein R⁰ is selected from hydrogen-C(O)R^{a′}, —C(O)NHR^{a′}, and (C₁-C₄)alkyl, wherein said (C₁-C₄)alkyl is optionally substituted with 1 to 3 groups selected from halo, CN, OH, and (C₃-C₆)cycloalkyl.

8. The compound of any one of claims 1 to 7, or a pharmaceutically acceptable salt thereof, wherein R^{a′} is selected from (C₁-C₄)alkyl, (C₁-C₄)alkenyl, each of which are optionally substituted with 1 to 3 groups selected from CN and halo.

9. The compound of any one of claims 1 to 8, or a pharmaceutically acceptable salt thereof, wherein R⁰ is selected from hydrogen, C(O)CH₂CH₃, C(O)CF₃, CH₃, CH₂CN, (CH₂)₂CN, CH₂CH(CH₃), CH₂CF₃, (CH₂)₂OH, CH₂ (cyclopropyl), C(O)CH₂CN, C(O)CH(CH₃)CN, C(O)CH(CH₂CH₃)CN, C(O)(CH₂)₂CN, C(O)CH₂CH(CH₃)CN, C(O)CH(CH₃)CH₂CN, C(O)CH(CN) CH₂CF₃, C(O)NHCH₂CN, C(O)NHCH₂CF₃, and C(O)CHCH₂.

10. The compound of any one of claims 1 to 6, or a pharmaceutically acceptable salt thereof, wherein R⁰ is selected from —CH₂CN, —CH₂CH₂OH, —CH₂CH₂OMe, or CH₂C(O)Me.

11. The compound of any one of claims 1 to 10, or a pharmaceutically acceptable salt thereof, wherein k is 0.

12. The compound of any one of claims 1 to 11, or a pharmaceutically acceptable salt thereof, wherein X is hydrogen or fluoro.

13. The compound of any one of claims 1 to 12, or a pharmaceutically acceptable salt thereof, wherein X is fluoro.

14. The compound of any one of claims 1 to 13, or a pharmaceutically acceptable salt thereof, wherein Ar is an optionally substituted phenyl or optionally substituted naphthyl.

15. The compound of any one of claims 1 to 14, or a pharmaceutically acceptable salt thereof, wherein Ar is phenyl or naphthalenyl, each of which are optionally substituted with one to three groups independently selected from R^{A}, wherein R^{A} is selected from halo, (C₁-C₄)alkyl, (C₂-C₄)alkynyl, (C₂-C₄)alkynylNH₂, (C₁-C₄)alkoxy, halo(C₁-C₄)alkoxy, (C₁-C₄)alkylOH, OH, NH₂, —NH(C₁-C₄)alkyl, —N[(C₁-C₄)alkyl]₂, C(O)NH₂, C(O)NH(C₁-C₄)alkyl, C(O)[(C₁-C₄)alkyl]₂, —NHC(O)(C₁-C₄)alkyl, —N(C₁-C₄)alkyIC(O)(C₁-C₄)alkyl, —NHC(O)O(C₁-C₄)alkyl, NHC(O)NH(C₁-C₄)alkyl, CN, —S(C₁-C₄)alkyl, —Shalo(C₁-C₄)alkyl, and (C₃-C₆)cycloalkyl, wherein said (C₁-C₄)alkyl and said (C₃-C₆)cycloalkyl are each optionally substituted with one to two groups selected from halo, (C₁-C₄)alkoxy, halo(C₁-C₄)alkoxy, OH, NH₂, —NH(C₁-C₄)alkyl, —N[(C₁-C₄)alkyl]₂, C(O)NH₂, C(O)NH(C₁-C₄)alkyl, C(O)[(C₁-C₄)alkyl]₂, —NHC(O)(C₁-C₄)alkyl, —N(C₁-C₄)alkylC(O)(C₁-C₄)alkyl, —NHC(O)O(C₁-C₄)alkyl, NHC(O)NH(C₁-C₄)alkyl, CN, —S(C₁-C₄)alkyl, and —S(C₁-C₄)haloalkyl.

16. The compound of any one of claims 1 to 15, or a pharmaceutically acceptable salt thereof, wherein Ar is naphthalenyl optionally substituted with one to three groups independently selected from R^{A}.

17. The compound of claim 15 or 16, or a pharmaceutically acceptable salt thereof, wherein R^{A} is selected from (C₂-C₄)alkynyl, halo, and OH.

18. The compound of any one of claims 1 to 17, or a or a pharmaceutically acceptable salt thereof, wherein Ar is

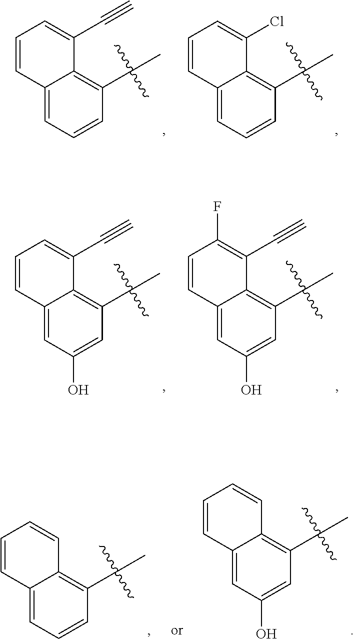 [SMILES: *c1cccc2cccc(C#C)c12 ; *c1cccc2cccc(Cl)c12 ; *c1cc(O)cc2cccc(C#C)c12 ; *c1cc(O)cc2ccc(F)c(C#C)c12 ; *c1cccc2ccccc12 ; *c1cc(O)cc2ccccc12]

19. The compound of any one of claims 1 to 18, or a or a pharmaceutically acceptable salt thereof, wherein Ar is

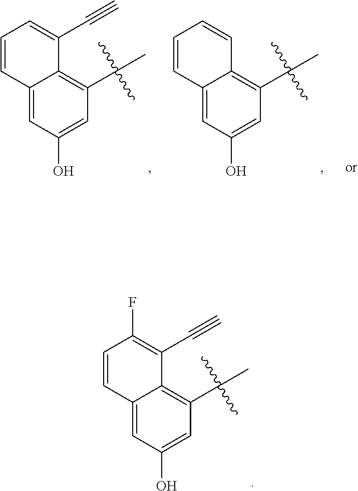 [SMILES: *c1cc(O)cc2cccc(C#C)c12 ; *c1cc(O)cc2ccccc12 ; *c1cc(O)cc2ccc(F)c(C#C)c12]

20. The compound of any one of claims 1 to 19, or a or a pharmaceutically acceptable salt thereof, wherein Ar is

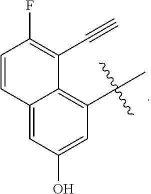 [SMILES: *c1cc(O)cc2ccc(F)c(C#C)c12]

21. The compound of any one of claims 1 to 20, or a pharmaceutically acceptable salt thereof, wherein E is selected from a cereblon (CRBN) modulator and a von Hippel-Lindau (VHL) ligand.

22. The compound of any one of claims 1 to 21, or a pharmaceutically acceptable salt thereof, wherein E is of the structural formula: 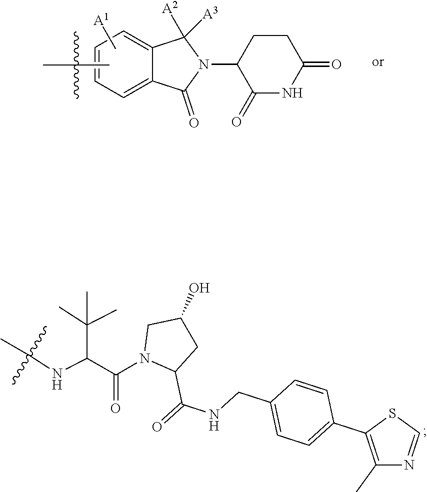
wherein A¹ is halo; and A² and A³ are both hydrogen or A² and A³ taken together form ═O.

23. The compound of any one of claims 1 to 22, or a pharmaceutically acceptable salt thereof, wherein E is of the structural formula:

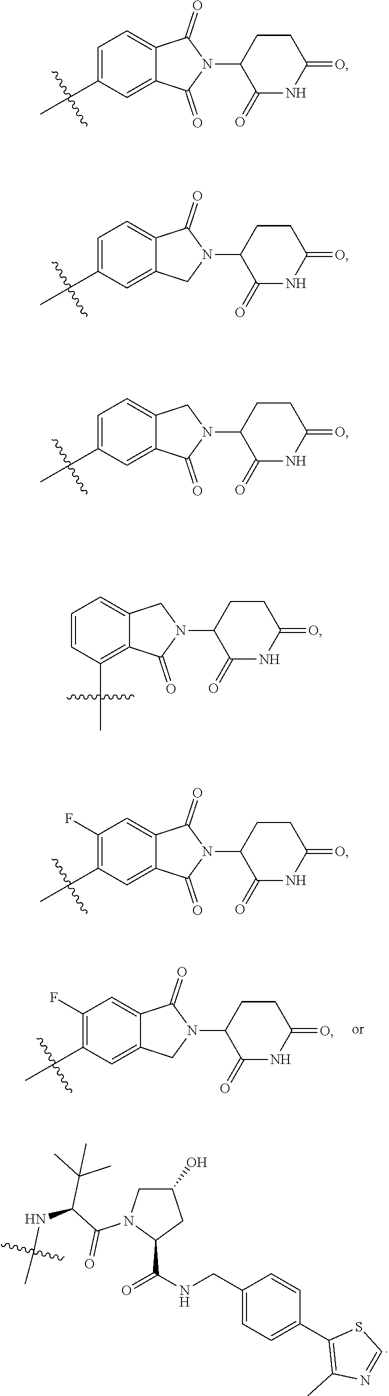

24. The compound of any one of claims 1 to 23, or a pharmaceutically acceptable salt thereof, wherein E is of the structural formula:

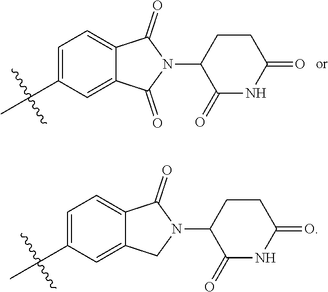 [SMILES: *c1ccc2c(c1)C(=O)N(C1CCC(=O)NC1=O)C2=O ; *c1ccc2c(c1)CN(C1CCC(=O)NC1=O)C2=O]

25. The compound of any one of claims 1 to 24, or a pharmaceutically acceptable salt thereof, wherein
L is -Het¹-X¹—*, *-Het¹-Het²-X¹—, *—NR^{c}—X¹-Het¹-Het²-X², *—X¹-Het¹-X²-Het²-(CH₂)ₘO—, —NR^{c}—(CH₂)ₘ—X¹-Het¹-X²—*, —NR^{c}—(CH₂)ₘ—X¹—NR^{c}—(CH₂CH₂O)ₙ—*, —NR^{c}—(CH₂)ₘ—X¹—NR^{c}—(CH₂)ₚ—*, —NR^{c}—(CH₂)ₘ—X¹-Het¹-X²-Het²-X³—*, —O(CH₂)ₘ—X¹-Het¹-X²-Het²-X³—*, —O(CH₂)ₘ—X¹—NR^{c}—(CH₂)ₚ-Het¹-X²-Het²-X³—*, *—X¹—NR^{c}—(CH₂)ₘ-Het¹-X²-Het²-X³—(CH₂)ₚ—NR^{d}—(CH₂)ₚ—, —NR^{c}—(CH₂)ₘ—X¹—(CH) CH₃-Het¹-X²-Het³-X³—*, —NR^{c}—(CH₂)ₘ—X¹—(CH₂)ₚ-Het¹-X²-Het²-X³—*, —NR^{c}—(CH₂)ₘ—X¹—NR^{d}—(CH₂)ₚ-Het¹-X²-Het²-X³—*, —NR^{c}—(CH₂)ₘ—NR^{d}—X¹-Het¹-X²—*, *Het¹-X¹-Het²-X²—, *X¹-Het¹-X²—, *(CH₂CH₂O)ₙ—NR^{c}—X¹-Het¹-Het²-X²—, *Het¹-X¹-Het²-X²-Het³-X³—, *X¹-Het¹-X²-Het²-X³—, *-Het¹-X¹—NR^{c}-Het²-X²—*X¹-Het¹-Het²-X²—, *X¹—(CH₂)ₘO-Het¹-X²—, *X¹—(CH₂)ₘNR^{c}—X²-Het¹-Het²-X², *-Het¹-X¹-Het²-X²—O—, *—O(CH₂)ₘ-Het¹-(CH₂)ₚ—O(CH₂)ₘ—NR^{c}—X²—*, *-Het¹-O—(CH₂)ₘ—X¹-Het²-X², *-Het¹-O—(CH₂)ₘ—X¹—NR^{c}—(CH₂CH₂O) n (CH₂)ₘ-Het²-X²—, *-Het¹-X¹—NR^{c}—(CH₂)ₘ—, *-Het¹-X¹-Het²-Het³-X², *-Het¹-X¹—NR^{c}—(CH₂CH₂O), (CH₂)ₘ—, *-Het¹-X¹—NR^{c}—(CH₂CH₂O)ₙHet²-(CH₂)ₘ—X²—, *-Het¹-X¹—NR^{c}—(CH₂CH₂O)ₙ—, *-Het¹-X¹—NR^{c}—(CH₂)ₘ-Het²-X²-Het³-(CH₂)ₘ—, *-Het¹-X¹-Het²-(CH₂)ₘ-Het³-X²—, *-Het¹-X¹-Het²-, *-Het¹-X¹—NR^{c}—, *-Het¹-X¹—NR^{c}—(CH₂)ₘ-Phe-X²-Het²-(CH₂)ₘ—, *-Het¹-X¹-Het²-Het³-, *-Het¹-X¹-Het²-(CH₂)ₘ-Het³-X²—(CH₂)ₚ—NR^{c}—(CH₂)ₘ—, *-Het¹-X¹-Het²-(CH₂)ₘ-Het³-(CH₂)ₘ—O—, *-Het¹-X¹-Het²-(CH₂)ₘ-Het³-(CH₂)ₚ—NR^{c}—(CH₂)ₘ—, *-Het¹-X¹-Het²-(CH₂CH₂O)ₙ—, *-Het¹-X¹—(CH₂)ₘ-Het²-X²—, *—(CH₂CH₂O)ₒ—(CH₂)ₚ-Het¹-X¹-Het²-(CH₂CH₂O)ₙ, *—(CH₂CH₂O)ₙ—(CH₂)ₘ-Het¹-X¹-Het²-X², *-Het¹-X¹-Phe-X²—NR^{c}—X³—, *—(CH₂CH₂O)ₒ—(CH₂)ₚ-Het¹-X¹-Phe-X²—NR^{c}—(CH₂CH₂O)ₙ—, *—(CH₂CH₂O)ₙ—(CH₂)ₘ—NR^{c}-Phe-X¹—, *—(CH₂CH₂O)ₒ—(CH₂)ₚ—NR^{c}-Phe-(CH₂CH₂O)ₙ—, *—(CH₂CH₂O)ₒ—(CH₂)ₚ—NR^{c}—(CH₂CH₂O)ₙ—(CH₂)ₘ—, *—(CH₂CH₂O)ₙ—(CH₂)ₘ—NR^{c}—(CH₂CH₂O)ₙ—(CH₂)ₘ—C(O)—NR^{d}—(CH₂CH₂O), —(CH₂)ₚ—, *—(CH₂CH₂O), —(CH₂)ₚ—NR^{c}—(CH₂CH₂O)ₙ—(CH₂)ₘ-Het¹-X¹-Het²-X², *—(CH₂CH₂O), —(CH₂)ₚ—NR^{c}—(CH₂CH₂O)ₙ—(CH₂)ₘ-Het¹-X¹-Het²-X²—(CH₂CH₂O)ₒ, *—NR^{c}—(CH₂CH₂O)ₙ—(CH₂)ₘ-Phe-NH—X¹-Het¹-X², *—NR^{c} (CH₂CH₂O)ₙ—(CH₂)ₘ-Phe-NH—X¹-Het¹-X²—(CH₂CH₂O)ₒ, *—(CH₂CH₂O)ₒ—(CH₂)ₚ—NR^{c}—(CH₂CH₂O)—(CH₂)ₘ-Phe-X¹—NR^{c}—(CH₂CH₂O)—(CH₂)ₚ—, *—(CH₂CH₂O)ₒ, —(CH₂)ₚ—NR^{c}—(CH₂CH₂O)ₙ—(CH₂)ₘ-Het¹-X¹—, *—(CH₂CH₂O)ₒ—(CH₂)ₚ—NR^{c}—(CH₂CH₂O)ₙ—(CH₂)ₘ-Het¹-X¹—(CH₂CH₂O)ₙ—, *—(CH₂CH₂O)ₙ—(CH₂)ₘ—NR^{c}—(CH₂)ₘ—C(O)—NR^{d}-Het¹-X¹-Het²-(CH₂CH₂O)ₒ—(CH₂)ₚ, or *—NR^{c}—(CH₂)ₘ—C(O)—NR^{d}—(CH₂)ₘ-Het¹-X¹-Het²-X²—;
* indicates the point of attachment to E;
Het¹, Het², and Het³ are each independently phenyl, a 5- to 8-membered heterocyclyl, 5- to 7-membered heteroaryl, or a 3- to 6-membered cycloalkyl, each of which are optionally substituted with (C₁-C₄)alkyl;
Phe is phenyl;
X¹, X², and X³, are each independently C(O) or (CH₂)ᵣ;
R^{c} and R^{d} are each independently hydrogen or (C₁-C₄)alkyl; and
m, n, o, p, and r are each independently integers selected from 0, 1, 2, 3, 4, 5, and 6.

26. The compound of any one of claims 1 to 25, or a pharmaceutically acceptable salt thereof, wherein L is *-Het¹-Het²-X¹—, *—NR^{c}—X¹-Het¹-Het²-X², *Het¹-X¹-Het²-X², *X¹-Het¹-X²—, *(CH₂CH₂O)ₙ—NR^{c}—X¹-Het¹-Het²-X², *Het¹-X¹-Het²-X²-Het³-X³, *X¹-Het¹-X²-Het²-X³, *-Het¹-X¹—NR^{c}-Het²-X², *X¹-Het¹-Het²-X², *X¹—(CH₂)ₘO-Het¹-X²—, *X¹—(CH₂)ₘNR^{c}—X²-Het¹-Het²-X², or *-Het¹-X¹-Het²-Het³-X².

27. The compound of claim 25 or 26, or a pharmaceutically acceptable salt thereof, wherein Het¹, Het², and Het³ are each independently a 5- to 8-membered heterocyclyl, or a 3- to 6-membered cycloalkyl, each of which are optionally substituted with (C₁-C₄)alkyl.

28. The compound of any one of claims 25 to 27, or a pharmaceutically acceptable salt thereof, wherein m, n, o, p, and r are each independently integers selected from 0, 1, 2, or 3.

29. The compound of any one of claims 1 to 28, or a pharmaceutically acceptable salt thereof, wherein L is selected from

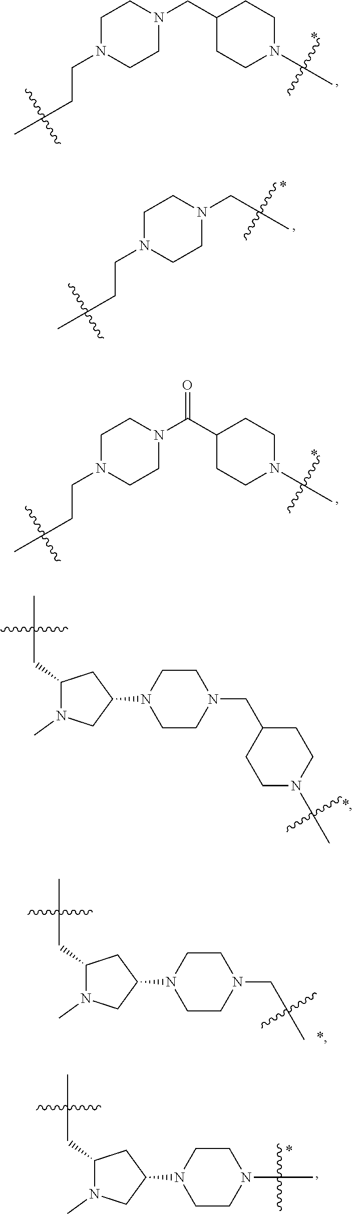 [SMILES: *CCN1CCN(CC2CCN(*)CC2)CC1 ; *CCN1CCN(C*)CC1 ; *CCN1CCN(C(=O)C2CCN(*)CC2)CC1 ; *C[C@@H]1C[C@H](N2CCN(CC3CCN(*)CC3)CC2)CN1C ; *C[C@@H]1C[C@H](N2CCN(C*)CC2)CN1C ; *C[C@@H]1C[C@H](N2CCN(*)CC2)CN1C]

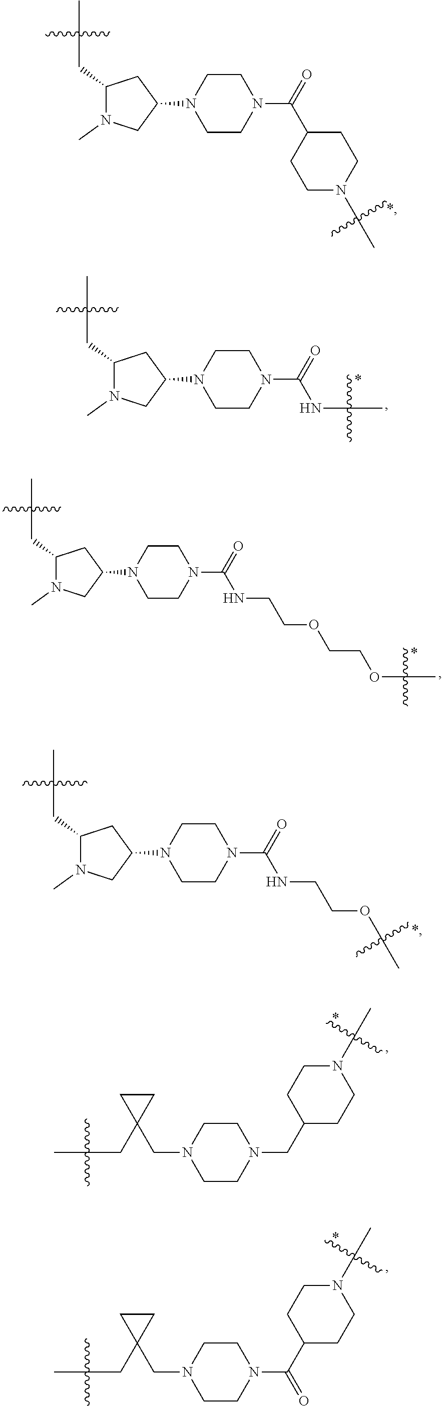 [SMILES: *C[C@@H]1C[C@H](N2CCN(C(=O)C3CCN(*)CC3)CC2)CN1C ; *C[C@@H]1C[C@H](N2CCN(C(=O)N*)CC2)CN1C ; *C[C@@H]1C[C@H](N2CCN(C(=O)NCCOCCO*)CC2)CN1C ; *C[C@@H]1C[C@H](N2CCN(C(=O)NCCO*)CC2)CN1C ; *CC1(CN2CCN(CC3CCN(*)CC3)CC2)CC1 ; *CC1(CN2CCN(C(=O)C3CCN(*)CC3)CC2)CC1]

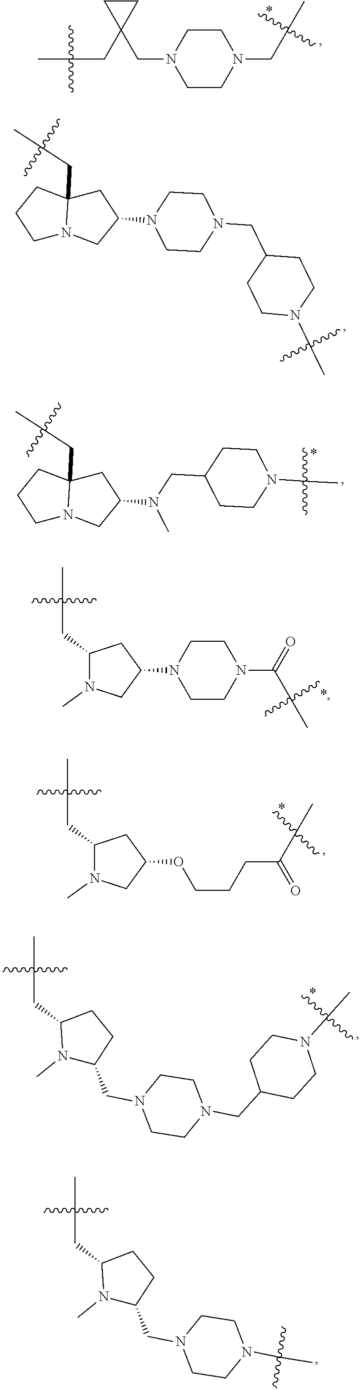

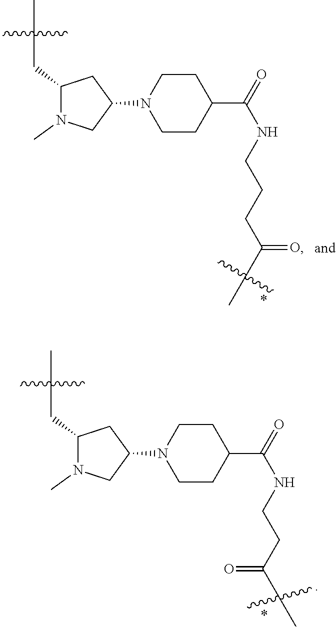 [SMILES: *C[C@@H]1C[C@H](N2CCC(C(=O)NCCCC(*)=O)CC2)CN1C ; "and" ; *C[C@@H]1C[C@H](N2CCC(C(=O)NCCC(*)=O)CC2)CN1C]

30. The compound of any one of claims 1 to 29, or a pharmaceutically acceptable salt thereof, wherein L is

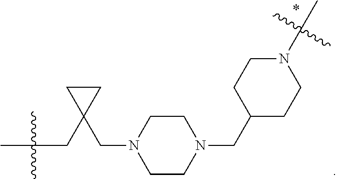 [SMILES: *CC1(CN2CCN(CC3CCN(*)CC3)CC2)CC1]

31. A compound selected from any one of compounds 1 to 125, or a pharmaceutically acceptable salt thereof.

32. A pharmaceutical composition comprising the compound of any one of claims 1 to 31, or a pharmaceutically acceptable salt thereof; and a pharmaceutically acceptable carrier.

33. A method of treating cancer comprising administering to a subject in need a therapeutically effective amount of a compound of any one of claims 1 to 31, or a pharmaceutically acceptable salt thereof, or the pharmaceutical composition of claim 32.
